(19) United States
(12) Patent Application Publication
(10) Pub. No.: US 2025/0170112 A1
(43) Pub. Date: May 29, 2025

(54) ADENOSINE LIGANDS FOR THE TREATMENT OF NEUROLOGICAL DISORDERS

(71) Applicant: Lucy Therapeutics, Inc., Waltham, MA (US)

(72) Inventors: Dario Doller, Waltham, MA (US); Guiying Li, Waltham, MA (US); Casey McComas, Waltham, MA (US); Amy Ripka, Waltham, MA (US); Allen T. Hopper, Waltham, MA (US); John E. Macor, Washington Crossing, PA (US)

(21) Appl. No.: 18/839,341

(22) PCT Filed: Feb. 16, 2023
(86) PCT No.: PCT/US2023/013193

§ 371 Date: Aug. 16, 2024

Related U.S. Application Data
(60) Provisional application No. 63/311,351, filed on Feb. 17, 2022.

(51) Int. Cl. A61K 31/4439; A61K 31/437; A61P 25/28; C07D 401/12; C07D 405/14; C07D 471/04

(52) U.S. Cl. CPC A61K 31/4439; A61K 31/437; A61P 25/28; C07D 401/12; C07D 405/14; C07D 471/04

(57) ABSTRACT

The present application relates to compounds of formula (I), and their pharmaceutical compositions/preparations. This application further relates to methods of treating Rett syndrome and/or one or more symptoms associated with Rett syndrome.

### RELATED APPLICATIONS

This application claims the benefit of and priority to U.S. Provisional Patent Application No. 63/311,351, filed Feb. 17, 2022, which application is hereby incorporated by reference in its entirety.

### BACKGROUND

Rett syndrome (RTT) is a neurological disorder caused by mutations in the X-linked gene methyl-CpG-binding protein 2 (MECP2), a ubiquitously expressed transcriptional regulator. The MECP2 gene contains instructions for the synthesis of a protein called methyl cytosine binding protein 2 (MeCP2), which is needed for brain development and acts as one of the many biochemical switches that can regulate gene expression. Because the MECP2 gene does not function properly in individuals with Rett syndrome, insufficient amounts or structurally abnormal forms of the MeCP2 protein are produced and can cause other genes to be abnormally expressed (Rett Syndrome Fact Sheet; www.ninds.nih.gov/Disorders/Patient-Caregiver-Education/Fact-Sheets/Rett-Syndrome-Fact-Sheet). The prevalence of RTT is high (1 in 10,000 births), and it is one of the most common causes of intellectual and developmental disabilities (IDD) in females. Lifespan and disease severity vary greatly.

RTT is characterized by progressive development of motor and neurological dysfunction. Girls affected with RTT acquire speech and movement skills on a normal timeline after birth but develop symptoms between 6 months and 2 years of age. Neurological manifestations of disease include loss of speech and motor skills, stereotypic hand movements, difficulty walking, sporadic rapid respiration and apnea, and seizures. Apraxia, the inability to perform motor functions, is perhaps the most severely disabling feature of RTT, interfering with every body movement, including eye gaze and speech. Children with Rett syndrome often exhibit autistic-like behaviors in the early stages. Other symptoms may include walking on the toes, sleep problems, a wide-based gait, teeth grinding and difficulty chewing, slowed growth, seizures, cognitive disabilities, and breathing difficulties while awake such as hyperventilation, apnea (breath holding), and air swallowing (Rett Syndrome Fact Sheet; www.ninds.nih.gov/Disorders/Patient-Caregiver-Education/Fact-Sheets/Rett-Syndrome-Fact-Sheet).

RTT was initially thought to be an entirely neurological disorder, and RTT research focused on the role of MeCP2 in the central nervous system. However, the variety of phenotypes identified in Mecp2 mutant mouse models and RTT patients also implicate important roles for MeCP2 in peripheral systems (Kyle, Vashi and Justice, Open Biol 2018, 8(2), 170216). More than 95% of RTT patients carry a mutation in the MECP2 gene. Mechanistically, MECP2 binds to methylated DNA to regulate gene transcription through repression or activation. When MECP2 represses gene transcription, it associates with chromatin-remodeling complexes that contain Type I histone deacetylases (HDACs) (Bienvenu and Chelly, Nat Rev. Genet 2006, 7, 415-426). Therefore, the elimination of MECP2 may result in the upregulation of genes that would normally be repressed. Notably, the severity of MECP2 mutation does not always correlate with disease severity, due at least in part to favorable patterns of X-chromosome inactivation in heterozygous females.

Many children diagnosed with RTT have reduced brain volume compared with healthy individuals, consistent with a smaller head circumference. Reduced brain volume is largely due to small neuronal body size and a denser packing of cells, particularly in layers III and V of the cerebral cortex, thalamus, substantia nigra, basal ganglia, amygdala, cerebellum and hippocampus. Patients also have reduced dendritic arborization, indicative of a delay in neuronal maturation. Furthermore, hypopigmentation of the substantia nigra suggests a dysfunction of dopaminergic neurons. RTT patients show evidence of dysregulated neurotransmitters, neuromodulators and transporters, indicating an important role in synaptic function (Kyle, Vashi and Justice, Open Biol 2018, 8(2), 170216).

Mouse models that carry Mecp2 mutations and recapitulate most of the symptoms of RTT are available. Mecp^{2/Y} male mutant mice are normal at birth and weaning, but develop symptoms that include hypo-activity, limb clasping, tremors, motor impairment and abnormal breathing as early as 4 weeks of age. Such symptoms progressively worsen, leading to their death at 6-16 weeks. Pronounced neuronal deficits are observed in Mecp^{2/Y} null mice, including delayed transition into mature stages, altered expression of presynaptic proteins and reduced dendritic spine density.

Remarkably, re-expression of Mecp2 in mutant male and female mice after the onset of symptoms rescues neurological deficits and mice recover normal movements to live a long life (Guy et al. Science 2007, 315, 1143-1147). These findings show that RTT is not caused by a permanent abnormal development of neurons during embryogenesis; instead, MECP2 is required for the maintenance of neurons after birth. Thus, RTT may be ameliorated or even reversed by genetic or pharmacological means after the onset of symptoms, providing tremendous hope for patients and families. Unfortunately, gene therapy using MECP2 is challenging, because brain function is exquisitely sensitive to levels of MECP2: increased MECP2 causes a progressive neurological disorder that leads to death as well (Bienvenu and Chelly, Nat Rev Genet 2006, 7, 415-426).

Adenosine is a purine nucleoside comprised of an adenine group attached to a ribose sugar by a glycosidic bond. Adenosine is present in all cells and released by certain physiological and pathophysiological stimuli. Adenosine is formed intracellularly as an intermediate during the degradation of adenosine 5′-phosphate (AMP) and S-adenosylhomocysteine, but it can be released from the cell, acting as a neurotransmitter by binding to specific receptors. Thus, adenosine may act as both a precursor and a metabolite of adenine nucleotides, and by providing the structural building block of adenosine triphosphate (ATP), it plays a central role in the basic energy transfer of all living organisms (Layland, J., Carrick, D., Lee, M., Oldroyd, K., & Berry, C. JACC. Cardiovascular Interventions 2014, 7, 581-591.)

Adenosine exerts a variety of physiological effects by interacting with cell surface G-protein-coupled receptor subtypes, namely A1, A2A, A2B, and A3 adenosine receptors (ARs). Each receptor is encoded by a different gene. “Adenosine receptor selective agonists” are substances that bind selectively to one or more subtypes of the adenosine receptors, thus mimicking the action of adenosine.

The actions of these adenosine receptors are mediated intracellularly by the messenger cAMP. In the case of A1 AR agonists (coupling preferably via Gi/o proteins), a decrease of the intracellular cAMP concentration is observed (preferably after direct pre-stimulation of adenylate cyclase by forskolin). Correspondingly, A2A and A2B AR agonists (coupling preferably via Gs proteins) leads to an increase of cAMP concentration in the cells, and A2A and A2B AR antagonists to a decrease of the cAMP concentration in the cells. As a classical neurotransmitter, adenosine is neither stored nor released and is thought to be formed inside cells or on their surface, mostly by breakdown of adenine nucleotides (Huang et al. Curr Top Med Chem 2011, 11(8), 1047-57).

Currently there is no cure for Rett Syndrome. Treatment for the disorder is symptomatic, i.e., focusing on the management of symptoms, and supportive, requiring a multidisciplinary approach. For example, medication may be used for breathing irregularities and motor difficulties, and anticonvulsant drugs may be used to control seizures. Also, occupational therapy is used to help children develop skills needed for performing self-directed activities (such as dressing, feeding, and practicing arts and crafts), while physical therapy and hydrotherapy may prolong mobility. The use of a brain penetrant A1, A2B and/or dual A1/A2B AR agonists may produce a medication for treating Rett syndrome.

There is a critical need for agents and methods for treating Rett Syndrome as well as related disorders. The compounds of the present application, agonists of adenosine receptors, provide promising agents for the treatment of Rett Syndrome and its related disorders.

### Summary of Application

The present application provides a compound of formula (I)

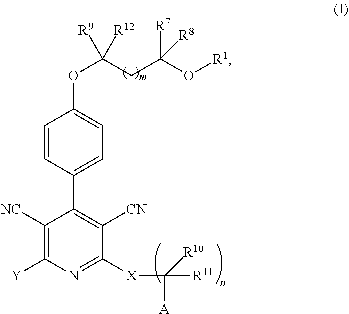

or a pharmaceutically acceptable salt thereof, wherein: Y is selected from hydrogen, C₁₋₆ alkyl, C₃₋₆ cycloalkyl, OR¹³, and NR²R³, R² and R³ each independently is selected from hydrogen and C₁₋₆alkyl; or R² and R³ taken together with the nitrogen to which they are attached form a 4- or 5-membered heterocyclyl ring, wherein the 4- or 5-membered heterocyclyl ring is optionally substituted with one or more substitutent independently selected from halogen or hydroxyl; R¹ is selected from hydrogen, C₁₋₆ alkyl, and C₁₋₆ haloalkyl; R⁷ and R⁸ each independently is selected from hydrogen, C₁₋₆alkyl, and C₁₋₆ haloalkyl; R⁹ is hydrogen or halogen, or R¹ and R⁹ taken together with the atoms to which they are attached form a 4-, 5-, or 6-membered heterocyclyl ring, or R⁹ and R⁸ taken together with the atoms to which they are attached form a cyclopropyl ring system when m is 0 or a cyclobutyl or cyclopentyl ring system when m is 1; R¹⁰ and R¹¹ each independently is selected from hydrogen, hydroxyl, halogen, C₁₋₆alkyl, and C₁₋₆ haloalkyl; R¹² is hydrogen or halogen; X is selected from a bond, S, O, and NR⁴, wherein R⁴ is hydrogen or C₁₋₆alkyl (e.g., methyl); R¹³ is selected from hydrogen, C₁₋₆ alkyl, and C₁₋₆ haloalkyl; A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, optionally substituted aryl, and optionally substituted heteroaryl; R⁵ and R⁶ each independently is selected from hydrogen and C₁₋₆alkyl; m is 0 or 1, provided that when m is 1, R⁸ and R⁹ are taken together with the carbons to which they are attached to form a cyclobutyl or cyclopentyl ring system; and n is 0, 1 or 2; provided that when Y is NH₂, X is S, n is 1, R¹ is hydrogen or methyl, and each of R⁷, R⁸ and R⁹ is hydrogen, then A is not substituted thiazole; when Y is NH₂, X is S, n is 1, R⁸ is methyl, and each of R¹, R⁷ and R⁹ is hydrogen, then A is not substituted thiazole; when Y is NR²R³, R² and R³ are both C₁₋₆alkyl (e.g., ethyl), X is S, n is 1, and each of R¹, R⁷, R⁸ and R⁹ is hydrogen, then A is not substituted thiazole; when Y is NR²R³, R² and R³ taken together with the nitrogen to which they are attached form a 4- or 5-membered heterocyclyl ring, X is S, n is 1, and each of R¹, R⁷, R⁸ and R⁹ is hydrogen, then A is not substituted thiazole; when Y is NH₂, X is S, n is 1, R¹ is methyl, and each of R⁷, R⁸ R⁹, R¹⁰, and R¹¹ is hydrogen, then A is not 2-pyridinyl or 3-pyridinyl; when Y is NH₂, X is S, n is 1, R⁷ is methyl, R⁸ is methyl, and each of R¹ and R⁹ is hydrogen, then A is not substituted 4-pyridyl; when Y is NH₂, X is S, n is 1, and each of R¹, R⁷, R⁸ and R⁹ is hydrogen, then A is not substituted 3-pyridyl; when Y is hydrogen or NR²R³, R² is hydrogen, R³ is hydrogen or C₁₋₆alkyl, X is S, n is 1, and each of R¹, R⁷, R⁸ and R⁹ is hydrogen, then A is not substituted 4-pyridyl; when Y is NH₂ or pyrrolidine, X is S, n is 1, and each of R¹, R⁷, R⁸, R⁹, R¹⁰ and R¹¹ is hydrogen, then A is not substituted phenyl; when Y is NH₂, X is S, n is 1, R¹ is hydrogen or methyl, each of R⁷, R⁸, R⁹, and R¹¹ is hydrogen, and R¹⁰ is methyl, then A is not substituted phenyl; when Y is H or NH₂, X is S, n is 0, and each of R¹, R⁷, R⁸ and R⁹ is hydrogen, then A is not optionally substituted aryl (e.g., aryl, such as phenyl); when Y is NH₂, X is S or O, n is 1, and each of R¹, R⁷, R⁸ and R⁹ is hydrogen, then A is not substituted oxazole; when Y is NH₂, X is 0, n is 1, and each of R¹, R⁷, R⁸ and R⁹ is hydrogen, then A is not substituted phenyl, or substituted 4-pyridyl; when Y is OR¹³, R¹³ is methyl, X is S, n is 1, and each of R¹, R⁷, R⁸, R⁹, R¹⁰ and R¹¹ is hydrogen, then A is not substituted phenyl, substituted oxazole, imidazole, or 2-pyridyl; when Y is OR¹³, R¹³ is ethyl, X is S, n is 1, and each of R¹, R⁷, R⁸, R⁹, R¹⁰ and R¹¹ is hydrogen, then A is not substituted oxazole; and when Y is OR¹³, R¹³ is methyl, ethyl, or sec-butyl, X is S, n is 1, and each of R¹, R⁷, R⁸, R⁹, R¹⁰ and R¹¹ is hydrogen, then A is not substituted thiazole.

In certain embodiments, A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, aryl (e.g., phenyl), and optionally substituted heteroaryl. In certain embodiments, A is selected from cyano, hydroxyl, optionally substituted C₁₋₆alkyl, optionally substituted C₁₋₆ alkoxy, —C(O)NR⁵R⁶, —NR⁵R⁶, —NR⁵C(O)C₁₋₆alkyl, optionally substituted heterocyclyl, optionally substituted aryl, optionally substituted indole, optionally substituted pyrazine, optionally substituted pyrimidine, optionally substituted pyrrolopyridine, optionally substituted pyridazine, optionally substituted indazole, optionally substituted benzofuran, optionally substituted benzoimidazole, optionally substituted benzothiazole, and optionally substituted benzothiophene. In certain embodiments, A is selected from cyano, hydroxyl, optionally substituted C₁₋₆alkyl, optionally substituted C₁₋₆ alkoxy, —C(O)NR⁵R⁶, —NR⁵R⁶, —NR⁵C(O)C₁₋₆alkyl, optionally substituted heterocyclyl, optionally substituted aryl, optionally substituted indole, optionally substituted pyrazine, optionally substituted pyrimidine, optionally substituted pyrrolopyridine, optionally substituted pyridazine, optionally substituted indazole, optionally substituted benzofuran, optionally substituted benzoimidazole, optionally substituted benzothiazole, optionally substituted benzothiophene, oxazole, and thiazole. In certain embodiments, A is selected from cyano, hydroxyl, optionally substituted C₁₋₆alkyl, optionally substituted C₁₋₆alkoxy, —C(O)NR⁵R⁶, —NR⁵R⁶, —NR⁵C(O)C₁₋₆alkyl, optionally substituted heterocyclyl, optionally substituted indole, optionally substituted pyrazine, optionally substituted pyrimidine, optionally substituted pyrrolopyridine, optionally substituted pyridazine, optionally substituted indazole, optionally substituted benzofuran, optionally substituted benzoimidazole, optionally substituted benzothiazole, and optionally substituted benzothiophene. In certain embodiments, A is selected from cyano, hydroxyl, optionally substituted C₁₋₆alkyl, optionally substituted C₁₋₆ alkoxy, —C(O)NR⁵R⁶, —NR⁵R⁶, —NR⁵C(O)C₁₋₆alkyl, and optionally substituted heterocyclyl. In certain embodiments, A is selected from cyano, hydroxyl, optionally substituted C₁₋₆alkyl, optionally substituted C₁₋₆ alkoxy, —C(O)NR⁵R⁶, —NR⁵R⁶, —NR⁵C(O)C₁₋₆alkyl, optionally substituted heterocyclyl, and optionally substituted heteroaryl. In certain embodiments, A is selected from cyano, hydroxyl, optionally substituted C₁₋₆alkyl, optionally substituted C₁₋₆alkoxy, —C(O)NR⁵R⁶, —NR⁵R⁶, —NR⁵C(O)C₁₋₆alkyl, optionally substituted heterocyclyl, aryl, and optionally substituted heteroaryl. In certain embodiments, A is optionally substituted heterocyclyl. In certain embodiments, A is optionally substituted oxetane. In certain embodiments, A is optionally substituted tetrahydrofuran.

In certain embodiments, Y is NR²R³ and R² and R³ each independently is selected from hydrogen and C₁₋₆alkyl. In certain embodiments, Y is NH₂. In certain embodiments, Y is NMe₂. In certain embodiments, Y is NHMe. In certain embodiments, Y is NR²R³ and R² and R³ taken together with the nitrogen to which they are attached form a 4- or 5-membered heterocyclyl ring. In certain embodiments, Y is hydrogen. In certain embodiments, Y is C₁₋₆ alkyl. In certain embodiments, Y is C₃₋₆ cycloalkyl.

In certain embodiments, X is S.

In certain embodiments, n is 1.

In certain embodiments, R¹⁰ and R¹¹ are both hydrogen.

In certain embodiments, R⁷ and R⁸ are both hydrogen. In certain embodiments, R⁷ and R⁸ are both C₁₋₆alkyl. In certain embodiments, R⁷ is hydrogen and and R⁸ is C₁₋₆alkyl or C₁₋₆ haloalkyl.

In certain embodiments, R⁹ is hydrogen.

In certain embodiments, R¹ is hydrogen.

In certain embodiments, R¹ and R⁹ taken together with the atoms to which they are attached form a 4- or 5-membered heterocyclyl ring. In certain embodiments, R¹ and R⁹ taken together with the atoms to which they are attached form oxetan-3-yl.

The present application provides a compound selected from any one of compounds 1-10, 12-18, 20-25, 27-37, 39, 40, 44-53, 56, 61-70, 74, 76-79, 83, 84, 89, 90, 93-96, 104, 106, 107, 110, 112-117, 119-123, 125, 127-129, 132, 136, 138-141, 146-147, 150-152, 159-162, 172, 174-178, 183, 191-195, 197, 198, 206-208, 212-230, and 232-339 (e.g., 1-10, 12-18, 20-25, 27-37, 39, 40, 44-53, 56, 61-70, 74, 76-79, 83, 84, 89, 90, 93-96, 104, 106, 107, 110, 112-117, 119-123, 125, 127-129, 132, 136, 138-141, 146-147, 150-152, 159-162, 172, 174-178, 183, 191-195, 197, 198, 206-208, and 212-230), and pharmaceutically acceptable salts thereof.

The present application provides a pharmaceutical composition comprising (a) a compound of formula (I), or a pharmaceutically acceptable salt thereof; and (b) a pharmaceutically acceptable excipient. The present application provides a pharmaceutical composition comprising (a) a compound selected from any one of compounds 1-10, 12-18, 20-25, 27-37, 39, 40, 44-53, 56, 61-70, 74, 76-79, 83, 84, 89, 90, 93-96, 104, 106, 107, 110, 112-117, 119-123, 125, 127-129, 132, 136, 138-141, 146-147, 150-152, 159-162, 172, 174-178, 183, 191-195, 197, 198, 206-208, 212-230, and 232-339 (e.g., 1-10, 12-18, 20-25, 27-37, 39, 40, 44-53, 56, 61-70, 74, 76-79, 83, 84, 89, 90, 93-96, 104, 106, 107, 110, 112-117, 119-123, 125, 127-129, 132, 136, 138-141, 146-147, 150-152, 159-162, 172, 174-178, 183, 191-195, 197, 198, 206-208, and 212-230), or a pharmaceutically acceptable salt thereof; and (b) a pharmaceutically acceptable excipient.

The present application provides a compound of formula (I), or a pharmaceutically acceptable salt thereof, or a pharmaceutical composition comprising (a) a compound of formula (I), or a pharmaceutically acceptable salt thereof for use as a medicament. The present application provides a compound selected from any one of compounds 1-10, 12-18, 20-25, 27-37, 39, 40, 44-53, 56, 61-70, 74, 76-79, 83, 84, 89, 90, 93-96, 104, 106, 107, 110, 112-117, 119-123, 125, 127-129, 132, 136, 138-141, 146-147, 150-152, 159-162, 172, 174-178, 183, 191-195, 197, 198, 206-208, 212-230, and 232-339 (e.g., 1-10, 12-18, 20-25, 27-37, 39, 40, 44-53, 56, 61-70, 74, 76-79, 83, 84, 89, 90, 93-96, 104, 106, 107, 110, 112-117, 119-123, 125, 127-129, 132, 136, 138-141, 146-147, 150-152, 159-162, 172, 174-178, 183, 191-195, 197, 198, 206-208, and 212-230), or a pharmaceutically acceptable salt thereof, for use as a medicament.

The present application provides a method of treating Rett syndrome and/or one or more symptoms associated with Rett syndrome in a subject, comprising administering to a subject in need thereof an effective amount of a compound of formula (I), or a pharmaceutically acceptable salt thereof, or a pharmaceutical composition comprising (a) a compound of formula (I), or a pharmaceutically acceptable salt thereof. The present application provides a method of treating Rett syndrome and/or one or more symptoms associated with Rett syndrome in a subject, comprising administering to a subject in need thereof an effective amount of a compound selected from any one of compounds 1-10, 12-18, 20-25, 27-37, 39, 40, 44-53, 56, 61-70, 74, 76-79, 83, 84, 89, 90, 93-96, 104, 106, 107, 110, 112-117, 119-123, 125, 127-129, 132, 136, 138-141, 146-147, 150-152, 159-162, 172, 174-178, 183, 191-195, 197, 198, 206-208, 212-230, and 232-339 (e.g., 1-10, 12-18, 20-25, 27-37, 39, 40, 44-53, 56, 61-70, 74, 76-79, 83, 84, 89, 90, 93-96, 104, 106, 107, 110, 112-117, 119-123, 125, 127-129, 132, 136, 138-141, 146-147, 150-152, 159-162, 172, 174-178, 183, 191-195, 197, 198, 206-208, and 212-230), or a pharmaceutically acceptable salt thereof. In certain embodiments, the one or more symptoms associated with Rett syndrome is selected from sleep disturbances; sleep apnea; seizures; breathing disorders; irregular heartbeat; intellectual disabilities; and autism.

The present application provides a method of treating Rett syndrome and/or one or more symptoms associated with Rett syndrome in a subject, comprising administering to a subject in need thereof an effective amount of a compound of formula (Ia)

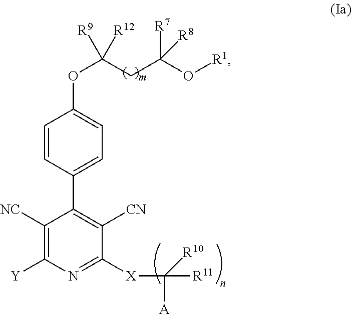

or a pharmaceutically acceptable salt thereof, wherein: Y is selected from hydrogen, C₁₋₆ alkyl, C₃₋₆ cycloalkyl, OR¹³, and NR²R³; R² and R³ each independently is selected from hydrogen and C₁₋₆alkyl; or R² and R³ taken together with the nitrogen to which they are attached form a 4- or 5-membered heterocyclyl ring, wherein the 4- or 5-membered heterocyclyl ring is optionally substituted with one or more substitutent independently selected from halogen or hydroxyl; R¹ is selected from hydrogen, C₁₋₆ alkyl, and C₁₋₆ haloalkyl; R⁷ and R⁸ each independently is selected from hydrogen, C₁₋₆alkyl, and C₁₋₆ haloalkyl; R⁹ is hydrogen or halogen, or R¹ and R⁹ taken together with the atoms to which they are attached form a 4-, 5-, or 6-membered heterocyclyl ring, or R⁹ and R⁸ taken together with the atoms to which they are attached form a cyclopropyl ring system when m is 0 or a cyclobutyl or cyclopentyl ring system when m is 1; R¹⁰ and R¹¹ each independently is selected from hydrogen, hydroxyl, halogen, C₁₋₆alkyl, and C₁₋₆ haloalkyl; R¹² is hydrogen or halogen; R¹³ is selected from hydrogen, C₁₋₆ alkyl, and C₁₋₆ haloalkyl; X is selected from a bond, S, O, and NR⁴, wherein R⁴ is hydrogen or C₁₋₆alkyl (e.g., methyl); A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, optionally substituted aryl, and optionally substituted heteroaryl; R⁵ and R⁶ each independently is selected from hydrogen and C₁₋₆alkyl; m is 0 or 1, provided that when m is 1, R⁸ and R⁹ are taken together with the carbons to which they are attached to form a cyclobutyl or cyclopentyl ring system; and n is 0, 1 or 2. In certain embodiments, the compound is selected from any one of compounds 1-10, 12-18, 20-25, 27-37, 39, 40, 44-53, 56, 61-70, 74, 76-79, 83, 84, 89, 90, 93-96, 104, 106, 107, 110, 112-117, 119-123, 125, 127-129, 132, 136, 138-141, 146-147, 150-152, 159-162, 172, 174-178, 183, 191-195, 197, 198, 206-208, 212-230, and 232-339 (e.g., 1-10, 12-18, 20-25, 27-37, 39, 40, 44-53, 56, 61-70, 74, 76-79, 83, 84, 89, 90, 93-96, 104, 106, 107, 110, 112-117, 119-123, 125, 127-129, 132, 136, 138-141, 146-147, 150-152, 159-162, 172, 174-178, 183, 191-195, 197, 198, 206-208, and 212-230), and pharmaceutically acceptable salts thereof. In certain embodiments, the one or more symptoms associated with Rett syndrome is selected from sleep disturbances; sleep apnea; seizures; breathing disorders; irregular heartbeat; intellectual disabilities; and autism.

The present application provides use of a compound of formula (I), or a pharmaceutically acceptable salt thereof, or a pharmaceutical composition comprising (a) a compound of formula (I), or a pharmaceutically acceptable salt thereof, in the preparation of a medicament for the treatment of Rett syndrome and/or one or more symptoms associated with Rett syndrome. In certain embodiments, the compound is selected from any one of compounds 1-10, 12-18, 20-25, 27-37, 39, 40, 44-53, 56, 61-70, 74, 76-79, 83, 84, 89, 90, 93-96, 104, 106, 107, 110, 112-117, 119-123, 125, 127-129, 132, 136, 138-141, 146-147, 150-152, 159-162, 172, 174-178, 183, 191-195, 197, 198, 206-208, 212-230, and 232-339 (e.g., 1-10, 12-18, 20-25, 27-37, 39, 40, 44-53, 56, 61-70, 74, 76-79, 83, 84, 89, 90, 93-96, 104, 106, 107, 110, 112-117, 119-123, 125, 127-129, 132, 136, 138-141, 146-147, 150-152, 159-162, 172, 174-178, 183, 191-195, 197, 198, 206-208, and 212-230), and pharmaceutically acceptable salts thereof. In certain embodiments, the one or more symptoms associated with Rett syndrome is selected from sleep disturbances; sleep apnea; seizures; breathing disorders; irregular heartbeat; intellectual disabilities; and autism.

The present application provides use of a compound of formula (Ia), or a pharmaceutically acceptable salt thereof, or a pharmaceutical composition comprising (a) a compound of formula (Ia), or a pharmaceutically acceptable salt thereof, in the preparation of a medicament for the treatment of Rett syndrome and/or one or more symptoms associated with Rett syndrome. In certain embodiments, the compound is selected from any one of compounds 1-10, 12-18, 20-25, 27-37, 39, 40, 44-53, 56, 61-70, 74, 76-79, 83, 84, 89, 90, 93-96, 104, 106, 107, 110, 112-117, 119-123, 125, 127-129, 132, 136, 138-141, 146-147, 150-152, 159-162, 172, 174-178, 183, 191-195, 197, 198, 206-208, 212-230, and 232-339 (e.g., 1-10, 12-18, 20-25, 27-37, 39, 40, 44-53, 56, 61-70, 74, 76-79, 83, 84, 89, 90, 93-96, 104, 106, 107, 110, 112-117, 119-123, 125, 127-129, 132, 136, 138-141, 146-147, 150-152, 159-162, 172, 174-178, 183, 191-195, 197, 198, 206-208, and 212-230), and pharmaceutically acceptable salts thereof. In certain embodiments, the one or more symptoms associated with Rett syndrome is selected from sleep disturbances; sleep apnea; seizures; breathing disorders; irregular heartbeat; intellectual disabilities; and autism.

### Detailed Description of the Application

The present application provides a compound of formula (I)

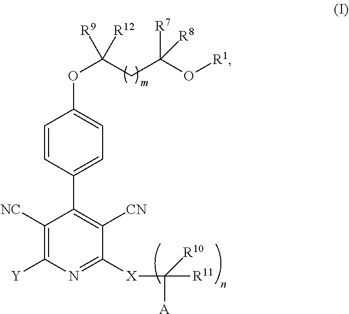

or a pharmaceutically acceptable salt thereof, wherein:
Y is selected from hydrogen, C₁₋₆ alkyl, C₃₋₆ cycloalkyl, OR¹³, and NR²R³,
R² and R³ each independently is selected from hydrogen and C₁₋₆alkyl; or R² and R³ taken together with the nitrogen to which they are attached form a 4- or 5-membered heterocyclyl ring, wherein the 4- or 5-membered heterocyclyl ring is optionally substituted with one or more substitutent independently selected from halogen or hydroxyl;
R¹ is selected from hydrogen, C₁₋₆ alkyl, and C₁₋₆ haloalkyl;
R⁷ and R⁸ each independently is selected from hydrogen, C₁₋₆alkyl, and C₁₋₆ haloalkyl;
R⁹ is hydrogen or halogen, or R¹ and R⁹ taken together with the atoms to which they are attached form a 4-, 5-, or 6-membered heterocyclyl ring, or R⁹ and R⁸ taken together with the atoms to which they are attached form a cyclopropyl ring system when m is 0 or a cyclobutyl or cyclopentyl ring system when m is 1;
R¹⁰ and R¹¹ each independently is selected from hydrogen, hydroxyl, halogen, C₁₋₆alkyl, and C₁₋₆ haloalkyl;
R¹² is hydrogen or halogen;
R¹³ is selected from hydrogen, C₁₋₆ alkyl, and C₁₋₆ haloalkyl;
X is selected from a bond, S, O, and NR⁴, wherein R⁴ is hydrogen or C₁₋₆alkyl (e.g., methyl);
A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, optionally substituted aryl, and optionally substituted heteroaryl;
R⁵ and R⁶ each independently is selected from hydrogen and C₁₋₆alkyl;
m is 0 or 1, provided that when m is 1, R⁸ and R⁹ are taken together with the carbons to which they are attached to form a cyclobutyl or cyclopentyl ring system; and
n is 0, 1 or 2;
provided that when Y is NH₂, m is 0, R¹² is hydrogen, X is S, n is 1, R¹ is hydrogen or methyl, and each of R⁷, R⁸ and R⁹ is hydrogen, then A is not substituted thiazole;
when Y is NH₂, m is 0, R¹² is hydrogen, X is S, n is 1, R⁸ is methyl, and each of R¹, R⁷ and R⁹ is hydrogen, then A is not substituted thiazole;
when Y is NR²R³, R² and R³ are both C₁₋₆alkyl (e.g., ethyl), m is 0, R¹² is hydrogen, X is S, n is 1, and each of R¹, R⁷, R⁸ and R⁹ is hydrogen, then A is not substituted thiazole;
when Y is NR²R³, R² and R³ taken together with the nitrogen to which they are attached form a 4- or 5-membered heterocyclyl ring, m is 0, R¹² is hydrogen, X is S, n is 1, and each of R¹, R⁷, R⁸ and R⁹ is hydrogen, then A is not substituted thiazole;
when Y is NH₂, m is 0, R¹² is hydrogen, X is S, n is 1, R¹ is methyl, and each of R⁷, R⁸, R⁹, R¹⁰, and R¹¹ is hydrogen, then A is not 2-pyridinyl or 3-pyridinyl;
when Y is NH₂, m is 0, R¹² is hydrogen, X is S, n is 1, R⁷ is methyl, R⁸ is methyl, and each of R¹ and R⁹ is hydrogen, then A is not substituted 4-pyridyl;
when Y is NH₂, m is 0, R¹² is hydrogen, X is S, n is 1, and each of R¹, R⁷, R⁸ and R⁹ is hydrogen, then A is not substituted 3-pyridyl;
when Y is hydrogen or NR²R³, R² is hydrogen, R³ is hydrogen or C₁₋₆alkyl, m is 0, R¹² is hydrogen, X is S, n is 1, and each of R¹, R⁷, R⁸ and R⁹ is hydrogen, then A is not substituted 4-pyridyl;
when Y is NH₂ or pyrrolidine, m is 0, R¹² is hydrogen, X is S, n is 1, and each of R¹, R⁷, R⁸, R⁹, R¹⁰ and R¹¹ is hydrogen, then A is not substituted phenyl;
when Y is NH₂, m is 0, R¹² is hydrogen, X is S, n is 1, R¹ is hydrogen or methyl, each of R⁷, R⁸, R⁹, and R¹¹ is hydrogen, and R¹⁰ is methyl, then A is not substituted phenyl;
when Y is H or NH₂, m is 0, R¹² is hydrogen, X is S, n is 0, and each of R¹, R⁷, R⁸ and R⁹ is hydrogen, then A is not optionally substituted aryl (e.g., aryl, such as phenyl);
when Y is NH₂, m is 0, R¹² is hydrogen, X is S or O, n is 1, and each of R¹, R⁷, R⁸ and R⁹ is hydrogen, then A is not substituted oxazole;
when Y is NH₂, m is 0, R¹² is hydrogen, X is 0, n is 1, and each of R¹, R⁷, R⁸ and R⁹ is hydrogen, then A is not substituted phenyl, or substituted 4-pyridyl;
when Y is OR¹³, R¹³ is methyl, m is 0, R¹² is hydrogen, X is S, n is 1, and each of R¹, R⁷, R⁸, R⁹, R¹⁰ and R¹¹ is hydrogen, then A is not substituted phenyl, substituted oxazole, imidazole, or 2-pyridyl;
when Y is OR¹³, R¹³ is ethyl, m is 0, R¹² is hydrogen, X is S, n is 1, and each of R¹, R⁷, R⁸, R⁹, R¹⁰ and R¹¹ is hydrogen, then A is not substituted oxazole; and
when Y is OR¹³, R¹³ is methyl, ethyl, or sec-butyl, m is 0, R¹² is hydrogen, X is S, n is 1, and each of R¹, R⁷, R⁸, R⁹, R¹⁰ and R¹¹ is hydrogen, then A is not substituted thiazole.

The present application provides a compound of formula (I)

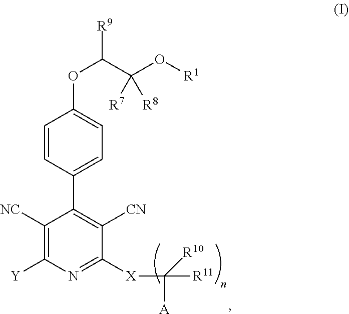

or a pharmaceutically acceptable salt thereof, wherein:
Y is selected from hydrogen, C₁₋₆ alkyl, C₃₋₆ cycloalkyl, and NR²R³,
R² and R³ each independently is selected from hydrogen and C₁₋₆alkyl; or R² and R³ taken together with the nitrogen to which they are attached form a 4- or 5-membered heterocyclyl ring;
R¹ is selected from hydrogen, C₁₋₆ alkyl, and C₁₋₆ haloalkyl;
R⁷ and R⁸ each independently is selected from hydrogen, C₁₋₆alkyl, and C₁₋₆ haloalkyl;
R⁹ is hydrogen, or R¹ and R⁹ taken together with the atoms to which they are attached form a 4- or 5-membered heterocyclyl ring;
R¹⁰ and R¹¹ each independently is selected from hydrogen, hydroxyl, halogen, C₁₋₆alkyl, and C₁₋₆ haloalkyl;
X is selected from a bond, S, O, and NR⁴, wherein R⁴ is hydrogen or C₁₋₆alkyl (e.g., methyl);
A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, optionally substituted aryl, and optionally substituted heteroaryl;
R⁵ and R⁶ each independently is selected from hydrogen and C₁₋₆alkyl; and
n is 0, 1 or 2;
provided that when Y is NH₂, X is S, n is 1, R¹ is hydrogen or methyl, and each of R⁷, R⁸ and R⁹ is hydrogen, then A is not substituted thiazole;
when Y is NH₂, X is S, n is 1, R⁸ is methyl, and each of R¹, R⁷ and R⁹ is hydrogen, then A is not substituted thiazole;
when Y is NR²R³, R² and R³ are both C₁₋₆alkyl (e.g., ethyl), X is S, n is 1, and each of R¹, R⁷, R⁸ and R⁹ is hydrogen, then A is not substituted thiazole;
when Y is NR²R³, R² and R³ taken together with the nitrogen to which they are attached form a 4- or 5-membered heterocyclyl ring, X is S, n is 1, and each of R¹, R⁷, R⁸ and R⁹ is hydrogen, then A is not substituted thiazole;
when Y is NH₂, X is S, n is 1, R¹ is methyl, and each of R⁷, R⁸ and R⁹ is hydrogen, then A is not 2-pyridinyl or 3-pyridinyl;
when Y is NH₂, X is S, n is 1, R⁷ is methyl, R⁸ is methyl, and each of R¹ and R⁹ is hydrogen, then A is not substituted 4-pyridyl;
when Y is NH₂, X is S, n is 1, and each of R¹, R⁷, R⁸ and R⁹ is hydrogen, then A is not substituted 3-pyridyl;
when Y is hydrogen or NR²R³, R² is hydrogen, R³ is hydrogen or C₁₋₆alkyl, X is S, n is 1, and each of R¹, R⁷, R⁸ and R⁹ is hydrogen, then A is not substituted 4-pyridyl;
when Y is NH₂ or pyrrolidine, X is S, n is 1, and each of R¹, R⁷, R⁸, R⁹, R¹⁰ and R¹¹ is hydrogen, then A is not substituted phenyl;
when Y is NH₂, X is S, n is 1, R¹ is hydrogen or methyl, each of R⁷, R¹, R⁹, and R¹¹ is hydrogen, and R¹⁰ is methyl, then A is not substituted phenyl;
when Y is H or NH₂, X is S, n is 0, and each of R¹, R⁷, R⁸ and R⁹ is hydrogen, then A is not optionally substituted aryl (e.g., aryl, such as phenyl);
when Y is NH₂, X is S or O, n is 1, and each of R¹, R⁷, R⁸ and R⁹ is hydrogen, then A is not substituted oxazole; and
when Y is NH₂, X is 0, n is 1, and each of R¹, R⁷, R⁸ and R⁹ is hydrogen, then A is not substituted phenyl, or substituted 4-pyridyl.

The present application provides a method of treating Rett syndrome and/or one or more symptoms associated with Rett syndrome comprising administering to a subject in need thereof a compound of formula (Ia)

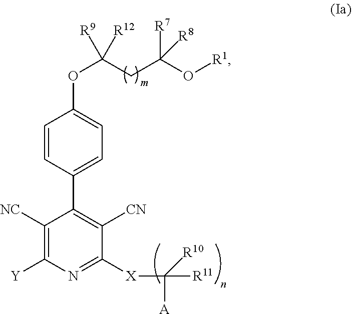

or a pharmaceutically acceptable salt thereof, wherein:
Y is selected from hydrogen, C₁₋₆ alkyl, C₃₋₆ cycloalkyl, OR¹³, and NR²R³,
R² and R³ each independently is selected from hydrogen and C₁₋₆alkyl; or R² and R³ taken together with the nitrogen to which they are attached form a 4- or 5-membered heterocyclyl ring, wherein the 4- or 5-membered heterocyclyl ring is optionally substituted with one or more substitutent independently selected from halogen or hydroxyl;
R¹ is selected from hydrogen, C₁₋₆ alkyl, and C₁₋₆ haloalkyl;
R⁷ and R⁸ each independently is selected from hydrogen, C₁₋₆alkyl, and C₁₋₆ haloalkyl;
R⁹ is hydrogen or halogen, or R¹ and R⁹ taken together with the atoms to which they are attached form a 4-, 5-, or 6-membered heterocyclyl ring, or R⁹ and R⁸ taken together with the atoms to which they are attached form a cyclopropyl ring system when m is 0 or a cyclobutyl or cyclopentyl ring system when m is 1;
R¹⁰ and R¹¹ each independently is selected from hydrogen, hydroxyl, halogen, C₁₋₆alkyl, and C₁₋₆ haloalkyl;
R¹² is hydrogen or halogen;
R¹³ is selected from hydrogen, C₁₋₆ alkyl, and C₁₋₆ haloalkyl;
X is selected from a bond, S, O, and NR⁴, wherein R⁴ is hydrogen or C₁₋₆alkyl (e.g., methyl);
A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, optionally substituted aryl, and optionally substituted heteroaryl;
R⁵ and R⁶ each independently is selected from hydrogen and C₁₋₆alkyl;
m is 0 or 1, provided that when m is 1, R⁸ and R⁹ are taken together with the carbons to which they are attached to form a cyclobutyl or cyclopentyl ring system; and
   n is 0, 1 or 2.

The present application provides a method of treating Rett syndrome and/or one or more symptoms associated with Rett syndrome comprising administering to a subject in need thereof a compound of formula (Ia)

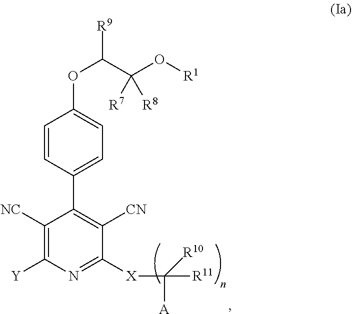

or a pharmaceutically acceptable salt thereof, wherein:
Y is selected from hydrogen, C₁₋₆ alkyl, C₃₋₆ cycloalkyl, and NR²R³, R² and R³ each independently is selected from hydrogen and C₁₋₆alkyl; or R² and R³ taken together with the nitrogen to which they are attached form a 4- or 5-membered heterocyclyl ring;
R¹ is selected from hydrogen, C₁₋₆ alkyl, and C₁₋₆ haloalkyl;
R⁷ and R⁸ each independently is selected from hydrogen, C₁₋₆alkyl, and C₁₋₆ haloalkyl;
R⁹ is hydrogen, or R¹ and R⁹ taken together with the atoms to which they are attached form a 4- or 5-membered heterocyclyl ring;
R¹⁰ and R¹¹ each independently is selected from hydrogen, hydroxyl, halogen, C₁₋₆alkyl, and C₁₋₆ haloalkyl;
X is selected from a bond, S, O, and NR⁴, wherein R⁴ is hydrogen or C₁₋₆alkyl (e.g., methyl);
A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, optionally substituted aryl, and optionally substituted heteroaryl;
R⁵ and R⁶ each independently is selected from hydrogen and C₁₋₆alkyl; and
n is 0, 1 or 2.

In certain embodiments of Formula (I) or (Ia), when Y is NR²R³, R² and R³ each independently is selected from hydrogen and C₁₋₆alkyl, X is S, n is 1, R¹ is hydrogen or methyl, and each of R⁷, R⁸ and R⁹ is hydrogen, then A is not substituted thiazole. In certain such embodiments, m is 0 and R¹² is hydrogen. In certain embodiments of Formula (I) or (Ia), when Y is NH₂, X is S, n is 1, R¹ is hydrogen or methyl, and each of R⁷, R⁸ and R⁹ is hydrogen, then A is not substituted thiazole. In certain such embodiments, m is 0 and R¹² is hydrogen.

In certain embodiments of Formula (I) or (Ia), when Y is NR²R³, R² and R³ each independently is selected from hydrogen and C₁₋₆alkyl, X is S, n is 1, R⁸ is methyl, and each of R¹, R⁷ and R⁹ is hydrogen, then A is not optionally substituted thiazole (e.g., substituted thiazole). In certain such embodiments, m is 0 and R¹² is hydrogen. In certain embodiments of Formula (I) or (Ia), when Y is NH₂, X is S, n is 1, R⁸ is methyl, and each of R¹, R⁷ and R⁹ is hydrogen, then A is not optionally substituted thiazole (e.g., substituted thiazole). In certain such embodiments, m is 0 and R¹² is hydrogen.

In certain embodiments of Formula (I) or (Ia), when Y is NR²R³, R² and R³ taken together with the nitrogen to which they are attached form a 4- or 5-membered heterocyclyl ring, X is S, n is 1, and each of R¹, R⁷, R⁸ and R⁹ is hydrogen, then A is not optionally substituted thiazole (e.g., substituted thiazole). In certain embodiments of Formula (I) or (Ia), when Y is NR²R³, R² and R³ taken together with the nitrogen to which they are attached form a 4- or 5-membered heterocyclyl ring, X is S, and n is 1, then A is not optionally substituted thiazole (e.g., substituted thiazole). In certain embodiments of Formula (I) or (Ia), when Y is NR²R³, R² and R³ taken together with the nitrogen to which they are attached form a 4- or 5-membered heterocyclyl ring, then A is not optionally substituted thiazole (e.g., substituted thiazole). In certain embodiments of the foregoing, m is 0 and R¹² is hydrogen.

In certain embodiments of Formula (I) or (Ia), when Y is NH₂, X is S, n is 1, R¹ is methyl, and each of R⁷, R⁸ and R⁹ is hydrogen, then A is not optionally substituted 2-pyridinyl (e.g., 2-pyridinyl) or optionally substituted 3-pyridinyl (e.g., 3-pyridinyl). In certain embodiments, when Y is NH₂, X is S, n is 1, R¹ is methyl, and each of R⁷, R⁸ and R⁹ is hydrogen, then A is not 2-pyridinyl or 3-pyridinyl. In certain embodiments of Formula (I) or (Ia), when Y is NR²R³, R² and R³ each independently is selected from hydrogen and C₁₋₆ alkyl, X is S, n is 1, R¹ is methyl, and each of R⁷, R⁸ and R⁹ is hydrogen, then A is not optionally substituted 2-pyridinyl (e.g., 2-pyridinyl). In certain embodiments, when Y is NR²R³, R² and R³ each independently is selected from hydrogen and C₁₋₆alkyl, X is S, and n is 1, then A is not optionally substituted 2-pyridinyl (e.g., 2-pyridinyl). In certain embodiments of Formula (I) or (Ia), when Y is NH₂, X is S, n is 1, R¹ is methyl, and each of R⁷, R⁸ and R⁹ is hydrogen, then A is not optionally substituted 2-pyridinyl (e.g., 2-pyridinyl). In certain embodiments of Formula (I) or (Ia), when Y is NH₂, X is S, and n is 1, then A is not optionally substituted 2-pyridinyl (e.g., 2-pyridinyl). In certain embodiments of Formula (I) or (Ia), when X is S, and n is 1, then A is not optionally substituted 2-pyridinyl (e.g., 2-pyridinyl). In certain embodiments of the foregoing, m is 0 and R¹² is hydrogen.

In certain embodiments of Formula (I) or (Ia), when Y is NR²R³, R² and R³ each independently is selected from hydrogen and C₁₋₆alkyl, X is S, n is 1, and each of R¹, R⁷, R⁸ and R⁹ is hydrogen, then A is not substituted pyridyl (e.g., substituted 3-pyridyl). In certain embodiments of Formula (I) or (Ia), when Y is NR²R³, R² and R³ each independently is selected from hydrogen and C₁₋₆alkyl, X is S, and n is 1, then A is not substituted pyridyl (e.g., substituted 3-pyridyl). In certain embodiments of Formula (I) or (Ia), when Y is NR²R³, and R² and R³ each independently is selected from hydrogen and C₁₋₆alkyl, then A is not substituted pyridyl (e.g., substituted 3-pyridyl). In certain embodiments of Formula (I) or (Ia), when Y is NH₂, X is S, n is 1, and each of R¹, R⁷, R⁸ and R⁹ is hydrogen, then A is not substituted 3-pyridyl. In certain embodiments of Formula (I) or (Ia), when Y is NH₂, X is S, and n is 1, then A is not substituted 3-pyridyl. In certain embodiments of Formula (I) or (Ia), when Y is NH₂, then A is not substituted 3-pyridyl. In certain embodiments of Formula (I) or (Ia), when Y is NH₂, X is S, n is 1, and each of R¹, R⁷, R⁸ and R⁹ is hydrogen, then A is not substituted pyridyl. In certain embodiment of Formula (I) or (Ia) s, when Y is NH₂, X is S, and n is 1, then A is not substituted pyridyl. In certain embodiments of Formula (I) or (Ia), when Y is NH₂, then A is not substituted pyridyl. In certain embodiments of the foregoing, m is 0 and R¹² is hydrogen.

In certain embodiments of Formula (I) or (Ia), when Y is NR²R³, X is S, n is 1, R⁷ is methyl, R⁸ is methyl, and each of R¹ and R⁹ is hydrogen, then A is not optionally substituted 4-pyridyl (e.g., substituted 4-pyridyl). In certain embodiments of Formula (I) or (Ia), when Y is NR²R³, X is S, n is 1, R⁷ is methyl, and R⁸ is methyl, then A is not optionally substituted 4-pyridyl (e.g., substituted 4-pyridyl). In certain embodiments of Formula (I) or (Ia), when Y is NR²R³, R² and R³ each independently is selected from hydrogen and C₁₋₆alkyl, X is S, n is 1, R⁷ is methyl, R⁸ is methyl, and each of R¹ and R⁹ is hydrogen, then A is not optionally substituted 4-pyridyl (e.g., substituted 4-pyridyl). In certain embodiments, when Y is NH₂, X is S, n is 1, R⁷ is methyl, R⁸ is methyl, and each of R¹ and R⁹ is hydrogen, then A is not optionally substituted 4-pyridyl (e.g., substituted 4-pyridyl). In certain embodiments of Formula (I) or (Ia), when Y is NR²R³, X is S, and n is 1, then A is not substituted 4-pyridyl. In certain embodiments of Formula (I) or (Ia), A is not substituted 4-pyridyl. In certain embodiments of the foregoing, m is 0 and R¹² is hydrogen.

In certain embodiments of Formula (I) or (Ia), when Y is hydrogen or NR²R³, R² is hydrogen, R³ is hydrogen or C₁₋₆alkyl, X is S, n is 1, and each of R¹, R⁷, R⁸ and R⁹ is hydrogen, then A is not substituted 4-pyridyl. In certain embodiments of Formula (I) or (Ia), when Y is hydrogen, then A is not optionally substituted 4-pyridyl (e.g., substituted 4-pyridyl). In certain embodiments of Formula (I) or (Ia), when Y is hydrogen, then A is not substituted pyridyl. In certain embodiments of the foregoing, m is 0 and R¹² is hydrogen.

In certain embodiments of Formula (I) or (Ia), when Y is NH₂, X is 0, n is 1, and each of R¹, R⁷, R⁸ and R⁹ is hydrogen, then A is not substituted 4-pyridyl. In certain embodiments of Formula (I) or (Ia), when X is 0, then A is not substituted 4-pyridyl. In certain embodiments of Formula (I) or (Ia), when X is 0, then A is not substituted pyridyl. In certain embodiments of the foregoing, m is 0 and R¹² is hydrogen.

In certain embodiments of Formula (I) or (Ia), when Y is NH₂ or pyrrolidine, X is S, n is 1, and each of R¹, R⁷, R⁸ and R⁹ is hydrogen, then A is not substituted phenyl. In certain embodiments of Formula (I) or (Ia), when Y is NR²R³, R² and R³ each independently is selected from hydrogen and C₁₋₆alkyl, X is S, n is 1, and each of R¹, R⁷, R⁸ and R⁹ is hydrogen, then A is not substituted aryl (e.g., substituted phenyl). In certain embodiments of Formula (I) or (Ia), when Y is NR²R³, R² and R³ each independently is selected from hydrogen and C₁₋₆alkyl, and each of R¹, R⁷, R⁸ and R⁹ is hydrogen, then A is not substituted aryl (e.g., substituted phenyl). In certain embodiments of Formula (I) or (Ia), when Y is NR²R³, R² and R³ taken together with the nitrogen to which they are attached form a 4- or 5-membered heterocyclyl ring (e.g., pyrollidine), X is S, n is 1, and each of R¹, R⁷, R⁸ and R⁹ is hydrogen, then A is not optionally substituted aryl (e.g., optionally substituted phenyl, such as substituted phenyl). In certain embodiments of Formula (I) or (Ia), when Y is NR²R³, R² and R³ taken together with the nitrogen to which they are attached form a 4- or 5-membered heterocyclyl ring (e.g., pyrollidine), X is S, and n is 1, then A is not optionally substituted aryl (e.g., optionally substituted phenyl, such as substituted phenyl). In certain embodiments of Formula (I) or (Ia), when Y is NR²R³, and R² and R³ taken together with the nitrogen to which they are attached form a 4- or 5-membered heterocyclyl ring (e.g., pyrollidine), then A is not optionally substituted aryl (e.g., optionally substituted phenyl, such as substituted phenyl). In certain embodiments of the foregoing, m is 0 and R¹² is hydrogen.

In certain embodiments of Formula (I) or (Ia), when Y is H or NR²R³ (e.g., NH₂), X is S, n is 0, and each of R¹, R⁷, R⁸ and R⁹ is hydrogen, then A is not optionally substituted aryl (e.g., aryl, such as phenyl). In certain embodiments of Formula (I) or (Ia), when Y is H or NR²R³ (e.g., NH₂), X is S, and n is 0, then A is not optionally substituted aryl (e.g., aryl, such as phenyl). In certain embodiments, when X is S, and n is 0, then A is not optionally substituted aryl (e.g., aryl, such as phenyl). In certain embodiments of Formula (I) or (Ia), when n is 0, then A is not optionally substituted aryl (e.g., aryl, such as phenyl). In certain embodiments of the foregoing, m is 0 and R¹² is hydrogen.

In certain embodiments of Formula (I) or (Ia), when Y is NR²R³, X is S or O, n is 1, and each of R¹, R⁷, R⁸ and R⁹ is hydrogen, then A is not optionally substituted oxazole (e.g., substituted oxazole). In certain embodiments of Formula (I) or (Ia), when Y is NH₂, X is S or O, n is 1, and each of R¹, R⁷, R⁸ and R⁹ is hydrogen, then A is not optionally substituted oxazole (e.g., substituted oxazole). In certain embodiments of Formula (I) or (Ia), when Y is NH₂, X is S or O, and n is 1, then A is not optionally substituted oxazole (e.g., substituted oxazole). In certain embodiments of Formula (I) or (Ia), when X is S or O, and n is 1, then A is not optionally substituted oxazole (e.g., substituted oxazole). In certain embodiments, when X is S or O, then A is not optionally substituted oxazole (e.g., substituted oxazole). In certain embodiments of Formula (I) or (Ia), when Y is NR²R³ (e.g., NH₂), then A is not optionally substituted oxazole (e.g., substituted oxazole). In certain embodiments of Formula (I) or (Ia), A is not optionally substituted oxazole (e.g., substituted oxazole). In certain embodiments of the foregoing, m is 0 and R¹² is hydrogen.

In certain embodiments of Formula (I) or (Ia), when Y is NR²R³ (e.g., NH₂), X is 0, n is 1, and each of R¹, R⁷, R⁸ and R⁹ is hydrogen, then A is not substituted phenyl. In certain embodiments of Formula (I) or (Ia), when Y is NR²R³ (e.g., NH₂), X is 0, and n is 1, then A is not substituted phenyl. In certain embodiments of Formula (I) or (Ia), when X is 0, and n is 1, then A is not substituted phenyl. In certain embodiments of Formula (I) or (Ia), when X is 0, then A is not substituted phenyl. In certain embodiments of the foregoing, m is 0 and R¹² is hydrogen.

In certain embodiments of Formula (I) or (Ia), when Y is OR¹³, R¹³ is C₁₋₆ alkyl, such as methyl, X is S, n is 1, and each of R¹, R⁷, R⁸, R⁹, R¹⁰ and R¹¹ is hydrogen, then A is not substituted phenyl, substituted oxazole, imidazole, or 2-pyridyl. In certain embodiments of Formula (I) or (Ia), when Y is OR¹³, R¹³ is C₁₋₆ alkyl, such as ethyl, X is S, n is 1, and each of R¹, R⁷, R⁸, R⁹, R¹⁰ and R¹¹ is hydrogen, then A is not substituted oxazole. In certain embodiments of Formula (I) or (Ia), when Y is OR¹³, R¹³ is C₁₋₆ alkyl, such as methyl, ethyl, or sec-butyl, X is S, n is 1, and each of R¹, R⁷, R⁸, R⁹, R¹⁰ and R¹¹ is hydrogen, then A is not substituted thiazole. In certain embodiments of the foregoing, m is 0 and R¹² is hydrogen.

In certain embodiments of Formula (I) or (Ia), X is O and A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, optionally substituted indole, optionally substituted pyrazine, optionally substituted pyrimidine, optionally substituted pyrrolopyridine, optionally substituted pyridazine, optionally substituted indazole, optionally substituted benzofuran, optionally substituted benzoimidazole, optionally substituted benzothiazole, optionally substituted benzothiophene, and optionally substituted thiazole. In certain embodiments of Formula (I) or (Ia), X is O and A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, aryl (e.g., phenyl), and heteroaryl (e.g., indole, pyrazine, pyrimidine, pyrrolopyridine, pyridazine, indazole, benzofuran, benzoimidazole, benzothiazole, benzothiophene, thiazole, oxazole, and pyridine). In certain embodiments of Formula (I) or (Ia), X is O and A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, and heteroaryl (e.g., indole, pyrazine, pyrimidine, pyrrolopyridine, pyridazine, indazole, benzofuran, benzoimidazole, benzothiazole, benzothiophene, thiazole, oxazole, and pyridine). In certain embodiments, X is O and A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, and aryl (e.g., phenyl). In certain embodiments of Formula (I) or (Ia), X is O and A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, aryl, and pyridyl. In certain embodiments of Formula (I) or (Ia), X is O and A is pyridyl. In certain embodiments of Formula (I) or (Ia), X is O and A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl.

In certain embodiments of Formula (I) or (Ia), n is 0 and A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, and optionally substituted heteroaryl.

In certain embodiments of Formula (I) or (Ia), Y is H, and A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, optionally substituted aryl, and heteroaryl (e.g., indole, oxazole, thiazole, pyrazine, pyrimidine, pyrrolopyridine, pyridazine, indazole, benzofuran, benzoimidazole, benzothiazole, benzothiophene, and pyridyl, such as oxazole, thiazole, indole, pyrazine, pyrimidine, pyrrolopyridine, pyridazine, benzofuran, benzoimidazole, benzothiazole, benzothiophene, 2-pyridyl, and 3-pyridyl). In certain embodiments of Formula (I) or (Ia), Y is H, and A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, optionally substituted aryl, optionally substituted oxazole, optionally substituted thiazole, optionally substituted indole, optionally substituted pyrazine, optionally substituted pyrimidine, optionally substituted pyrrolopyridine, optionally substituted pyridazine, optionally substituted indazole, optionally substituted benzofuran, optionally substituted benzoimidazole, optionally substituted benzothiazole, and optionally substituted benzothiophene.

In certain embodiments of Formula (I) or (Ia), when Y is NR²R³, then A is not substituted thiazole, such as phenyl-substituted thiazole. In certain embodiments of Formula (I) or (Ia), when Y is NR²R³, then A is not optionally substituted pyridyl. In certain embodiments of Formula (I) or (Ia), when Y is NR²R³, then A is selected from cyano, hydroxyl, optionally substituted C₁₋₆alkyl, optionally substituted C₁₋₆ alkoxy, —C(O)NR⁵R⁶, —NR⁵R⁶, —NR⁵C(O)C₁₋₆alkyl, optionally substituted heterocyclyl, phenyl, optionally substituted indole, optionally substituted pyrazine, optionally substituted pyrimidine, optionally substituted pyrrolopyridine, optionally substituted pyridazine, optionally substituted indazole, optionally substituted benzofuran, optionally substituted benzoimidazole, optionally substituted benzothiazole, optionally substituted benzothiophene, oxazole, and thiazole. In certain embodiments of Formula (I) or (Ia), when X is S, and n is 1, then A is not substituted thiazole. In certain embodiments of Formula (I) or (Ia), when X is S, and n is 1, then A is not optionally substituted pyridyl. In certain embodiments of Formula (I) or (Ia), when X is S and n is 1, then A is selected from cyano, hydroxyl, optionally substituted C₁₋₆alkyl, optionally substituted C₁₋₆ alkoxy, —C(O)NR⁵R⁶, —NR⁵R⁶, —NR⁵C(O)C₁₋₆alkyl, optionally substituted heterocyclyl, phenyl, optionally substituted indole, optionally substituted pyrazine, optionally substituted pyrimidine, optionally substituted pyrrolopyridine, optionally substituted pyridazine, optionally substituted indazole, optionally substituted benzofuran, optionally substituted benzoimidazole, optionally substituted benzothiazole, optionally substituted benzothiophene, oxazole, and thiazole. In certain embodiments of Formula (I) or (Ia), A is not substituted thiazole, such as phenyl-substituted thiazole. In certain embodiments of Formula (I) or (Ia), A is not optionally substituted pyridyl.

In certain embodiments of Formula (I) or (Ia), A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, aryl (e.g., phenyl), and optionally substituted heteroaryl. In certain embodiments of Formula (I) or (Ia), A is selected from cyano, hydroxyl, optionally substituted C₁₋₆alkyl, optionally substituted C₁₋₆ alkoxy, —C(O)NR⁵R⁶, —NR⁵R⁶, —NR⁵C(O)C₁₋₆ alkyl, optionally substituted heterocyclyl, optionally substituted aryl, optionally substituted indole, optionally substituted pyrazine, optionally substituted pyrimidine, optionally substituted pyrrolopyridine, optionally substituted pyridazine, optionally substituted indazole, optionally substituted benzofuran, optionally substituted benzoimidazole, optionally substituted benzothiazole, and optionally substituted benzothiophene. In certain embodiments of Formula (I) or (Ia), A is selected from cyano, hydroxyl, optionally substituted C₁₋₆alkyl, optionally substituted C₁₋₆ alkoxy, —C(O)NR⁵R⁶, —NR⁵R⁶, —NR⁵C(O)C₁₋₆alkyl, optionally substituted heterocyclyl, optionally substituted aryl, optionally substituted indole, optionally substituted pyrazine, optionally substituted pyrimidine, optionally substituted pyrrolopyridine, optionally substituted pyridazine, optionally substituted indazole, optionally substituted benzofuran, optionally substituted benzoimidazole, optionally substituted benzothiazole, optionally substituted benzothiophene, oxazole, and thiazole. In certain embodiments of Formula (I) or (Ia), A is selected from cyano, hydroxyl, optionally substituted C₁₋₆alkyl, optionally substituted C₁₋₆alkoxy, —C(O)NR⁵R⁶, —NR⁵R⁶, —NR⁵C(O)C₁₋₆alkyl, optionally substituted heterocyclyl, optionally substituted indole, optionally substituted pyrazine, optionally substituted pyrimidine, optionally substituted pyrrolopyridine, optionally substituted pyridazine, optionally substituted indazole, optionally substituted benzofuran, optionally substituted benzoimidazole, optionally substituted benzothiazole, and optionally substituted benzothiophene. In certain embodiments of Formula (I) or (Ia), A is selected from cyano, hydroxyl, optionally substituted C₁₋₆alkyl, optionally substituted C₁₋₆ alkoxy, —C(O)NR⁵R⁶, —NR⁵R⁶, —NR⁵C(O)C₁₋₆alkyl, and optionally substituted heterocyclyl. In certain embodiments, A is selected from cyano, hydroxyl, optionally substituted C₁₋₆alkyl, optionally substituted C₁₋₆ alkoxy, —C(O)NR⁵R⁶, —NR⁵R⁶, —NR⁵C(O)C₁₋₆alkyl, optionally substituted heterocyclyl, and optionally substituted heteroaryl. In certain embodiments of Formula (I) or (Ia), A is selected from cyano, hydroxyl, optionally substituted C₁₋₆alkyl, optionally substituted C₁₋₆alkoxy, —C(O)NR⁵R⁶, —NR⁵R⁶, —NR⁵C(O)C₁₋₆alkyl, optionally substituted heterocyclyl, aryl, and optionally substituted heteroaryl. In certain embodiments of Formula (I) or (Ia), A is optionally substituted heterocyclyl, such as oxetane, tetrahydrofuran or tetrahydropyran. In certain embodiments of Formula (I) or (Ia), A is optionally substituted oxetane, such as oxetan-3-yl. In certain embodiments, A is optionally substituted tetrahydrofuran, such as tetrahydrofuran-3-yl. In certain embodiments of Formula (I) or (Ia), A is optionally substituted C₁₋₆alkoxy, such as methoxy or ethoxy. In certain embodiments of Formula (I) or (Ia), A is optionally substituted C₁₋₆alkyl, such as methyl, trifluoromethyl, or hydroxyalkyl.

In certain embodiments of Formula (I) or (Ia), Y is selected from hydrogen, C₁₋₆ alkyl, C₃₋₆ cycloalkyl, and NR²R³. In certain embodiments of Formula (I) or (Ia), Y is O. In certain embodiments of Formula (I) or (Ia), Y is NR²R³ and R² and R³ each independently is selected from hydrogen and C₁₋₆alkyl, such as methyl. In certain such embodiments of Formula (I) or (Ia), Y is NH₂. In certain such embodiments of Formula (I) or (Ia), Y is NHMe. In certain such embodiments of Formula (I) or (Ia), Y is NMe₂. In certain embodiments of Formula (I) or (Ia), Y is NR²R³ and R² and R³ taken together with the nitrogen to which they are attached form a 4- or 5-membered heterocyclyl ring (e.g., pyrrolidine or azetidine). In certain such embodiments, the 4- or 5-membered heterocyclyl ring is optionally substituted with one or more substitutent independently selected from halogen or hydroxyl. In other embodiments, the 4- or 5-membered heterocyclyl ring is unsubstituted. In other embodiments of Formula (I) or (Ia), Y is hydrogen. In certain embodiments, Y is C₁₋₆ alkyl, such as methyl. In certain embodiments of Formula (I) or (Ia), Y is C₃₋₆ cycloalkyl, such as cyclopropyl.

In certain embodiments of Formula (I) or (Ia), R⁷ and R⁸ are both hydrogen. In certain embodiments of Formula (I) or (Ia), R⁷ is hydrogen and and R⁸ is C₁₋₆alkyl, or C₁₋₆ haloalkyl, such as methyl or trifluoromethyl. In certain embodiments of Formula (I) or (Ia), R⁷ and R⁸ are both C₁₋₆alkyl, such as methyl.

In certain embodiments of Formula (I) or (Ia), R⁹ is hydrogen. In certain embodiments of Formula (I) or (Ia), R⁹ is halogen, such as F. In certain embodiments of Formula (I) or (Ia), R¹ is hydrogen. In certain embodiments of Formula (I) or (Ia), R¹ and R⁹ taken together with the atoms to which they are attached form a 4- or 5-membered heterocyclyl ring, such as oxetane or tetrahydrofuran. In certain embodiments of Formula (I) or (Ia), R¹ and R⁹ taken together with the atoms to which they are attached form oxetan-3-yl. In certain embodiments of Formula (I) or (Ia), R¹ and R⁹ taken together with the atoms to which they are attached form a 6-membered heterocyclyl ring, such as oxetane or tetrahydropyran.

In certain embodiments, m is 0. In certain such embodiments, R⁹ and R⁸ taken together with the atoms to which they are attached form a cyclopropyl ring system. In certain embodiments, m is 1 and R⁸ and R⁹ are taken together with the carbons to which they are attached to form a cyclobutyl or cyclopentyl ring system.

In certain embodiments of Formula (I) or (Ia), R⁷ and R⁸ are both hydrogen and R¹ and R⁹ taken together with the atoms to which they are attached form oxetan-3-yl. In certain embodiments of Formula (I) or (Ia), R¹, R⁷, R⁸, and R⁹ are each hydrogen.

In certain embodiments of Formula (I) or (Ia), R¹⁰ and R¹¹ are both hydrogen.

In certain embodiments of Formula (I) or (Ia), n is 0 and A is optionally substituted heterocyclyl.

In certain embodiments of Formula (I) or (Ia), R¹³ is C₁₋₆ alkyl, such as methyl.

In certain embodiments of Formula (I) or (Ia), X is S. In certain embodiments of Formula (I) or (Ia), X is S, and n is 0. In certain embodiments of Formula (I) or (Ia), X is S, and n is 1. In certain embodiments of Formula (I) or (Ia), X is S, and n is 2.

In certain embodiments of Formula (I) or (Ia), X is O. In certain embodiments of Formula (I) or (Ia), X is 0, and n is 1.

In certain embodiments of Formula (I) or (Ia), X is a bond. In certain embodiments of Formula (I) or (Ia), X is a bond, and n is 0. In certain embodiments of Formula (I) or (Ia), X is a bond, and n is 2.

In certain embodiments of Formula (I) or (Ia), X is NR⁴, wherein R⁴ is hydrogen or C₁₋₆alkyl (e.g., methyl). In certain embodiments of Formula (I) or (Ia), X is NR⁴, wherein R⁴ is hydrogen or C₁₋₆alkyl (e.g., methyl), and n is 1.

In certain embodiments of Formula (I) or (Ia), n is 1 or 2.

In certain embodiments of Formula (I) or (Ia), R¹ is haloalkyl (e.g., difluoromethyl). In certain embodiments, R¹ is hydrogen. In certain embodiments of Formula (I) or (Ia), R¹ is C₁₋₆ alkyl (e.g., methyl or ethyl).

In certain embodiments of Formula (I) or (Ia), X is S, n is 1, R¹⁰ and R¹¹ are both hydrogen, and A is optionally substituted heterocyclyl, such as oxetane (e.g., oxetan-3-yl), tetrahydrofuran (e.g., tetrahydrofuran-3-yl) or tetrahydropyran. In certain embodiments of Formula (I) or (Ia), X is S, n is 1, R¹⁰ and R¹¹ are both hydrogen, and A is optionally substituted oxetane, such as oxetan-3-yl. In certain embodiments of Formula (I) or (Ia), X is S, n is 1, R¹⁰ and R¹¹ are both hydrogen, and A is optionally substituted tetrahydrofuran, such as tetrahydrofuran-3-yl. In certain embodiments of Formula (I) or (Ia), Y is NR²R³, R² and R³ each independently is selected from hydrogen and C₁₋₆alkyl (e.g., methyl), X is S, n is 1, R¹⁰ and R¹¹ are both hydrogen, and A is optionally substituted heterocyclyl, such as oxetane (e.g., oxetan-3-yl), tetrahydrofuran (e.g., tetrahydrofuran-3-yl) or tetrahydropyran. In certain such embodiments of Formula (I) or (Ia), Y is NH₂. In certain such embodiments, Y is NHMe. In certain such embodiments of Formula (I) or (Ia), Y is NMe₂. In certain embodiments of the foregoing, R¹ and R⁹ taken together with the atoms to which they are attached form a 4- or 5-membered heterocyclyl ring, such as oxetane or tetrahydrofuran.

The present application provides a compound selected from any one of the following, and pharmaceutically acceptable salts thereof:

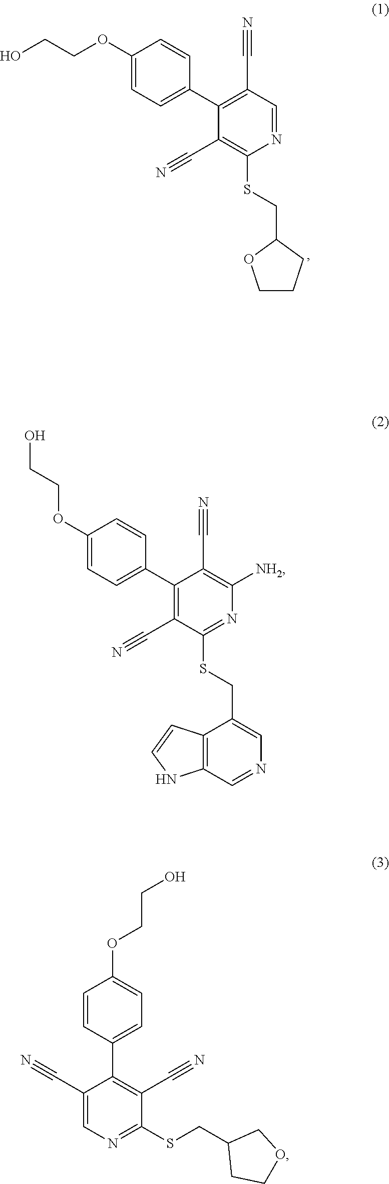 [SMILES: N#Cc1cnc(SCC2CCCO2)c(C#N)c1-c1ccc(OCCO)cc1 ; N#Cc1c(N)nc(SCc2cncc3[nH]ccc23)c(C#N)c1-c1ccc(OCCO)cc1 ; N#Cc1cnc(SCC2CCOC2)c(C#N)c1-c1ccc(OCCO)cc1]

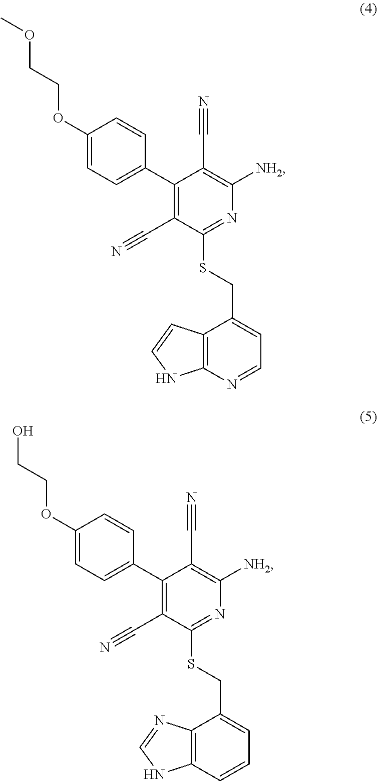 [SMILES: COCCOc1ccc(-c2c(C#N)c(N)nc(SCc3ccnc4[nH]ccc34)c2C#N)cc1 ; N#Cc1c(N)nc(SCc2cccc3[nH]cnc23)c(C#N)c1-c1ccc(OCCO)cc1]

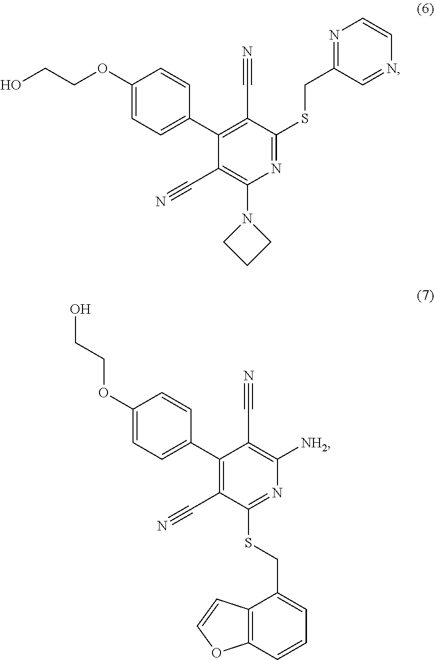 [SMILES: N#Cc1c(SCc2cnccn2)nc(N2CCC2)c(C#N)c1-c1ccc(OCCO)cc1 ; N#Cc1c(N)nc(SCc2cccc3occc23)c(C#N)c1-c1ccc(OCCO)cc1]

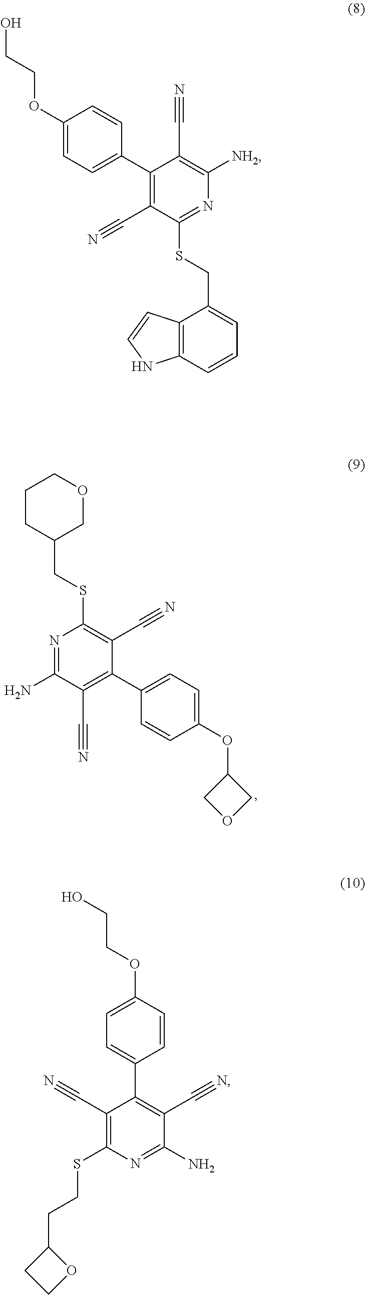 [SMILES: N#Cc1c(N)nc(SCc2cccc3[nH]ccc23)c(C#N)c1-c1ccc(OCCO)cc1 ; N#Cc1c(N)nc(SCC2CCCOC2)c(C#N)c1-c1ccc(OC2COC2)cc1 ; N#Cc1c(N)nc(SCCC2CCO2)c(C#N)c1-c1ccc(OCCO)cc1]

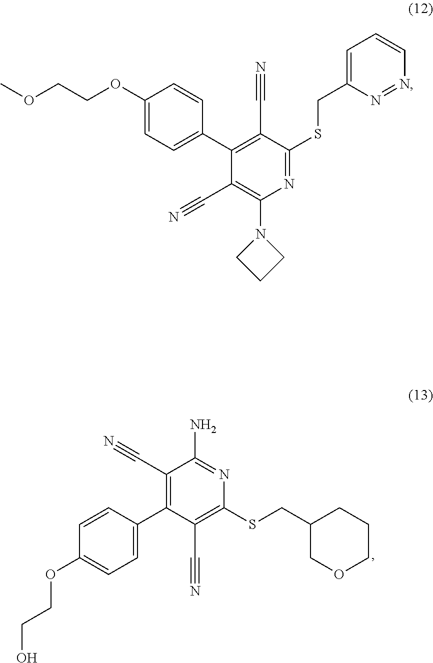 [SMILES: COCCOc1ccc(-c2c(C#N)c(SCc3cccnn3)nc(N3CCC3)c2C#N)cc1 ; N#Cc1c(N)nc(SCC2CCCOC2)c(C#N)c1-c1ccc(OCCO)cc1]

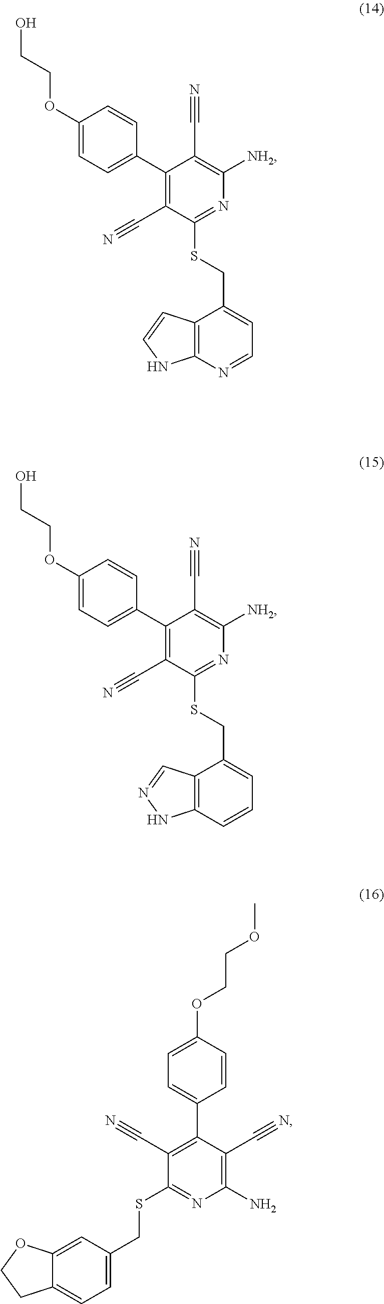 [SMILES: N#Cc1c(N)nc(SCc2ccnc3[nH]ccc23)c(C#N)c1-c1ccc(OCCO)cc1 ; N#Cc1c(N)nc(SCc2cccc3[nH]ncc23)c(C#N)c1-c1ccc(OCCO)cc1 ; COCCOc1ccc(-c2c(C#N)c(N)nc(SCc3ccc4c(c3)OCC4)c2C#N)cc1]

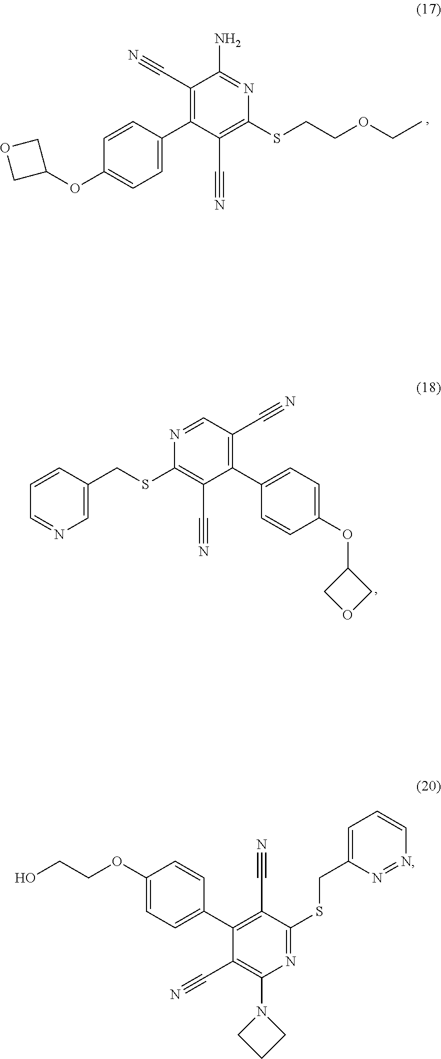 [SMILES: CCOCCSc1nc(N)c(C#N)c(-c2ccc(OC3COC3)cc2)c1C#N ; N#Cc1cnc(SCc2cccnc2)c(C#N)c1-c1ccc(OC2COC2)cc1 ; N#Cc1c(SCc2cccnn2)nc(N2CCC2)c(C#N)c1-c1ccc(OCCO)cc1]

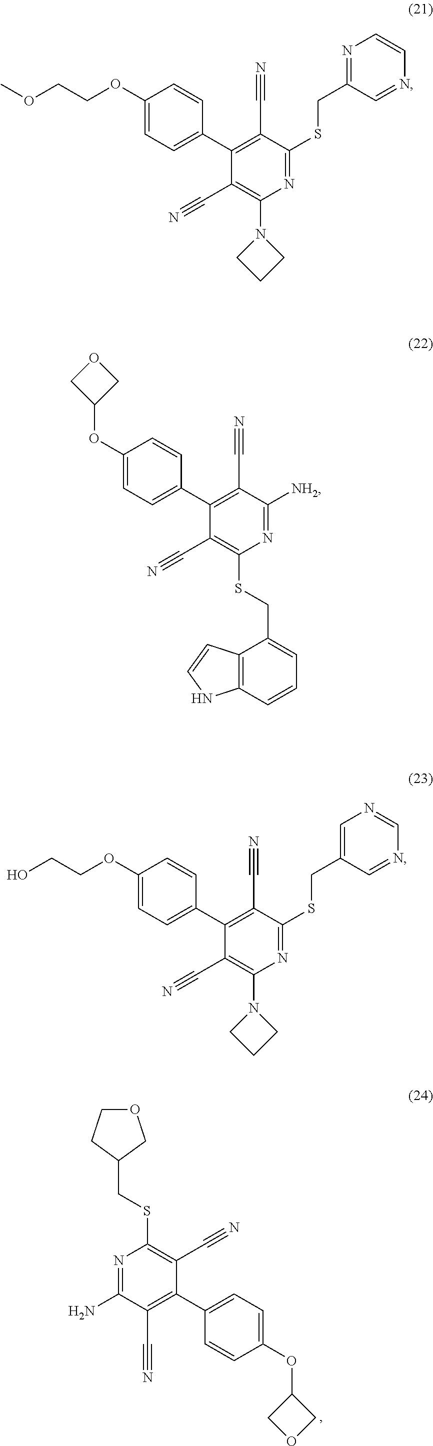 [SMILES: COCCOc1ccc(-c2c(C#N)c(SCc3cnccn3)nc(N3CCC3)c2C#N)cc1 ; N#Cc1c(N)nc(SCc2cccc3[nH]ccc23)c(C#N)c1-c1ccc(OC2COC2)cc1 ; N#Cc1c(SCc2cncnc2)nc(N2CCC2)c(C#N)c1-c1ccc(OCCO)cc1 ; N#Cc1c(N)nc(SCC2CCOC2)c(C#N)c1-c1ccc(OC2COC2)cc1]

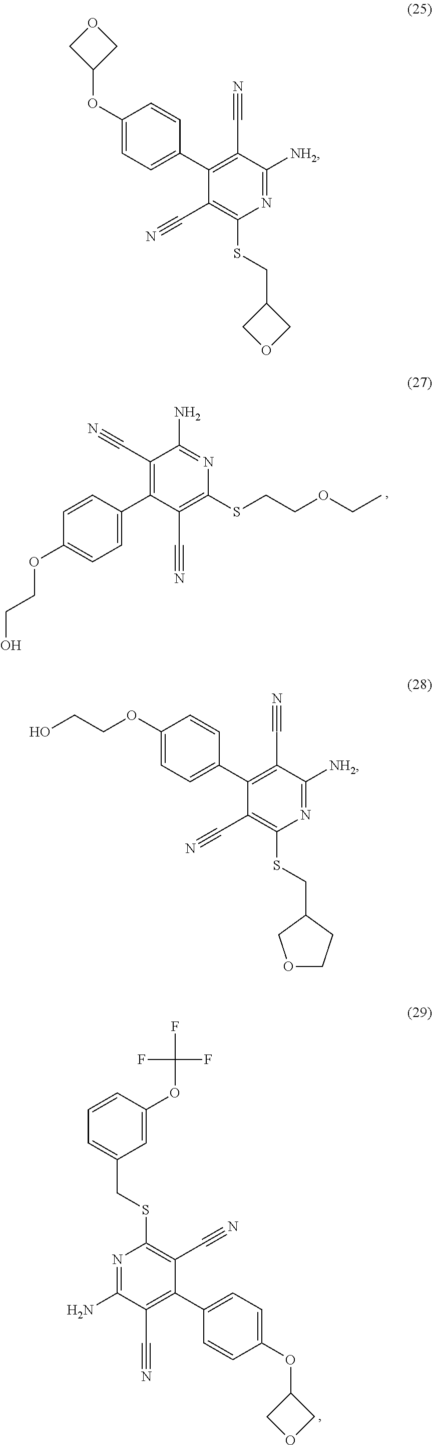 [SMILES: N#Cc1c(N)nc(SCC2COC2)c(C#N)c1-c1ccc(OC2COC2)cc1 ; CCOCCSc1nc(N)c(C#N)c(-c2ccc(OCCO)cc2)c1C#N ; N#Cc1c(N)nc(SCC2CCOC2)c(C#N)c1-c1ccc(OCCO)cc1 ; N#Cc1c(N)nc(SCc2cccc(OC(F)(F)F)c2)c(C#N)c1-c1ccc(OC2COC2)cc1]

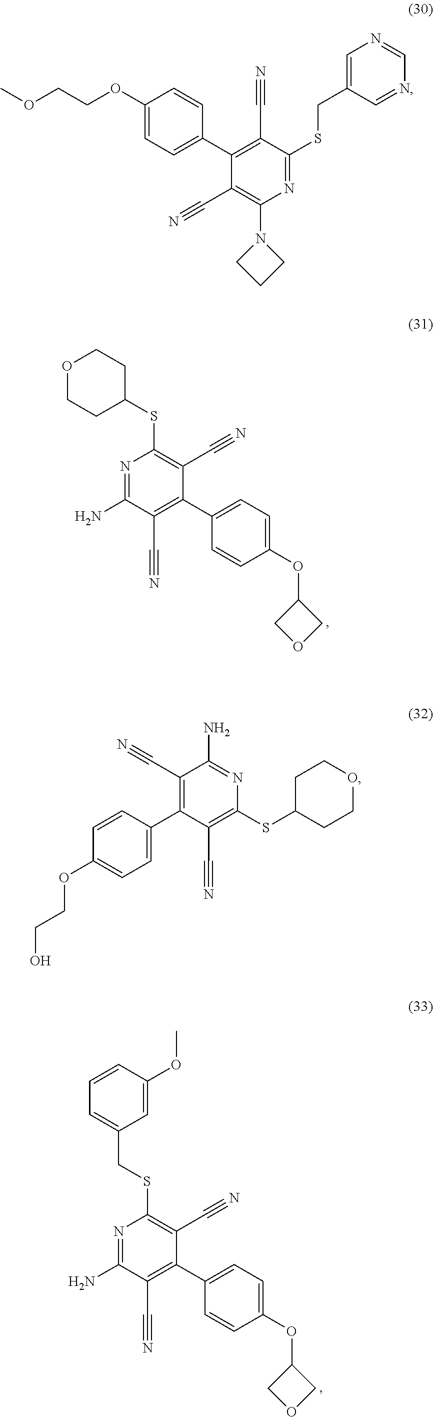 [SMILES: COCCOc1ccc(-c2c(C#N)c(SCc3cncnc3)nc(N3CCC3)c2C#N)cc1 ; N#Cc1c(N)nc(SC2CCOCC2)c(C#N)c1-c1ccc(OC2COC2)cc1 ; N#Cc1c(N)nc(SC2CCOCC2)c(C#N)c1-c1ccc(OCCO)cc1 ; COc1cccc(CSc2nc(N)c(C#N)c(-c3ccc(OC4COC4)cc3)c2C#N)c1]

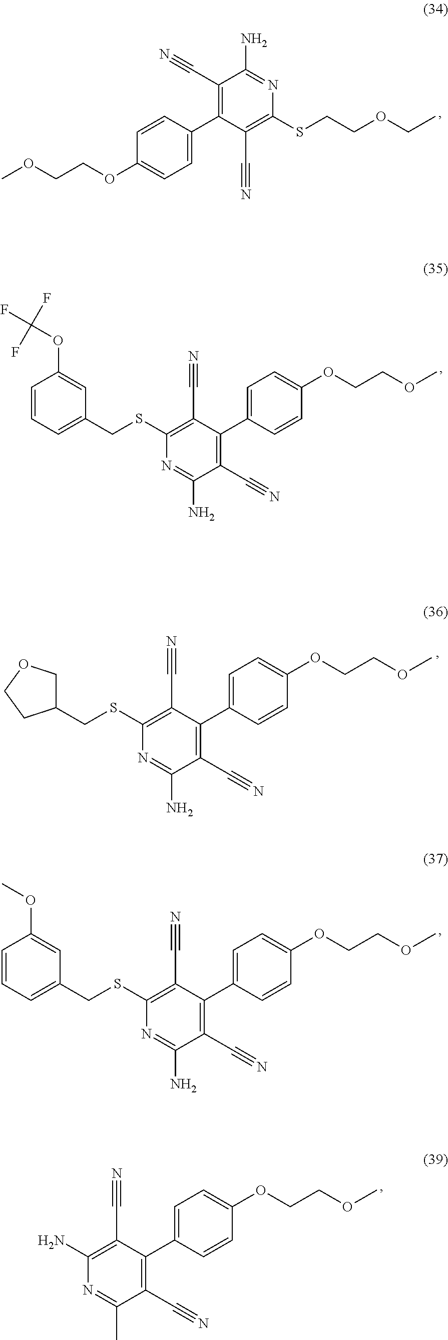 [SMILES: CCOCCSc1nc(N)c(C#N)c(-c2ccc(OCCOC)cc2)c1C#N ; COCCOc1ccc(-c2c(C#N)c(N)nc(SCc3cccc(OC(F)(F)F)c3)c2C#N)cc1 ; COCCOc1ccc(-c2c(C#N)c(N)nc(SCC3CCOC3)c2C#N)cc1 ; COCCOc1ccc(-c2c(C#N)c(N)nc(SCc3cccc(OC)c3)c2C#N)cc1 ; COCCOc1ccc(-c2c(C#N)c(C)nc(N)c2C#N)cc1]

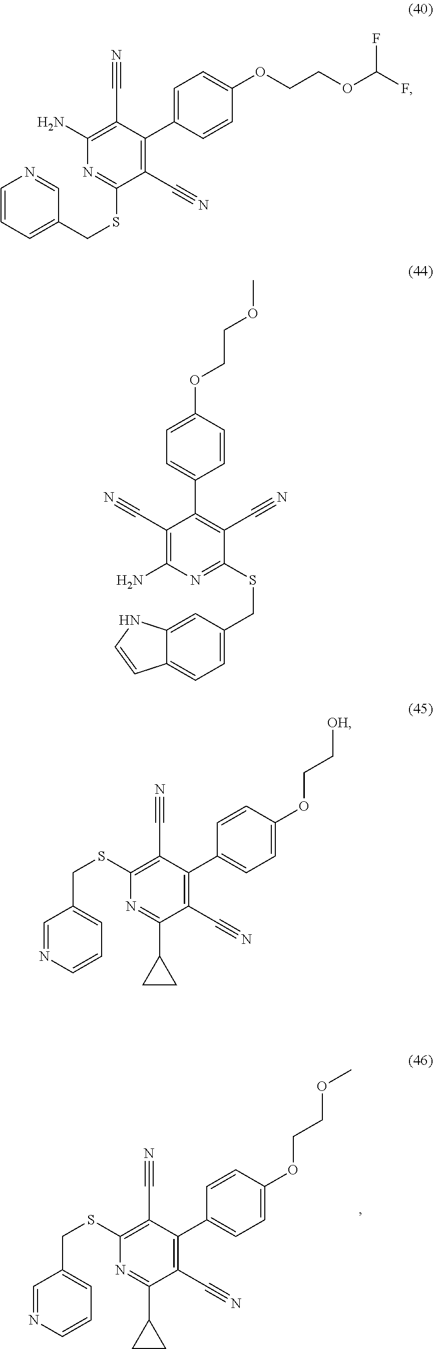 [SMILES: N#Cc1c(N)nc(SCc2cccnc2)c(C#N)c1-c1ccc(OCCOC(F)F)cc1 ; COCCOc1ccc(-c2c(C#N)c(N)nc(SCc3ccc4cc[nH]c4c3)c2C#N)cc1 ; N#Cc1c(SCc2cccnc2)nc(C2CC2)c(C#N)c1-c1ccc(OCCO)cc1 ; COCCOc1ccc(-c2c(C#N)c(SCc3cccnc3)nc(C3CC3)c2C#N)cc1]

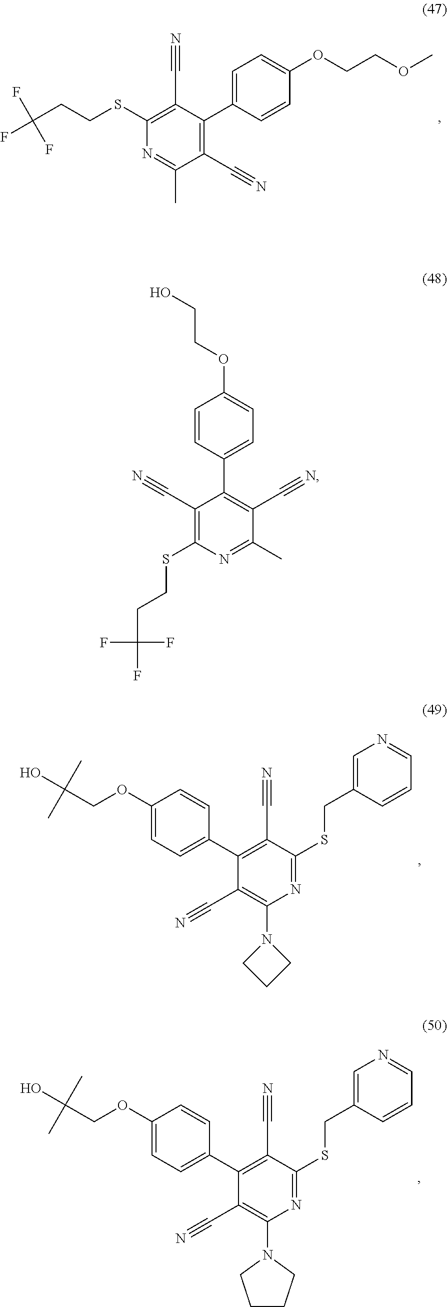 [SMILES: COCCOc1ccc(-c2c(C#N)c(C)nc(SCCC(F)(F)F)c2C#N)cc1 ; Cc1nc(SCCC(F)(F)F)c(C#N)c(-c2ccc(OCCO)cc2)c1C#N ; CC(C)(O)COc1ccc(-c2c(C#N)c(SCc3cccnc3)nc(N3CCC3)c2C#N)cc1 ; CC(C)(O)COc1ccc(-c2c(C#N)c(SCc3cccnc3)nc(N3CCCC3)c2C#N)cc1]

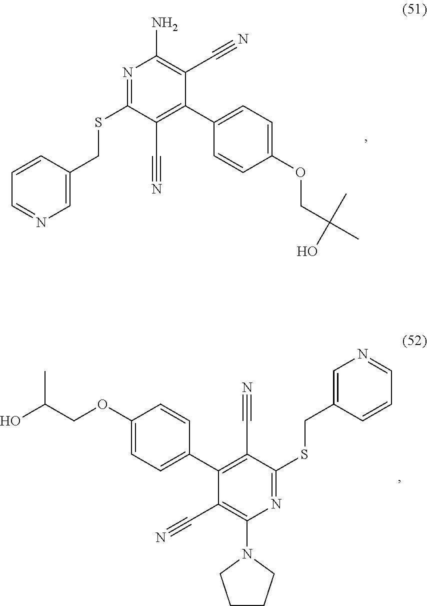 [SMILES: CC(C)(O)COc1ccc(-c2c(C#N)c(N)nc(SCc3cccnc3)c2C#N)cc1 ; CC(O)COc1ccc(-c2c(C#N)c(SCc3cccnc3)nc(N3CCCC3)c2C#N)cc1]

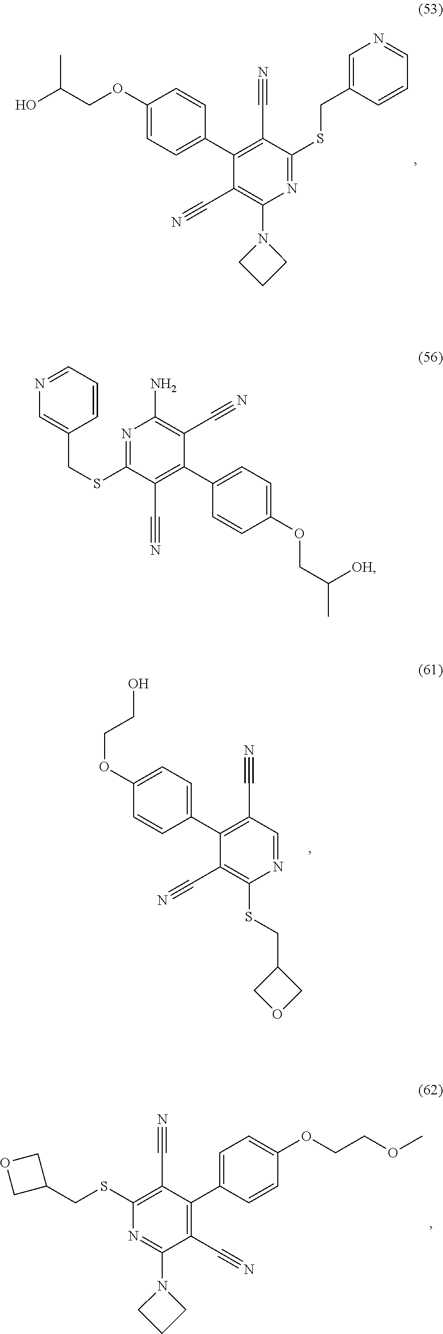 [SMILES: CC(O)COc1ccc(-c2c(C#N)c(SCc3cccnc3)nc(N3CCC3)c2C#N)cc1 ; CC(O)COc1ccc(-c2c(C#N)c(N)nc(SCc3cccnc3)c2C#N)cc1 ; N#Cc1cnc(SCC2COC2)c(C#N)c1-c1ccc(OCCO)cc1 ; COCCOc1ccc(-c2c(C#N)c(SCC3COC3)nc(N3CCC3)c2C#N)cc1]

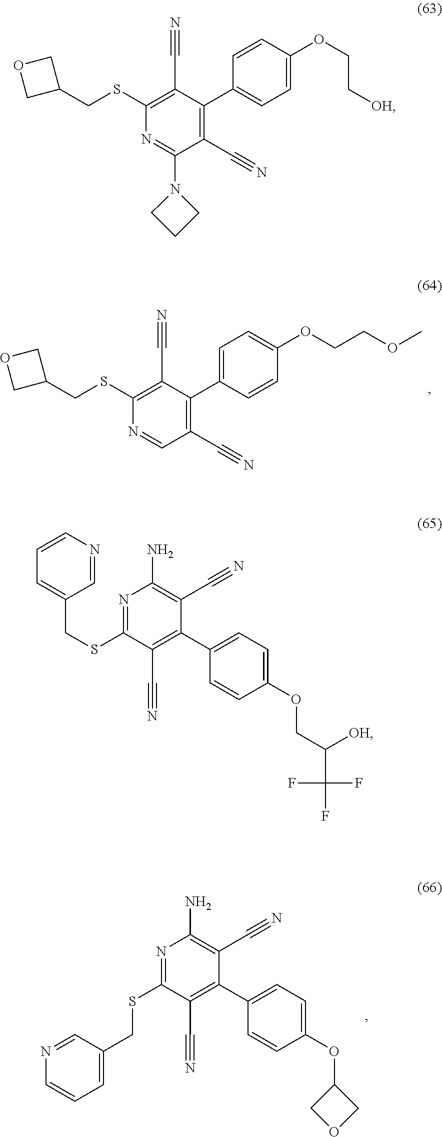 [SMILES: N#Cc1c(SCC2COC2)nc(N2CCC2)c(C#N)c1-c1ccc(OCCO)cc1 ; COCCOc1ccc(-c2c(C#N)cnc(SCC3COC3)c2C#N)cc1 ; N#Cc1c(N)nc(SCc2cccnc2)c(C#N)c1-c1ccc(OCC(O)C(F)(F)F)cc1 ; N#Cc1c(N)nc(SCc2cccnc2)c(C#N)c1-c1ccc(OC2COC2)cc1]

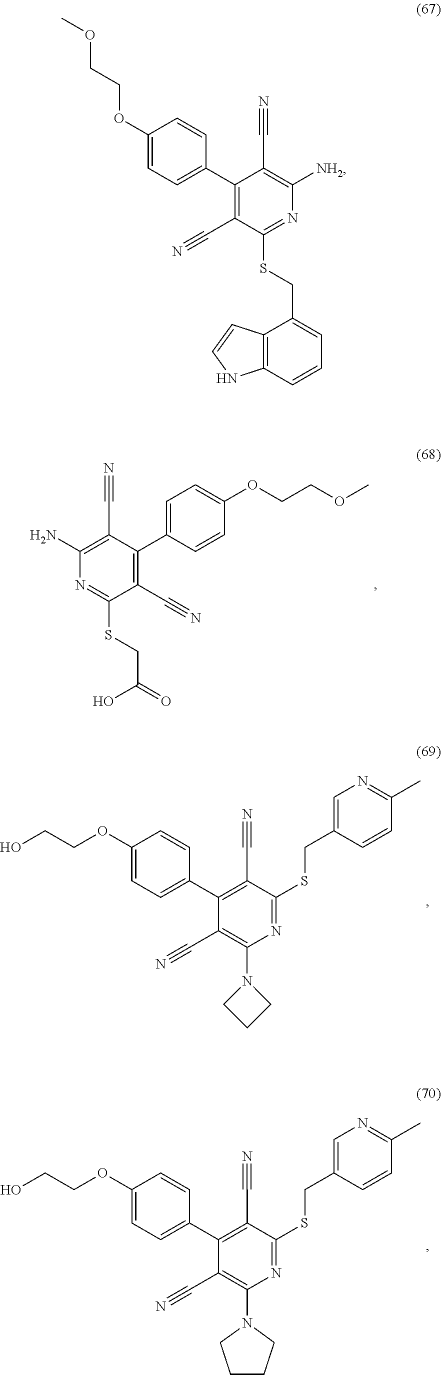 [SMILES: COCCOc1ccc(-c2c(C#N)c(N)nc(SCc3cccc4[nH]ccc34)c2C#N)cc1 ; COCCOc1ccc(-c2c(C#N)c(N)nc(SCC(=O)O)c2C#N)cc1 ; Cc1ccc(CSc2nc(N3CCC3)c(C#N)c(-c3ccc(OCCO)cc3)c2C#N)cn1 ; Cc1ccc(CSc2nc(N3CCCC3)c(C#N)c(-c3ccc(OCCO)cc3)c2C#N)cn1]

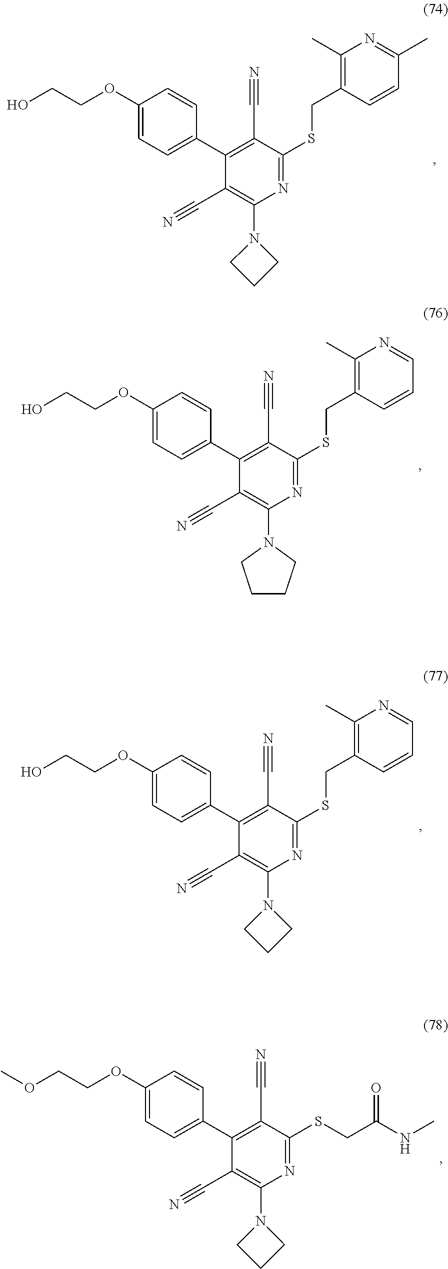

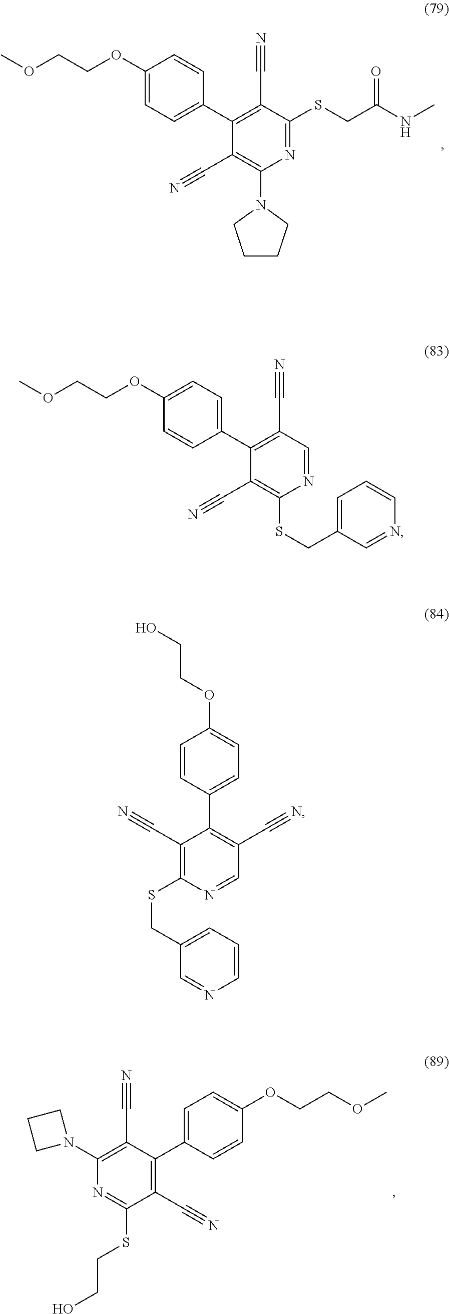

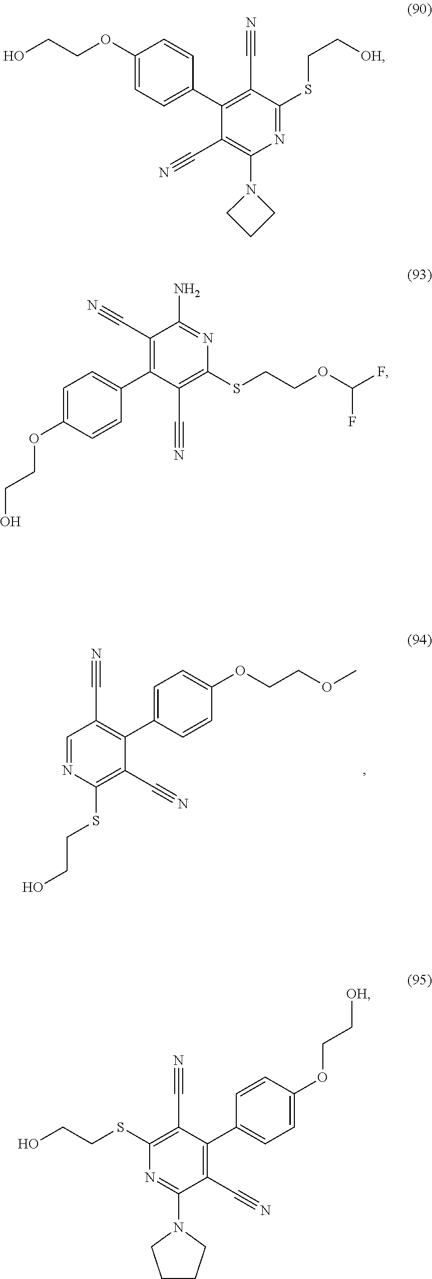 [SMILES: N#Cc1c(SCCO)nc(N2CCC2)c(C#N)c1-c1ccc(OCCO)cc1 ; N#Cc1c(N)nc(SCCOC(F)F)c(C#N)c1-c1ccc(OCCO)cc1 ; COCCOc1ccc(-c2c(C#N)cnc(SCCO)c2C#N)cc1 ; N#Cc1c(SCCO)nc(N2CCCC2)c(C#N)c1-c1ccc(OCCO)cc1]

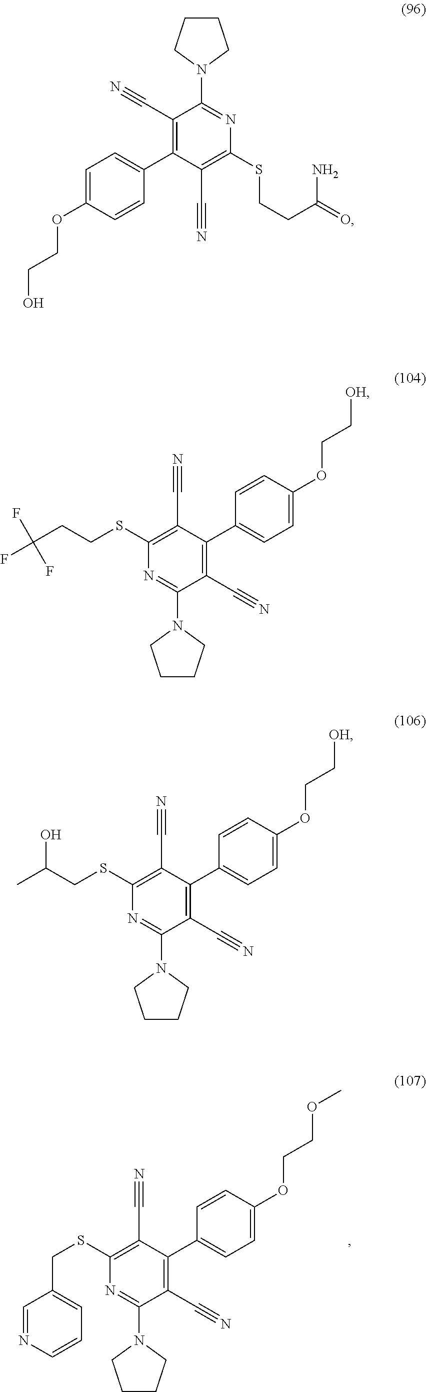 [SMILES: N#Cc1c(SCCC(N)=O)nc(N2CCCC2)c(C#N)c1-c1ccc(OCCO)cc1 ; N#Cc1c(SCCC(F)(F)F)nc(N2CCCC2)c(C#N)c1-c1ccc(OCCO)cc1 ; CC(O)CSc1nc(N2CCCC2)c(C#N)c(-c2ccc(OCCO)cc2)c1C#N ; COCCOc1ccc(-c2c(C#N)c(SCc3cccnc3)nc(N3CCCC3)c2C#N)cc1]

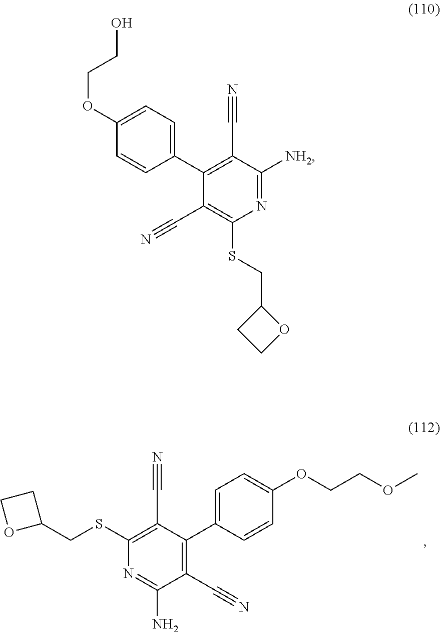 [SMILES: N#Cc1c(N)nc(SCC2CCO2)c(C#N)c1-c1ccc(OCCO)cc1 ; COCCOc1ccc(-c2c(C#N)c(N)nc(SCC3CCO3)c2C#N)cc1]

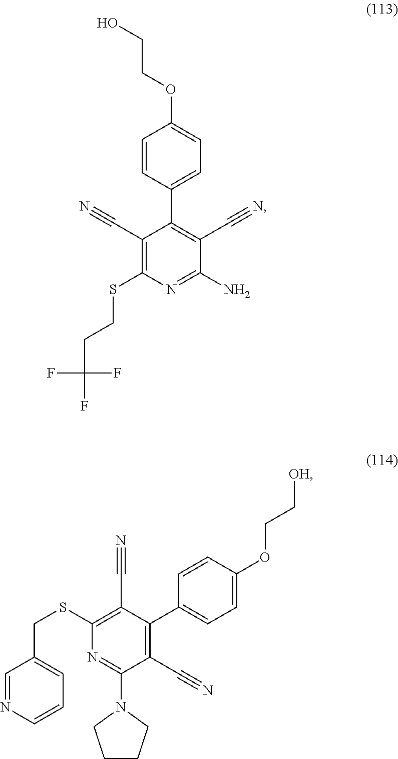 [SMILES: N#Cc1c(N)nc(SCCC(F)(F)F)c(C#N)c1-c1ccc(OCCO)cc1 ; N#Cc1c(SCc2cccnc2)nc(N2CCCC2)c(C#N)c1-c1ccc(OCCO)cc1]

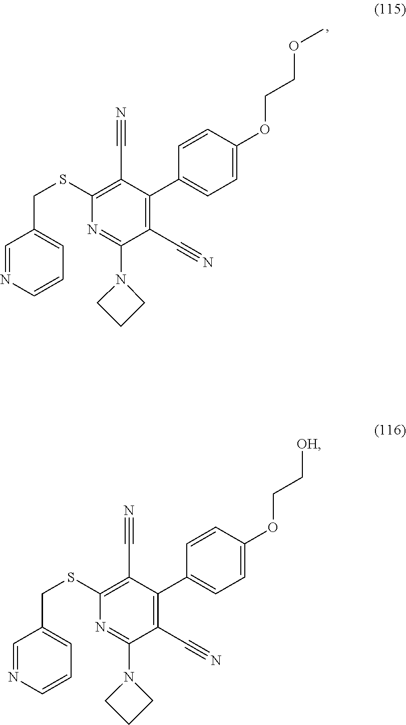 [SMILES: COCCOc1ccc(-c2c(C#N)c(SCc3cccnc3)nc(N3CCC3)c2C#N)cc1 ; N#Cc1c(SCc2cccnc2)nc(N2CCC2)c(C#N)c1-c1ccc(OCCO)cc1]

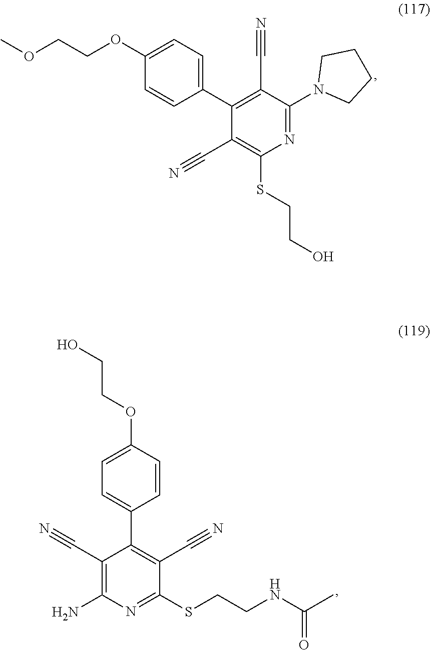 [SMILES: COCCOc1ccc(-c2c(C#N)c(SCCO)nc(N3CCCC3)c2C#N)cc1 ; CC(=O)NCCSc1nc(N)c(C#N)c(-c2ccc(OCCO)cc2)c1C#N]

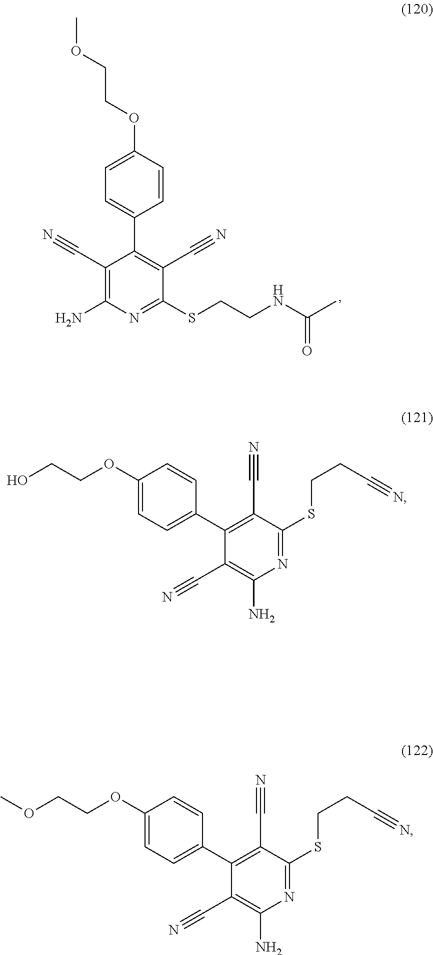

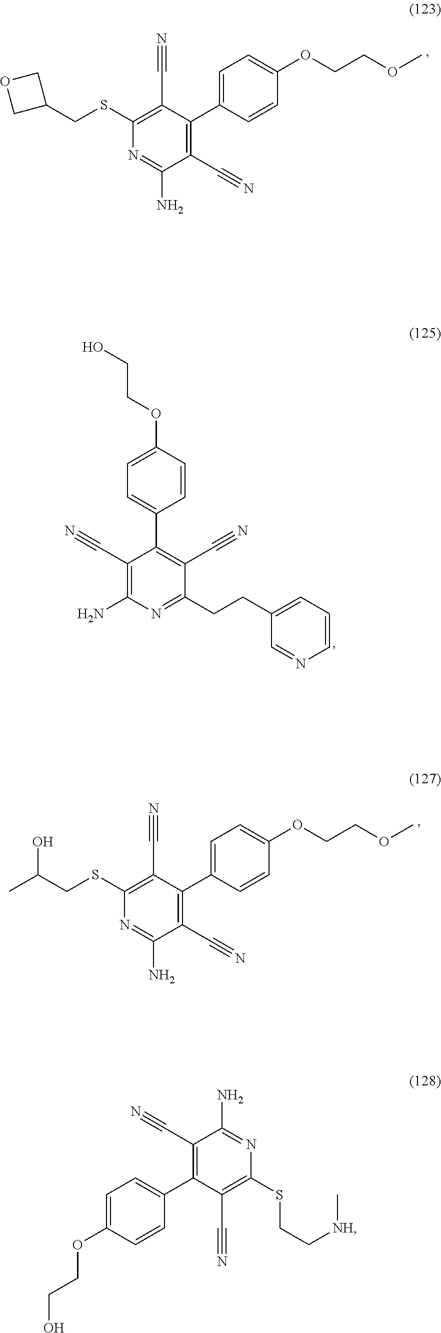

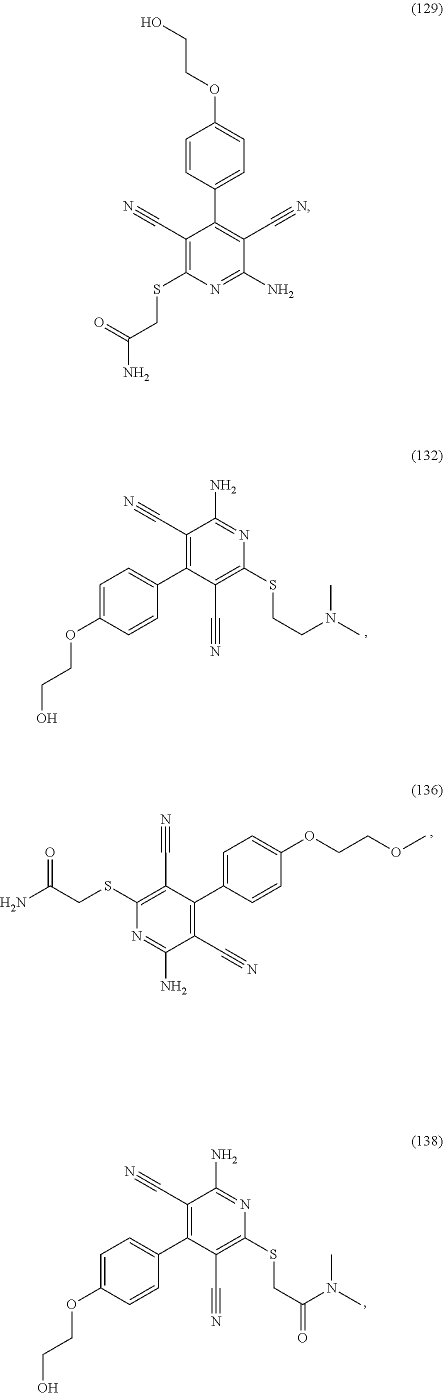 [SMILES: N#Cc1c(N)nc(SCC(N)=O)c(C#N)c1-c1ccc(OCCO)cc1 ; CN(C)CCSc1nc(N)c(C#N)c(-c2ccc(OCCO)cc2)c1C#N ; COCCOc1ccc(-c2c(C#N)c(N)nc(SCC(N)=O)c2C#N)cc1 ; CN(C)C(=O)CSc1nc(N)c(C#N)c(-c2ccc(OCCO)cc2)c1C#N]

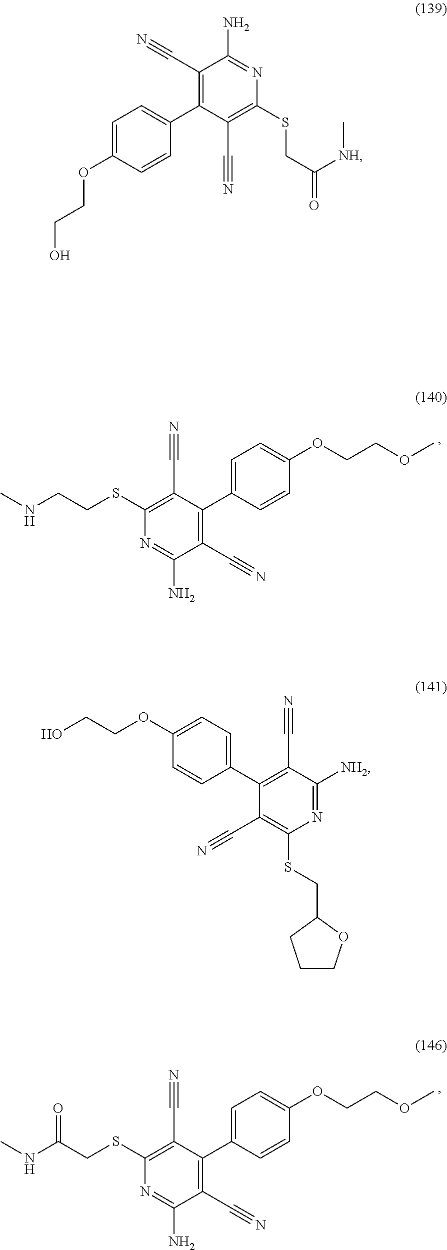

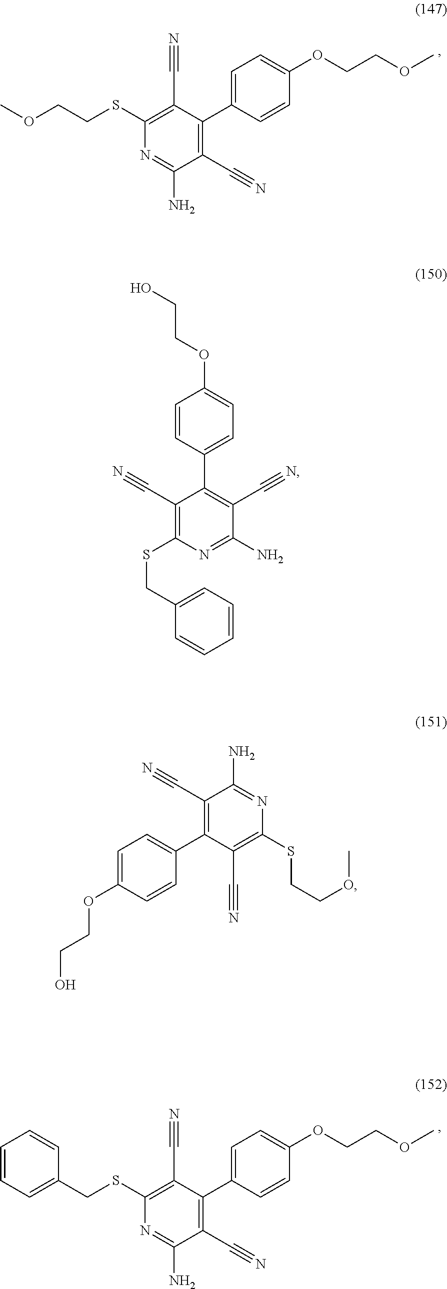

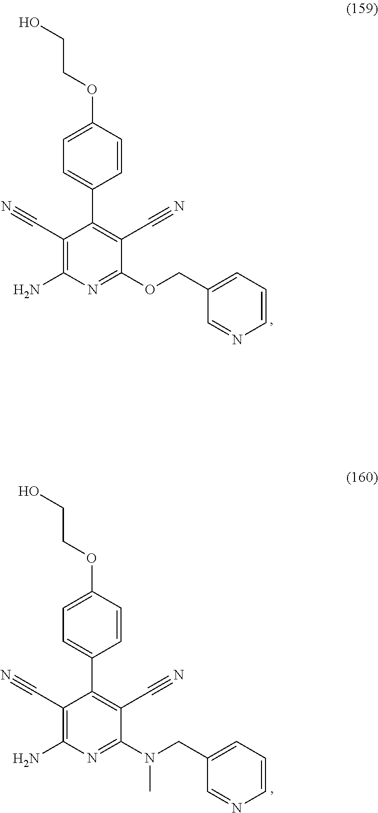 [SMILES: N#Cc1c(N)nc(OCc2cccnc2)c(C#N)c1-c1ccc(OCCO)cc1 ; CN(Cc1cccnc1)c1nc(N)c(C#N)c(-c2ccc(OCCO)cc2)c1C#N]

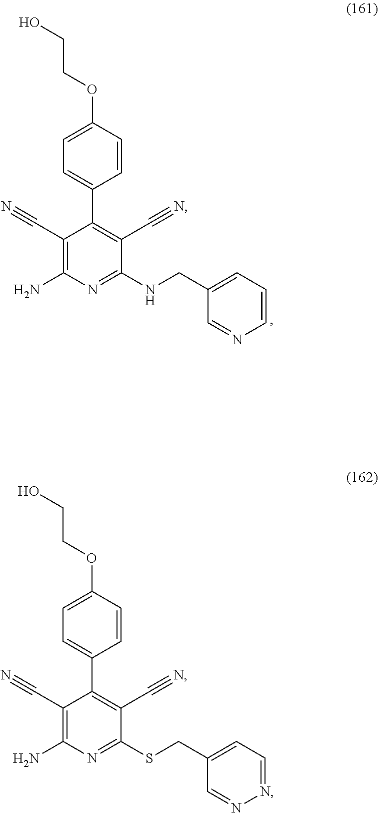 [SMILES: N#Cc1c(N)nc(NCc2cccnc2)c(C#N)c1-c1ccc(OCCO)cc1 ; N#Cc1c(N)nc(SCc2ccnnc2)c(C#N)c1-c1ccc(OCCO)cc1]

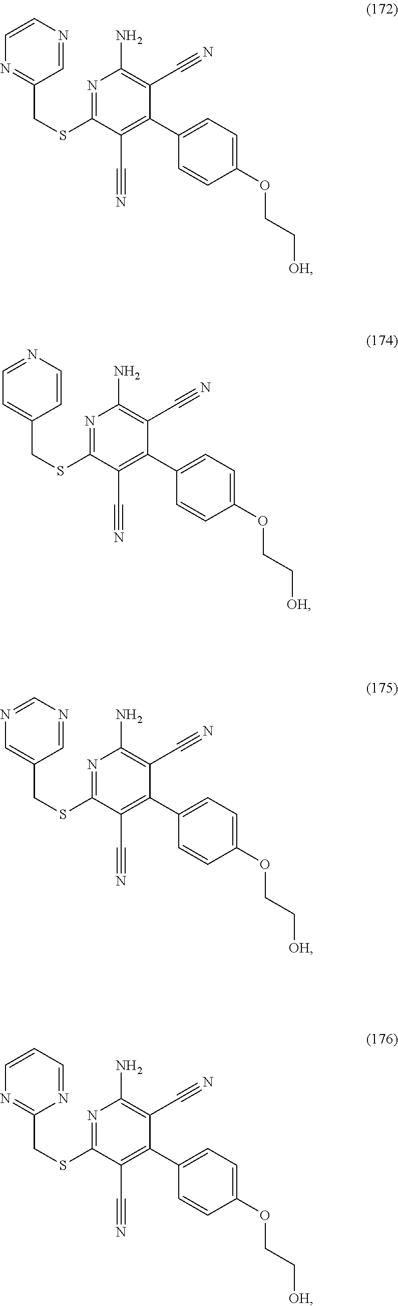 [SMILES: N#Cc1c(N)nc(SCc2cnccn2)c(C#N)c1-c1ccc(OCCO)cc1 ; N#Cc1c(N)nc(SCc2ccncc2)c(C#N)c1-c1ccc(OCCO)cc1 ; N#Cc1c(N)nc(SCc2cncnc2)c(C#N)c1-c1ccc(OCCO)cc1 ; N#Cc1c(N)nc(SCc2ncccn2)c(C#N)c1-c1ccc(OCCO)cc1]

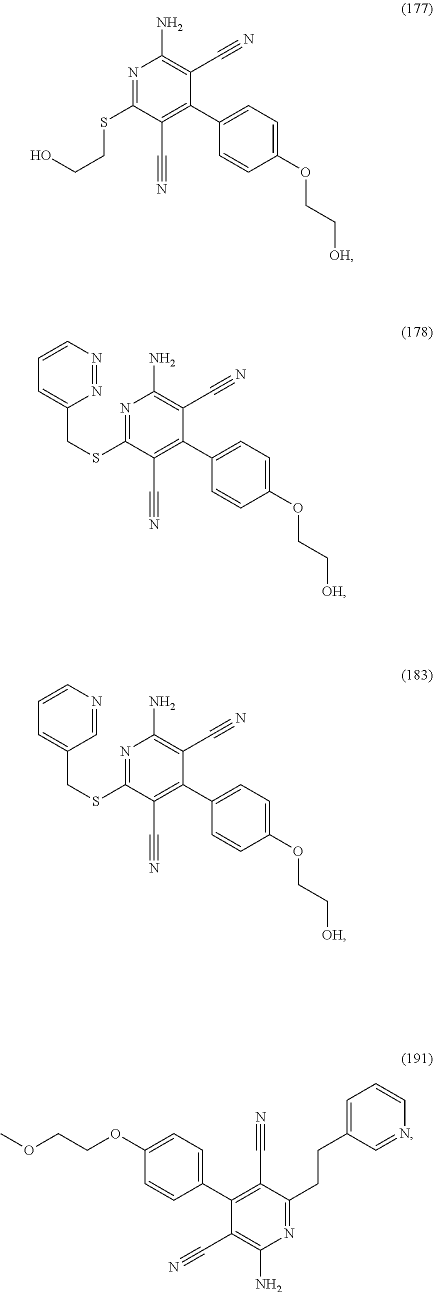 [SMILES: N#Cc1c(N)nc(SCCO)c(C#N)c1-c1ccc(OCCO)cc1 ; N#Cc1c(N)nc(SCc2cccnn2)c(C#N)c1-c1ccc(OCCO)cc1 ; N#Cc1c(N)nc(SCc2cccnc2)c(C#N)c1-c1ccc(OCCO)cc1 ; COCCOc1ccc(-c2c(C#N)c(N)nc(CCc3cccnc3)c2C#N)cc1]

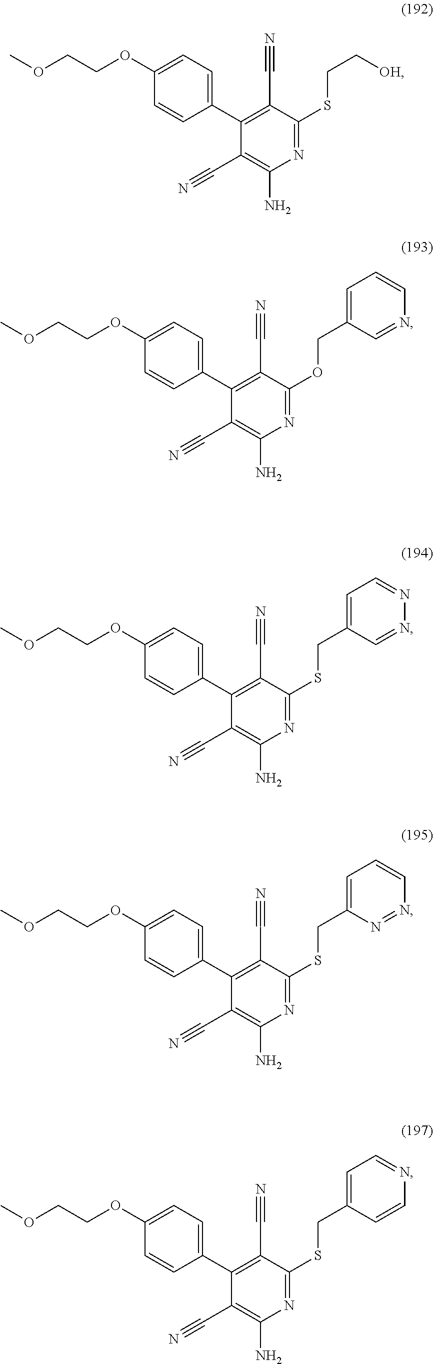 [SMILES: COCCOc1ccc(-c2c(C#N)c(N)nc(SCCO)c2C#N)cc1 ; COCCOc1ccc(-c2c(C#N)c(N)nc(OCc3cccnc3)c2C#N)cc1 ; COCCOc1ccc(-c2c(C#N)c(N)nc(SCc3ccnnc3)c2C#N)cc1 ; COCCOc1ccc(-c2c(C#N)c(N)nc(SCc3cccnn3)c2C#N)cc1 ; COCCOc1ccc(-c2c(C#N)c(N)nc(SCc3ccncc3)c2C#N)cc1]

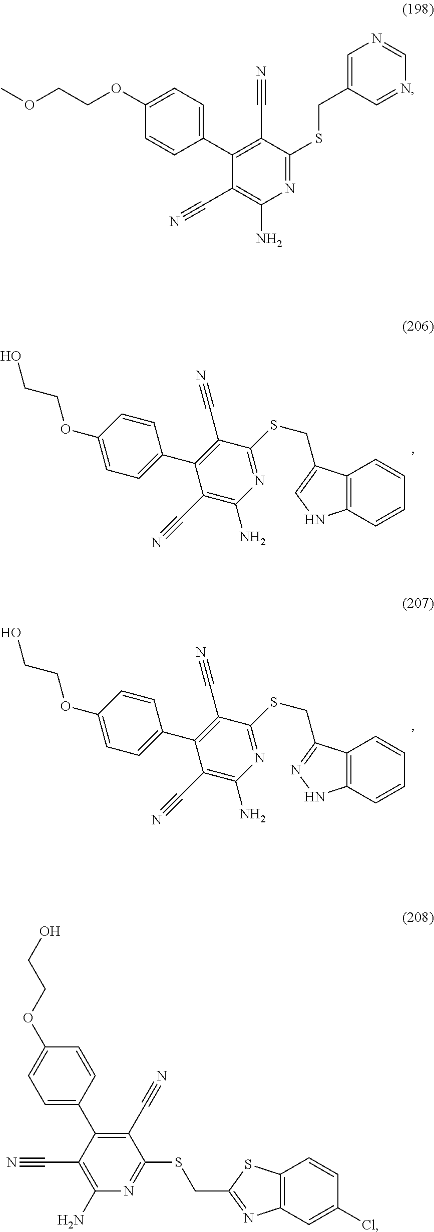 [SMILES: COCCOc1ccc(-c2c(C#N)c(N)nc(SCc3cncnc3)c2C#N)cc1 ; N#Cc1c(N)nc(SCc2c[nH]c3ccccc23)c(C#N)c1-c1ccc(OCCO)cc1 ; N#Cc1c(N)nc(SCc2n[nH]c3ccccc23)c(C#N)c1-c1ccc(OCCO)cc1 ; N#Cc1c(N)nc(SCc2nc3cc(Cl)ccc3s2)c(C#N)c1-c1ccc(OCCO)cc1]

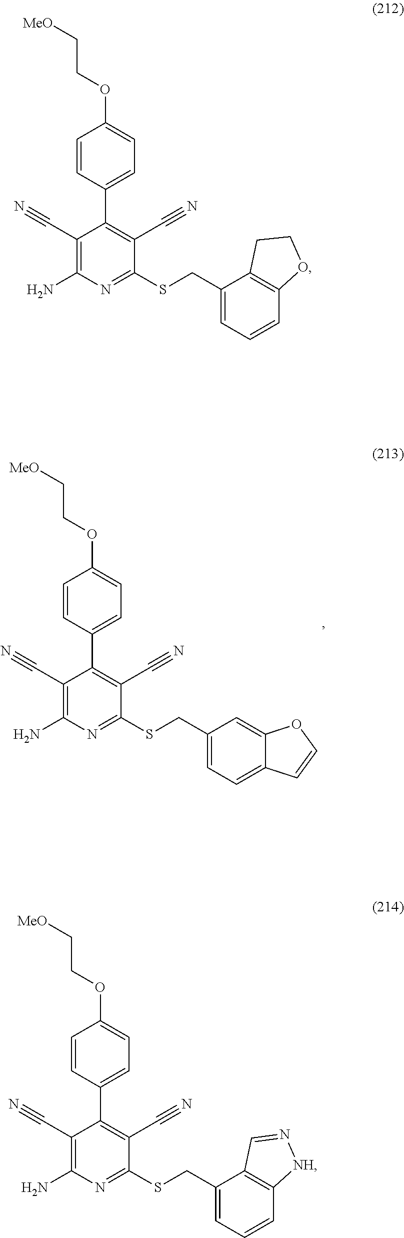 [SMILES: COCCOc1ccc(-c2c(C#N)c(N)nc(SCc3cccc4c3CCO4)c2C#N)cc1 ; COCCOc1ccc(-c2c(C#N)c(N)nc(SCc3ccc4ccoc4c3)c2C#N)cc1 ; COCCOc1ccc(-c2c(C#N)c(N)nc(SCc3cccc4[nH]ncc34)c2C#N)cc1]

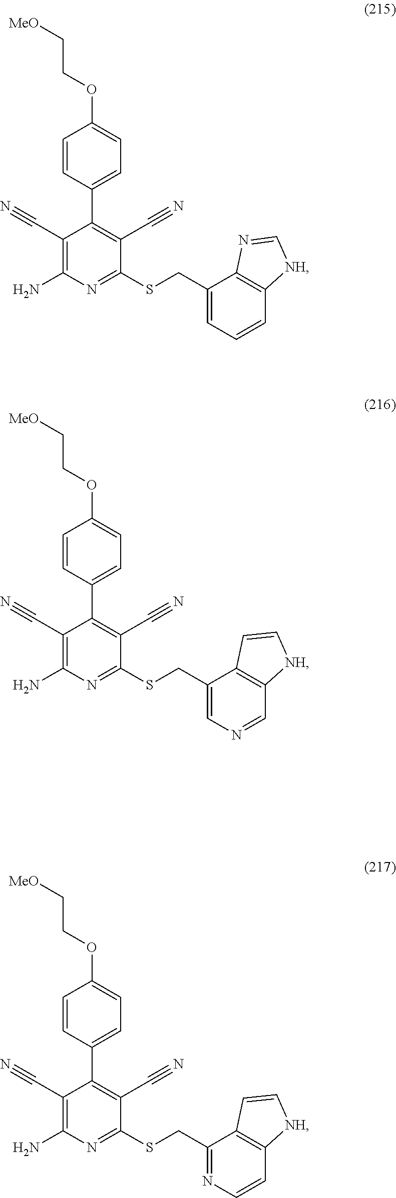 [SMILES: COCCOc1ccc(-c2c(C#N)c(N)nc(SCc3cccc4[nH]cnc34)c2C#N)cc1 ; COCCOc1ccc(-c2c(C#N)c(N)nc(SCc3cncc4[nH]ccc34)c2C#N)cc1 ; COCCOc1ccc(-c2c(C#N)c(N)nc(SCc3nccc4[nH]ccc34)c2C#N)cc1]

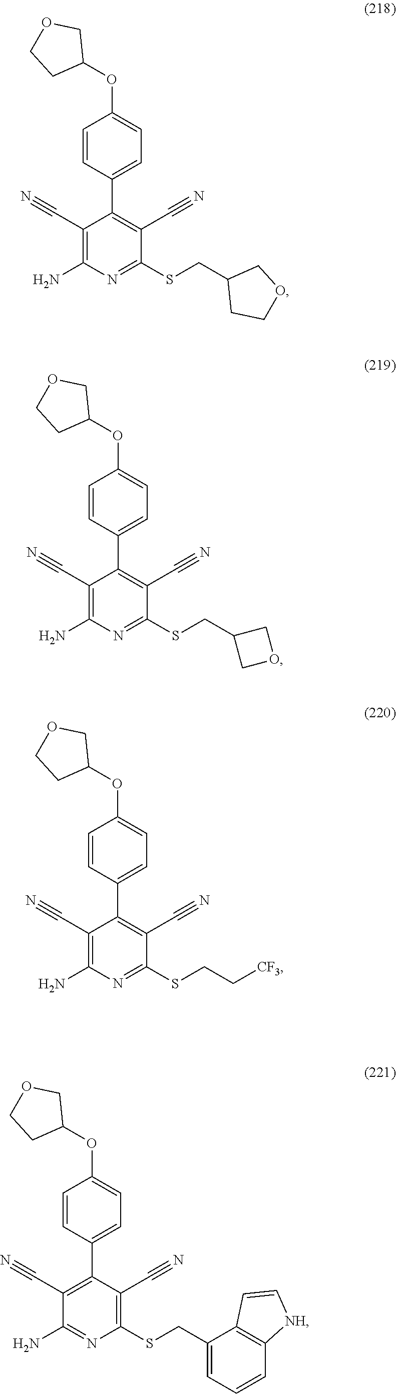 [SMILES: N#Cc1c(N)nc(SCC2CCOC2)c(C#N)c1-c1ccc(OC2CCOC2)cc1 ; N#Cc1c(N)nc(SCC2COC2)c(C#N)c1-c1ccc(OC2CCOC2)cc1 ; N#Cc1c(N)nc(SCCC(F)(F)F)c(C#N)c1-c1ccc(OC2CCOC2)cc1 ; N#Cc1c(N)nc(SCc2cccc3[nH]ccc23)c(C#N)c1-c1ccc(OC2CCOC2)cc1]

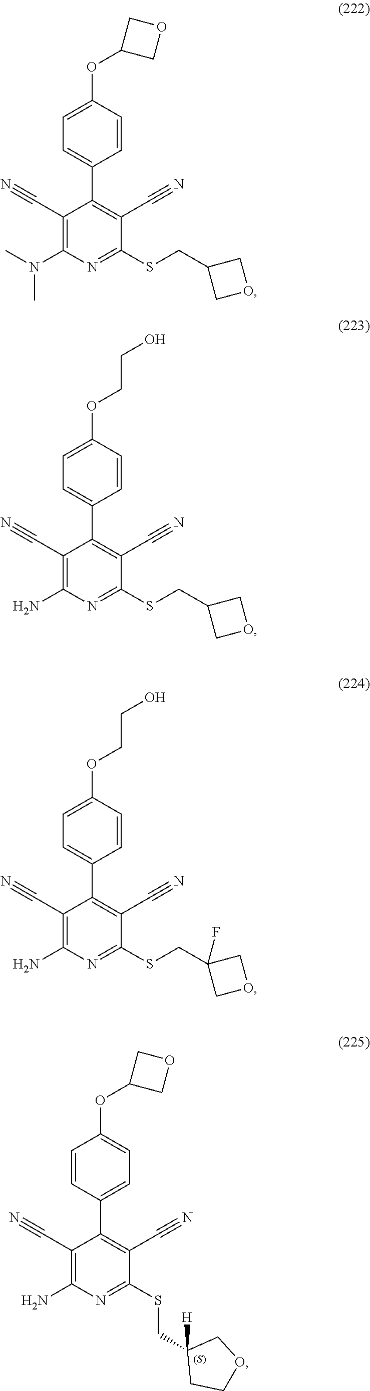

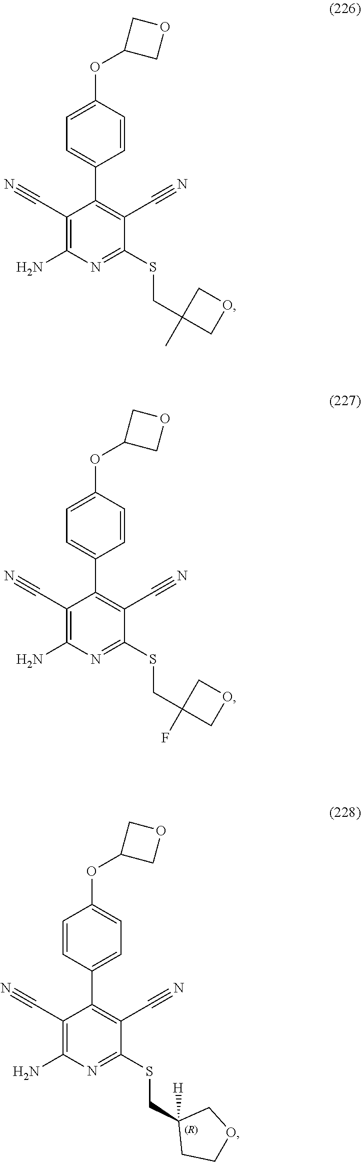

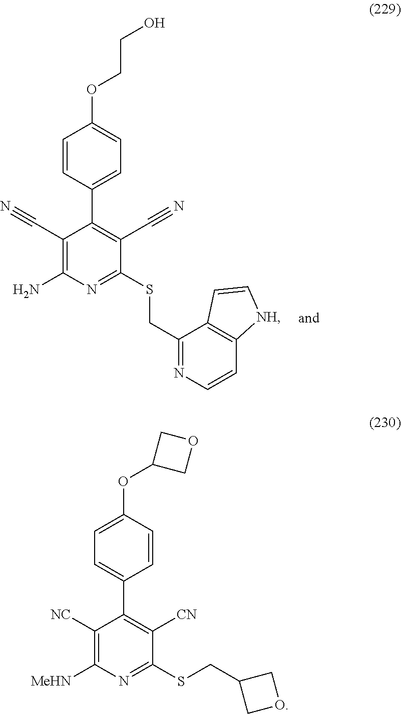 [SMILES: N#Cc1c(N)nc(SCc2nccc3[nH]ccc23)c(C#N)c1-c1ccc(OCCO)cc1 ; CNc1nc(SCC2COC2)c(C#N)c(-c2ccc(OC3COC3)cc2)c1C#N]

The present application further provides a compound selected from any one of the following, and pharmaceutically acceptable salts thereof:

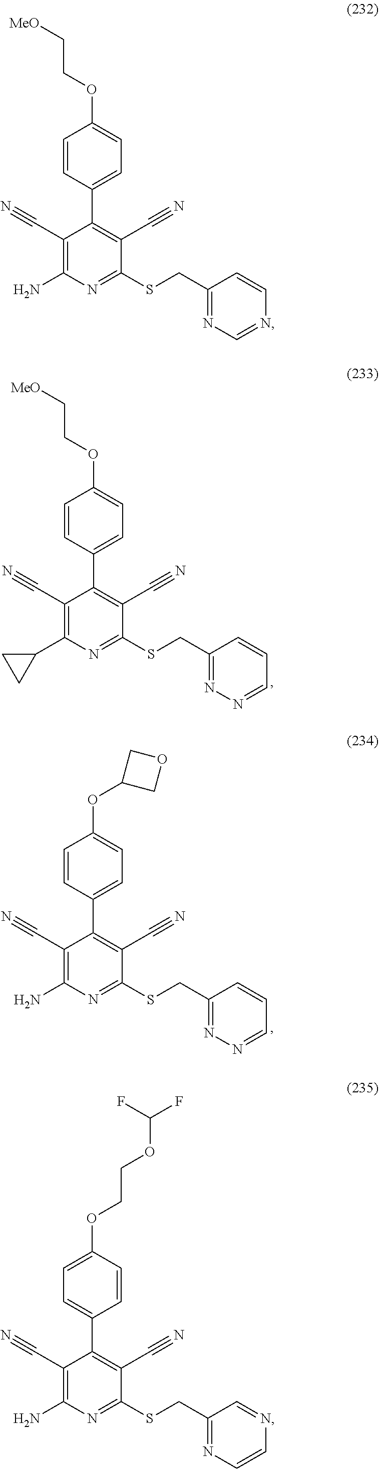 [SMILES: COCCOc1ccc(-c2c(C#N)c(N)nc(SCc3ccncn3)c2C#N)cc1 ; COCCOc1ccc(-c2c(C#N)c(SCc3cccnn3)nc(C3CC3)c2C#N)cc1 ; N#Cc1c(N)nc(SCc2cccnn2)c(C#N)c1-c1ccc(OC2COC2)cc1 ; N#Cc1c(N)nc(SCc2cnccn2)c(C#N)c1-c1ccc(OCCOC(F)F)cc1]

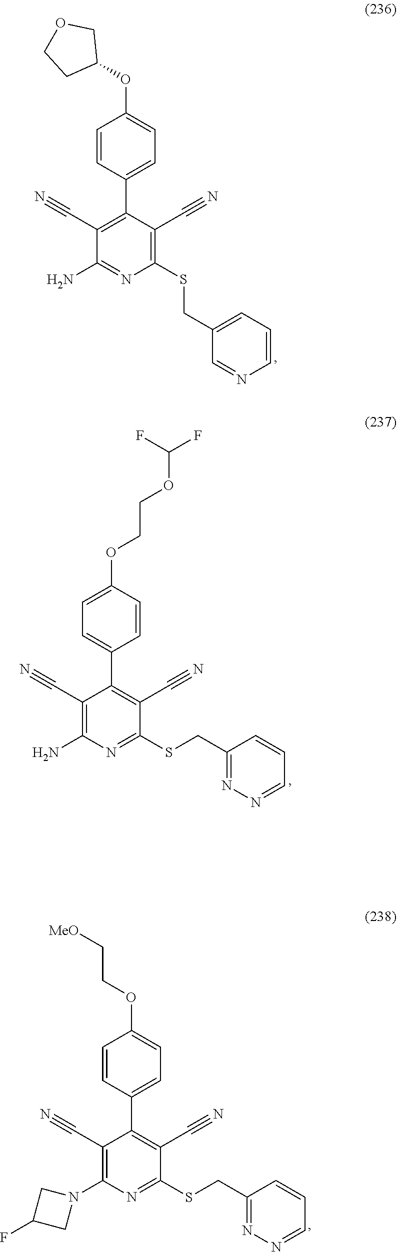 [SMILES: N#Cc1c(N)nc(SCc2cccnc2)c(C#N)c1-c1ccc(O[C@@H]2CCOC2)cc1 ; N#Cc1c(N)nc(SCc2cccnn2)c(C#N)c1-c1ccc(OCCOC(F)F)cc1 ; COCCOc1ccc(-c2c(C#N)c(SCc3cccnn3)nc(N3CC(F)C3)c2C#N)cc1]

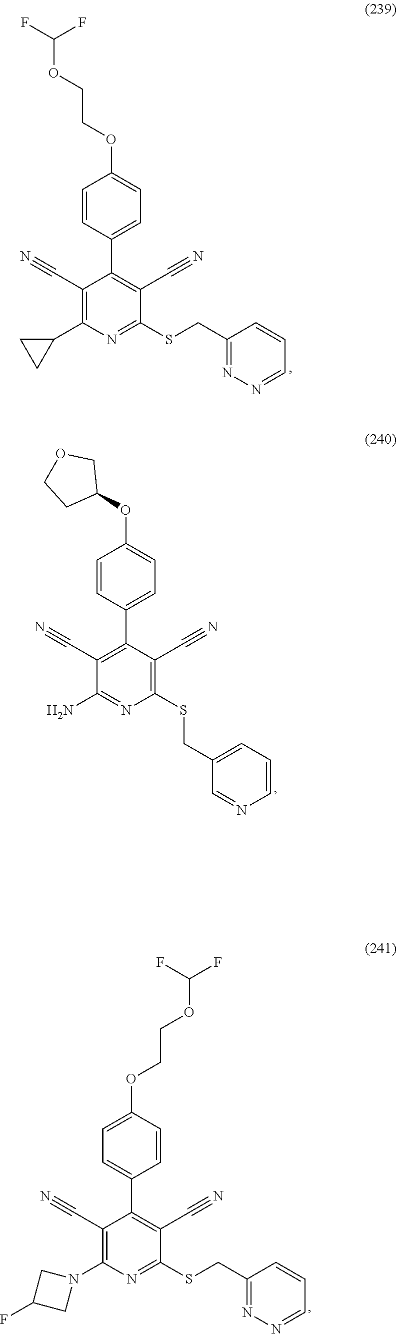 [SMILES: N#Cc1c(SCc2cccnn2)nc(C2CC2)c(C#N)c1-c1ccc(OCCOC(F)F)cc1 ; N#Cc1c(N)nc(SCc2cccnc2)c(C#N)c1-c1ccc(O[C@H]2CCOC2)cc1 ; N#Cc1c(SCc2cccnn2)nc(N2CC(F)C2)c(C#N)c1-c1ccc(OCCOC(F)F)cc1]

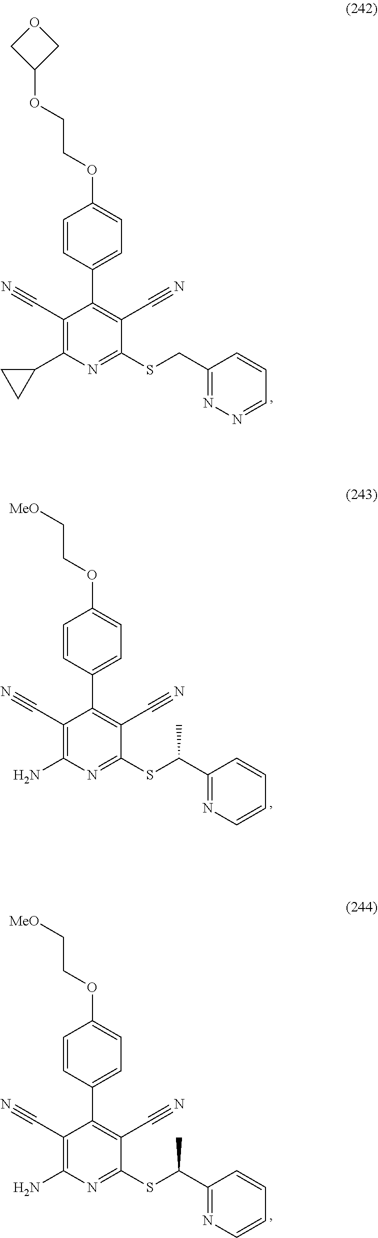 [SMILES: N#Cc1c(SCc2cccnn2)nc(C2CC2)c(C#N)c1-c1ccc(OCCOC2COC2)cc1 ; COCCOc1ccc(-c2c(C#N)c(N)nc(S[C@H](C)c3ccccn3)c2C#N)cc1 ; COCCOc1ccc(-c2c(C#N)c(N)nc(S[C@@H](C)c3ccccn3)c2C#N)cc1]

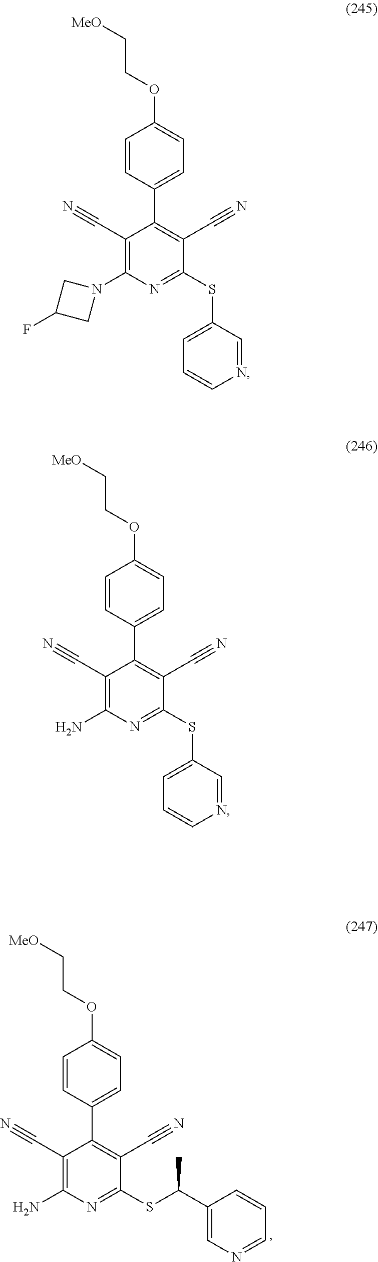 [SMILES: COCCOc1ccc(-c2c(C#N)c(Sc3cccnc3)nc(N3CC(F)C3)c2C#N)cc1 ; COCCOc1ccc(-c2c(C#N)c(N)nc(Sc3cccnc3)c2C#N)cc1 ; COCCOc1ccc(-c2c(C#N)c(N)nc(S[C@@H](C)c3cccnc3)c2C#N)cc1]

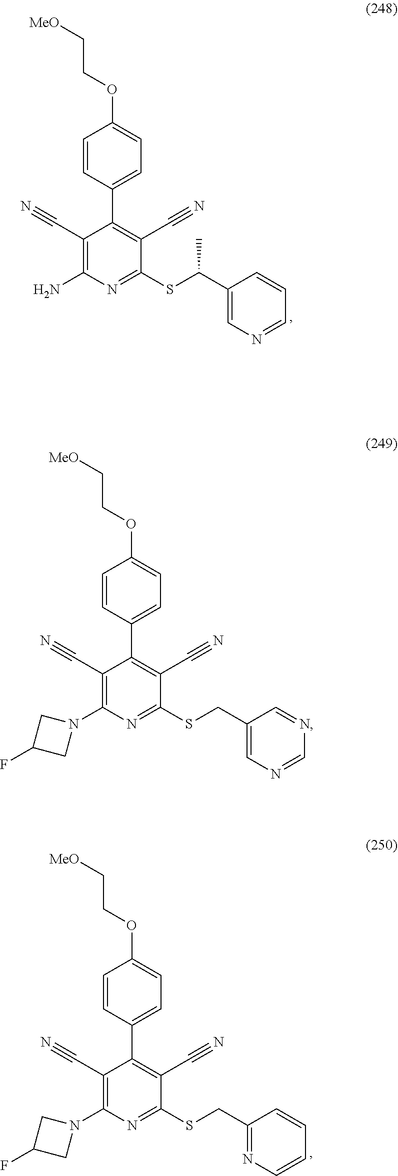 [SMILES: COCCOc1ccc(-c2c(C#N)c(N)nc(S[C@H](C)c3cccnc3)c2C#N)cc1 ; COCCOc1ccc(-c2c(C#N)c(SCc3cncnc3)nc(N3CC(F)C3)c2C#N)cc1 ; COCCOc1ccc(-c2c(C#N)c(SCc3ccccn3)nc(N3CC(F)C3)c2C#N)cc1]

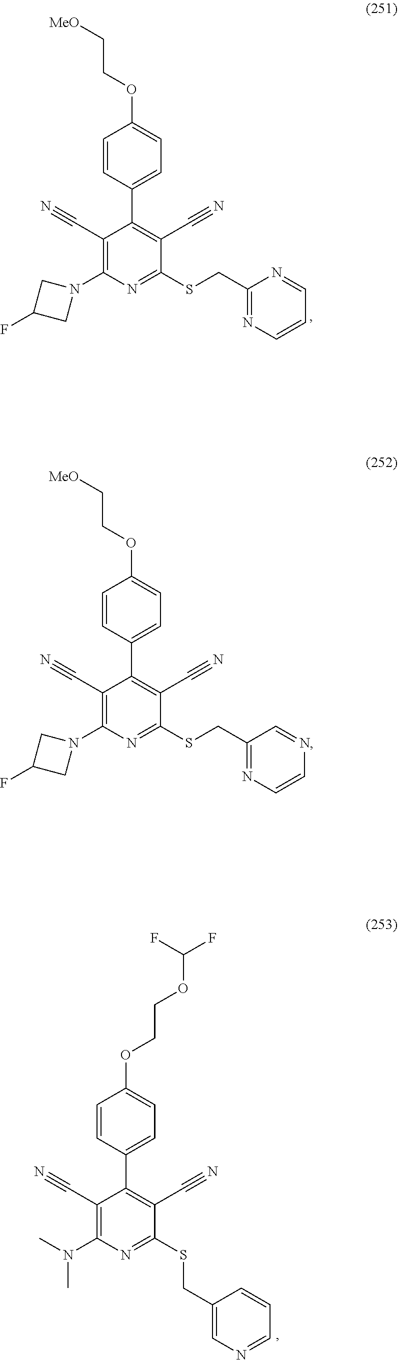 [SMILES: COCCOc1ccc(-c2c(C#N)c(SCc3ncccn3)nc(N3CC(F)C3)c2C#N)cc1 ; COCCOc1ccc(-c2c(C#N)c(SCc3cnccn3)nc(N3CC(F)C3)c2C#N)cc1 ; CN(C)c1nc(SCc2cccnc2)c(C#N)c(-c2ccc(OCCOC(F)F)cc2)c1C#N]

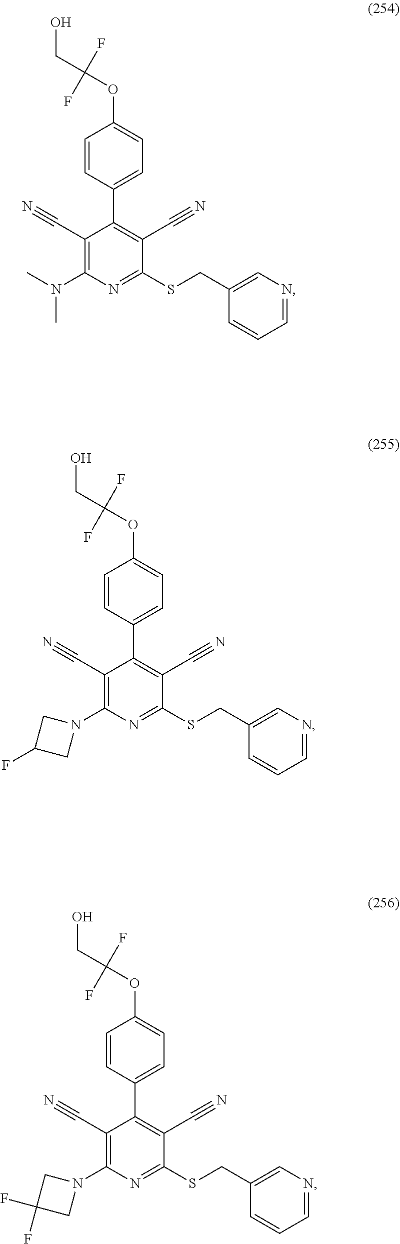 [SMILES: CN(C)c1nc(SCc2cccnc2)c(C#N)c(-c2ccc(OC(F)(F)CO)cc2)c1C#N ; N#Cc1c(SCc2cccnc2)nc(N2CC(F)C2)c(C#N)c1-c1ccc(OC(F)(F)CO)cc1 ; N#Cc1c(SCc2cccnc2)nc(N2CC(F)(F)C2)c(C#N)c1-c1ccc(OC(F)(F)CO)cc1]

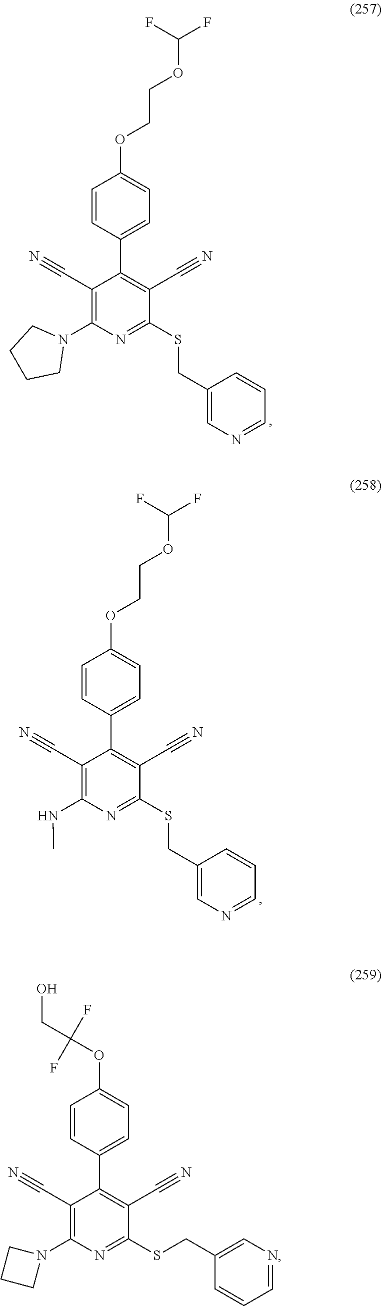 [SMILES: N#Cc1c(SCc2cccnc2)nc(N2CCCC2)c(C#N)c1-c1ccc(OCCOC(F)F)cc1 ; CNc1nc(SCc2cccnc2)c(C#N)c(-c2ccc(OCCOC(F)F)cc2)c1C#N ; N#Cc1c(SCc2cccnc2)nc(N2CCC2)c(C#N)c1-c1ccc(OC(F)(F)CO)cc1]

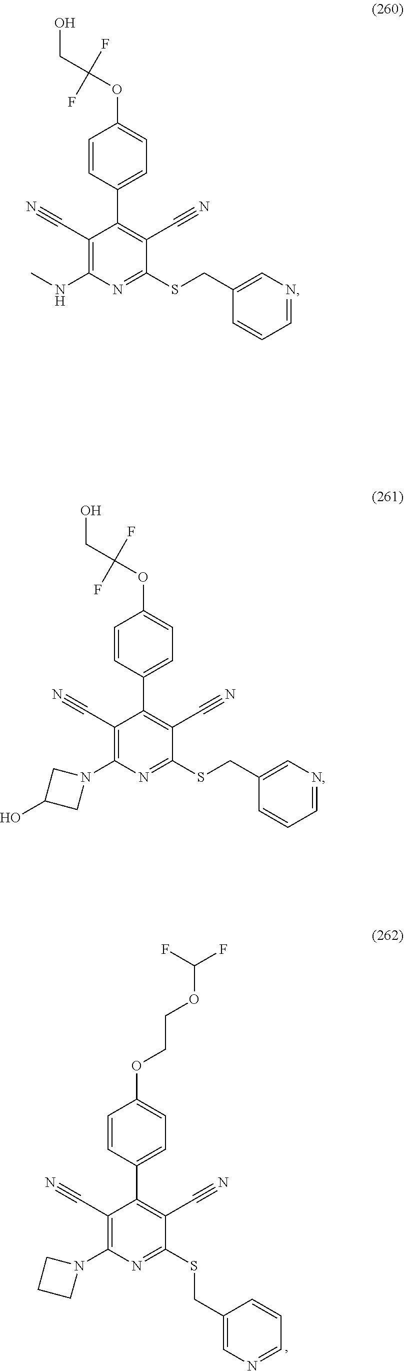 [SMILES: CNc1nc(SCc2cccnc2)c(C#N)c(-c2ccc(OC(F)(F)CO)cc2)c1C#N ; N#Cc1c(SCc2cccnc2)nc(N2CC(O)C2)c(C#N)c1-c1ccc(OC(F)(F)CO)cc1 ; N#Cc1c(SCc2cccnc2)nc(N2CCC2)c(C#N)c1-c1ccc(OCCOC(F)F)cc1]

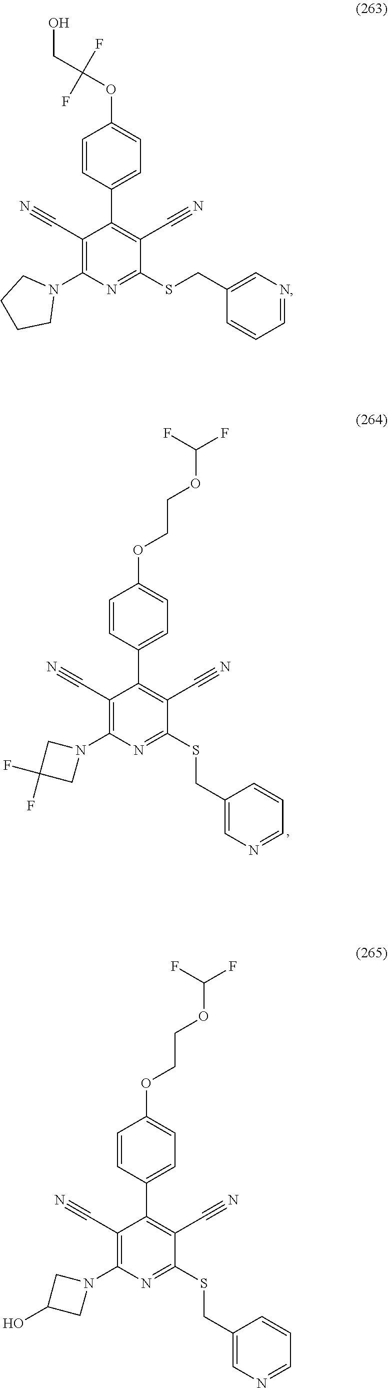 [SMILES: N#Cc1c(SCc2cccnc2)nc(N2CCCC2)c(C#N)c1-c1ccc(OC(F)(F)CO)cc1 ; N#Cc1c(SCc2cccnc2)nc(N2CC(F)(F)C2)c(C#N)c1-c1ccc(OCCOC(F)F)cc1 ; N#Cc1c(SCc2cccnc2)nc(N2CC(O)C2)c(C#N)c1-c1ccc(OCCOC(F)F)cc1]

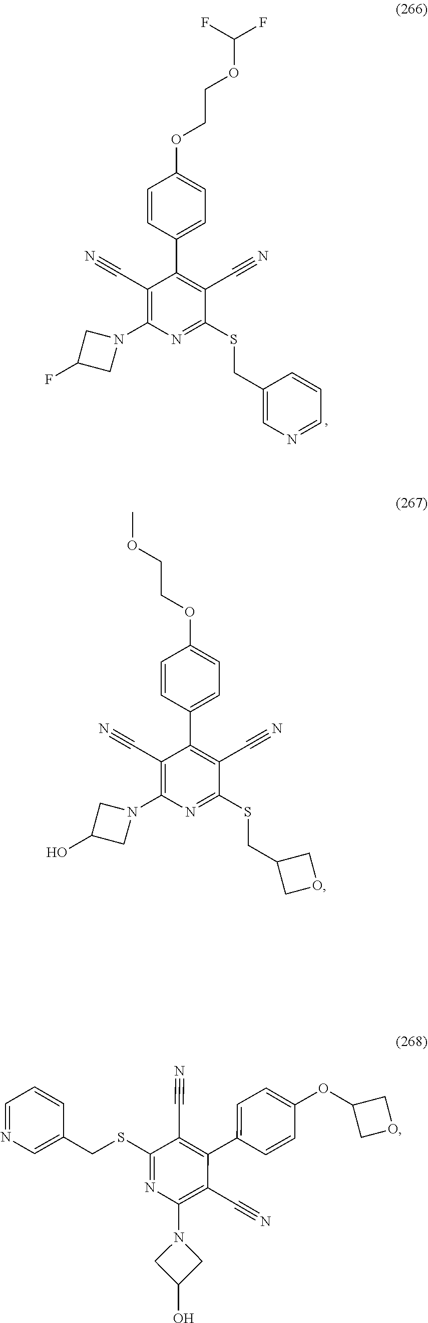 [SMILES: N#Cc1c(SCc2cccnc2)nc(N2CC(F)C2)c(C#N)c1-c1ccc(OCCOC(F)F)cc1 ; COCCOc1ccc(-c2c(C#N)c(SCC3COC3)nc(N3CC(O)C3)c2C#N)cc1 ; N#Cc1c(SCc2cccnc2)nc(N2CC(O)C2)c(C#N)c1-c1ccc(OC2COC2)cc1]

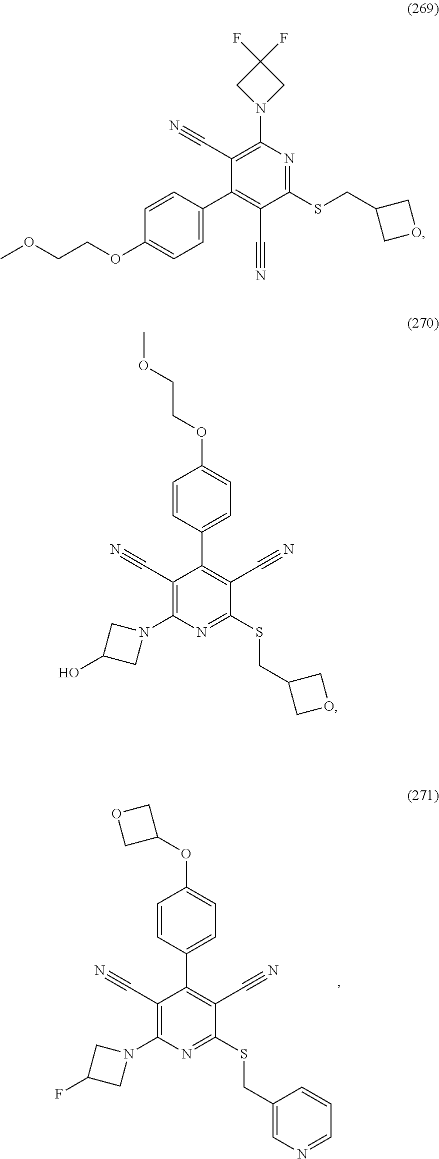 [SMILES: COCCOc1ccc(-c2c(C#N)c(SCC3COC3)nc(N3CC(F)(F)C3)c2C#N)cc1 ; COCCOc1ccc(-c2c(C#N)c(SCC3COC3)nc(N3CC(O)C3)c2C#N)cc1 ; N#Cc1c(SCc2cccnc2)nc(N2CC(F)C2)c(C#N)c1-c1ccc(OC2COC2)cc1]

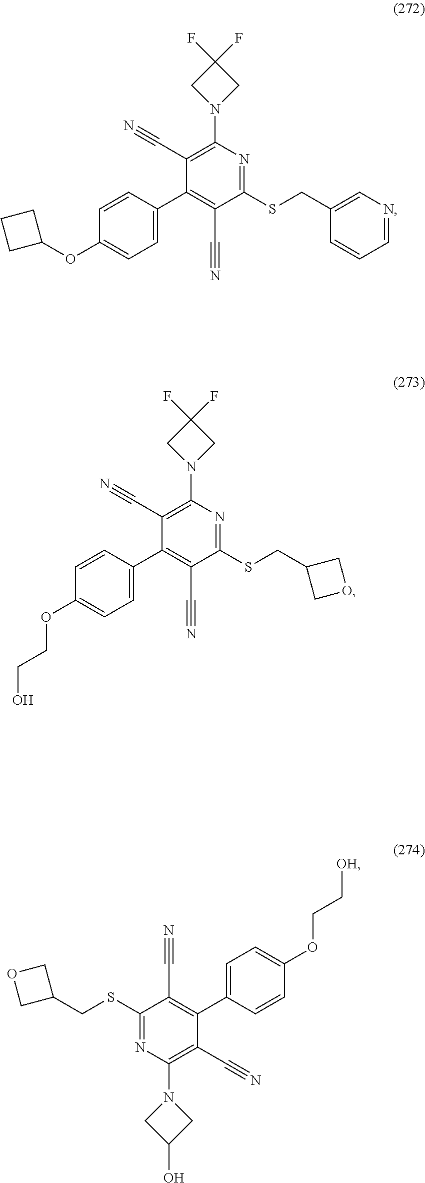 [SMILES: N#Cc1c(SCc2cccnc2)nc(N2CC(F)(F)C2)c(C#N)c1-c1ccc(OC2CCC2)cc1 ; N#Cc1c(SCC2COC2)nc(N2CC(F)(F)C2)c(C#N)c1-c1ccc(OCCO)cc1 ; N#Cc1c(SCC2COC2)nc(N2CC(O)C2)c(C#N)c1-c1ccc(OCCO)cc1]

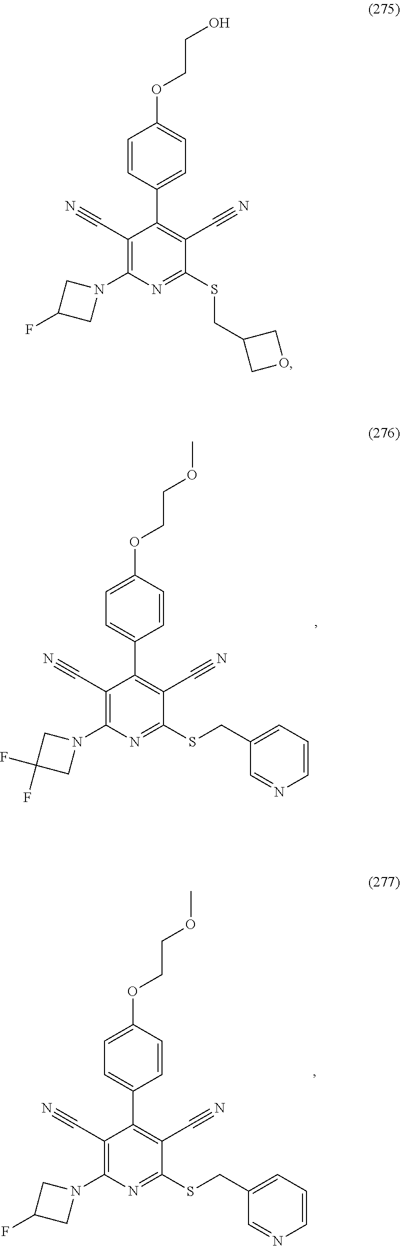 [SMILES: N#Cc1c(SCC2COC2)nc(N2CC(F)C2)c(C#N)c1-c1ccc(OCCO)cc1 ; COCCOc1ccc(-c2c(C#N)c(SCc3cccnc3)nc(N3CC(F)(F)C3)c2C#N)cc1 ; COCCOc1ccc(-c2c(C#N)c(SCc3cccnc3)nc(N3CC(F)C3)c2C#N)cc1]

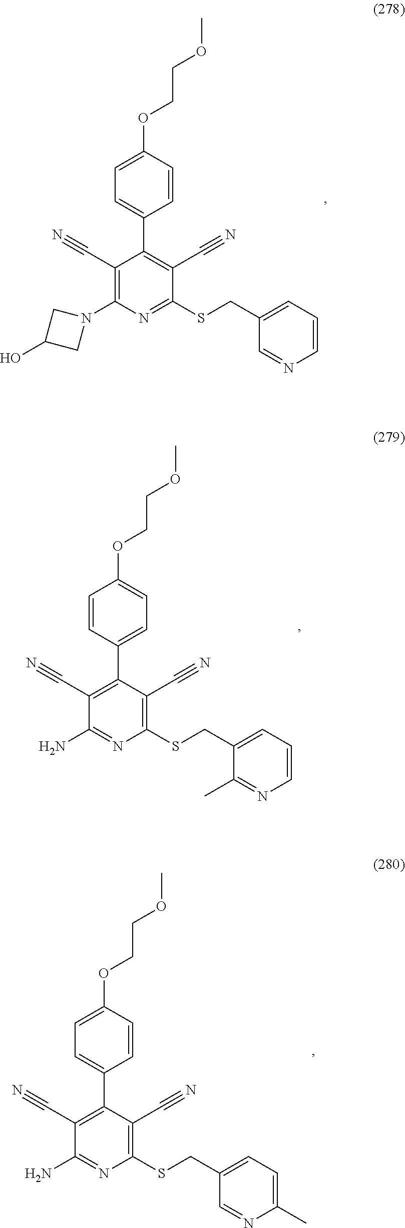 [SMILES: COCCOc1ccc(-c2c(C#N)c(SCc3cccnc3)nc(N3CC(O)C3)c2C#N)cc1 ; COCCOc1ccc(-c2c(C#N)c(N)nc(SCc3cccnc3C)c2C#N)cc1 ; COCCOc1ccc(-c2c(C#N)c(N)nc(SCc3ccc(C)nc3)c2C#N)cc1]

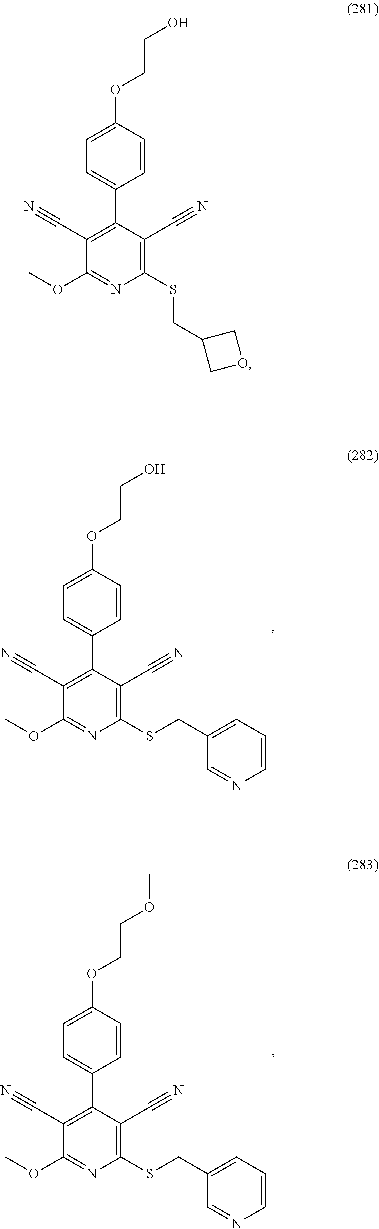 [SMILES: COc1nc(SCC2COC2)c(C#N)c(-c2ccc(OCCO)cc2)c1C#N ; COc1nc(SCc2cccnc2)c(C#N)c(-c2ccc(OCCO)cc2)c1C#N ; COCCOc1ccc(-c2c(C#N)c(OC)nc(SCc3cccnc3)c2C#N)cc1]

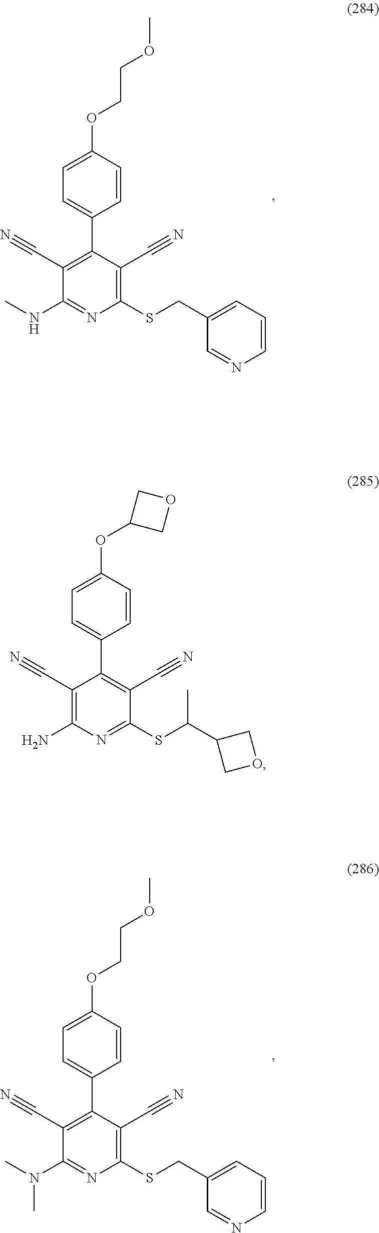 [SMILES: CNc1nc(SCc2cccnc2)c(C#N)c(-c2ccc(OCCOC)cc2)c1C#N ; CC(Sc1nc(N)c(C#N)c(-c2ccc(OC3COC3)cc2)c1C#N)C1COC1 ; COCCOc1ccc(-c2c(C#N)c(SCc3cccnc3)nc(N(C)C)c2C#N)cc1]

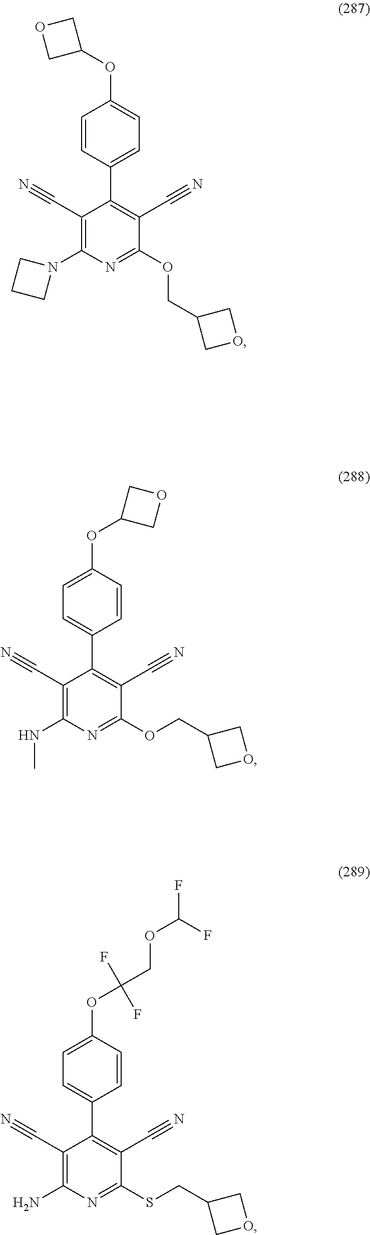 [SMILES: N#Cc1c(OCC2COC2)nc(N2CCC2)c(C#N)c1-c1ccc(OC2COC2)cc1 ; CNc1nc(OCC2COC2)c(C#N)c(-c2ccc(OC3COC3)cc2)c1C#N ; N#Cc1c(N)nc(SCC2COC2)c(C#N)c1-c1ccc(OC(F)(F)COC(F)F)cc1]

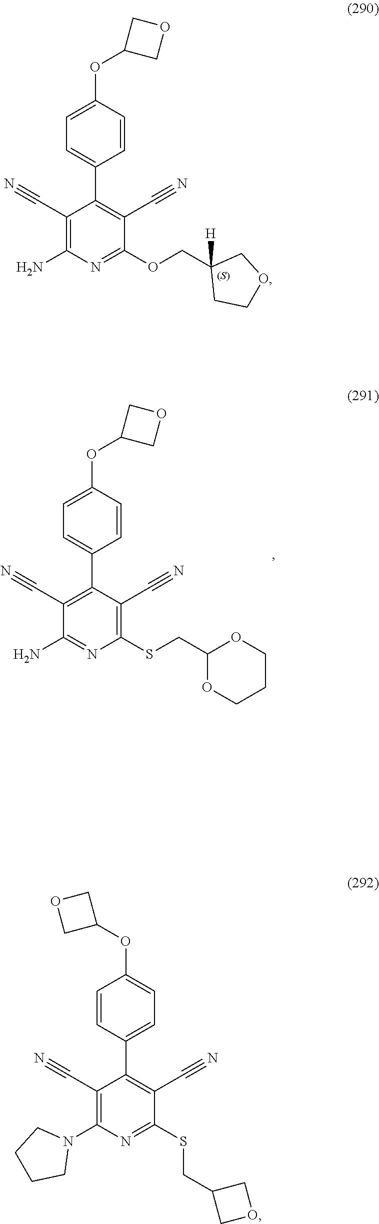

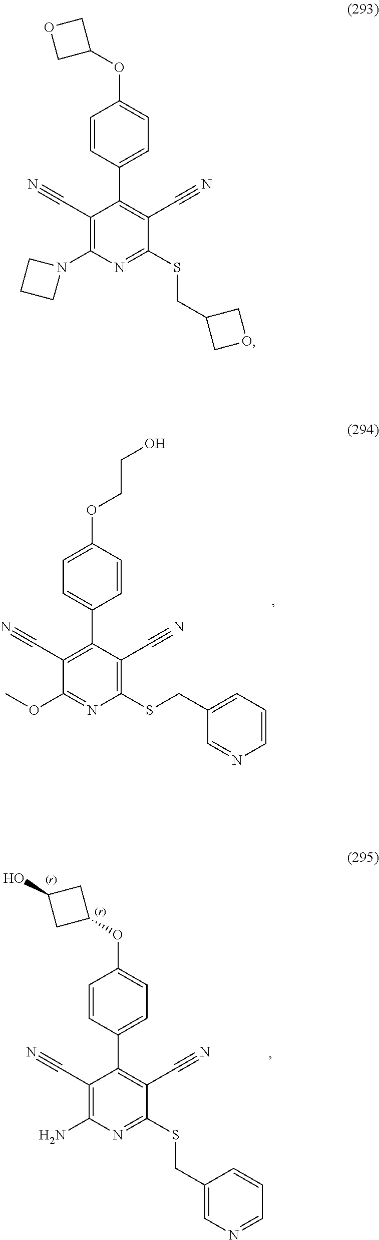 [SMILES: N#Cc1c(SCC2COC2)nc(N2CCC2)c(C#N)c1-c1ccc(OC2COC2)cc1 ; COc1nc(SCc2cccnc2)c(C#N)c(-c2ccc(OCCO)cc2)c1C#N ; N#Cc1c(N)nc(SCc2cccnc2)c(C#N)c1-c1ccc(O[C@H]2C[C@H](O)C2)cc1]

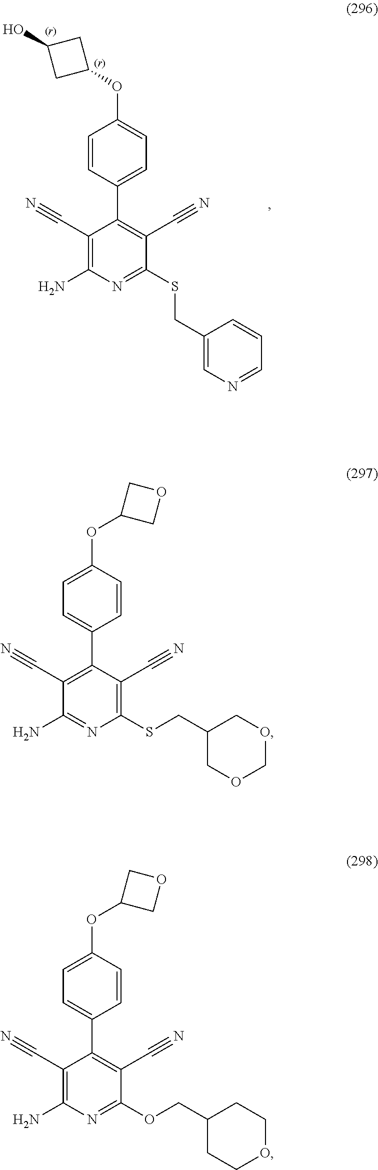 [SMILES: N#Cc1c(N)nc(SCc2cccnc2)c(C#N)c1-c1ccc(O[C@H]2C[C@H](O)C2)cc1 ; N#Cc1c(N)nc(SCC2COCOC2)c(C#N)c1-c1ccc(OC2COC2)cc1 ; N#Cc1c(N)nc(OCC2CCOCC2)c(C#N)c1-c1ccc(OC2COC2)cc1]

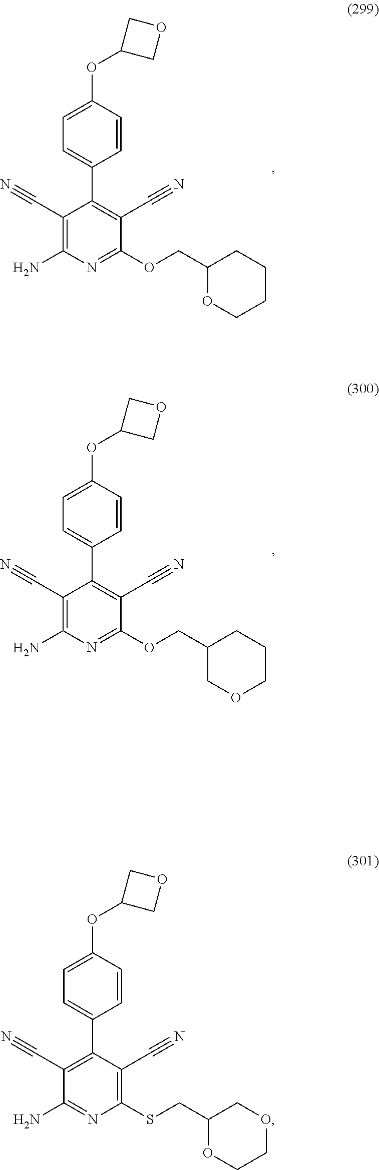 [SMILES: N#Cc1c(N)nc(OCC2CCCCO2)c(C#N)c1-c1ccc(OC2COC2)cc1 ; N#Cc1c(N)nc(OCC2CCCOC2)c(C#N)c1-c1ccc(OC2COC2)cc1 ; N#Cc1c(N)nc(SCC2COCCO2)c(C#N)c1-c1ccc(OC2COC2)cc1]

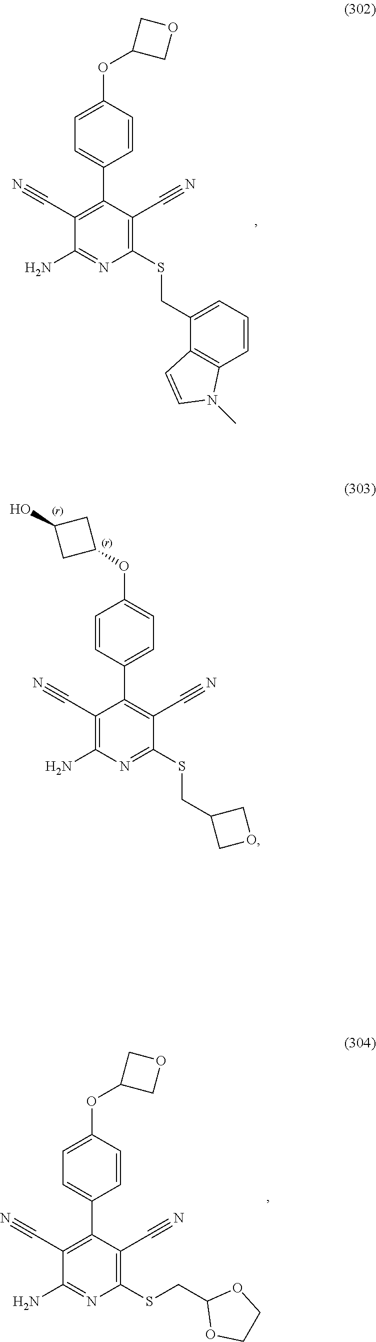 [SMILES: Cn1ccc2c(CSc3nc(N)c(C#N)c(-c4ccc(OC5COC5)cc4)c3C#N)cccc21 ; N#Cc1c(N)nc(SCC2COC2)c(C#N)c1-c1ccc(O[C@H]2C[C@H](O)C2)cc1 ; N#Cc1c(N)nc(SCC2OCCO2)c(C#N)c1-c1ccc(OC2COC2)cc1]

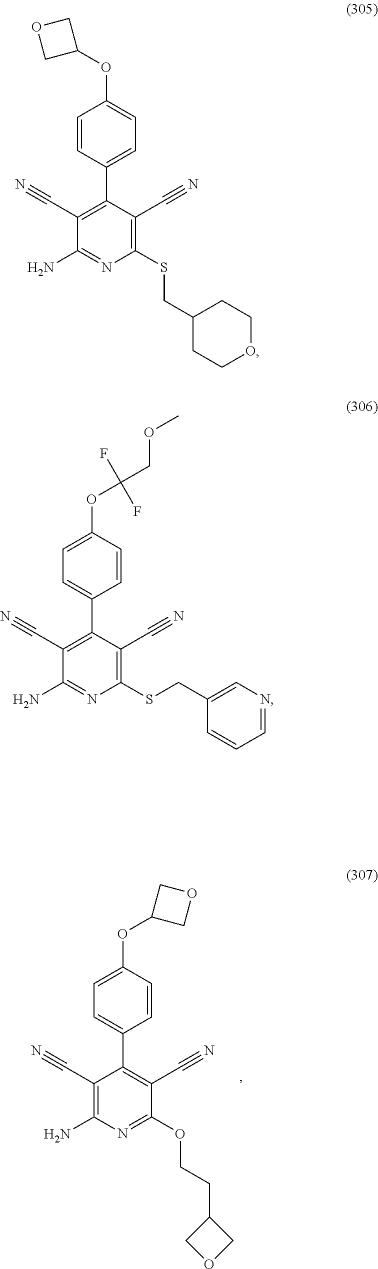 [SMILES: N#Cc1c(N)nc(SCC2CCOCC2)c(C#N)c1-c1ccc(OC2COC2)cc1 ; COCC(F)(F)Oc1ccc(-c2c(C#N)c(N)nc(SCc3cccnc3)c2C#N)cc1 ; N#Cc1c(N)nc(OCCC2COC2)c(C#N)c1-c1ccc(OC2COC2)cc1]

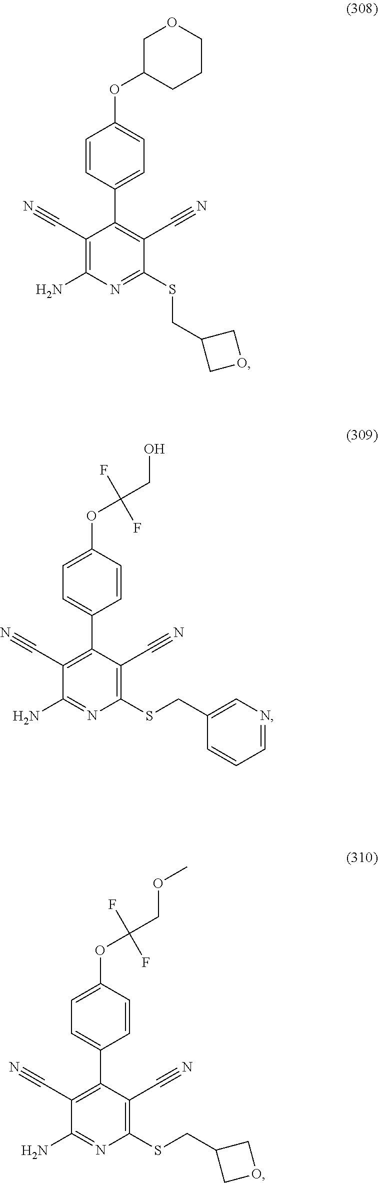 [SMILES: N#Cc1c(N)nc(SCC2COC2)c(C#N)c1-c1ccc(OC2CCCOC2)cc1 ; N#Cc1c(N)nc(SCc2cccnc2)c(C#N)c1-c1ccc(OC(F)(F)CO)cc1 ; COCC(F)(F)Oc1ccc(-c2c(C#N)c(N)nc(SCC3COC3)c2C#N)cc1]

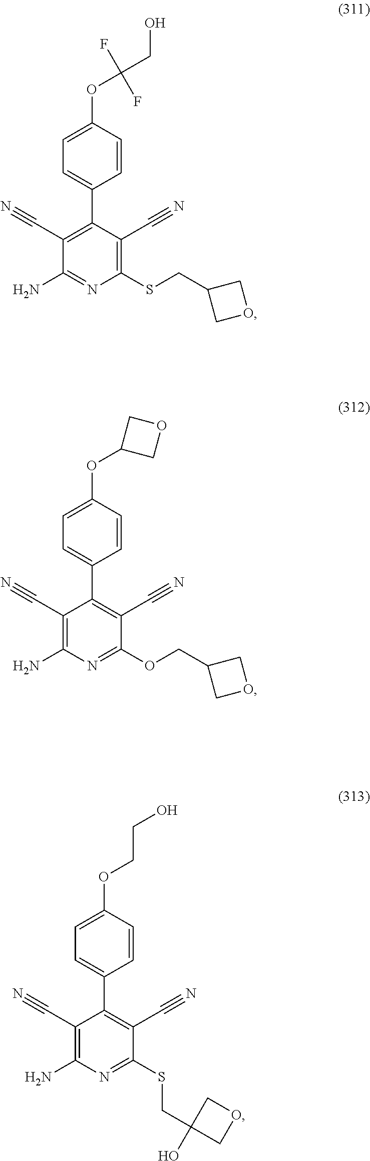 [SMILES: N#Cc1c(N)nc(SCC2COC2)c(C#N)c1-c1ccc(OC(F)(F)CO)cc1 ; N#Cc1c(N)nc(OCC2COC2)c(C#N)c1-c1ccc(OC2COC2)cc1 ; N#Cc1c(N)nc(SCC2(O)COC2)c(C#N)c1-c1ccc(OCCO)cc1]

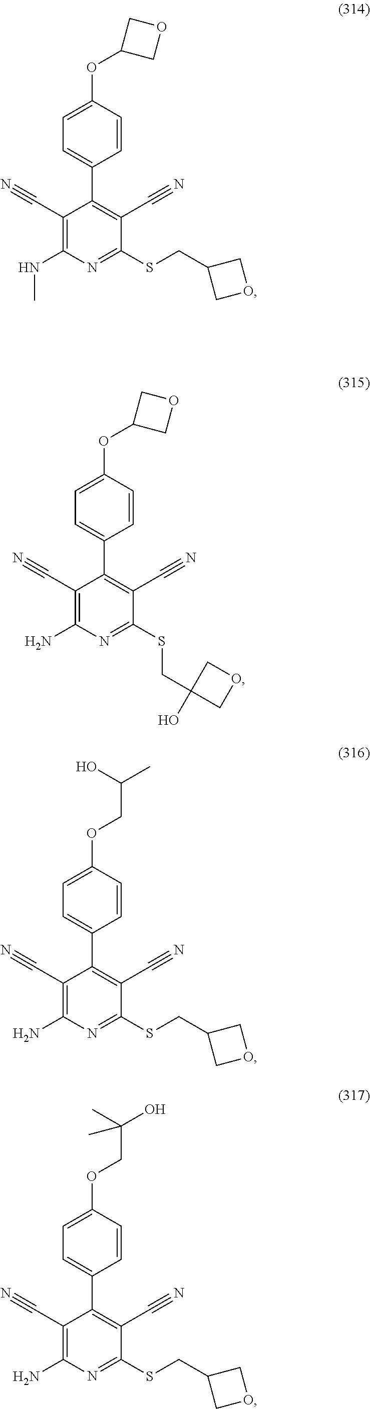 [SMILES: CNc1nc(SCC2COC2)c(C#N)c(-c2ccc(OC3COC3)cc2)c1C#N ; N#Cc1c(N)nc(SCC2(O)COC2)c(C#N)c1-c1ccc(OC2COC2)cc1 ; CC(O)COc1ccc(-c2c(C#N)c(N)nc(SCC3COC3)c2C#N)cc1 ; CC(C)(O)COc1ccc(-c2c(C#N)c(N)nc(SCC3COC3)c2C#N)cc1]

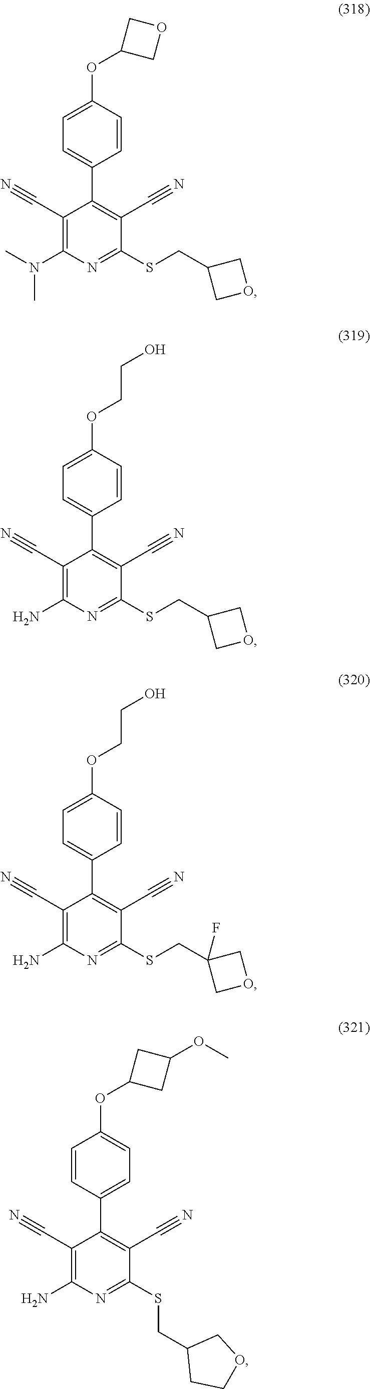 [SMILES: CN(C)c1nc(SCC2COC2)c(C#N)c(-c2ccc(OC3COC3)cc2)c1C#N ; N#Cc1c(N)nc(SCC2COC2)c(C#N)c1-c1ccc(OCCO)cc1 ; N#Cc1c(N)nc(SCC2(F)COC2)c(C#N)c1-c1ccc(OCCO)cc1 ; COC1CC(Oc2ccc(-c3c(C#N)c(N)nc(SCC4CCOC4)c3C#N)cc2)C1]

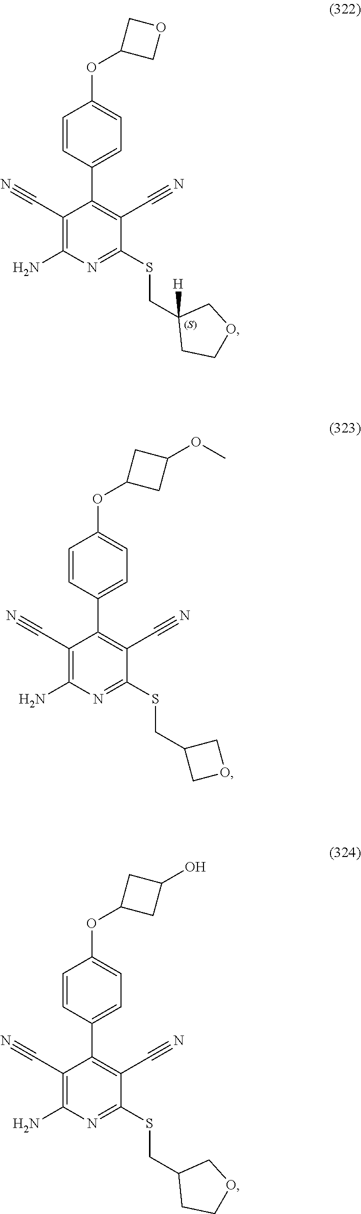

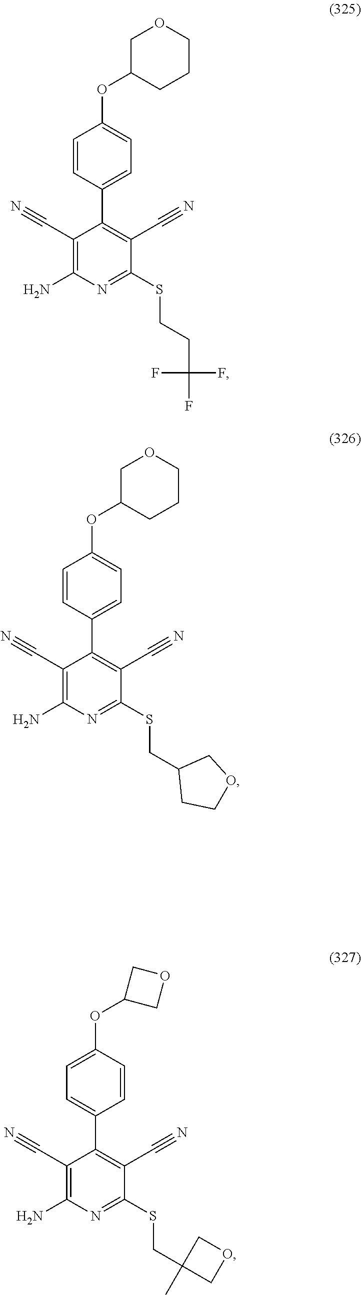 [SMILES: N#Cc1c(N)nc(SCCC(F)(F)F)c(C#N)c1-c1ccc(OC2CCCOC2)cc1 ; N#Cc1c(N)nc(SCC2CCOC2)c(C#N)c1-c1ccc(OC2CCCOC2)cc1 ; CC1(CSc2nc(N)c(C#N)c(-c3ccc(OC4COC4)cc3)c2C#N)COC1]

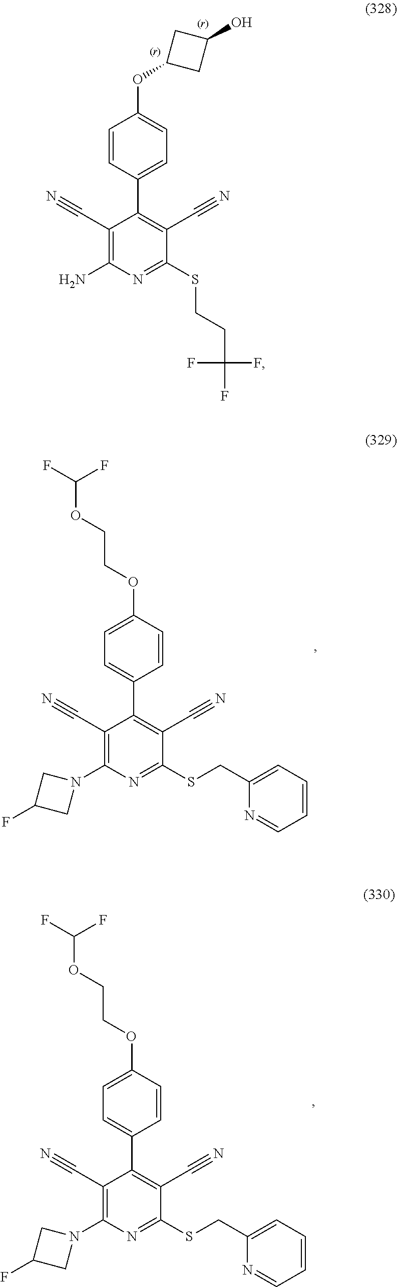

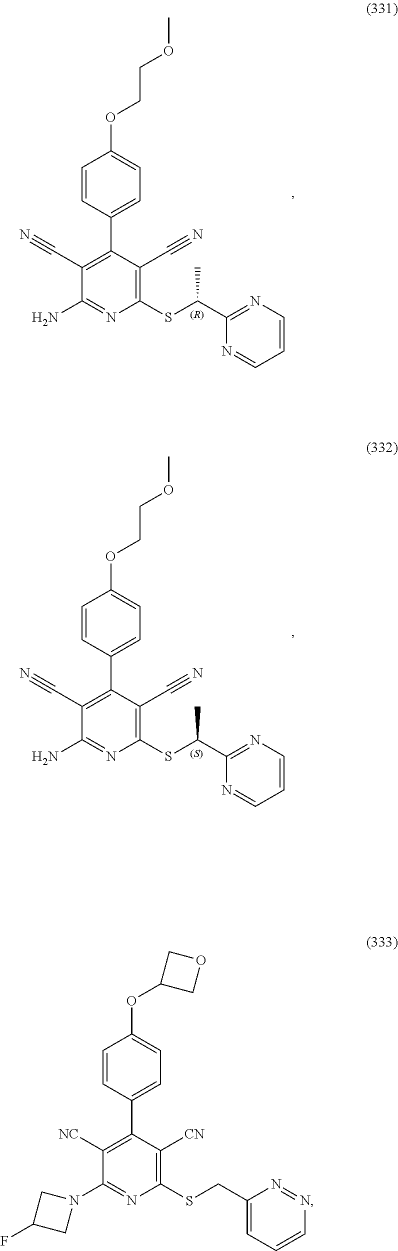

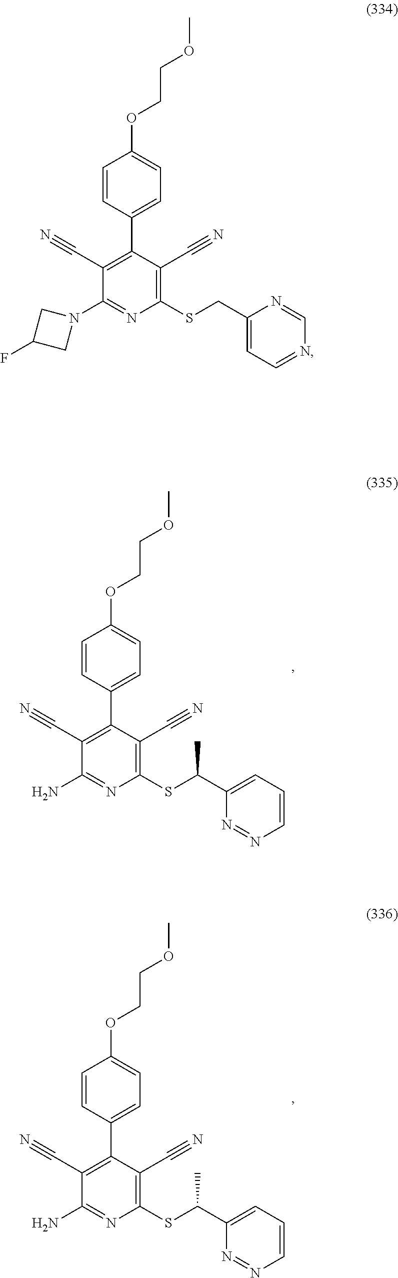 [SMILES: COCCOc1ccc(-c2c(C#N)c(SCc3ccncn3)nc(N3CC(F)C3)c2C#N)cc1 ; COCCOc1ccc(-c2c(C#N)c(N)nc(S[C@@H](C)c3cccnn3)c2C#N)cc1 ; COCCOc1ccc(-c2c(C#N)c(N)nc(S[C@H](C)c3cccnn3)c2C#N)cc1]

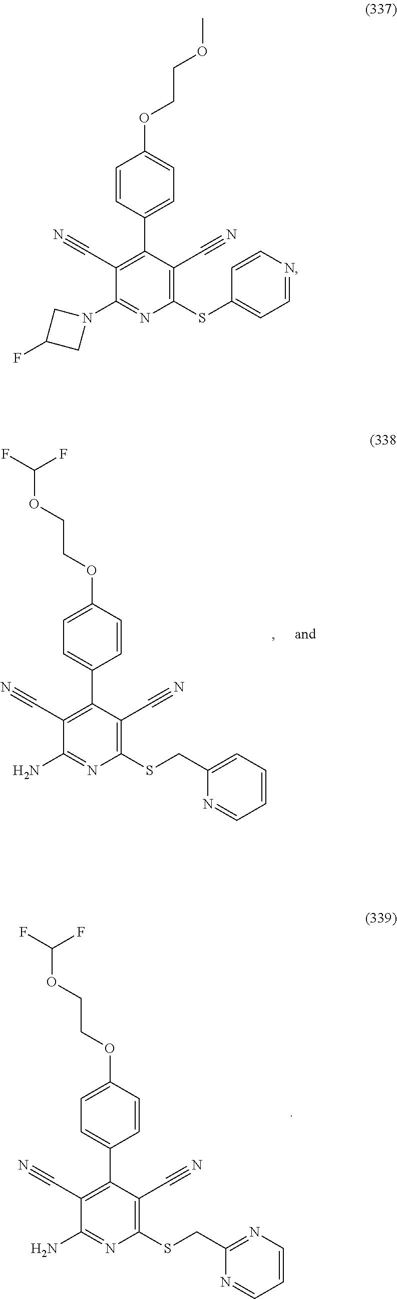 [SMILES: COCCOc1ccc(-c2c(C#N)c(Sc3ccncc3)nc(N3CC(F)C3)c2C#N)cc1 ; N#Cc1c(N)nc(SCc2ccccn2)c(C#N)c1-c1ccc(OCCOC(F)F)cc1 ; N#Cc1c(N)nc(SCc2ncccn2)c(C#N)c1-c1ccc(OCCOC(F)F)cc1]

In certain embodiments wherein alkyl, alkenyl, alkynyl, alkoxy, aryl, heteroaryl, cycloalkyl, heterocyclyl, or oxime are substituted, they are substituted, valency permitting, with one or more substituents selected from substituted or unsubstituted alkyl, such as perfluoroalkyl (e.g., trifluoromethyl), alkenyl, alkoxy, alkoxyalkyl, aryl, aralkyl, arylalkoxy, aryloxy, aryloxyalkyl, hydroxyl, halo, alkoxy, such as perfluoroalkoxy (e.g., trifluoromethoxy), alkoxyalkoxy, hydroxyalkyl, hydroxyalkylamino, hydroxyalkoxy, amino, aminoalkyl, alkylamino, aminoalkylalkoxy, aminoalkoxy, acylamino, acylaminoalkyl, such as perfluoro acylaminoalkyl (e.g., trifluoromethylacylaminoalkyl), acyloxy, cycloalkyl, cycloalkylalkyl, cycloalkylalkoxy, heterocyclyl, heterocyclylalkyl, heterocyclyloxy, heterocyclylalkoxy, heteroaryl, heteroarylalkyl, heteroarylalkoxy, heteroaryloxy, heteroaryloxyalkyl, heterocyclylaminoalkyl, heterocyclylaminoalkoxy, amido, amidoalkyl, amidine, imine, oxo, carbonyl (such as carboxyl, alkoxycarbonyl, formyl, or acyl, including perfluoroacyl (e.g., C(O)CF₃)), carbonylalkyl (such as carboxyalkyl, alkoxycarbonylalkyl, formylalkyl, or acylalkyl, including perfluoroacylalkyl (e.g., -alkylC(O)CF₃)), carbamate, carbamatealkyl, urea, ureaalkyl, sulfate, sulfonate, sulfamoyl, sulfone, sulfonamide, sulfonamidealkyl, cyano, nitro, azido, sulfhydryl, alkylthio, thiocarbonyl (such as thioester, thioacetate, or thioformate), phosphoryl, phosphate, phosphonate or phosphinate.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents, positions of substituents and/or variables are permissible only if such combinations result in chemically stable compounds.

As used in this application, the term “optionally substituted” means that substitution is optional and therefore it is possible for the designated atom or moiety to be unsubstituted.

Compounds of the present application containing one or multiple asymmetrically substituted atoms may be isolated in optically active or racemic forms. It is well known in the art how to prepare optically active forms, such as by resolution of racemic forms, by synthesis from optically active starting materials, or by synthesis using optically active reagents.

In certain embodiments, compounds of the application may be racemic. For example, in embodiments of the application wherein a compound (e.g., a compound of formula (I) or (Ia), or a pharmaceutically acceptable salts thereof) is disclosed herein as a particular enantiomer, the application further contemplates the compound in its racemic form. In certain embodiments, compounds of the application may be enriched in one enantiomer. For example, a compound of the application may have greater than 30% ee, 40% ee, 50% ee, 60% ee, 70% ee, 80% ee, 90% ee, or even 95% or greater ee.

In certain embodiments, the therapeutic preparation may be enriched to provide predominantly one enantiomer of a compound (e.g., of formula (I) or (Ia), or a pharmaceutically acceptable salt thereof). An enantiomerically enriched mixture may comprise, for example, at least 60 mol percent of one enantiomer, or more preferably at least 75, 90, 95, or even 99 mol percent. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

In certain embodiments, compounds of the application may have more than one stereocenter. In certain such embodiments, compounds of the application may be enriched in one or more diastereomer. For example, a compound of the application may have greater than 30% de, 40% de, 50% de, 60% de, 70% de, 80% de, 90% de, or even 95% or greater de.

In certain embodiments, the therapeutic preparation may be enriched to provide predominantly one diastereomer of a compound (e.g., of formula (I) or (Ia), or a pharmaceutically acceptable salt thereof). A diastereomerically enriched mixture may comprise, for example, at least 60 mol percent of one diastereomer, or more preferably at least 75, 90, 95, or even 99 mol percent.

A variety of compounds in the present application may exist in particular geometric or stereoisomeric forms. The present application takes into account all such compounds, including tautomers, cis- and trans-isomers, R- and S-enantiomers, diastereomers, (D)-isomers, (L)-isomers, the racemic mixtures thereof, and other mixtures thereof, as being covered within the scope of this application. All tautomeric forms are encompassed in the present application. Additional asymmetric carbon atoms may be present in a substituent such as an alkyl group. All such isomers, as well as mixtures thereof, are intended to be included in this application, unless the stereochemistry or isomeric form is specifically indicated.

The present application further includes all pharmaceutically acceptable isotopically labelled compounds (e.g., of formula (I) or (Ia), or pharmaceutically acceptable salts thereof). An “isotopically” or “radio-labelled” compound is a compound where one or more atoms are replaced or substituted by an atom having an atomic mass or mass number different from the atomic mass or mass number typically found in nature (i.e., naturally occurring). For example, in certain embodiments, in compounds (e.g., of formula (I) or (Ia), or pharmaceutically acceptable salts thereof), hydrogen atoms are replaced or substituted by one or more deuterium or tritium (e.g., hydrogen atoms on a C₁₋₆ alkyl or a C₁₋₆ alkoxy are replaced with deuterium, such as d₃-methoxy or 1,1,2,2-d₄-3-methylbutyl).

Certain isotopically labelled compounds (e.g., of formula (I) or (Ia), or pharmaceutically acceptable salts thereof), in the application, for example, those incorporating a radioactive isotope, are useful in drug and/or substrate tissue distribution studies. The radioactive isotopes tritium, i.e. ³H, and carbon 14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵, and ¹³N, can be useful in Positron Emission Topography (PET) studies for examining substrate receptor occupancy.

Isotopically labelled compounds (e.g., of formula (I) or (Ia), or pharmaceutically acceptable salts thereof) can generally be prepared by conventional techniques known to those skilled in the art or by processes analogous to those described in the accompanying examples using an appropriate isotopically labelled reagent in place of the non-labelled reagent previously employed. Suitable isotopes that may be incorporated in compounds of the present application include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I, and ¹³¹I. In certain embodiments, the present application provides a pharmaceutical preparation suitable for use in a human patient, comprising any of the compounds shown above (e.g., a compound of the application, such as a compound of formula (I) or (Ia), or pharmaceutically acceptable salts thereof) and one or more pharmaceutically acceptable excipients. In certain embodiments, the pharmaceutical preparations may be for use in treating or preventing a condition or disease as described herein. In certain embodiments, the pharmaceutical preparations have a low enough pyrogen activity to be suitable for use in a human patient.

Compounds of any of the above structures may be used in the manufacture of medicaments for the treatment of any diseases or conditions disclosed herein.

### Uses of the Compounds

Compounds of the present application may be administered orally, parenteral, buccal, vaginal, rectal, inhalation, insufflation, sublingually, intramuscularly, subcutaneously, topically, intranasally, intraperitoneally, intrathoracically, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient. The quantity of the compound to be administered will vary for the patient being treated and will vary from about 100 ng/kg of body weight to 100 mg/kg of body weight per day. For instance, dosages can be readily ascertained by those skilled in the art from this disclosure and the knowledge in the art. This, the skilled artisan can readily determine the amount of compound and optional additives, vehicles, and/or carrier in compositions and to be administered in methods of the application. In certain embodiments, the application relates to a compound according to formula (I) or (Ia), or a pharmaceutically acceptable salt of the compound according to formula (I) or (Ia), for use as a medicament, e.g., for treatment of any of the disorders disclosed herein.

In certain embodiments, the application relates to a compound according to formula (I) or (Ia), or a pharmaceutically acceptable salt of the compound according to formula (I) or (Ia), for use as a medicament.

In certain embodiments, the application relates to the use of a compound according to formula (I) or (Ia), or a pharmaceutically acceptable salt of the compound according to formula (I) or (Ia), in the manufacture of a medicament for treatment of Rett syndrome and/or one or more symptoms associated with Rett syndrome in a subject. In certain such embodiments, the one or more symptoms associated with Rett syndrome is selected from sleep disturbances, including irregular sleep times, falling asleep during the day and being awake at night, or waking in the night with crying or screaming; sleep apnea; seizures, including those accompanied by an abnormal electroencephalogram (EEG); breathing disorders, including breath-holding, abnormally rapid breathing (hyperventilation), forceful exhalation of air or saliva, and swallowing air; irregular heartbeat, including problems with heart rhythm such as abnormally long pauses between heartbeats (e.g., as measured by an electrocardiogram), and other types of arrhythmia; intellectual disabilities; and autism.

In certain embodiments, the application relates to a method of treating or preventing Rett syndrome and/or one or more symptoms associated with Rett syndrome in a mammal, such as a human being, comprising administering to said patient a therapeutically effective amount of a compound according to formula (I) or (Ia), or a pharmaceutically acceptable salt of the compound according to formula (I) or (Ia). In certain such embodiments, the one or more symptoms associated with Rett syndrome is selected from sleep disturbances, including irregular sleep times, falling asleep during the day and being awake at night, or waking in the night with crying or screaming; sleep apnea; seizures, including those accompanied by an abnormal electroencephalogram (EEG); breathing disorders, including breath-holding, abnormally rapid breathing (hyperventilation), forceful exhalation of air or saliva, and swallowing air; irregular heartbeat, including problems with heart rhythm such as abnormally long pauses between heartbeats (e.g., as measured by an electrocardiogram), and other types of arrhythmia; intellectual disabilities; and autism.

In certain embodiments, a compound according to formula (I) or (Ia), or a pharmaceutically acceptable salt of the compound according to formula (I) or (Ia), as disclosed herein is a brain penetrant. In certain embodiments, a compound according to formula (I) or (Ia), or a pharmaceutically acceptable salt of the compound according to formula (I) or (Ia), as disclosed herein is capable of crossing the blood brain barrier.

In certain embodiments of the methods a disclosed herein, the compound is selected from any one of compounds 1-10, 12-18, 20-25, 27-37, 39, 40, 44-53, 56, 61-70, 74, 76-79, 83, 84, 89, 90, 93-96, 104, 106, 107, 110, 112-117, 119-123, 125, 127-129, 132, 136, 138-141, 146-147, 150-152, 159-162, 172, 174-178, 183, 191-195, 197, 198, 206-208, and 212-230, and pharmaceutically acceptable salts thereof. In certain embodiments of the methods a disclosed herein, the compound is selected from any one of compounds 1-10, 12-18, 20-25, 27-37, 39, 40, 44-53, 56, 61-70, 74, 76-79, 83, 84, 89, 90, 93-96, 104, 106, 107, 110, 112-117, 119-123, 125, 127-129, 132, 136, 138-141, 146-147, 150-152, 159-162, 172, 174-178, 183, 191-195, 197, 198, 206-208, 212-230, and 232-339, and pharmaceutically acceptable salts thereof.

In certain embodiments, the application relates to a pharmaceutical composition comprising as active ingredient a therapeutically effective amount of a compound according to formula (I) or (Ia), or a pharmaceutically acceptable salt of the compound according to formula (I) or (Ia), in association with at least one pharmaceutically acceptable excipient, carrier or diluent. In certain such embodiments, the pharmaceutical composition is for treating a disease or disorder in a patient in need thereof, such as a disease or disorder as disclosed herein.

In certain embodiments, the application relates to a pharmaceutical composition comprising (1) a compound according to formula (I) or (Ia), or a pharmaceutically acceptable salt of the compound of formula (I) or (Ia), (2) an additional therapeutic agent, or a pharmaceutically acceptable salt thereof, and (3) pharmaceutically acceptable excipients, carriers or diluents.

In the treatment of any of the disorders disclosed herein, different compounds of the application may be (e.g., conjointly) administered with one or more other compounds of the application. Moreover, compounds of formula (I) or (Ia), or a pharmaceutically acceptable salt of the compound of formula (I) or (Ia), or certain combinations thereof, may be conjointly administered with other conventional therapeutic agents in treating one or more disease conditions referred to herein.

In certain embodiments, compounds of the application may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either simultaneously, sequentially, or by separate dosing of the individual components of the treatment. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of compounds of the application with one or more additional therapeutic agent(s) provides improved efficacy relative to each individual administration of the compound of the application (e.g., compound of formula (I) or (Ia), or a pharmaceutically acceptable salt of the compound of formula (I) or (Ia)) or the one or more additional therapeutic agent(s). In certain such embodiments, the conjoint administration provides an additive effect, wherein an additive effect refers to the sum of each of the effects of individual administration of the compound of the application and the one or more additional therapeutic agent(s).

Such conventional therapeutics may include one or more of the following categories of agents:
anticonvulsants including: for example carbamazepine, clonazepam, ethosuximide, felbamate, fosphenytoin, gabapentin, lacosamide, lamotrogine, levetiracetam, oxcarbazepine, phenobarbital, phenytoin, pregabaline, rufinamide, topiramate, valproate, vigabatrine, zonisamide and equivalents and pharmaceutically active isomer(s) and metabolite(s) thereof;
insomnia therapies including: for example agomelatine, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, ramelteon, roletamide, triclofos, secobarbital, zaleplon, zolpidem and equivalents and pharmaceutically active isomer(s) and metabolite(s) thereof.

Such combination products employ the compounds of this application within the dosage range described herein and the other pharmaceutically active compound or compounds within approved dosage ranges and/or the dosage described in the publication reference.

### Definitions

The definitions set forth in this application are intended to clarify terms used throughout this application.

The term “herein” means the entire application.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. Co alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS-.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

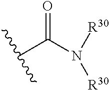

wherein each R³⁰ independently represent a hydrogen or hydrocarbyl group, or two R³⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

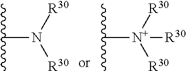

wherein each R³⁰ independently represents a hydrogen or a hydrocarbyl group, or two R³⁰ are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

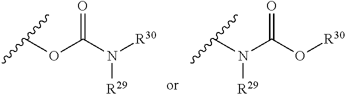

wherein R²⁹ and R³⁰ independently represent hydrogen or a hydrocarbyl group, such as an alkyl group, or R²⁹ and R³⁰ taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated, and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated, and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R³⁰, wherein R³⁰ represents a hydrocarbyl group.

The term “carboxy”, as used herein, refers to a group represented by the formula —CO₂H.

The term “ester”, as used herein, refers to a group —C(O)OR³⁰ wherein R³⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Exemplary heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or =S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this application, the heteroatoms such as nitrogen may have hydrogen substituents and/or any permissible substituents of organic compounds described herein which satisfy the valences of the heteroatoms.

Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

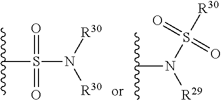

wherein R²⁹ and R³⁰ independently represents hydrogen or hydrocarbyl, such as alkyl, or R²⁹ and R³⁰ taken together with the intervening atom(s) complete a heterocycle having from 4 to 8 atoms in the ring structure.

The term “sulfoxide” is art-recognized and refers to the group —S(O)—R³⁰, wherein R³⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R³⁰, wherein R³⁰ represents a hydrocarbyl.

The term “thioalkyl”, as used herein, refers to an alkyl group substituted with a thiol group.

The term “thioester”, as used herein, refers to a group —C(O)SR³⁰ or —SC(O)R³⁰ wherein R³⁰ represents a hydrocarbyl.

The term “thioether”, as used herein, is equivalent to an ether, wherein the oxygen is replaced with a sulfur.

The term “urea” is art-recognized and may be represented by the general formula

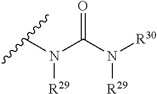

wherein R²⁹ and R³¹ independently represent hydrogen or a hydrocarbyl, such as alkyl, or either occurrence of R²⁹ taken together with R^{3′} and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxylprotecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

The term “healthcare providers” refers to individuals or organizations that provide healthcare services to a person, community, etc. Examples of “healthcare providers” include doctors, hospitals, continuing care retirement communities, skilled nursing facilities, subacute care facilities, clinics, multispecialty clinics, freestanding ambulatory centers, home health agencies, and HMO's.

The present application includes prodrugs of the compounds formula (I) or (Ia), or pharmaceutically acceptable salts thereof. The term “prodrug” is intended to encompass compounds which, under physiologic conditions, are converted into the therapeutically active agents of the present application (e.g., a compound of formula (I) or (Ia), or pharmaceutically acceptable salts thereof). A common method for making a prodrug is to include one or more selected moieties which are hydrolyzed under physiologic conditions to yield the desired molecule. In certain embodiments, the prodrug is converted by an enzymatic activity of the host animal. For example, a prodrug with a nitro group on an aromatic ring could be reduced by reductase to generate the desired amino group of the corresponding active compound in vivo. In another example, functional groups such as a hydroxyl, carbonate, or carboxylic acid in the parent compound are presented as an ester, which could be cleaved by esterases. Additionally, amine groups in the parent compounds are presented in, but not limited to, carbamate, N-alkylated or N-acylated forms (Simplicio et al, “Prodrugs for Amines,” Molecules, (2008), 13:519-547). In certain embodiments, some or all of the compounds of formula (I) or (Ia), or pharmaceutically acceptable salts thereof, in a formulation represented above can be replaced with the corresponding suitable prodrug.

The present application includes metabolites of the compounds of formula (I) or (Ia), or pharmaceutically acceptable salts thereof. The term “metabolite” is intended to encompass compounds that are produced by metabolism/biochemical modification of the parent compound under physiological conditions, e.g. through certain enzymatic pathway. For example, an oxidative metabolite is formed by oxidation of the parent compound during metabolism, such as the oxidation of a pyridine ring to pyridine-N-oxide. In another example, an oxidative metabolite is formed by demethylation of a methoxy group to result in a hydroxyl group.

### Pharmaceutical Compositions

The compositions and methods of the present application may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the application and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the application. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the application. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the application, with the carrier and, optionally, one or more accessory ingredients. In general, the formulations are prepared by uniformly and intimately bringing into association a compound of the present application with liquid carriers, or finely divided solid carriers, or both, and then, if necessary, shaping the product.

Formulations of the application suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of a compound of the present application as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that releases the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present application to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this application. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the application include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this application, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician having ordinary skill in the art can readily determine and prescribe the therapeutically effective amount of the pharmaceutical composition required. For example, the physician could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the application. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the application will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present application, the active compound may be administered two or three times daily. In preferred embodiments, the active compound will be administered once daily.

The patient receiving this treatment is any animal in need, including primates, in particular humans.

This application includes the use of pharmaceutically acceptable salts of compounds of the application in the compositions and methods of the present application. The term “pharmaceutically acceptable salts” includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, such as an amine, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, trifluoroacetic, propionic, isobutyric, maleic, malonic, benzoic, succinic, suberic, fumaric, lactic, mandelic, phthalic, benzensulfonic, p-tolylsulfonic, citric, tartaric, methanesulfonic, camphorsulfonic and the like. In certain embodiments, the pharmaceutically acceptable salt is a hydrochloride salt. In certain embodiments, the pharmaceutically acceptable salt is a camsylate salt. In certain embodiments, contemplated salts of the compounds include, but are not limited to, alkyl, dialkyl, trialkyl or tetra-alkyl ammonium salts. In certain embodiments, contemplated salts of compounds include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of compounds include, but are not limited to, Li, Na, Ca, K, Mg, Zn or other metal salts. Also included are the salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention may contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

The compounds of the application, including their pharmaceutically acceptable salts, can also exist as various solvates, such as with water (also known as hydrates), methanol, ethanol, dimethylformamide, diethyl ether, acetamide, and the like. Mixtures of such solvates can also be prepared. The source of such solvate can be from the solvent of crystallization, inherent in the solvent of preparation or crystallization, or adventitious to such solvent.

The compounds of the application, including their pharmaceutically acceptable salts, can also exist as various polymorphs, pseudo-polymorphs, or in amorphous state. As used herein, the term “polymorph” refers to different crystalline forms of the same compound and other solid state molecular forms including pseudo-polymorphs, such as hydrates, solvates, or salts of the same compound. Different crystalline polymorphs have different crystal structures due to a different packing of molecules in the lattice, as a result of changes in temperature, pressure, or variations in the crystallization process. Polymorphs differ from each other in their physical properties, such as x-ray diffraction characteristics, stability, melting points, solubility, or rates of dissolution in certain solvents. Thus, crystalline polymorphic forms are important aspects in the development of suitable dosage forms in pharmaceutical industry.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

In certain embodiments, the application comprises a method for conducting a pharmaceutical business, by determining an appropriate formulation and dosage of a compound of the application for treating or preventing any of the diseases or conditions as described herein, conducting therapeutic profiling of identified formulations for efficacy and toxicity in animals, and providing a distribution network for selling an identified preparation as having an acceptable therapeutic profile. In certain embodiments, the method further includes providing a sales group for marketing the preparation to healthcare providers.

In certain embodiments, the application relates to a method for conducting a pharmaceutical business by determining an appropriate formulation and dosage of a compound of the application for treating or preventing any of the disease or conditions as described herein, and licensing, to a third party, the rights for further development and sale of the formulation.

### EXAMPLES

### Example 1: Synthetic Protocols

Below follows a number of non-limiting examples of compounds of the application.

Unless specifically stated otherwise, the experimental procedures were performed under the following conditions. All operations were carried out at room temperature (about 18° C. to about 25° C.) under nitrogen atmosphere. Evaporation of solvent was carried out using a rotary evaporator under reduced pressure. The course of a reaction was followed by thin layer chromatography (TLC), high-pressure liquid chromatography (HPLC) or liquid chromatography-mass spectrometry (LC-MS), and reaction times are given for illustration only. The compounds provided herein may be isolated and purified by known standard procedures. Such procedures include (but are not limited to) recrystallization, column chromatography, HPLC, or supercritical fluid chromatography (SFC). Silica gel chromatography was performed on silica gel 60 (230-400 mesh). Chromatography columns available for use in the separation/purification of the enantiomers/diastereoisomers provided herein include, but are not limited to, Waters Xbridge Prep OBD®, Waters Xbridge BEH®, Kromasil®, Phenomenex Luna®, etc.

The structure and purity of all final products was assured by at least one of the following analytical methods: nuclear magnetic resonance (NMR) and LC-MS. NMR spectra was recorded on a Bruker 400 MHz NMR Spectrometer using the indicated solvent. Chemical shift (6) is given in parts per million (ppm) relative to tetramethylsilane (TMS) as an internal standard. Coupling constants (J) are expressed in hertz (Hz), and conventional abbreviations used for signal shape are: s=singlet; d=doublet; t=triplet; m=multiplet; br=broad; etc.

The following abbreviations have been used:
BOC tert-butoxycarbonyl
DEAD diethyl azodicarboxylate
DCCI 1,3-dicyclohexyl carbodiimide
DCM Dichloromethane
DMA N,N-dimethylacetamide
DMAP 4-dimethyl-aminopyridine
DME 1,2-dimethoxyethane
DMF N,N-dimethylformamide
DMSO dimethylsulfoxide
EDCl 1-(3-dimethylaminopropyl)-3-ethyl-carbodiimide
EEDQ 2-ethoxy-1-ethoxycarbonyl-1,2-dihydroquinoline
EtOAc Ethyl acetate
HOBT hydroxybenzotriazole
MeCN acetonitrile
mCPBA m-chloroperoxybenzoic acid
NMM N-methylmorpholine
NMMO N-methylmorpholine-N-oxide
PE Petroleum ether
TBAF tetra-n-butylammonium fluoride
TFA trifluoroacetic acid
THF tetrahydrofuran
TMEDA N,N,N′,N′-tetramethylethylenediamine
TMSI trimethylsilyliodide
TPAP tetrapropylammonium perruthenate

### General Synthetic Methods for the Preparation of the Compounds of the Present Invention

The general synthetic routes to prepare diversely substituted amino-3,5-dicyanopyridine derivatives are depicted in the schemes below. Compounds were synthesized starting from the suitable commercially available 4-hydroxybenzaldehyde properly alkylated, followed by a one-pot cyclization with cyanothioacetamide in the presence of a base such as triethylamine or piperidine. Upon treatment with alkylating agents, the thiolate gave new 2-(alkylthio)pyridines (Scheme 1). (Matrosova, S. V.; Zav'yalova, V. K.; Litvinov, V. P.; Sharanin, Yu. A. Izvestiya Akademii Nauk SSSR, Seriya Khimicheskaya (1991), (7), 1643-6.)

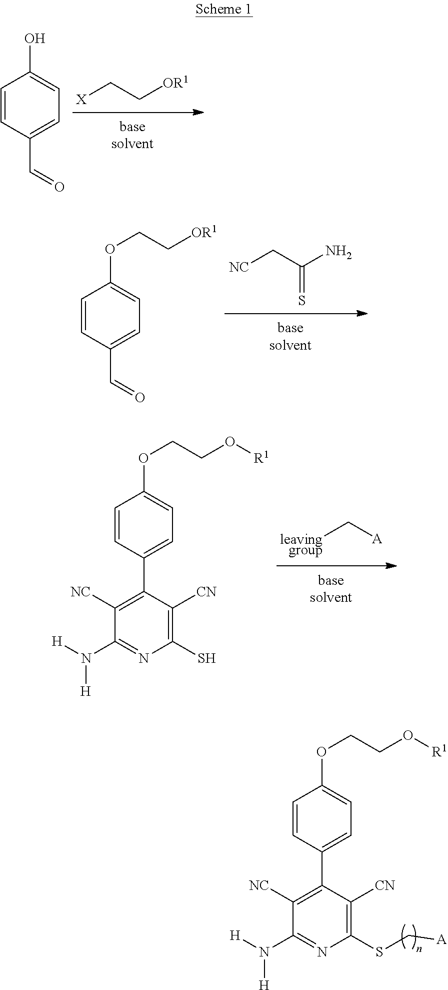

Preparation of 6-amino-3,5-dicyanopyridines diversely substituted on C-6 with mono- or dialkylamino groups may then be carried out by reaction with t-butyl nitrite and a copper salt in a solvent such as acetonitrile, followed by nucleophilic substitution of the halogen by a mono- or dialkyl-substituted amine (Scheme 2).

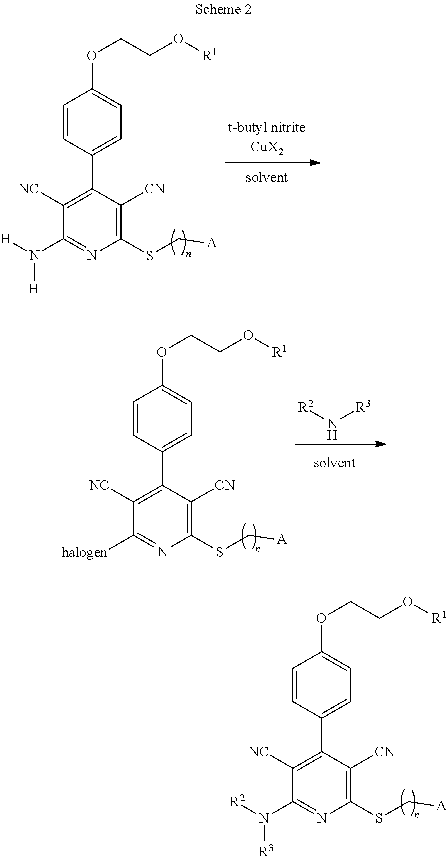

### Preparation of Compounds of Formula I

Compounds of formula I

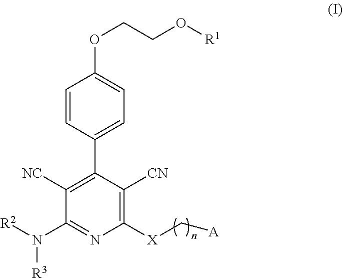

i.e., compounds of Formula I where X is S

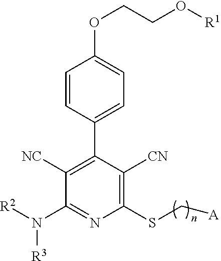

and R² and R³ are H

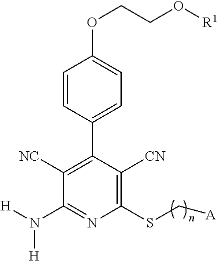

are prepared by treatment of a phenol with an alkylating agent such as 1-bromo-2-methoxyethane or 1-bromo-hydroxyethane and a base such as K₂CO₃ in a solvent such as DMF or acetonitrile to give an intermediate aldehyde.

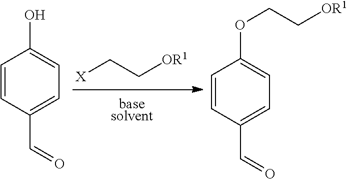

The aromatic aldehyde can be condensed with 2-cyanothioacetamide in the presence of a base such as N-methylmorpholine or K₂CO₃ in a solvent such as ethanol to give a 4-aryl-2-mercaptopyridine.

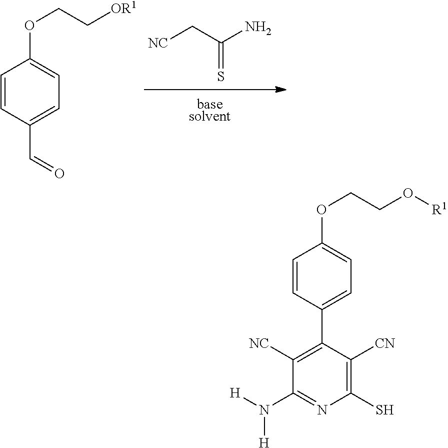

The 2-mercaptopyridine can be reacted with an alkylating agent containing a leaving group commonly used in the art such as chloride, bromide, or mesylate in the presence of base in a solvent such as DMF to give a compound of Formula I where A is cyano, hydroxyl, optionally substituted C₁₋₆alkyl, optionally substituted C₁₋₆ alkoxy, —C(O)NR⁵R⁶, —NR⁵R⁶, —NR⁵C(O)C₁₋₆alkyl, optionally substituted heterocycloalkyl, aryl, or optionally substituted heteroaryl.

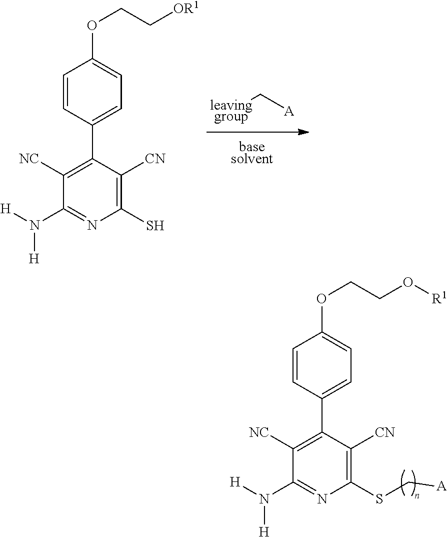

A compound of Formula I where R² and R³ are H can be further modified to give a compound of Formula I where R² and R³ each independently can be C₁₋₆alkyl or R² and R³ taken together with the nitrogen to which they are attached form a 4- or 5-membered heterocycloalkyl ring by conversion of the amine to a chloride or bromide using a t-butyl nitrite and a copper salt in a solvent such as acetonitrile. The resulting 2-halopyridine can be treated with an amine such as pyrrolidine or azetidine in a solvent such as methanol or dichloromethane to give a compound of Formula I where R² and R³ are each independently C₁₋₆alkyl or R² and R³ taken together with the nitrogen to which they are attached form a 4- or 5-membered heterocycloalkyl ring.

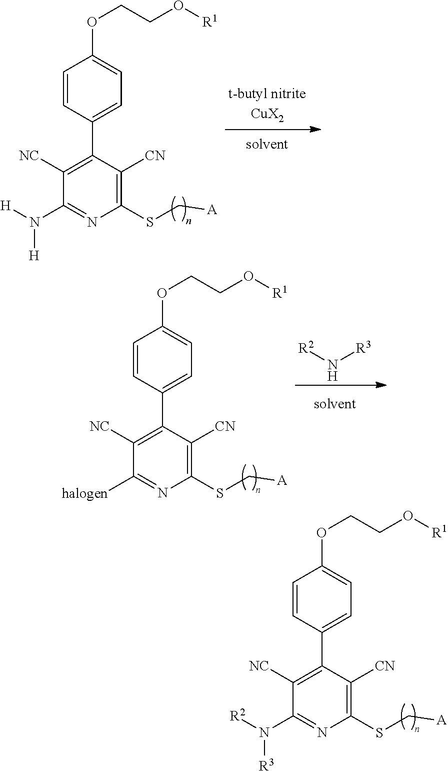

### Protocol 1. 2-Amino-4-[4-(2-hydroxy-propoxy)phenyl]-6-(3-pyridylmethylsulfanyl)pyridine-3,5-dicarbonitrile (56) and 2-(azetidin-1-yl)-4-[4-(2-hydroxypropoxy)phenyl]-6-(3-pyridylmethylsulfanyl)-pyridine-3,5-dicarbonitrile (53)

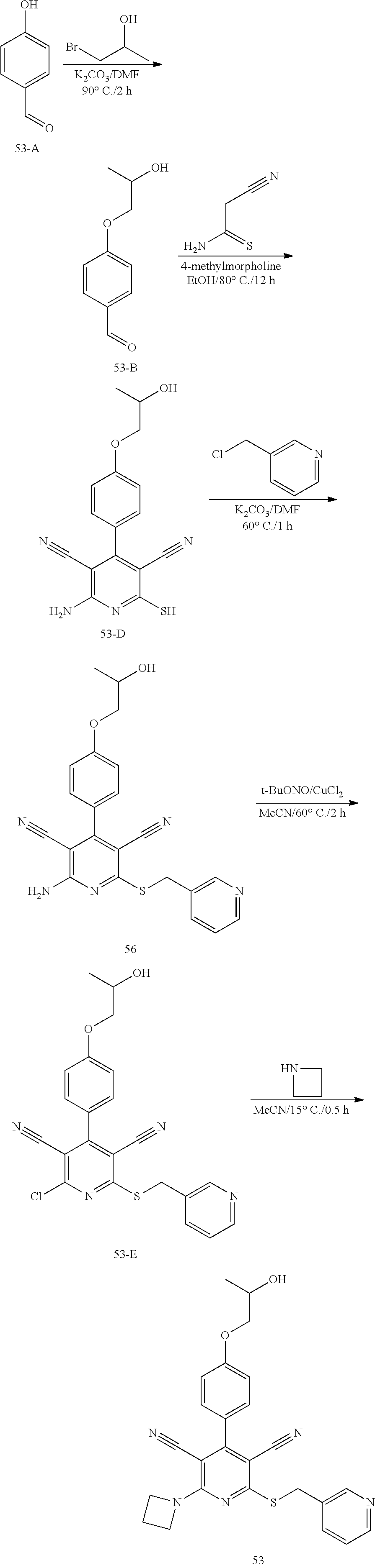

Step 1: Preparation of 4-(2-hydroxypropoxy)benzaldehyde (53-B). A mixture of 4-hydroxybenzaldehyde (4 g, 32.75 mmol, 1 eq), 1-bromopropan-2-ol (5.01 g, 36.03 mmol, 3.27 mL, 1.1 eq), K₂CO₃ (9.05 g, 65.51 mmol, 2 eq) in DMF (1 mL) was stirred at 90° C. for 2 h. The reaction mixture was filtered, and the filtrate was concentrated under reduced pressure to give a residue. The residue was purified by column chromatography (SiO₂, PE:EtOAc=20:1 to 2:1) to give 4-(2-hydroxypropoxy)benzaldehyde (2.8 g, 15.54 mmol, 47.44% yield) as a yellow oil.

¹H-NMR (400 MHz, DMSO-d₆) δ=1.18 (d, J=6.0 Hz, 3H), 3.89-3.97 (m, 2H), 4.00-4.04 (m, 1H), 7.14 (m, J=8.8 Hz, 2H), 7.84-7.89 (m, 2H), 9.86-9.89 (m, 1H). [M+H]=180.9

Step 2. Preparation of 2-amino-4-[4-(2-hydroxypropoxy)phenyl]-6-sulfanyl-pyridine-3,5-dicarbonitrile (53-D). A mixture of 4-(2-hydroxypropoxy)benzaldehyde (1 g, 5.55 mmol, 1 eq), 2-cyanothioacetamide (1.22 g, 12.21 mmol, 2.2 eq), NMM (1.18 g, 11.65 mmol, 1.28 mL, 2.1 eq) in EtOH (10 mL) was stirred at 80° C. for 12 h. To the reaction mixture was added H₂O (15 mL) and extracted with EtOAc (3×50 mL). The combined organic layers were washed with brine (50 mL), dried over Na₂SO₄, filtered, and concentrated under reduced pressure to give 2-amino-4-[4-(2-hydroxypropoxy)phenyl]-6-sulfanyl-pyridine-3,5-dicarbonitrile (800 mg, 2.45 mmol, 44.17% yield) as a white solid. [M−H]=324.9.

Step 3. Preparation of 2-amino-4-[4-(2-hydroxypropoxy)phenyl]-6-(3-pyridylmethylsulfanyl)pyridine-3,5-dicarbonitrile (56). A mixture of 2-amino-4-[4-(2-hydroxypropoxy)phenyl]-6-sulfanyl-pyridine-3,5-dicarbonitrile (53-D, 750 mg, 2.30 mmol, 1 eq), 3-(chloromethyl)pyridine (322.47 mg, 2.53 mmol, 1.1 eq), K₂CO₃ (635.19 mg, 4.60 mmol, 2 eq) in DMF (10 mL) was stirred at 60° C. for 1 h. The reaction mixture was filtered, and the filtrate was concentrated under reduced pressure to give a residue. The residue was purified by prep-HPLC (column: Waters Xbridge Prep OBD C18 150 mm×40 mm×10 μm; mobile phase A: 10 mM NH₄HCO₃ in water, mobile phase B: ACN; 25%-55% over 8 min, to give 2-amino-4-[4-(2-hydroxypropoxy)phenyl]-6-(3-pyridylmethylsulfanyl)pyridine-3,5-dicarbonitrile (540 mg, 1.23 mmol, 53.42% yield, 94.9% purity) as white solid. ¹H-NMR (400 MHz, DMSO-d₆) δ=1.13 (d, J=6.4 Hz, 3H), 3.79-3.88 (m, 2H) 3.90-4.00 (m, 1H) 4.43-4.49 (m, 2H) 4.91 (d, J=4.8 Hz, 1H) 7.05 (d, J=8.4 Hz, 2H) 7.31 (dd, J=7.6, 5.2 Hz, 1H) 7.47 (s, 2H) 7.91 (br d, J=7.6 Hz, 1H) 8.42 (br d, J=4.4 Hz, 1H), 8.79 (br d, J=1.6 Hz, 1H). [M+H]=418.0

Step 4. Preparation of 2-chloro-4-[4-(2-hydroxypropoxy)phenyl]-6-(3-pyridylmethylsulfanyl)pyridine-3,5-dicarbonitrile (53-E). A mixture of 2-amino-4-[4-(2-hydroxypropoxy)phenyl]-6-(3-pyridylmethylsulfanyl)-pyridine-3,5-dicarbonitrile (56, 520 mg, 1.25 mmol, 1 eq), tert-butyl nitrite (192.66 mg, 1.87 mmol, 222.22 μL, 1.5 eq), CuCl₂ (334.94 mg, 2.49 mmol, 2 eq) in ACN (6 mL) was stirred at 60° C. for 2 h under N₂ atmosphere. The reaction mixture was concentrated under reduced pressure to give 2-chloro-4-[4-(2-hydroxypropoxy)phenyl]-6-(3-pyridylmethylsulfanyl)pyridine-3,5-dicarbonitrile (500 mg, 1.14 mmol, 91.88% yield) as a black solid. [M+H]=437.1.

Step 5. Preparation of 2-(azetidin-1-yl)-4-[4-(2-hydroxypropoxy)phenyl]-6-(3-pyridylmethylsulfanyl)-pyridine-3,5-dicarbonitrile (53). A mixture of 2-chloro-4-[4-(2-hydroxypropoxy)phenyl]-6-(3-pyridylmethylsulfanyl)pyridine-3,5-dicarbonitrile (250 mg, 572.20 μmol, 1 eq), azetidine (326.69 mg, 5.72 mmol, 386.16 μL 10 eq) in MeOH (3 mL) was stirred at 15° C. for 0.5 h. The reaction mixture was filtered, and filtrate was collected. The residue was purified by prep-HPLC (column: Waters Xbridge Prep OBD C18 150×40 mm×10 μm mobile phase A: 10 mM NH₄HCO₃ in water, mobile phase B: ACN; 25%-55% over 8 min, to give 2-(azetidin-1-yl)-4-[4-(2-hydroxypropoxy)phenyl]-6-(3-pyridylmethylsulfanyl)pyridine-3,5-dicarbonitrile (119.2 mg, 260.52 μmol, 45.53% yield, 100% purity) as white solid. ¹H-NMR (400 MHz, DMSO-d₆) δ=9.11-8.78 (br s, 2H), 7.86 (br s, 1H), 7.44 (br s, 3H), 7.10 (br s, 2H), 4.92 (br s, 1H), 4.55 (br s, 6H), 3.98-3.89 (br s, 3H), 2.18-2.44 (m, 2H), 1.17 (br s, 3H). [M+H]=161.9

TABLE 1
The following compounds were synthesized in a similar manner, but with
different starting materials.

| Compound | Structure | Spectroscopic data |
|---|---|---|
| 52 | 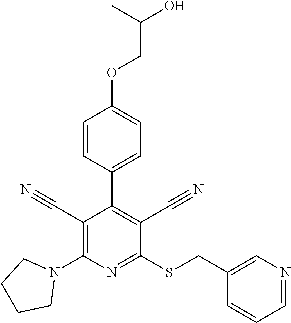 [SMILES: CC(O)COc1ccc(-c2c(C#N)c(SCc3cccnc3)nc(N3CCCC3)c2C#N)cc1] | ¹H-NMR (400 MHz, DMSO-d₆) δ = 9.31- 8.92 (m, 1 H), 7.88 (br s, 1 H), 7.48 (br s, 3H), 7.10 (br s, 2H), 4.93 (br s, 1H), 4.62 (br s, 2 H), 3.98-3.80 (m, 7H), 1.95 (br s, 4H), 1.18 (br s, 3H). [M + H] = 472.2 |
| 51 | 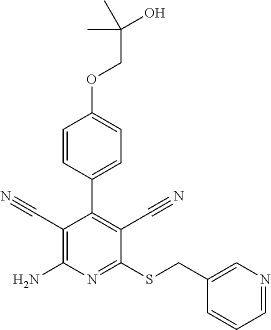 [SMILES: CC(C)(O)COc1ccc(-c2c(C#N)c(N)nc(SCc3cccnc3)c2C#N)cc1] | ¹H-NMR (400 MHz, CDCl₃) δ = 8.71 (d, J = 1.6 Hz, 1H), 8.55 (dd, J = 4.8 1.2, Hz, 1H), 7.77 (br d, J = 8.0 Hz, 1H), 7.51 (d, J = 8.8 Hz, 2H), 7.29 (s, 1H), 7.08 (d, J = 8.8 Hz, 2H), 5.75 (s, 2H), 4.45 (s, 2H), 3.88 (s, 2H), 1.39 (s, 6H). [M + H] = 432.2 |
| 49 | 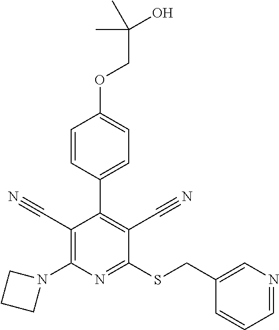 [SMILES: CC(C)(O)COc1ccc(-c2c(C#N)c(SCc3cccnc3)nc(N3CCC3)c2C#N)cc1] | ¹H-NMR (400 MHz, CD₃OD) δ = 9.55- 8.49 (m, 3H), 7.95-7.91(m, 1H), 7.44 (d, J = 8.8 Hz, 2H), 7.14 (d, J = 8.8 Hz, 2H), 4.69 (s, 2H), 4.54-4.39 (m, 4H), 3.90 (s, 2H), 2.48-2.40 (quin, J = 7.6 Hz, 2H), 1.34 (s, 6H). [M + H] = 472.2 |
| 50 | 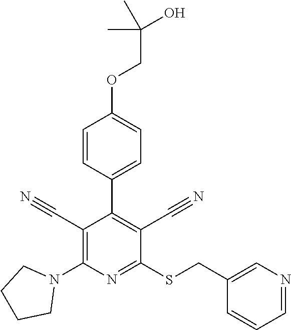 [SMILES: CC(C)(O)COc1ccc(-c2c(C#N)c(SCc3cccnc3)nc(N3CCCC3)c2C#N)cc1] | ¹H-NMR (400 MHz, CD₃OD) δ = 8.43 (br d, J = 6.0 Hz, 2H), 8.25-7.61 (m, 1H), 7.39 (br d, J = 8.4 Hz, 2H), 7.05 (br d, J = 8.4 Hz, 2H), 4.70 (br s, 2H), 4.23-3.33 (m, 6H), 1.95 (br s, 4H), 1.28 (s, 6H). [M + H] = 486.2. |
| (+)-24 | 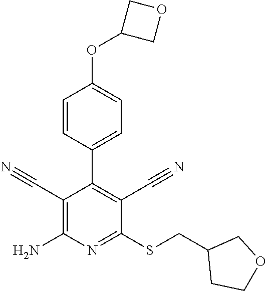 [SMILES: N#Cc1c(N)nc(SCC2CCOC2)c(C#N)c1-c1ccc(OC2COC2)cc1] | ¹H-NMR (400 MHz, CD₃OD) δ = 7.56- 7.48 (m, 2H), 7.01-6.94 (m, 2H), 5.41 (quin, J = 5.6 Hz, 1H), 5.08 (t, J = 6.8 Hz, 2H), 4.77 (dd, J = 7.6, 5.2 Hz, 2H), 4.00- 3.90 (m, 2H), 3.84-3.75 (m, 1H), 3.66- 3.58 (m, 1H), 3.43-3.36 (m, 2H), 2.74- 2.63 (m, 1H), 2.20 (dtd, J = 12.8, 7.6, 5.6 Hz, 1H), 1.86-1.73 (m, 1H). [M + H] = 409.2. |
| 225 | 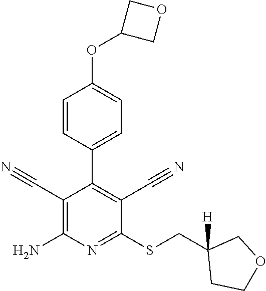 [SMILES: N#Cc1c(N)nc(SC[C@H]2CCOC2)c(C#N)c1-c1ccc(OC2COC2)cc1] | ¹H-NMR (400 MHz, CD₃OD) δ = 7.55- 7.45 (m, 2H), 7.02-6.89 (m, 2H), 5.48- 5.32 (m, 1H), 5.07 (t, J = 7.2 Hz, 2H), 4.82-4.69 (m, 2H), 3.99-3.88 (m, 2H), 3.84-3.74 (m, 1H), 3.60 (dd, J = 8.4, 6.0 Hz, 1H), 3.40-3.38 (m, 1H), 3.37 (d, J = 1.2 Hz, 1H), 2.76-2.63 (m, 1H), 2.19 (dd, J = 7.6, 5.6 Hz, 1H), 1.85-1.71 (m, 1H). [M + H] = 409.2. |
| 228 | 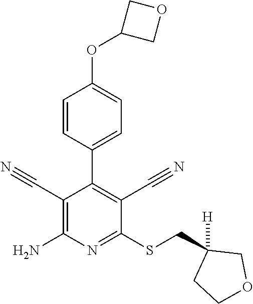 [SMILES: N#Cc1c(N)nc(SC[C@@H]2CCOC2)c(C#N)c1-c1ccc(OC2COC2)cc1] | ¹H-NMR (400 MHz, CD₃OD) δ = 7.55- 7.45 (m, 2H), 7.02-6.89 (m, 2H), 5.48- 5.32 (m, 1H), 5.07 (t, J = 7.2 Hz, 2H), 4.82- 4.69 (m, 2H), 3.99-3.88 (m, 2H), 3.84- 3.74 (m, 1H), 3.60 (dd, J = 8.4, 6.0 Hz, 1H), 3.40-3.38 (m, 1H), 3.37 (d, J = 1.2 Hz, 1H), 2.76-2.63 (m, 1H), 2.19 (dd, J = 7.6, 5.6 Hz, 1H), 1.85-1.71 (m, 1H). [M + H] = 409.2. |
| 25 | 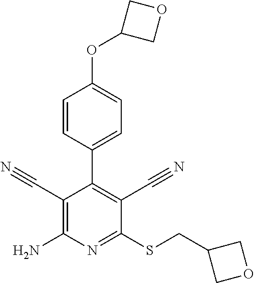 [SMILES: N#Cc1c(N)nc(SCC2COC2)c(C#N)c1-c1ccc(OC2COC2)cc1] | ¹H-NMR (400 MHz, DMSO-d₆) δ = 3.30- 3.34 (m, 1H) 3.53-3.59 (m, 2H) 4.33-4.40 (m, 2H) 4.56-4.63 (m, 2 H) 4.64-4.71 (m, 2H) 4.93-5.02 (m, 2H) 5.35-5.44 (m, 1H) 6.95-7.01 (m, 2H) 7.47-7.55 (m, 2H). [M + H] = 395.0. |
| 7 | 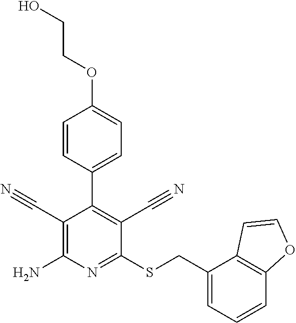 [SMILES: N#Cc1c(N)nc(SCc2cccc3occc23)c(C#N)c1-c1ccc(OCCO)cc1] | ¹H-NMR (400 MHz, DMSO-d₆) δ = 8.02 (d, J = 2.4 Hz, 1H), 7.54-7.43 (m, 4H), 7.29-7.22 (m, 1H), 7.19 (dd, J = 2.0, 1.6 Hz, 1H), 7.09 (d, J = 8.8 Hz, 2H), 4.90 (t, J = 5.6 Hz, 1H), 4.80 (s, 2H), 4.07 (t, J = 4.8 Hz, 2H), 3.74 (d, J = 5.2 Hz, 2H). ESI [M + H] = 433.3 |
| 69 | 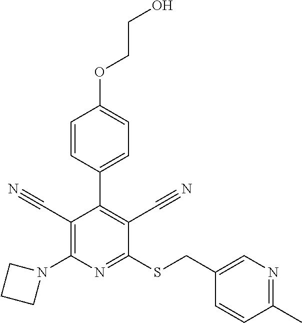 [SMILES: Cc1ccc(CSc2nc(N3CCC3)c(C#N)c(-c3ccc(OCCO)cc3)c2C#N)cn1] | ¹H-NMR (400 MHz, DMSO-d₆) δ = 8.50 (br. S, 1H), 7.41 (d, J = 8.8 Hz, 2H), 7.22 (br. S, 1H), 7.17-7.27 (m, 1H), 7.06 (d, J = 8.8 Hz, 2H), 4.89 (t, J = 5.2 Hz, 1H), 4.30-4.51 (m, 6H), 4.03 (t, J = 4.8 Hz, 2H), 3.70 (q, J = 4.8 Hz, 2H), 2.41 (s, 3H), 2.32- 2.38 (m, 2H). ESI [M + H] = 458.3. |
| 70 | 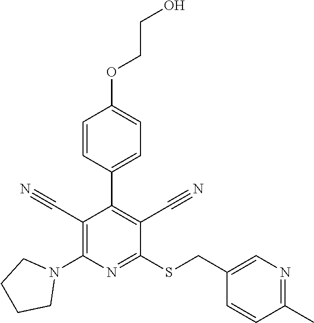 [SMILES: Cc1ccc(CSc2nc(N3CCCC3)c(C#N)c(-c3ccc(OCCO)cc3)c2C#N)cn1] | ¹H-NMR (400 MHz, DMSO-d₆) δ = 8.43- 8.57 (m, 1H), 7.69 (d, J = 7.2 Hz, 1H), 7.44 (d, J = 8.8 Hz, 2H), 7.20 (d, J = 7.2 Hz, 1H), 7.07 (d, J = 8.4 Hz, 2H), 4.89 (t, J = 5.6 Hz, 1H), 4.51 (s, 2H), 4.04 (t, J = 4.8 Hz, 2H), 3.66-3.85 (m, 7H), 2.40-2.42 (m, 1H), 2.41 (s, 3H), 1.92 (br. S., 5H). |
| 212 | 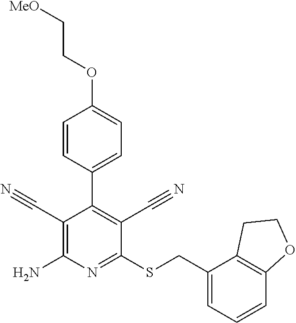 [SMILES: COCCOc1ccc(-c2c(C#N)c(N)nc(SCc3cccc4c3CCO4)c2C#N)cc1] | ¹H-NMR (400 MHz, DMSO-d₆) δ = 7.84- 8.30 (m, 2H), 7.44-7.52 (m, 2H), 7.08- 7.12 (m, 2H), 6.98-7.08 (m, 2H), 6.68 (d, J = 7.6 Hz, 1H), 4.55 (t, J = 8.8 Hz, 2H), 4.40 (s, 2H), 4.18 (dd, J = 5.4, 3.6 Hz, 2H), 3.67-3.71 (m, 2H), 3.32 (s, 3H), 3.22 (t, J = 8.6 Hz, 2H). [M + H] = 459.1. |
| 16 | 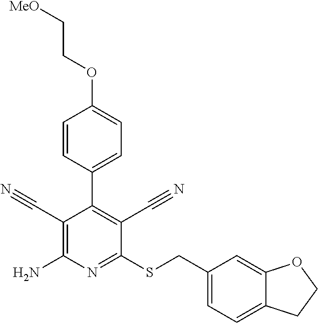 [SMILES: COCCOc1ccc(-c2c(C#N)c(N)nc(SCc3ccc4c(c3)OCC4)c2C#N)cc1] | ¹H-NMR (400 MHz, CDCl₃) δ = 7.46- 7.50 (m, 2H), 7.14 (d, J = 7.6 Hz, 1H), 7.06 (d, J = 8.8 Hz, 2H), 6.84-6.90 (m, 1 H), 6.83 (s, 1H), 5.65 (s, 2H), 4.58 (t, J = 8.6 Hz, 2H), 4.40 (s, 2H), 4.16-4.22 (m, 2H), 3.76-3.82 (m, 2H), 3.47 (s, 3H), 3.20 (t, J = 8.4 Hz, 2H). [M + H] = 459.1. |
| 213 | 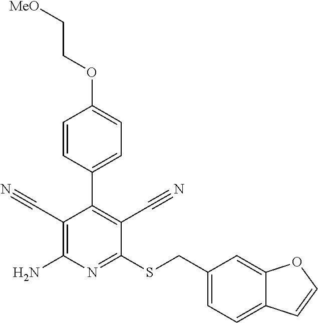 [SMILES: COCCOc1ccc(-c2c(C#N)c(N)nc(SCc3ccc4ccoc4c3)c2C#N)cc1] | ¹HNMR (400 MHz, DMSO-d₆) δ = 7.98- 8.02 (m, 1H), 7.84-7.88 (m, 1H), 7.62- 7.64 (m, 1H), 7.44-7.52 (m, 2H), 7.40- 7.46 (m, 1H), 7.08-7.16 (m, 2H), 6.92- 6.98 (m, 1 H), 4.60-4.68 (m, 2H), 4.14- 4.24 (m, 2H), 3.70 (dd, J = 5.2, 3.6 Hz, 2H), 3.34 (s, 3H). [M + H] = 457.1. |
| 231 | 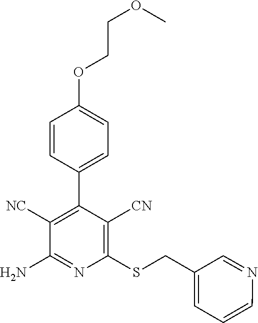 [SMILES: COCCOc1ccc(-c2c(C#N)c(N)nc(SCc3cccnc3)c2C#N)cc1] | [M + H] = 418.1 |
| 115 | 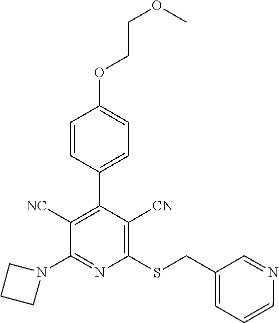 [SMILES: COCCOc1ccc(-c2c(C#N)c(SCc3cccnc3)nc(N3CCC3)c2C#N)cc1] | ¹H NMR (400 MHz, DMSO-d6) δ = 7.86 (br d, J = 7.4 Hz, 1H), 7.45 (br d, J = 8.5 Hz, 4H), 7.10 (br d, J = 8.6 Hz, 3H), 4.55 (s, 3H), 4.43 (br s, 4H), 4.21-4.14 (m, 3H), 3.72-3.64 (m, 3H), 2.39-2.33 (m, 2H). [M + H] = 458.2 |
| 123 | 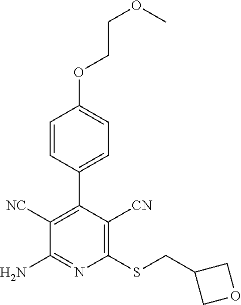 [SMILES: COCCOc1ccc(-c2c(C#N)c(N)nc(SCC3COC3)c2C#N)cc1] | ¹H-NMR (400 MHz, DMSO-d6) δ = 7.97 (br s, 1 H) 7.44 (d, J = 8.8 Hz, 2 H) 7.07 (d, J = 8.4 Hz, 2 H) 4.57-4.66 (m, 2 H) 4.32 (t, J = 6.4 Hz, 2 H) 4.11-4.19 (m, 2 H) 3.58- 3.73 (m, 2 H) 3.36-3.55 (m, 2 H) 3.27- 3.31 (m, 3 H). [M + H] = 397.2 |
| 218 | 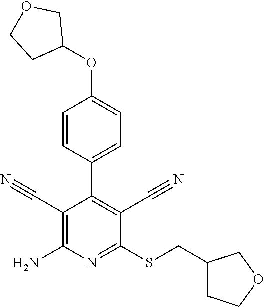 [SMILES: N#Cc1c(N)nc(SCC2CCOC2)c(C#N)c1-c1ccc(OC2CCOC2)cc1] | ¹H-NMR (400 MHz, DMSO-d₆) δ = 8.01 (br.s, 2H), 7.51 (d, J = 8.8 Hz, 2H), 7.11 (d, J = 8.8 Hz, 2H), 5.24-5.08 (m, 1H), 3.96-3.92 (m, 1H), 3.92-3.84 (m, 3H), 3.84-3.76 (m, 2H), 3.67 (q, J = 7.6 Hz, 1H), 3.44 (dd, J = 8.4, 6.4 Hz, 1H), 3.31 (d, J = 7.6 Hz, 2H), 2.64-2.56 (m, 1H), 2.36-2.24 (m, 1H), 2.14-1.96 (m, 2H), 1.72-1.60 (m, 1H). ESI [M + H] = 423.3. |
| 219 | 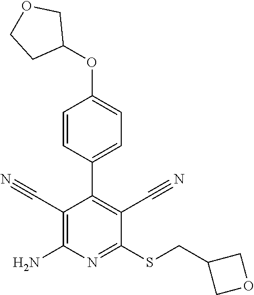 [SMILES: N#Cc1c(N)nc(SCC2COC2)c(C#N)c1-c1ccc(OC2CCOC2)cc1] | ¹H-NMR (400 MHz, DMSO-d₆) δ = 8.03 (br s, 2H), 7.52-7.48 (m, 2H), 7.12-7.08 (m, 2H), 5.20-5.04 (m, 1H), 4.66 (dd, J = 7.6, 6.4 Hz, 2H), 4.37 (t, J = 6.4 Hz, 2H), 3.96-3.92 (m, 1H), 3.88-3.80 (m, 2H), 3.80-3.76 (m, 1H), 3.56 (d, J = 7.2 Hz, 2H), 3.44-3.36 (m, 1H), 2.36-2.24 (m, 1H), 2.04-1.96 (m, 1H). ESI [M + H] = 409.2. |
| 220 | 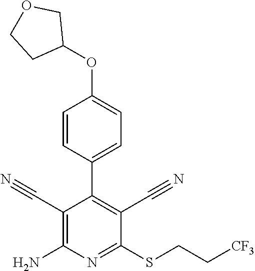 [SMILES: N#Cc1c(N)nc(SCCC(F)(F)F)c(C#N)c1-c1ccc(OC2CCOC2)cc1] | ¹H-NMR (400 MHz, DMSO-d₆) δ = 8.05 (br s, 2H), 7.50 (d, J = 8.8 Hz, 2H), 7.11 (d, J = 8.8 Hz, 2H), 5.14 (dd, J = 6.0, 4.4 Hz, 1H), 3.94 (dd, J = 10.4, 8.8 Hz, 1H), 3.92- 3.84 (m, 2H), 3.79 (d, J = 8.4, 4.0 Hz, 1H), 3.40-3.36 (m, 2H), 2.84-2.68 (m, 2H), 2.36-2.24 (m, 1H), 2.04-1.96 (m, 1H). ESI [M + H] = 435.12. |
| 232 | 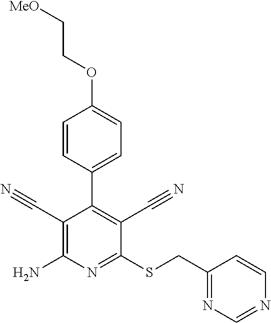 [SMILES: COCCOc1ccc(-c2c(C#N)c(N)nc(SCc3ccncn3)c2C#N)cc1] | ¹H NMR (400 MHz, DMSO-d₆) δ = 9.14 (dd, J = 4.8, 1.6 Hz, 1H), 8.46-7.97 (m, 2H), 7.95 (dd, J = 8.4, 1.6 Hz, 1H), 7.66 (dd, J = 8.4, 4.8 Hz, 1H), 7.50-7.46 (m, 2H), 7.13-7.09 (m, 2H), 4.77 (s, 2H), 4.18 (dd, J = 5.2, 3.6 Hz, 2H), 3.70-3.67 (m, 2H), 3.32 (s, 3H). [M + H] = 419.1 |
| 234 | 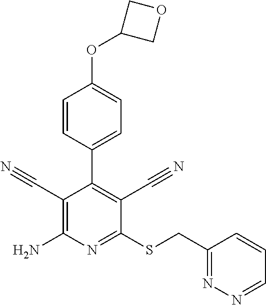 [SMILES: N#Cc1c(N)nc(SCc2cccnn2)c(C#N)c1-c1ccc(OC2COC2)cc1] | ¹H NMR (400 MHz, DMSO-d₆) δ 9.17- 9.13 (m, 1H), 7.98-7.95 (m, 1H), 7.70- 7.66 (m, 1H), 7.50 (d, J = 8.8 Hz, 2H), 6.97 (d, J = 8.8 Hz, 2H), 5.38 (t, J = 5.6 Hz, 1H), 4.96 (t, J = 6.8 Hz, 2H), 4.77 (s, 2H), 4.61-4.58 (m, 2H). [M + H] = 417.1 |
| 236 | 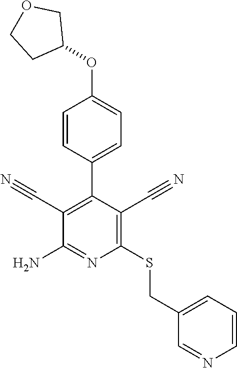 | ¹H NMR (400 MHz, DMSO-d₆) δ = 8.78 (d, J = 2.0 Hz, 1H), 8.47-8.43 (m, 1H), 7.94 (br d, J = 8.0 Hz, 1H), 7.48 (d, J = 8.8 Hz, 2H), 7.37-7.32 (m, 1H), 7.08 (d, J = 8.8 Hz, 2H), 5.11 (br t, J = 5.4 Hz, 1H), 4.49 (s, 2H), 3.94-3.89 (m, 1H), 3.86-3.73 (m, 3H), 2.33-2.21 (m, 1H), 2.03-1.94 (m, 1H) [M + H] = 430.1 |
| 333 | 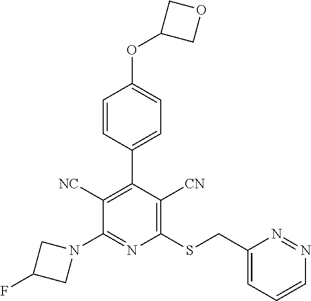 [SMILES: N#Cc1c(SCc2cccnn2)nc(N2CC(F)C2)c(C#N)c1-c1ccc(OC2COC2)cc1] | ¹H NMR (400 MHz, CD₃OD) δ = 9.12- 8.90 (m, 1H), 7.80 (d, J = 7.6 Hz, 1H), 7.61 (dd, J = 4.8, 8.8 Hz, 1H), 7.36 (d, J = 8.8 Hz, 2H), 6.88-6.76 (m, 2H), 5.41-5.20 (m, 2H), 4.94 (t, J = 6.4 Hz, 2H), 4.73 (s, 2H), 4.63 (br d, J = 2.0 Hz, 2H), 4.61-4.53 (m, 2H), 4.38-4.24 (m, 2H) |
| 238 | 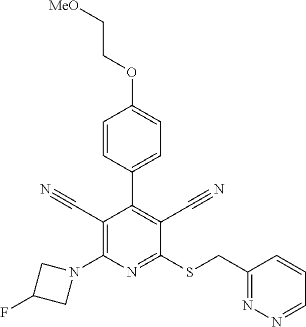 [SMILES: COCCOc1ccc(-c2c(C#N)c(SCc3cccnn3)nc(N3CC(F)C3)c2C#N)cc1] | ¹H NMR (400 MHz, DMSO-d₆) δ = 9.16 (dd, J = 4.8, 1.2 Hz, 1H), 7.82 (dd, J = 8.4, 1.2 Hz, 1H), 7.70 (dd, J = 8.4, 4.8 Hz, 1H), 7.48 (d, J = 8.8 Hz, 2H), 7.12 (d, J = 8.8 Hz, 2H), 5.61-5.37 (m, 1H), 4.83 (s, 2H), 4.75-4.61 (m, 2H), 4.47- 4.32 (m, 2H), 4.21-4.17 (m, 2H), 3.71- 3.68 (m, 2H), 3.34-3.33 (m, 3H) [M + H] = 477.2 |
| 240 | 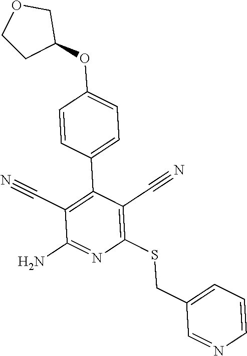 [SMILES: N#Cc1c(N)nc(SCc2cccnc2)c(C#N)c1-c1ccc(O[C@H]2CCOC2)cc1] | ¹H NMR (400 MHz, DMSO-d₆) δ = 8.81- 8.74 (m, 1H), 8.45 (dd, J = 1.2, 4.8 Hz, 1H), 7.95 (br d, J = 7.6 Hz, 1H), 7.49- 7.44 (m, 1H), 7.37-7.32 (m, 1H), 7.11- 7.04 (m, 2H), 5.15-5.07 (m, 1H), 4.53- 4.42 (m, 2H), 3.93-3.69 (m, 4H), 2.33- 2.21 (m, 1H), 2.06-1.92 (m, 1H) |
| ^{b}243 | 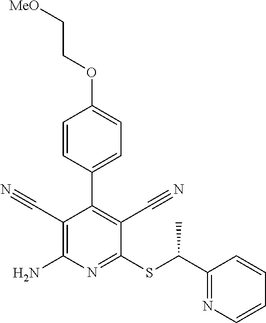 [SMILES: COCCOc1ccc(-c2c(C#N)c(N)nc(S[C@H](C)c3ccccn3)c2C#N)cc1] | ¹H NMR (400 MHz, CD₃OD) δ = 8.50 (d, J = 4.4 Hz, 1H), 7.86-7.78 (m, 1H), 7.65 (d, J = 8.0 Hz, 1H), 7.45 (d, J = 8.8 Hz, 2H), 7.35-7.28 (m, 1H), 7.14-7.05 (m, 2H), 5.44-5.30 (m, 1H), 4.25-4.15 (m, 2H), 3.84-3.69 (m, 2H), 3.43 (s, 3H), 1.78 (d, J = 7.2 Hz, 3H) |
| ^{b}244 | 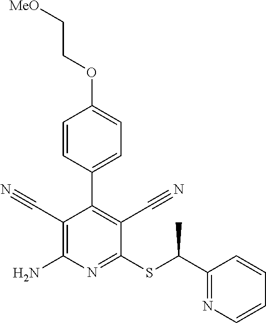 [SMILES: COCCOc1ccc(-c2c(C#N)c(N)nc(S[C@@H](C)c3ccccn3)c2C#N)cc1] | ¹H NMR (400 MHz, CD₃OD) δ = 8.52- 8.48 (m, 1H), 7.85-7.78 (m, 1H), 7.65 (d, J = 8.0 Hz, 1H), 7.48-7.42 (m, 2H), 7.35-7.29 (m, 1H), 7.12-7.05 (m, 2H), 5.41-5.32 (m, 1H), 4.24-4.16 (m, 2H), 3.82-3.74 (m, 2H), 3.43 (s, 3H), 1.77 (d, J = 7.2 Hz, 3H) |
| a245 | 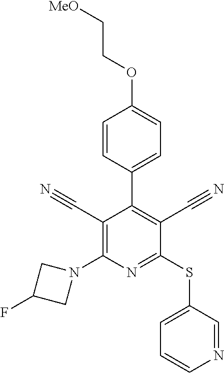 [SMILES: COCCOc1ccc(-c2c(C#N)c(Sc3cccnc3)nc(N3CC(F)C3)c2C#N)cc1] | ¹H NMR (400 MHz, DMSO-d₆) δ 8.73 (d, J = 2.4 Hz, 2H), 8.08 (td, J = 8.0, 2.0 Hz, 1H), 7.59 (dd, J = 8.0, 4.8 Hz, 1H), 7.51 (d, J = 8.8 Hz, 2H), 7.16 (d, J = 8.8 Hz, 2H), 5.48-5.28 (m, 1H), 4.54-4.22 (m, 2H), 4.21 (dd, J = 5.2, 3.6 Hz, 2H), 4.08 (br s, 2H), 3.71 (dd, J = 5.2, 4.0 Hz, 2H), 3.34 (s, 3H). [M + H] = 462.2 |
| ^{a}246 | 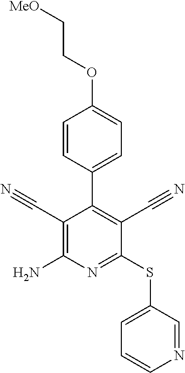 [SMILES: COCCOc1ccc(-c2c(C#N)c(N)nc(Sc3cccnc3)c2C#N)cc1] | ¹H NMR (400 MHz, DMSO-d₆) δ 8.77- 8.64 (m, 2H), 8.06 (td, J = 8.0, 1.6 Hz, 1H), 7.82 (br s, 2H), 7.58-7.49 (m, 3H), 7.17-7.12 (m, 2H), 4.20 (dd, J = 5.2, 3.6 Hz, 2H), 3.73-3.68 (m, 2H), 3.34 (s, 3H). [M + H] = 404.1 |
| ^{a}337 | 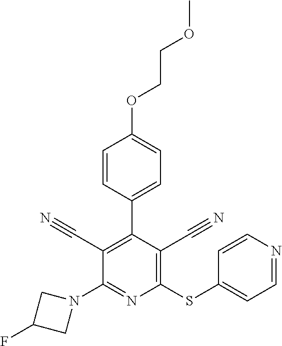 [SMILES: COCCOc1ccc(-c2c(C#N)c(Sc3ccncc3)nc(N3CC(F)C3)c2C#N)cc1] | ¹H NMR (400 MHz, DMSO-d₆) δ = 8.69 (br d, J = 4.0 Hz, 1H), 8.66-8.60 (m, 1H), 7.67 (d, J = 6.0 Hz, 2H), 7.51 (d, J = 8.8 Hz, 2H), 7.15 (d, J = 8.8 Hz, 2H), 5.47 (qd, J = 5.6, 2.8 Hz, 1H), 5.33 (td, J = 5.6, 2.8 Hz, 1H), 4.58-4.33 (m, 2H), 4.27-4.09 (m, 4H), 3.72-3.67 (m, 2H). [M + H] = 462.2. |
| ^{b}247 | 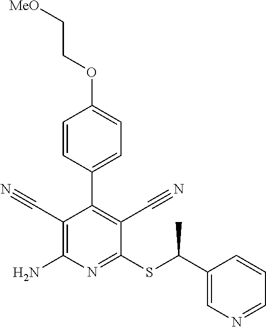 [SMILES: COCCOc1ccc(-c2c(C#N)c(N)nc(S[C@@H](C)c3cccnc3)c2C#N)cc1] | ¹H NMR (400 MHz, CD₃OD ) δ = 8.74 (d, J = 2.0 Hz, 1H), 8.42 (dd, J = 4.8, 1.6 Hz, 1H), 8.04 (td, J = 8.0, 2.0 Hz, 1H), 7.47-7.39 (m, 3H), 7.08 (d, J = 8.8 Hz, 2H), 5.29 (q, J = 7.2 Hz, 1H), 4.19 (dd, J = 5.2, 3.2 Hz, 2H), 3.81 (s, 2H), 3.43 (s, 3H), 1.78 (d, J = 7.2 Hz, 3H) |
| ^{b}248 | 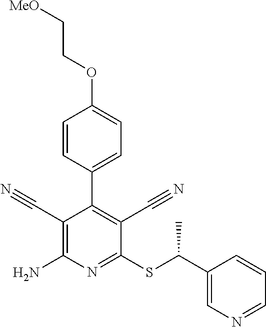 [SMILES: COCCOc1ccc(-c2c(C#N)c(N)nc(S[C@H](C)c3cccnc3)c2C#N)cc1] | ¹H NMR (400 MHz, CD₃OD) δ = 8.76- 8.72 (m, 1H), 8.41 (dd, J = 4.8, 1.2 Hz, 1H), 8.08-8.00 (m, 1H), 7.47-7.38 (m, 3H), 7.08 (d, J = 8.8 Hz, 2H), 5.29 (q, J = 7.2 Hz, 1H), 4.18 (d, J = 4.8 Hz, 2H), 3.79- 3.72 (m, 2H), 3.43 (s, 3H), 1.82-1.73 (m, 3H) |
| ^{b}331 | 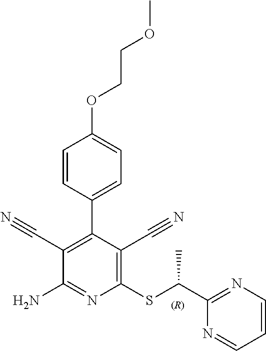 | ¹H NMR (400 MHz, CD₃OD) δ = 8.79 (d, 4.8 Hz, 2H), 7.46 (d, 8.8 Hz, 2H), 7.40 (t, 4.4 Hz, 1H), 7.10 (d, 8.4 Hz, 2H), 5.45 (q, 7.2 Hz, 1H), 4.23-4.18 (m, 2H), 3.80-3.76 (m, 2H), 3.44 (s, 3H), 1.81 (d, 7.2 Hz, 3H) |
| ^{b}332 | 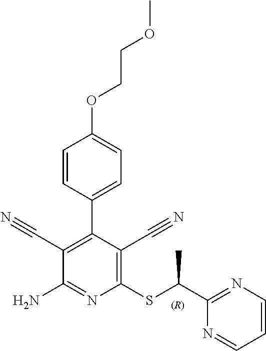 | ¹H NMR (400 MHz, CD₃OD) δ = 8.79 (d, 5.2 Hz, 2H), 7.46 (d, 8.8 Hz, 2H), 7.40 (t, 4.8 Hz, 1H), 7.10 (d, 8.8 Hz, 2H), 5.45 (q, 7.2 Hz, 1H), 4.22-4.19 (m, 2H), 3.80-3.76 (m, 2H), 3.44 (s, 3H), 1.81 (d, 7.2 Hz, 3H) |
| ^{b}335 | 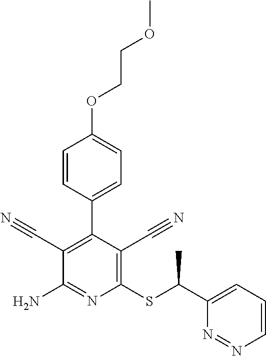 [SMILES: COCCOc1ccc(-c2c(C#N)c(N)nc(S[C@@H](C)c3cccnn3)c2C#N)cc1] | ¹H NMR (400 MHz, CD₃OD) δ = 9.09 (dd, 4.8, 1.2 Hz, 1H), 8.00 (dd, 8.4, 1.2 Hz, 1H), 7.71 (dd, 8.4, 5.2 Hz, 1H), 7.45 (d, 8.4 Hz, 2H), 7.09 (d, 8.4 Hz, 2H), 5.52 (q, 7.2 Hz, 1H), 4.22-4.18 (m, 2H), 3.79-3.76 (m, 2H), 3.43 (s, 3H), 1.85 (d, 7.2 Hz, 3H) |
| b336 | 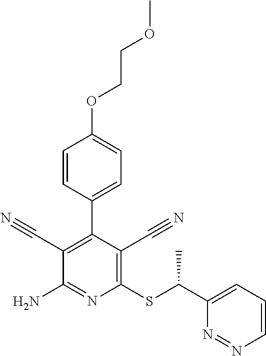 [SMILES: COCCOc1ccc(-c2c(C#N)c(N)nc(S[C@H](C)c3cccnn3)c2C#N)cc1] | ¹H NMR (400 MHz, CD₃OD) δ = 9.09 (dd, 4.8, 1.2 Hz, 1H), 8.00 (dd, 8.4, 1.2 Hz, 1H), 7.71 (dd, 8.4, 5.2 Hz, 1H), 7.45 (d, 8.8 Hz, 2H), 7.09 (d, 8.8 Hz, 2H), 5.52 (q, 7.2 Hz, 1H), 4.21-4.18 (m, 2H), 3.79-3.76 (m, 2H), 3.43 (s, 3H), 1.85 (d, 7.2 Hz, 3H) |
| 249 | 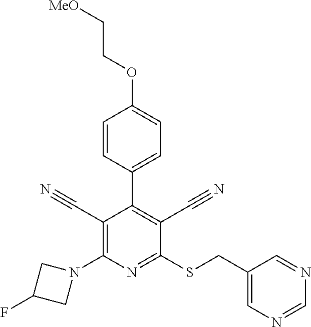 [SMILES: COCCOc1ccc(-c2c(C#N)c(SCc3cncnc3)nc(N3CC(F)C3)c2C#N)cc1] | ¹H NMR (400 MHz, DMSO-d₆) δ = 9.15- 9.04 (m, 1H), 8.93-8.81 (m, 2H), 7.52- 7.38 (m, 2H), 7.18-7.03 (m, 2H), 5.64- 5.33 (m, 1H), 4.80-4.66 (m, 2H), 4.56 (br s, 2H), 4.50-4.38 (m, 2H), 4.21- 4.15 (m, 2H), 3.71-3.65 (m, 2H), 3.33 (br s, 3H). [M + H] = 477.2 |
| 250 | 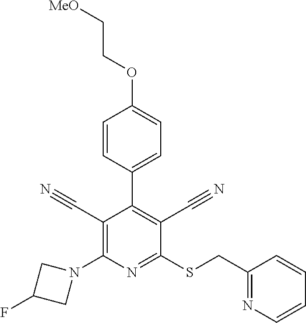 [SMILES: COCCOc1ccc(-c2c(C#N)c(SCc3ccccn3)nc(N3CC(F)C3)c2C#N)cc1] | ¹H NMR (400 MHz, DMSO-d₆) δ = 8.54 (d, J = 4.2 Hz, 1H), 7.79 (dt, J = 1.6, 7.6 Hz, 1H), 7.53 (d, J = 7.6 Hz, 1H), 7.48 (d, J = 8.8 Hz, 2H), 7.31 (dd, J = 5.2, 7.2 Hz, 1H), 7.12 (d, J = 8.8 Hz, 2H), 5.59- 5.40 (m, 1H), 4.79-4.67 (m, 2H), 4.64 (s, 2H), 4.49-4.37 (m, 2H), 4.21-4.17 (m, 2H), 3.72-3.68 (m, 2H), 3.34-3.33 (m, 3H). [M + H] = 476.2 |
| 251 | 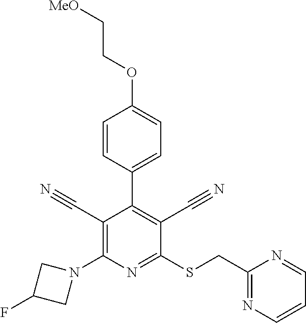 [SMILES: COCCOc1ccc(-c2c(C#N)c(SCc3ncccn3)nc(N3CC(F)C3)c2C#N)cc1] | ¹H NMR (400 MHz, DMSO-d₆) δ = 8.81 (d, J = 5.2 Hz, 2H), 7.48 (d, J = 8.8 Hz, 2H), 7.45 (t, J = 4.8 Hz, 1H), 7.13 (d, J = 8.8 Hz, 2H), 5.55-5.37 (m, 1H), 4.75 (s, 2H), 4.70-4.57 (m, 2H), 4.39-4.26 (m, 2H), 4.22-4.18 (m, 2H), 3.71-3.67 (m, 2H), 3.35-3.33 (m, 3H) [M + H] = 477.2 |
| 252 | 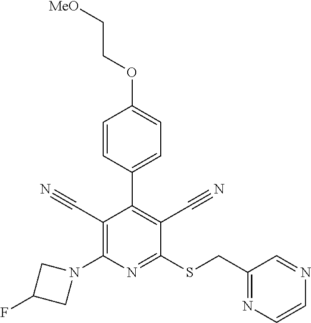 [SMILES: COCCOc1ccc(-c2c(C#N)c(SCc3cnccn3)nc(N3CC(F)C3)c2C#N)cc1] | ¹H NMR (400 MHz, DMSO-d₆) δ = 8.81 (s, 1H), 8.67-8.59 (m, 1H), 8.56 (d, J = 2.4 Hz, 1H), 7.50-7.44 (m, 2H), 7.11 (d, J = 8.8 Hz, 2H), 5.60-5.38 (m, 1H), 4.70 (s, 4H), 4.47-4.32 (m, 2H), 4.18 (dd, J = 5.4, 3.6 Hz, 2H), 3.71-3.66 (m, 2H), 3.32 (s, 3H) [M + H] = 477.2 |
| 267 | 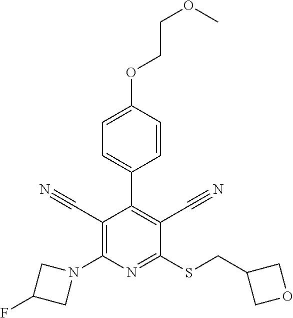 [SMILES: COCCOc1ccc(-c2c(C#N)c(SCC3COC3)nc(N3CC(F)C3)c2C#N)cc1] | ¹H NMR (400 MHz, DMSO-d₆) δ = 7.49 (d, J = 8.8 Hz, 2 H), 7.19-7.11 (m, 2 H), 5.67-5.33 (m, 1 H), 5.02-4.61 (m, 4 H), 4.57-4.41 (m, 2 H), 4.37 (t, J = 6.0 Hz, 2 H), 4.23-4.18 (m, 2 H), 3.78-3.66 (m, 2 H), 3.58 (d, J = 7.2 Hz, 2 H), 3.43-3.36 (m, 1 H), 3.34-3.33 (m, 3 H). [M + H] = 455.2 |
| 268 | 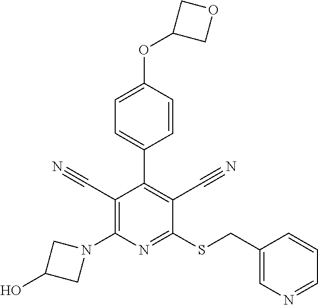 [SMILES: N#Cc1c(SCc2cccnc2)nc(N2CC(O)C2)c(C#N)c1-c1ccc(OC2COC2)cc1] | ¹H-NMR (400 MHz, DMSO-d₆) δ 8.66 (s, 1H), 8.48 (d, J = 4.0 Hz, 1H), 7.85 (br d, J = 8.0 Hz, 1H), 7.48 (d, J = 8.8 Hz, 2H), 7.38 (dd, J = 7.6, 4.8 Hz, 1H), 6.97 (d, J = 8.8 Hz, 2H), 5.90 (d, J = 5.6 Hz, 1H), 5.42-5.34 (m, 1H), 4.96 (t, J = 6.8 Hz, 2H), 4.65 (br s, 1H), 4.59 (dd, J = 7.2, 5.2 Hz, 4H), 4.55-4.49 (m, 2H), 4.18- 4.06 (m, 2H). [M + H] = 472.5 |
| 269 | 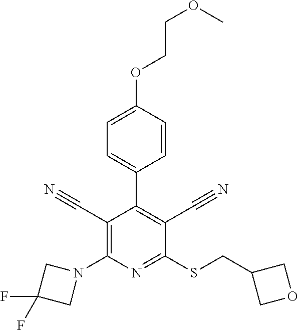 [SMILES: COCCOc1ccc(-c2c(C#N)c(SCC3COC3)nc(N3CC(F)(F)C3)c2C#N)cc1] | ¹H NMR (400 MHz, DMSO-d₆) δ = 7.49 (br d, J = 8.4 Hz, 2H), 7.14 (br d, J = 8.8 Hz, 2H), 4.86 (br t, J = 12.0 Hz, 4H), 4.68 (brt, J = 6.8 Hz, 2H), 4.36 (t, J = 6.0 Hz, 2H), 4.24-4.16 (m, 2H), 3.73-3.67 (m, 2H), 3.59 (br d, J = 7.6 Hz, 2H), 3.41- 3.37 (m, 1H), 3.36-3.34 (m, 3H), 2.54- 2.51 (m, 3H). [M + H] = 473.5 |
| 270 | 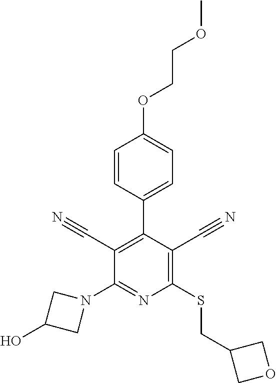 [SMILES: COCCOc1ccc(-c2c(C#N)c(SCC3COC3)nc(N3CC(O)C3)c2C#N)cc1] | ¹H NMR (400 MHz, DMSO) δ = 7.47 (d, J = 8.8 Hz, 2H), 7.12 (d, J = 8.8 Hz, 2H), 5.89 (d, J = 5.6 Hz, 1H), 4.67 (dd, J = 7.6, 6.0 Hz, 3H), 4.62 (br s, 2H), 4.36 (t, J = 6.4 Hz, 2H), 4.19 (br d, J = 4.4 Hz, 2H), 4.13 (br s, 2H), 3.72-3.67 (m, 2H), 3.56 (d, J = 7.2 Hz, 2H), 3.37 (br d, J = 7.2 Hz, 1H), 3.33 (s, 3H). [M + H] = 453.2 |
| 271 | 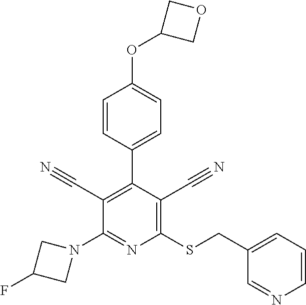 [SMILES: N#Cc1c(SCc2cccnc2)nc(N2CC(F)C2)c(C#N)c1-c1ccc(OC2COC2)cc1] | ¹H NMR (400 MHz, DMSO) δ = 8.96- 8.22 (m, 2H), 7.87 (br d, J = 7.2 Hz, 1H), 7.49 (d, J = 8.8 Hz, 2H), 7.47-7.35 (m, 1H), 6.98 (d, J = 8.8 Hz, 2H), 5.61-5.37 (m, 2H), 4.96 (t, J = 6.8 Hz, 2H), 4.83- 4.70 (m, 2H), 4.61-4.57 (m, 4H), 4.53- 4.42 (m, 2H). [M + H] = 474.1 |
| 272 | 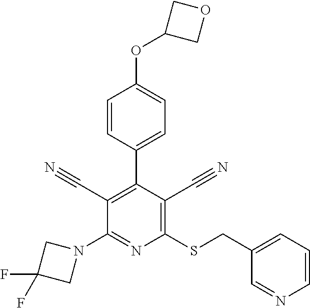 [SMILES: N#Cc1c(SCc2cccnc2)nc(N2CC(F)(F)C2)c(C#N)c1-c1ccc(OC2COC2)cc1] | ¹H NMR (400 MHz, DMSO) δ = 8.68 (br s, 1H), 8.49 (br d, J = 3.6 Hz, 1H), 7.87 (br d, J = 7.2 Hz, 1H), 7.51 (br d, J = 8.4 Hz, 2H), 7.39 (br dd, J = 4.8, 7.2 Hz, 1H), 7.00 (br d, J = 8.8 Hz, 2H), 5.44- 5.36 (m, 1H), 4.97 (br t, J = 6.4 Hz, 2H), 4.86 (br t, J = 12.4 Hz, 4H), 4.62-4.55 (m, 4H). [M + H] = 492.1 M + H] = 512.2 |
| 273 | 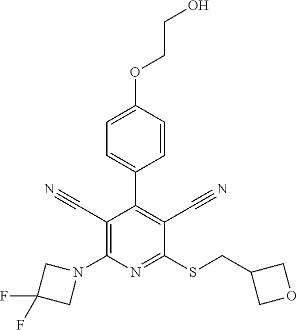 [SMILES: N#Cc1c(SCC2COC2)nc(N2CC(F)(F)C2)c(C#N)c1-c1ccc(OCCO)cc1] | ¹H NMR (400 MHz, DMSO-d₆) δ = 7.49 (br d, J = 8.0 Hz, 2H), 7.13 (br d, J = 8.4 Hz, 2H), 4.94-4.82 (m, 5H), 4.68 (br t, J = 6.8 Hz, 2H), 4.36 (br t, J = 5.6 Hz, 2H), 4.09 (br s, 2H), 3.75 (br d, J = 4.4 Hz, 2H), 3.59 (br d, J = 7.2 Hz, 2H), 3.40- 3.37 (m, 1H). [M + H] = 459.1 |
| 274 | 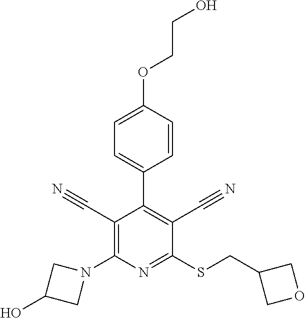 [SMILES: N#Cc1c(SCC2COC2)nc(N2CC(O)C2)c(C#N)c1-c1ccc(OCCO)cc1] | ¹H NMR (400 MHz, DMSO-d₆) δ = 7.53- 7.34 (m, 2H), 7.10 (d, J = 8.8 Hz, 2H), 5.89 (d, J = 5.6 Hz, 1H), 4.91 (t, J = 5.6 Hz, 1H), 4.75-4.51 (m, 5H), 4.35 (t, J = 6.0 Hz, 2H), 4.20-4.00 (m, 4H), 3.74 (q, J = 5.2 Hz, 2H), 3.55 (d, J = 7.6 Hz, 2H), 3.36 (br s, 1H) [M + H] = 439.1 |
| 275 | 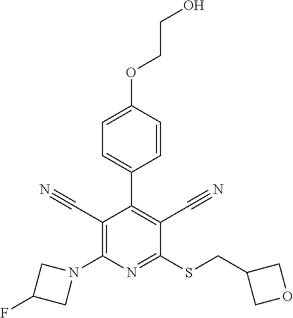 [SMILES: N#Cc1c(SCC2COC2)nc(N2CC(F)C2)c(C#N)c1-c1ccc(OCCO)cc1] | ¹H NMR (400 MHz, DMSO-d₆) δ 7.48 (d, J = 8.8 Hz, 2 H), 7.12 (d, J = 8.8 Hz, 2 H), 5.62-5.43 (m, 1 H), 4.92 (br s, 1 H), 4.83- 4.70 (m, 2 H), 4.68 (dd, J = 7.6, 6.0 Hz, 2 H), 4.54-4.41 (m, 2 H), 4.36 (t, J = 6.0 Hz, 2 H), 4.09 (t, J = 4.8 Hz, 2 H), 3.75 (br s, 2 H), 3.58 (d, J = 7.6 Hz, 2 H), 3.43- 3.37 (m, 1 H). [M + H] = 441.1 |
| 276 | 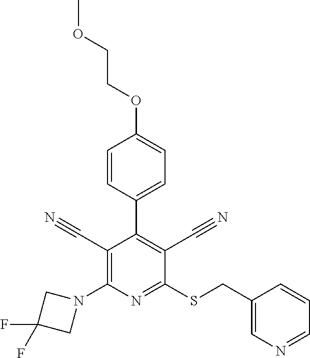 [SMILES: COCCOc1ccc(-c2c(C#N)c(SCc3cccnc3)nc(N3CC(F)(F)C3)c2C#N)cc1] | ¹H-NMR (400 MHz, CD₃OD) δ = 8.66 (br s, 1 H), 8.48 (br d, J = 3.2 Hz, 1 H), 7.97 (br d, J = 8.0 Hz, 1 H), 7.50 (d, J = 8.8 Hz, 2 H), 7.45 (dd, J = 8.0, 4.8 Hz, 1 H), 7.13 (d, J = 8.8 Hz, 2 H), 4.83 (t, J = 12.0 Hz, 4 H), 4.61 (s, 2 H), 4.23 (dd, J = 5.2, 4.0 Hz, 2 H), 3.82-3.78 (m, 2 H), 3.46 (s, 3 H). [M + H] = 494.2 |
| 277 | 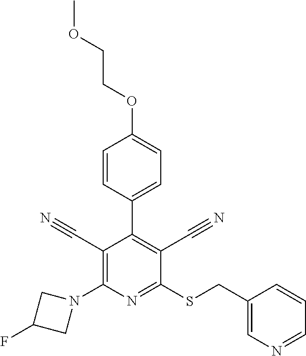 [SMILES: COCCOc1ccc(-c2c(C#N)c(SCc3cccnc3)nc(N3CC(F)C3)c2C#N)cc1] | ¹H-NMR (400 MHz, DMSO-d₆) δ = 8.67 (d, J = 2.0 Hz, 1H), 8.48 (dd, J = 4.8, 1.6 Hz, 1H), 7.89-7.83 (m, 1H), 7.47 (d, J = 8.8 Hz, 2H), 7.39 (dd, J = 8.0, 4.8 Hz, 1H), 7.12 (d, J = 8.8 Hz, 2H), 5.62-5.42 (m, 1H), 4.82-4.68 (m, 2H), 4.57 (s, 2H), 4.53-4.41 (m, 2H), 4.20-4.17 (m, 2H), 3.71-3.67 (m, 2H), 3.33-3.32 (m, 3H). [M + H] = 476.2 |
| 278 | 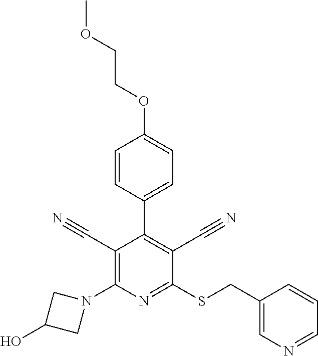 [SMILES: COCCOc1ccc(-c2c(C#N)c(SCc3cccnc3)nc(N3CC(O)C3)c2C#N)cc1] | ¹H-NMR (400 MHz, DMSO-d₆) δ = 8.73- 8.41 (m, 2 H), 7.85 (br d, J = 7.6 Hz, 1 H), 7.48-7.44 (m, 2 H), 7.41-7.36 (m, 1 H), 7.13-7.09 (m, 2 H), 5.91 (d, J = 5.6 Hz, 1 H), 4.69-4.63 (m, 1 H), 4.63-4.56 (m, 2 H), 4.55 (s, 2 H), 4.20-4.10 (m, 4 H), 3.71-3.67 (m, 2 H), 3.32 (s, 3 H). [M + H] = 474.2 |
| 279 | 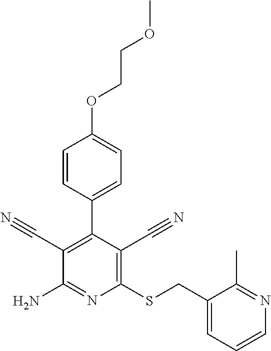 [SMILES: COCCOc1ccc(-c2c(C#N)c(N)nc(SCc3cccnc3C)c2C#N)cc1] | ¹H NMR (400 MHz, DMSO-d₆) δ = 8.35 (dd, J = 4.8, 1.6 Hz, 1H), 7.97 (dd, J = 7.6, 1.6 Hz, 1H), 7.51-7.43 (m, 2H), 7.17 (dd, J = 7.6, 4.8 Hz, 1H), 7.13-7.06 (m, 2H), 4.54 (s, 2H), 4.17 (dd, J = 5.2, 3.6 Hz, 2H), 3.71-3.65 (m, 2H), 3.32 (s, 3H), 2.55 (s, 3H). [M + H] = 432.2 |
| 280 | 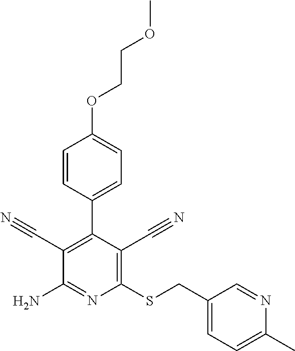 [SMILES: COCCOc1ccc(-c2c(C#N)c(N)nc(SCc3ccc(C)nc3)c2C#N)cc1] | ¹H-NMR (400 MHz, DMSO-d₆) δ = 8.64 (d, J = 2.0 Hz, 1 H), 7.82 (dd, J = 8.0, 2.4 Hz, 1 H), 7.47 (d, J = 8.8 Hz, 2 H), 7.20 (d, J = 8.0 Hz, 1 H), 7.10 (d, J = 8.8 Hz, 2 H), 4.45 (s, 2 H), 4.22-4.14 (m, 2 H), 3.72- 3.66 (m, 2 H), 3.32 (s, 3 H), 2.43 (s, 3 H). [M + H] = 432.2 |
| 284 | 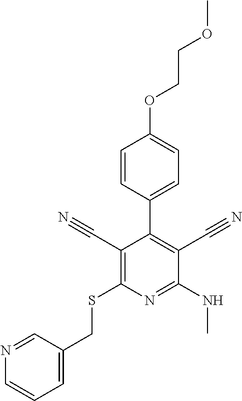 [SMILES: CNc1nc(SCc2cccnc2)c(C#N)c(-c2ccc(OCCOC)cc2)c1C#N] | ¹H NMR (400 MHz, CD₃OD) δ = 8.67 (s, 1H), 8.46 (br d, J = 4.4 Hz, 1H), 8.00- 7.95 (m, 1H), 7.97 (br d, J = 7.2 Hz, 1H), 7.48 (d, J = 8.8 Hz, 2H), 7.44 (dd, J = 7.2, 5.2 Hz, 1H), 7.12 (d, J = 8.8 Hz, 1H), 7.14- 7.09 (m, 1H), 4.65 (s, 2H), 4.24-4.20 (m, 2H), 3.82-3.77 (m, 2H), 3.45 (s, 3H), 3.09 (s, 3H). [M + H] = 432.2 |
| 285 | 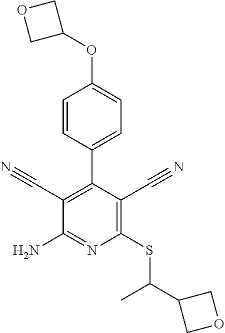 [SMILES: CC(Sc1nc(N)c(C#N)c(-c2ccc(OC3COC3)cc2)c1C#N)C1COC1] | ¹H NMR (400 MHz, CDCl₃) δ ppm 7.41 (d, J = 8.4 Hz, 2H), 6.77 (d, J = 8.4 Hz, 2H), 5.62 (s, 2H), 5.20 (t, J = 5.6 Hz, 1H), 4.93 (t, J = 6.4 Hz, 2H), 4.80-4.69 (m, 4H), 4.44 (t, J = 6.4 Hz, 2H), 4.40- 4.29 (m, 1H), 3.21-3.09 (m, 1H), 1.34 (d, J = 6.4 Hz, 3H) [M + H] = 409.1 |
| 286 | 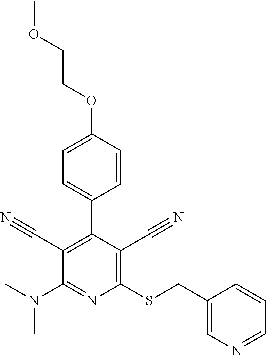 [SMILES: COCCOc1ccc(-c2c(C#N)c(SCc3cccnc3)nc(N(C)C)c2C#N)cc1] | ¹H-NMR (400 MHz, CD₃OD) δ = 8.47- 9.03 (m, 2H), 7.98 (br d, J = 8.4 Hz, 1H), 7.50 (br d, J = 8.8 Hz, 3H), 7.12 (d, J = 8.8 Hz, 2H), 4.65 (s, 2H), 4.21-4.24 (m, 2H), 3.79-3.82 (m, 2H), 3.46 (s, 3H), 3.40 (s, 6H). [M + H] = 446.2 |
| 291 | 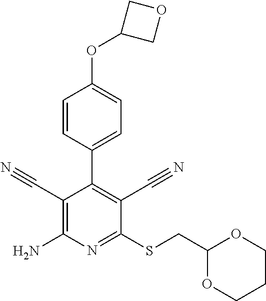 [SMILES: N#Cc1c(N)nc(SCC2OCCCO2)c(C#N)c1-c1ccc(OC2COC2)cc1] | ¹H NMR (400 MHz, DMSO-d₆) δ = 8.40- 7.66 (m, 2H), 7.50 (d, J = 8.8 Hz, 2H), 6.97 (d, J = 8.8 Hz, 2H), 5.38 (m, J = 5.6 Hz, 1H), 4.96 (t, J = 6.8 Hz, 2H), 4.79 (t, J = 4.8 Hz, 1H), 4.62-4.57 (m, 2H), 4.03 (br dd, J = 11.2, 4.4 Hz, 2H), 3.76 (br t, J = 11.1 Hz, 2H), 3.45 (d, J = 4.8 Hz, 2H), 3.33 (s, 3H), 1.95-1.83 (m, 1H), 1.36 (br d, J = 13.4 Hz, 1H). [M + H] = 425.2 |
| 292 | 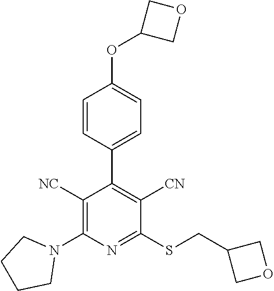 [SMILES: N#Cc1c(SCC2COC2)nc(N2CCCC2)c(C#N)c1-c1ccc(OC2COC2)cc1] | ¹H-NMR (400 MHz, DMSO-d₆) δ = 7.49 (d, J = 8.8 Hz, 2H) 6.97 (d, J = 8.8 Hz, 2 H) 5.39 (t, J = 5.2 Hz, 1H) 4.97 (t, J = 6.4 Hz, 2H) 4.67 (dd, J = 7.6, 6.0 Hz, 2H) 4.60 (dd, J = 7.2, 5.2 Hz, 2H) 4.35 (t, J = 6.0 Hz, 2H) 3.86-3.76 (m, 1H) 3.81 (br s, 3H) 3.58 (d, J = 7.6 Hz, 2H) 3.42-3.35 (m, 1H) 3.30 (s, 1H) 1.97 (br s, 4H). [M + H] = 449.2 |
| 293 | 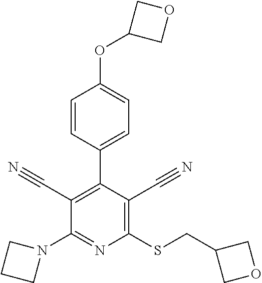 [SMILES: N#Cc1c(SCC2COC2)nc(N2CCC2)c(C#N)c1-c1ccc(OC2COC2)cc1] | ¹H-NMR (400 MHz, CDCl₃) δ = 7.34 (d, J = 8.8 Hz, 2H), 6.73 (d, J = 8.8 Hz, 2H), 5.18 (q, J = 5.6 Hz, 1H), 4.92 (t, J = 6.8 Hz, 2H), 4.74-4.78 (m, 2H), 4.70- 4.74 (m, 2H), 4.42-4.57 (m, 3H), 4.38 (t, J = 6.0 Hz, 3H), 3.47 (d, J = 7.6 Hz, 2H), 3.29 (s, 1H), 2.40 (br t, J = 7.6 Hz, 2H). [M + H] = 435.5 |
| 297 | 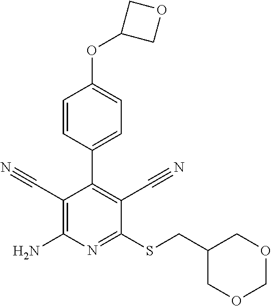 [SMILES: N#Cc1c(N)nc(SCC2COCOC2)c(C#N)c1-c1ccc(OC2COC2)cc1] | ¹H NMR (400 MHz, CD₃OD) δ = 7.52 (d, J = 8.8 Hz, 2 H) 6.87 (d, J = 8.4 Hz, 2 H) 5.69 (s, 2 H) 5.30 (t, J = 5.2 Hz, 1 H) 5.04 (t, J = 6.4 Hz, 2 H) 4.93-4.90 (m, 1 H) 4.89-4.86 (m, 1 H) 4.86-4.82 (m, 2 H) 4.11 (dd, J = 11.2, 3.2 Hz, 2 H) 3.82 (dd, J = 11.2, 6.0 Hz, 2 H) 3.37 (d, J = 7.2 Hz, 2 H) 2.15 (dt, J = 6.4, 3.2 Hz, 1 H); [M + H] = 425.1 |
| 301 | 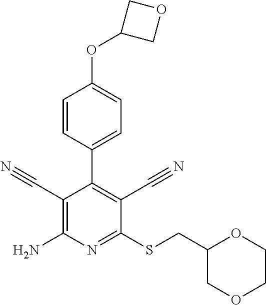 [SMILES: N#Cc1c(N)nc(SCC2COCCO2)c(C#N)c1-c1ccc(OC2COC2)cc1] | ¹H NMR (400 MHz, DMSO-d₆) δ 7.61- 7.40 (m, 2 H), 7.13-6.74 (m, 2 H), 5.80- 5.19 (m, 1 H), 4.97 (t, J = 6.8 Hz, 2 H), 4.60 (dd, J = 7.6, 5.2 Hz, 2 H), 3.88 (dd, J = 11.2, 2.4 Hz, 1 H), 3.80-3.68 (m, 2 H), 3.68-3.61 (m, 1 H), 3.60-3.53 (m, 1 H), 3.48 (dd, J = 10.8, 2.8 Hz, 1 H), 3.42 (dd, J = 14.0, 4.0 Hz, 1 H), 3.31- 3.21 (m, 1 H), 3.16 (dd, J = 14.0, 7.6 Hz, 1 H). [M + H] = 425.1 |
| 302 | 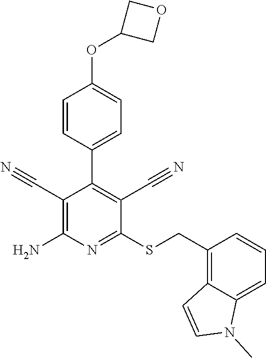 [SMILES: Cn1ccc2c(CSc3nc(N)c(C#N)c(-c4ccc(OC5COC5)cc4)c3C#N)cccc21] | ¹H-NMR (400 MHz, DMSO-d₆) δ = 7.45 (d, J = 8.8 Hz, 2H), 7.31-7.36 (m, 2H), 7.18 (d, J = 7.2 Hz, 1H), 7.03-7.09 (m, 1H), 6.92 (d, J = 8.8 Hz, 2H), 6.53 (d, J = 3.2 Hz, 1H), 5.30-5.38 (m, 1H), 4.92 (t, J = 6.8 Hz, 2H), 4.75 (s, 2H), 4.55 (dd, J = 7.2, 5.2 Hz, 2H), 3.76 (s, 3H). [M + H] = 468.0 |
| 304 | 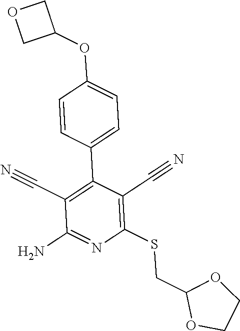 [SMILES: N#Cc1c(N)nc(SCC2OCCO2)c(C#N)c1-c1ccc(OC2COC2)cc1] | ¹H NMR (400₃OD) δ = 7.52 (d, 2H), 6.98 (d, 2H), 5.41 (s, 1H), 5.20 (t, 1H), 5.09 (t, 2H), 4.79-4.74 (m, 2H), 4.09-4.02 (m, 2H), 3.96-3.95 (m, 1H), 3.98-3.98 (m, 1H), 3.61 (d, 2H). [M + H] = 411.1 |
| 305 | 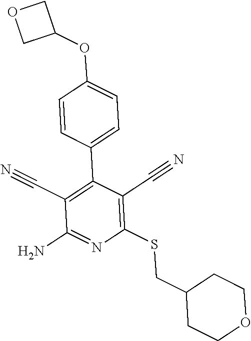 [SMILES: N#Cc1c(N)nc(SCC2CCOCC2)c(C#N)c1-c1ccc(OC2COC2)cc1] | ¹H NMR (400 MHz, CD₃OD) δ = 7.52 (d, J = 8.8 Hz, 2H), 6.98 (d, J = 8.8 Hz, 2H), 5.46-5.35 (m, 1H), 5.09 (t, J = 6.4 Hz, 2H), 4.77 (dd, J = 7.2, 5.2 Hz, 2H), 3.99 (dd, J = 10.4, 3.6 Hz, 2H), 3.44 (td, J = 11.2, 1.6 Hz, 2H), 3.29 (d, J = 6.4 Hz, 2H), 1.94 (m, J = 11.2, 7.2, 4.8 Hz, 1H), 1.86 (d, J = 13.2 Hz, 2H), 1.43 (qd, J = 12.4, 4.8 Hz, 2H). [M + H] = 423.1 |
| 308 | 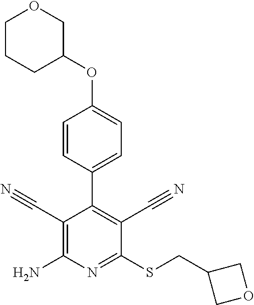 [SMILES: N#Cc1c(N)nc(SCC2COC2)c(C#N)c1-c1ccc(OC2CCCOC2)cc1] | ¹H-NMR (400 MHz, CD₃OD) δ = 7.49 (br d, J = 8.4 Hz, 2H), 7.13 (br d, J = 8.8 Hz, 2H), 4.83-4.80 (m, 1H), 4.62 (br d, J = 1.6 Hz, 1H), 4.52 (br t, J = 5.6 Hz, 3H), 3.95 (br d, J = 11.2 Hz, 1H), 3.79- 3.72 (m, 1H), 3.72-3.66 (m, 2H), 3.64 (br d, J = 7.6 Hz, 2H), 3.52-3.37 (m, 2H), 2.21-2.08 (m, 1H), 2.00-1.84 (m, 2H), 1.72-1.59 (m, 1H). [M + H] = 423.2 |
| 313 | 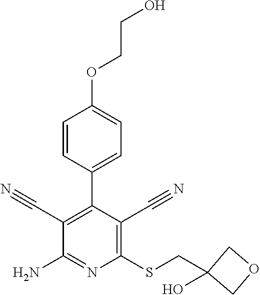 [SMILES: N#Cc1c(N)nc(SCC2(O)COC2)c(C#N)c1-c1ccc(OCCO)cc1] | ¹H NMR (400 MHz, CD₃OD) δ = 7.54- 7.48 (m, 2H), 7.19-7.11 (m, 2H), 4.66- 4.62 (m, 2H), 4.61-4.56 (m, 2H), 4.19- 4.14 (m, 2H), 3.96-3.91 (m, 2H), 3.90- 3.87 (m, 2H). [M + H] = 399.1 |
| 314 | 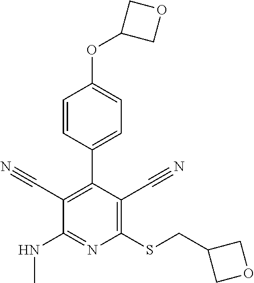 [SMILES: CNc1nc(SCC2COC2)c(C#N)c(-c2ccc(OC3COC3)cc2)c1C#N] | ¹H-NMR (400 = 3.10 (d, J = 4.8 Hz, 3H), 3.40-3.28 (m, 1H), 3.56 (d, J = 7.6 Hz, 2H), 4.41 (t, J = 6.0 Hz, 2H), 4.76 (m, 4H), 4.93 (t, J = 6.8 Hz, 2H), 5.19 (t, J = 5.6 Hz, 1H), 5.73 (br d, J = 4.4 Hz, 1H), 6.83-6.69 (m, 2H), 7.46-7.34 (m, 2H). [M + H] = 409.5 |
| 315 | 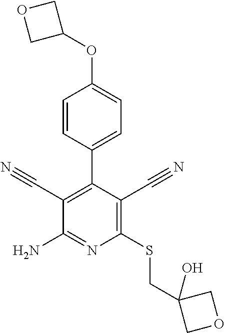 [SMILES: N#Cc1c(N)nc(SCC2(O)COC2)c(C#N)c1-c1ccc(OC2COC2)cc1] | ¹H NMR (400 MHz, DMSO-d₆) δ = 7.55- 7.47 (m, 2H), 7.02-6.93 (m, 2H), 6.19 (s, 1H), 5.42-5.34 (m, 1H), 5.01-4.93 (m, 2H), 4.64-4.57 (m, 2H), 4.51-4.45 (m, 2H), 4.44-4.38 (m, 2H), 3.82-3.74 (m, 2H), 3.32 (s, 2H), 3.18 (d, J = 5.6 Hz, 1H). [M + H] = 411.1 |
| 316 | 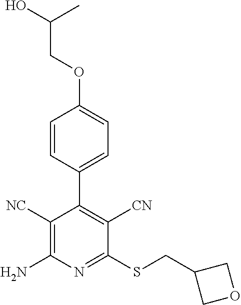 [SMILES: CC(O)COc1ccc(-c2c(C#N)c(N)nc(SCC3COC3)c2C#N)cc1] | ¹H-NMR (400 MHz, DMSO-d₆) δ = 1.23- 1.14 (m, 1H), 1.19 (d, J = 6.4 Hz, 2H), 3.57 (d, J = 7.2 Hz, 2H), 3.96-3.75 (m, 2H), 4.00 (dt, J = 11.2, 5.6 Hz, 1H), 4.38 (t, J = 6.0 Hz, 2H), 4.67 (dd, J = 7.6, 6.4 Hz, 2H), 4.96 (d, J = 4.8 Hz, 1H), 7.12 (d, J = 8.4 Hz, 2H), 7.49 (d, J = 8.8 Hz, 2H). [M + H] = 397.2 |
| 317 | 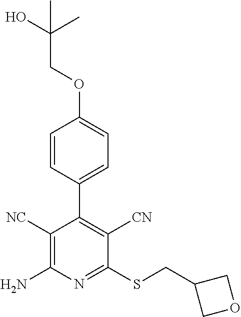 [SMILES: CC(C)(O)COc1ccc(-c2c(C#N)c(N)nc(SCC3COC3)c2C#N)cc1] | ¹H-NMR (400 MHz, DMSO-d6) δ = 8.15- 7.82 (m, 2H), 7.52-7.42 (m, 2H), 7.10 (d, J = 8.8 Hz, 2H), 4.72-4.61 (m, 3H), 4.36 (t, J = 6.0 Hz, 2H), 3.80 (s, 2 H), 3.55 (d, J = 7.2 Hz, 2H), 1.23 (s, 6H). [M + H] = 411.0 |
| 318 | 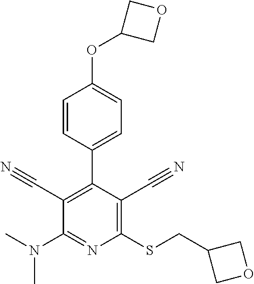 [SMILES: CN(C)c1nc(SCC2COC2)c(C#N)c(-c2ccc(OC3COC3)cc2)c1C#N] | ¹H NMR (400 MHz, DMSO-d₆) δ = 3.32 (br s, 1H), 3.36 (s, 6H), 3.59 (d, J = 7.2 Hz, 2H), 4.36 (t, J = 6.0 Hz, 2H), 4.60 (dd, J = 7.6, 5.2 Hz, 2H), 4.67 (dd, J = 7.6, 6.4 Hz, 2H), 4.97 (t, J = 6.8 Hz, 2H), 5.44-5.34 (m, 1H), 7.00-6.93 (m, 2H), 7.56-7.48 (m, 2H). [M + H] = 423.5 |
| 319 | 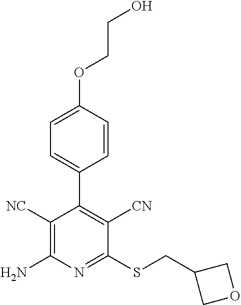 [SMILES: N#Cc1c(N)nc(SCC2COC2)c(C#N)c1-c1ccc(OCCO)cc1] | ¹H NMR (400 MHz, CD₃OD) δ = 7.51 (d, J = 8.8 Hz, 2 H) 7.15 (d, J = 8.8 Hz, 2 H) 4.85 (dd, J = 7.6, 6.4 Hz, 2 H) 4.53 (t, J = 6.4 Hz, 2 H) 4.17 (t, J = 4.4 Hz, 2 H) 3.94 (t, J = 4.4 Hz, 2 H) 3.65 (d, J = 7.6 Hz, 2 H) 3.50-3.42 (m, 1 H); [M + H] = 383.2 |
| 320 | 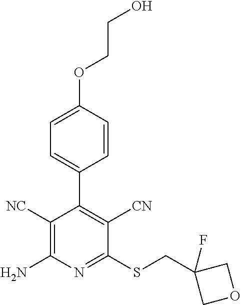 [SMILES: N#Cc1c(N)nc(SCC2(F)COC2)c(C#N)c1-c1ccc(OCCO)cc1] | ¹H NMR (400 MHz, CD₃OD) δ = 7.53 (d, J = 8.8 Hz, 2 H) 7.16 (d, J = 8.4 Hz, 2 H) 4.85-4.76 (m, 2 H) 4.76-4.67 (m, 2 H) 4.18 (t, J = 4.4 Hz, 2 H) 4.09-4.00 (m, 2 H) 3.95 (t, J = 4.4 Hz, 2 H) |
| 322 | 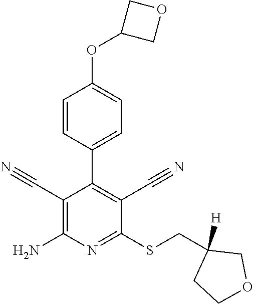 [SMILES: N#Cc1c(N)nc(SC[C@H]2CCOC2)c(C#N)c1-c1ccc(OC2COC2)cc1] | ¹H-NMR (400 MHz, CD₃OD) δ ppm 7.61-7.44 (m, 2H), 6.96 (d, J = 8.8 Hz, 2H), 5.53-5.30 (m, 1H), 5.07 (t, J = 6.8 Hz, 2H), 4.80-4.69 (m, 2H), 4.01-3.88 (m, 2H), 3.85-3.74 (m, 1H), 3.60 (dd, J = 8.8, 6.0 Hz, 1H), 3.38 (dd, J = 7.2, 1.2 Hz, 2H), 2.69 (dt, J = 13.6, 6.8 Hz, 1H), 2.19 (m, J = 7.6 Hz, 1H), 1.87-1.72 (m, 1H); [M + H] = 409.2 |
| 325 | 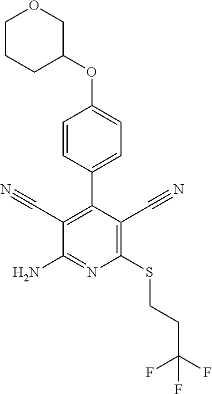 [SMILES: N#Cc1c(N)nc(SCCC(F)(F)F)c(C#N)c1-c1ccc(OC2CCCOC2)cc1] | ¹H-NMR (400 MHz, CD₃OD) δ = 7.57- 7.44 (m, 2H), 7.22-7.08 (m, 2H), 4.53 (dt, J = 6.4, 3.2 Hz, 1H), 3.96 (dd, J = 11.2, 2.8 Hz, 1H), 3.83-3.63 (m, 3H), 3.52-3.42 (m, 2H), 2.82-2.61 (m, 2H), 2.22-2.10(m, 1H), 2.03-1.84 (m, 2H), 1.76-1.59 (m, 1H). [M + H] = 449.2 |
| 326 | 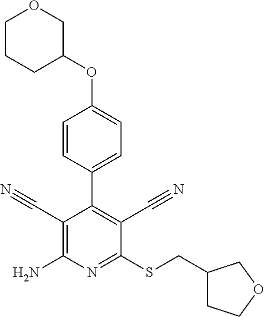 [SMILES: N#Cc1c(N)nc(SCC2CCOC2)c(C#N)c1-c1ccc(OC2CCCOC2)cc1] | ¹H-NMR (400 MHz, CD₃OD) δ = 7.48 (d, J = 8.8 Hz, 2H), 7.11 (d, J = 8.8 Hz, 2H), 4.49 (tt, J = 6.4, 3.2 Hz, 1H), 3.97-3.86 (m, 3H), 3.81-3.70 (m, 2H), 3.69-3.62 (m, 2H), 3.59 (dd, J = 8.8, 6.0 Hz, 1H), 3.36 (dd, J = 7.2, 1.6 Hz, 2H), 2.68 (dt, J = 14.0, 6.8 Hz, 1H), 2.23-2.07 (m, 2H), 1.98-1.73 (m, 3H), 1.69-1.59 (m, 1H). [M + H] = 437.2 |
| 327 | 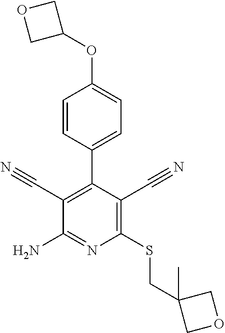 [SMILES: CC1(CSc2nc(N)c(C#N)c(-c3ccc(OC4COC4)cc3)c2C#N)COC1] | ¹H NMR (400 MHz, DMSO-d₆) δ = 7.56- 7.49 (m, 2H), 7.02-6.95 (m, 2H), 5.44- 5.34 (m, 1H), 5.02-4.95 (m, 2H), 4.65- 4.57 (m, 2H), 4.45-4.39 (m, 2H), 4.31- 4.23 (m, 2H), 3.68-3.61 (m, 2H), 1.41- 1.34 (m, 3 H). [M + H] = 409.1 |
| 258 | 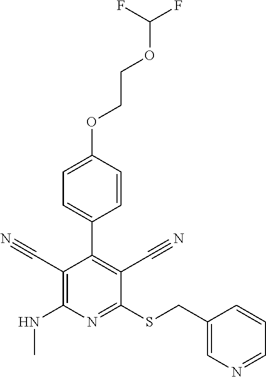 [SMILES: CNc1nc(SCc2cccnc2)c(C#N)c(-c2ccc(OCCOC(F)F)cc2)c1C#N] | ¹H NMR (400 MHz, CD₃OD) δ = 8.75- 8.40 (m, 2H), 7.98 (d, J = 8.0 Hz, 1H), 7.53-7.48 (m, 2H), 7.47-7.40 (m, 1H), 7.15-7.11 (m, 2H), 6.49 (t, J = 75.2 Hz, 1H), 4.66 (s, 2H), 4.60 (s, 1H), 4.31- 4.28 (m, 2H), 4.25-4.22 (m, 2H), 3.09 (s, 3H) [M + H] = 468.2 |
| 266 | 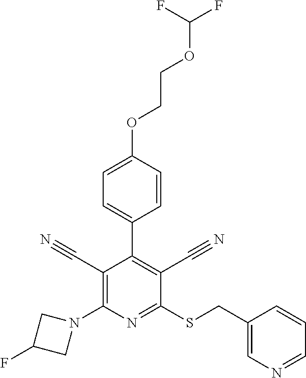 [SMILES: N#Cc1c(SCc2cccnc2)nc(N2CC(F)C2)c(C#N)c1-c1ccc(OCCOC(F)F)cc1] | ¹H NMR (400 MHz, CD₃OD) δ = 8.78- 8.60 (m, 1H), 8.51-8.38 (m, 1H), 7.96 (br d, J = 8.0 Hz, 1H), 7.51-7.49 (m, 1H), 7.48-7.47 (m, 1H), 7.46-7.42 (m, 1H), 7.15-7.13 (m, 1H), 7.13-7.10 (m, 1H), 6.73-6.21 (m, 1H), 5.56-5.36 (m, 1H), 4.84-4.72 (m, 2H), [M + H] = 512.24 |
| 241 | 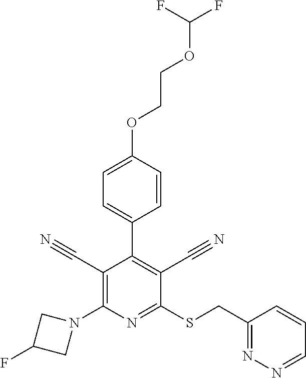 [SMILES: N#Cc1c(SCc2cccnn2)nc(N2CC(F)C2)c(C#N)c1-c1ccc(OCCOC(F)F)cc1] | ¹H NMR (400 MHz, DMSO-d₆) δ = 9.16 (dd, J = 4.8, 1.6 Hz, 1H), 7.81 (dd, J = 8.8, 1.6 Hz, 1H), 7.69 (dd, J = 8.8, 4.8 Hz, 1H), 7.49 (d, J = 8.8 Hz, 2H), 7.14 (d, J = 8.8 Hz, 2H), 6.77 (t, J = 75.6 Hz, 1H), 5.62-5.36 (m, 1H), 4.83 (s, 2H), 4.75-4.60 (m, 2H), 4.46-4.31 (m, 2H), 4.29-4.18 (m, 4H) [M + H] = 513.2 |
| 235 | 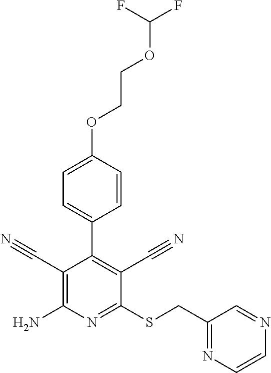 [SMILES: N#Cc1c(N)nc(SCc2cnccn2)c(C#N)c1-c1ccc(OCCOC(F)F)cc1] | ¹H NMR (400 MHz, DMSO-d₆) δ = 8.99 (d, J = 1.0 Hz, 1H), 8.63-8.61 (m, 1H), 8.54 (d, J = 2.4 Hz, 1H), 7.48 (d, J = 8.8 Hz, 2H), 7.12 (d, J = 8.8 Hz, 2H), 6.76 (t, J = 7.6 Hz, 1H), 4.61 (s, 2H), 4.28-4.25 (m, 2H), 4.21-4.18 (m, 2H) [M + H] = 455.2 |
| 338 | 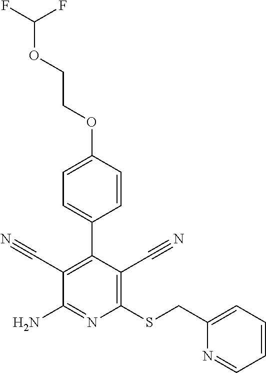 [SMILES: N#Cc1c(N)nc(SCc2ccccn2)c(C#N)c1-c1ccc(OCCOC(F)F)cc1] | ¹H NMR (400 MHz, CD₃OD) δ = 8.49 (br d, J = 4.4 Hz, 1H), 7.81 (dt, J = 7.6, 1.6 Hz, 1H), 7.67 (d, J = 7.2 Hz, 1H), 7.48 (d, J = 8.8 Hz, 2H), 7.33 (dd, J = 7.2, 5.2 Hz, 1H), 7.11 (d, J = 8.8 Hz, 2H), 6.47 (t, J = 7.6 Hz, 1H), 4.63 (s, 2H), 4.28-4.25 (m, 2H), 4.23-4.20 (m, 2H) |
| 330 | 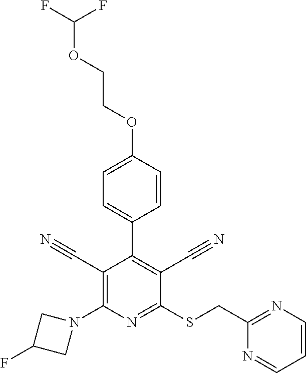 [SMILES: N#Cc1c(SCc2ncccn2)nc(N2CC(F)C2)c(C#N)c1-c1ccc(OCCOC(F)F)cc1] | ¹H NMR (400 MHz, DMSO-d₆) δ = 8.80 (d, J = 5.2 Hz, 2H), 7.49 (d, J = 8.8 Hz, 2H), 7.46-7.41 (m, 1H), 7.14 (d, J = 8.8 Hz, 2H), 6.77 (t, J = 75.6 Hz, 1H), 5.62- 5.29 (m, 1H), 4.75 (s, 2H), 4.70-4.56 (m, 2H), 4.44-4.29 (m, 2H), 4.27 (br d, J = 4.8 Hz, 2H), 4.21-4.17 (m, 2H) |
| 329 | 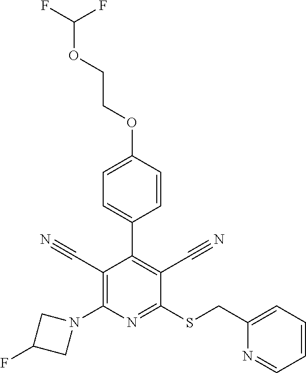 [SMILES: N#Cc1c(SCc2ccccn2)nc(N2CC(F)C2)c(C#N)c1-c1ccc(OCCOC(F)F)cc1] | ¹H NMR (400 MHz, CD₃OD) δ = 8.86- 8.11 (m, 1H), 7.85 (br t, J = 7.6 Hz, 1H), 7.70-7.52 (m, 1H), 7.47 (d, J = 8.8 Hz, 2H), 7.43-7.28 (m, 1H), 7.11 (d, J = 8.8 Hz, 2H), 6.47 (t, J = 7.2 Hz, 1H), 5.42 (td, J = 3.2, 5.6 Hz, 1H), 4.79-4.62 (m, 4H), 4.44 (br dd, J = 11.2, 22.8 Hz, 2H), 4.29-4.25 (m, 2H), 4.24-4.18 (m, 2H) [M + H] = 512.1 |
| 237 | 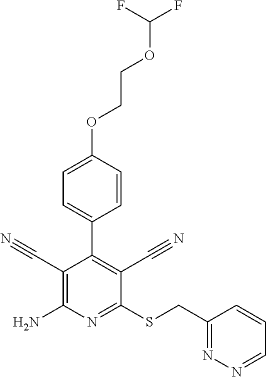 [SMILES: N#Cc1c(N)nc(SCc2cccnn2)c(C#N)c1-c1ccc(OCCOC(F)F)cc1] | ¹H NMR (400 MHz, DMSO-d₆) δ = 9.15 (dd, J = 4.8, 1.6 Hz, 1H), 8.43-7.97 (m, 2H), 7.96 (dd, J = 8.4, 1.6 Hz, 1H), 7.67 (dd, J = 8.4, 4.8 Hz, 1H), 7.54-7.46 (m, 2H), 7.14 (d, J = 8.8 Hz, 2H), 6.77 (t, J = 7.6 Hz, 1H), 4.78 (s, 2H), 4.29-4.18 (m, 4H) [M + H] = 455.1 |
| 339 | 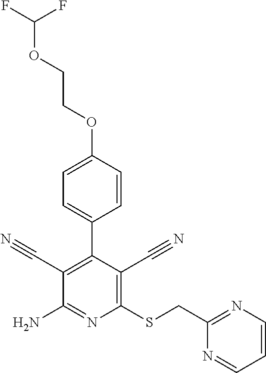 [SMILES: N#Cc1c(N)nc(SCc2ncccn2)c(C#N)c1-c1ccc(OCCOC(F)F)cc1] | ¹H NMR (400 MHz, DMSO-d₆) δ = 8.81 (d, J = 4.8 Hz, 2H), 8.25-7.66 (m, 2H), 7.51 (d, J = 8.8 Hz, 2H), 7.45 (t, J = 4.8 Hz, 1H), 7.14 (d, J = 8.8 Hz, 2H), 6.97- 6.57 (m, 1H), 4.83 (s, 2H), 4.30-4.25 (m, 2H), 4.21-4.18 (m, 2H) [M + H] = 455.2 |
| 262 | 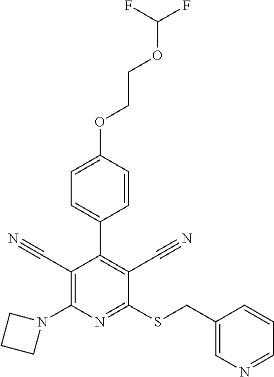 [SMILES: N#Cc1c(SCc2cccnc2)nc(N2CCC2)c(C#N)c1-c1ccc(OCCOC(F)F)cc1] | ¹H NMR (400 MHz, CD₃OD) δ = 8.66 (s, 1H), 8.51-8.45 (m, 1H), 7.97 (br d, J = 8.0 Hz, 1H), 7.48 (d, J = 8.8 Hz, 3H), 7.13 (d, J = 8.8 Hz, 2H), 6.50 (t, J = 75.2 Hz, 1H), 4.67-4.45 (m, 6H), 4.31-4.23 (m, 4H), 2.48 (br t, J = 7.6 Hz, 2H). [M + H] = 494.1 |
| 264 | 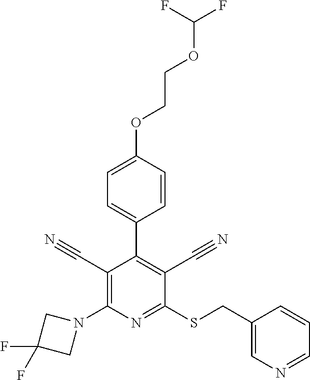 [SMILES: N#Cc1c(SCc2cccnc2)nc(N2CC(F)(F)C2)c(C#N)c1-c1ccc(OCCOC(F)F)cc1] | ¹H NMR (400 MHz, CD₃OD) δ = 8.64 (s, 1H), 8.45 (d, J = 4.0 Hz, 1H), 7.94 (br d, J = 8.0 Hz, 1H), 7.49 (d, J = 8.8 Hz, 2H), 7.43 (dd, J = 7.6, 4.8 Hz, 1H), 7.12 (d, J = 8.8 Hz, 2H), 6.47 (t, J = 75.2 Hz, 1H), 4.81 (t, J = 12.0 Hz, 4H), 4.59 (s, 2H), 4.29-4.25 (m, 2H), 4.24-4.19 (m, 2H). [M + H] = 530.1 |
| 257 | 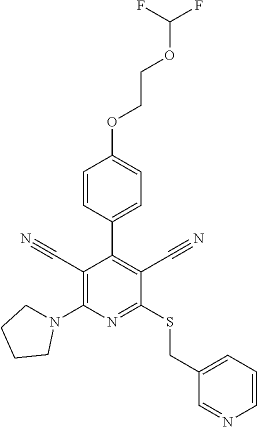 [SMILES: N#Cc1c(SCc2cccnc2)nc(N2CCCC2)c(C#N)c1-c1ccc(OCCOC(F)F)cc1] | ¹H NMR (400 MHz, CD₃OD) δ = 8.65 (br s, 2H), 7.94 (br d, J = 8.0 Hz, 1H), 7.52-7.38 (m, 3H), 7.11 (d, J = 8.8 Hz, 2H), 6.48 (t, J = 7.2 Hz, 1H), 4.61 (s, 2H), 4.29-4.21 (m, 4H), 3.86 (br s, 4H), 2.02 (br t, J = 6.4 Hz, 4H) [M + H] = 508.1 |
| 265 | 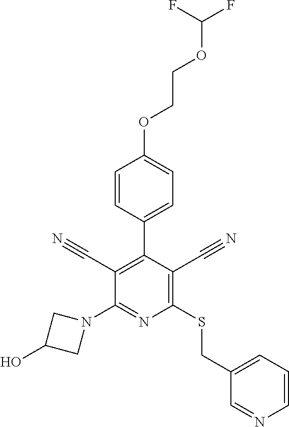 [SMILES: N#Cc1c(SCc2cccnc2)nc(N2CC(O)C2)c(C#N)c1-c1ccc(OCCOC(F)F)cc1] | ¹H NMR (400 MHz, CD₃OD) δ = 8.71- 8.46 (m, 2H), 8.90-8.29 (m, 1H), 7.94 (d, J = 8.0 Hz, 1H), 7.43 (br s, 3H), 7.10 (d, J = 8.8 Hz, 2H), 6.65 (s, 1H), 4.68 (br s, 3H), 4.56 (s, 2H), 4.28-4.25 (m, 2H), 4.23-4.18 (m, 4H). [M + H] = 510.1 |

^{a}Step 3 was altered by reacting the thiolpyridine with iodopyridine in DMSO in the presence of CuI (1 eq), (2S)-pyrrolidine-2-carboxylic acid (0.4 eq) and t-BuONa (2 eq).
^{b}The enantiomers were separated by SFC (Instrument: Waters SFC80 preparative SFC; Column: DAICEL CHIRALPAK IG (250 mm x 30 mm, 10 um); mobile phase: [CO₂-IPA(0.1% NH₃H₂O)]; B %:35%, isocratic elution mode). Absolute configuration assignments require further analysis to confirm the preliminary designations provided, as one of ordinary skill will readily appreciate.

### Protocol 2. 2-Amino-6-(1H-indol-4-ylmethylsulfanyl)-4-[4-(oxetan-3-yloxy)phenyl]pyridine-3,5-dicarbonitrile (22)

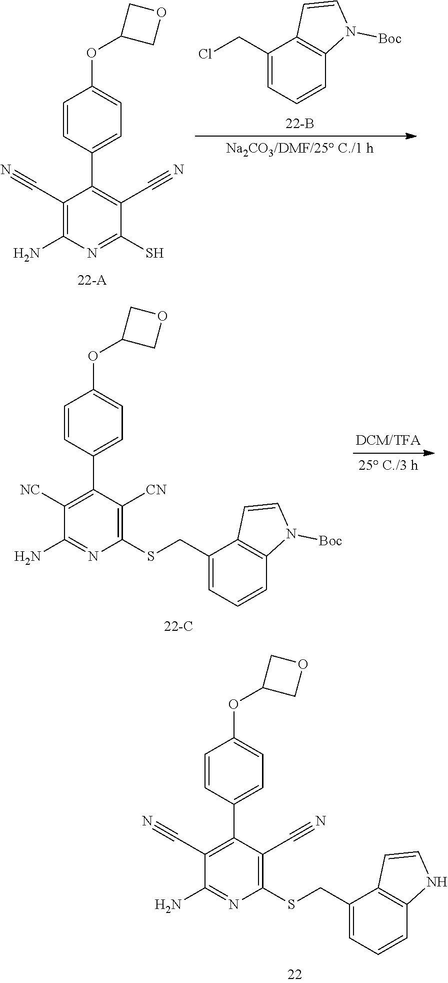 [SMILES: N#Cc1c(N)nc(S)c(C#N)c1-c1ccc(OC2COC2)cc1 ; CC(C)(C)OC(=O)n1ccc2c(CCl)cccc21 ; CC(C)(C)OC(=O)n1ccc2c(CSc3nc(N)c(C#N)c(-c4ccc(OC5COC5)cc4)c3C#N)cccc21 ; N#Cc1c(N)nc(SCc2cccc3[nH]ccc23)c(C#N)c1-c1ccc(OC2COC2)cc1]

Step 1. tert-Butyl 4-(chloromethyl)indole-1-carboxylate (22-B) To a mixture of tert-butyl 4-(hydroxymethyl)indole-1-carboxylate (180 mg, 727.89 μmol, 1 eq) in CHCl₃ (2.5 mL) was added SOCl₂ (173.19 mg, 1.46 mmol, 105.61 μL, 2 eq) at 0° C., the mixture was stirred at 65° C. for 1 h. The reaction mixture was concentrated under reduced pressure to give tert-butyl 4-(chloromethyl)indole-1-carboxylate (180 mg, crude) as brown solid. [M+H]=265.7

Step 2. tert-Butyl 4-[[6-amino-3,5-dicyano-4-[4-(oxetan-3-yloxy)phenyl]-2-pyridyl]-sulfanylmethyl]indole-1-carboxylate (22-C). To a mixture of 2-amino-4-[4-(oxetan-3-yloxy)phenyl]-6-sulfanyl-pyridine-3,5-dicarbonitrile (150 mg, 462.45 μmol, 1 eq) and tert-butyl 4-(chloromethyl)indole-1-carboxylate (122.89 mg, 462.45 μmol, 1 eq) in DMF (2 mL) was added K₂CO₃ (191.74 mg, 1.39 mmol, 3 eq) at 25° C., the mixture was stirred at 25° C. for 1 h. The reaction mixture was quenched by addition saturated aqueous NaCl (20 mL), and then extracted with EtOAc (3×20 mL). The combined organic layers were dried over Na₂SO₄, filtered and concentrated under reduced pressure to give a residue. The residue was purified by prep-TLC (PE:EtOAc=1:1) to give tert-butyl 4-[[6-amino-3,5-dicyano-4-[4-(oxetan-3-yloxy)phenyl]-2-pyridyl]sulfanylmethyl]indole-1-carboxylate (70 mg, 126.44 μmol, 27.34% yield) as brown solid. [M+H]=554.3

Step 3. 2-Amino-6-(1H-indol-4-ylmethylsulfanyl)-4-[4-(oxetan-3-yloxy)phenyl]-pyridine-3,5-dicarbonitrile (22). A mixture of tert-butyl 4-[[6-amino-3,5-dicyano-4-[4-(oxetan-3-yloxy)phenyl]-2-pyridyl]-sulfanylmethyl]indole-1-carboxylate (50 mg, 90.31 μmol, 1 eq) in DCM (3 mL) and TFA (0.1 mL) was stirred at 25° C. for 72 h. The reaction mixture was concentrated under reduced pressure to give a residue. The residue was purified by prep-HPLC (column: Phenomenex C18 75 mm×30 mm×3 μm; mobile phase A: 10 mM NH₄HCO₃ in water, mobile phase B: ACN; 30%-60% over 8 min, to give 2-amino-6-(1H-indol-4-ylmethylsulfanyl)-4-[4-(oxetan-3-yloxy)phenyl]pyridine-3,5-dicarbonitrile (10.1 mg, 22.27 μmol, 24.66% yield, 100% purity) as a white solid. ¹H-NMR (400 MHz, CDCl₃) δ=1.71-1.82 (m, 1H), 2.14-2.27 (m, 1H), 2.67 (dt, J=13.2, 6.8 Hz, 1H), 3.41 (dd, J=7.2, 2.0 Hz, 2H), 3.63 (dd, J=8.8, 5.6 Hz, 1H), 3.81 (q, J=7.6 Hz, 1H), 3.89-3.97 (m, 2H), 4.76-4.90 (m, 2H), 5.02 (br t, J=6.8 Hz, 2H), 5.30 (quin, J=5.6 Hz, 1H), 6.89 (br d, J=8.4 Hz, 2H), 7.53 (br d, J=8.4 Hz, 2H), 8.78 (s, 1H). [M+H]=454.2.

TABLE 2
The following compounds were synthesized in a similar manner, but with
different starting materials.

| Compound | Structure | Spectroscopic data |
|---|---|---|
| 15 | 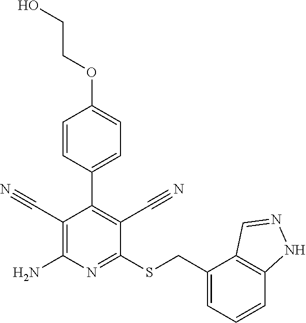 [SMILES: N#Cc1c(N)nc(SCc2cccc3[nH]ncc23)c(C#N)c1-c1ccc(OCCO)cc1] | ¹H-NMR (400 MHz, DMSO-d₆) δ = 13.14 (s, 1H), 8.25 (s, 1H), 7.42-7.49 (m, 3H), 7.24-7.32 (m, 2H), 7.08 (d, J = 8.8 Hz, 2H), 4.88-4.92 (m, 1H), 4.86 (s, 2H), 4.06 (t, J = 4.8 Hz, 2H), 3.73 (q, J = 5.6 Hz, 2H). [M + H] = 443.3. |
| 5 | 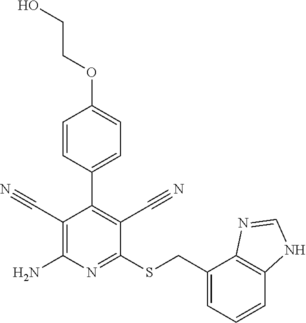 [SMILES: N#Cc1c(N)nc(SCc2cccc3[nH]cnc23)c(C#N)c1-c1ccc(OCCO)cc1] | ¹H-NMR (400 MHz, DMSO-d₆) δ = 9.17 (s, 1H), 7.75 (d, J = 7.2 Hz, 1H), 7.68 (d, J = 8.0 Hz, 1H), 7.45 (d, J = 8.8 Hz, 2H), 7.38 (t, J = 7.6 Hz, 1H), 7.08 (d, J = 8.8 Hz, 2H), 4.82 (s, 2H), 4.06 (t, J = 4.8 Hz, 2H), 3.73 (t, J = 4.8 Hz, 2H). [M + H] = 443.0. |
| 14 | 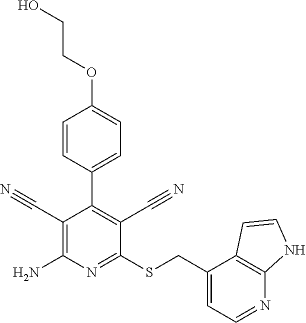 [SMILES: N#Cc1c(N)nc(SCc2ccnc3[nH]ccc23)c(C#N)c1-c1ccc(OCCO)cc1] | ¹H-NMR (400 MHz, DMSO-d₆) δ = 11.97 (s, 1H), 8.20 (d, J = 5.2 Hz, 1H), 7.53-7.57 (m, 1H), 7.43-7.48 (m, 2H), 7.38 (d, J = 5.2 Hz, 1H), 7.05-7.11 (m, 2H), 6.75 (dd, J = 3.2, 1.6 Hz, 1H), 4.83 (s, 2H), 4.06 (t, J = 4.8 Hz, 2H), 3.73 (t, J = 4.8 Hz, 2H). [M + H] = 443.0. |
| 2 | 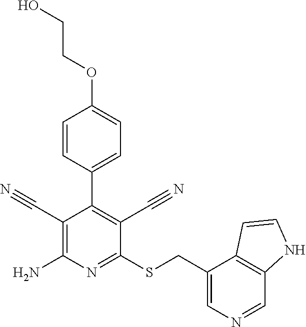 [SMILES: N#Cc1c(N)nc(SCc2cncc3[nH]ccc23)c(C#N)c1-c1ccc(OCCO)cc1] | ¹H-NMR (400 MHz, DMSO-d₆) δ = 9.09 (s, 1H), 8.66 (s, 1H), 8.32 (s, 1H), 7.45 (d, J = 8.8 Hz, 2H), 7.17 (d, J = 2.4 Hz, 1H), 7.09 (d, J = 8.8 Hz, 2H), 4.90 (s, 2H), 4.07 (t, J = 4.8 Hz, 2H), 3.74 (t, J = 4.8 Hz, 2H). [M + H] = 443.2. |
| 8 | 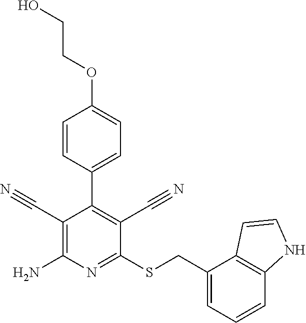 [SMILES: N#Cc1c(N)nc(SCc2cccc3[nH]ccc23)c(C#N)c1-c1ccc(OCCO)cc1] | ¹H-NMR (400 MHz, DMSO-d₆) δ = 11.20 (s, 1H), 7.46 (d, J = 8.8 Hz, 2H), 7.37 (t, J = 2.4 Hz, 1H), 7.33 (d, J = 8.4 Hz, 1H), 7.17 (d, J = 7.2 Hz, 1H), 7.08 (d, J = 8.8 Hz, 2H), 6.99-7.05 (m, 1 H), 6.56 (brs, 1H), 4.79 (s, 2H), 4.06 (t, J = 4.8 Hz, 2H), 3.73 (t, J = 4.8 Hz, 2H). [M + H] = 442.0. |
| 67 | 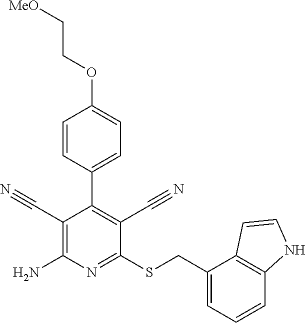 [SMILES: COCCOc1ccc(-c2c(C#N)c(N)nc(SCc3cccc4[nH]ccc34)c2C#N)cc1] | ¹H-NMR (400 MHz, DMSO-d₆) δ = 11.20 (br s, 1H), 7.47 (d, J = 8.6 Hz, 2H), 7.37 (t, J = 2.6 Hz, 1H), 7.33 (d, J = 8.0 Hz, 1H), 7.17 (d, J = 7.2 Hz, 1H), 7.10 (d, J = 8.8 Hz, 2H), 7.00-7.05 (m, 1H), 6.56 (br s, 1H), 4.79 (s, 2H), 4.14-4.20 (m, 2H), 3.64-3.72 (m, 2H), 3.32 (s, 3 H). [M + H] = 456.0 |
| 214 | 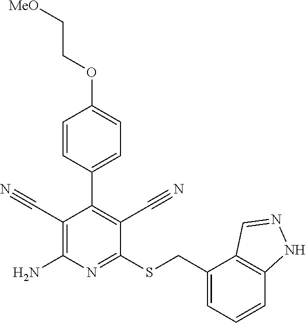 [SMILES: COCCOc1ccc(-c2c(C#N)c(N)nc(SCc3cccc4[nH]ncc34)c2C#N)cc1] | ¹H-NMR (400 MHz, DMSO-d₆) δ = 13.15 (s, 1H), 8.25 (s, 1H), 7.44-7.50 (m, 3H), 7.24-7.34 (m, 2H), 7.09 (d, J = 8.8 Hz, 2H), 4.87 (s, 2H), 4.12-4.20 (m, 2H), 3.64-3.72 (m, 2H), 3.30-3.32 (m, 3H). [M + H] = 456.9. |
| 4 | 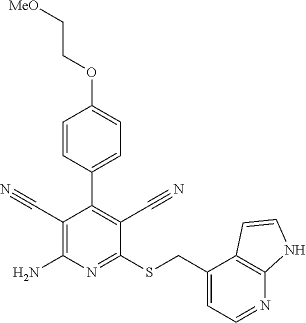 [SMILES: COCCOc1ccc(-c2c(C#N)c(N)nc(SCc3ccnc4[nH]ccc34)c2C#N)cc1] | ¹H-NMR (400 MHz, DMSO-d₆) δ = 11.71 (br s, 1H), 8.15 (d, J = 4.8 Hz, 1H), 7.44-7.52 (3 H, m), 7.27 ( d, J = 4.8 Hz, 1H), 7.10 (d, J = 8.8 Hz, 2H), 6.65 (dd, J = 3.2, 1.6 Hz, 1H), 4.81 (s, 2H), 4.15-4.20 (m, 2H), 3.66-3.73 (m, 2H), 3.32 (s, 3H). [M + H] = 457.5. |
| 215 | 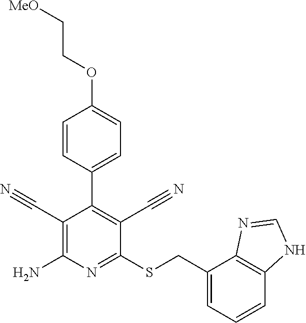 [SMILES: COCCOc1ccc(-c2c(C#N)c(N)nc(SCc3cccc4[nH]cnc34)c2C#N)cc1] | ¹H-NMR (400 MHz, DMSO-d₆) δ = 9.24 (br s, 1H), 7.78 (d, J = 7.2 Hz, 1H), 7.70 (d, J = 7.6 Hz, 1H), 7.40-7.44 (m, 2H), 7.40 (t, J = 7.8 Hz, 1H), 7.08-7.12 (m, 2H), 4.82 (s, 2H), 4.12-4.16 (m, 2H), 3.68-3.72 (m, 2H), 3.32 (s, 3H). |
| 216 | 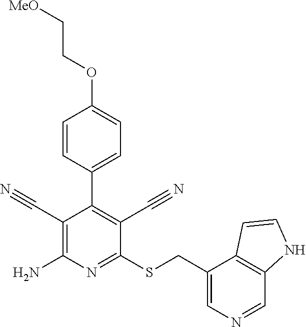 [SMILES: COCCOc1ccc(-c2c(C#N)c(N)nc(SCc3cncc4[nH]ccc34)c2C#N)cc1] | ¹H-NMR (400 MHz, DMSO-d₆) δ = 13.00 (br s, 1H), 9.10 (s, 1 H), 8.66 (s, 1H), 8.32 (t, J = 2.4 Hz, 1H), 7.43-7.47 (m, 2H), 7.17 (d, J = 2.8 Hz, 1H), 7.07- 7.12 (m, 2H), 4.90 (s, 2H), 4.15-4.20 (m, 2H), 3.65-3.71 (m, 2H), 3.32 (s, 3H). [M + H] = 456.9. |
| 217^{a} | 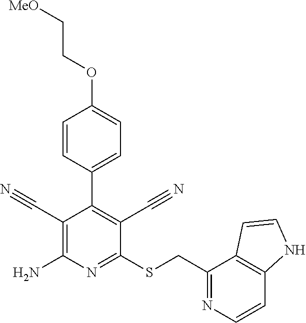 [SMILES: COCCOc1ccc(-c2c(C#N)c(N)nc(SCc3nccc4[nH]ccc34)c2C#N)cc1] | ¹H-NMR (400 MHz, CD₃OD) δ = 8.12 (d, J = 5.2 Hz, 1H), 7.50 (d, J = 8.8 Hz, 2H), 7.45 (d, J = 3.2 Hz, 1H), 7.41 (d, J = 5.6 Hz, 1H), 7.13 (d, J = 8.8 Hz, 2H), 6.84 (d, J = 3.2 Hz, 1H), 4.99 (s, 2H), 4.21-4.25 (m, 2H), 3.79-3.83 (m, 2H), 3.47 (s, 3H). [M + H] = 457.2. |
| 221 | 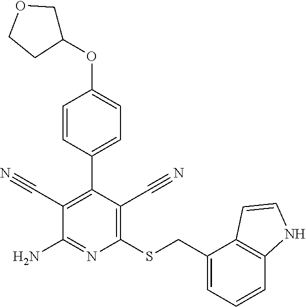 [SMILES: N#Cc1c(N)nc(SCc2cccc3[nH]ccc23)c(C#N)c1-c1ccc(OC2CCOC2)cc1] | ¹H-NMR (400 MHz, MeOD) δ = 7.52- 7.48 (m, 2H), 7.36 (d, J = 8.0 Hz, 1H), 7.29 (d, J = 3.2 Hz, 1H), 7.16-7.12 (m, 1H), 7.12-7.08 (m, 2H), 7.08-7.06 (m, 1H), 6.60 (dd, J = 3.2, 0.8 Hz, 1H), 5.13 (dd, J = 6.4, 4.0 Hz, 1H), 4.60 (s, 2H), 4.04-4.00 (m, 1H), 4.00-3.96 (m, 2H), 3.96-3.88 (m, 1H), 2.40-2.28 (m, 1H), 2.17 (dd, J = 12.0, 6.4 Hz, 1H). ESI [M + H] = 468.1. |

^{a}The azaindole was protected with a tosyl group and deprotected with KOH in MeOH/THF at RT for 1h.

### Protocol 3. 2-(Azetidin-1-yl)-4-[4-(2-hydroxyethoxy)phenyl]-6-(oxetan-3-ylmethylsulfanyl)pyridine-3,5-dicarbonitrile (63)

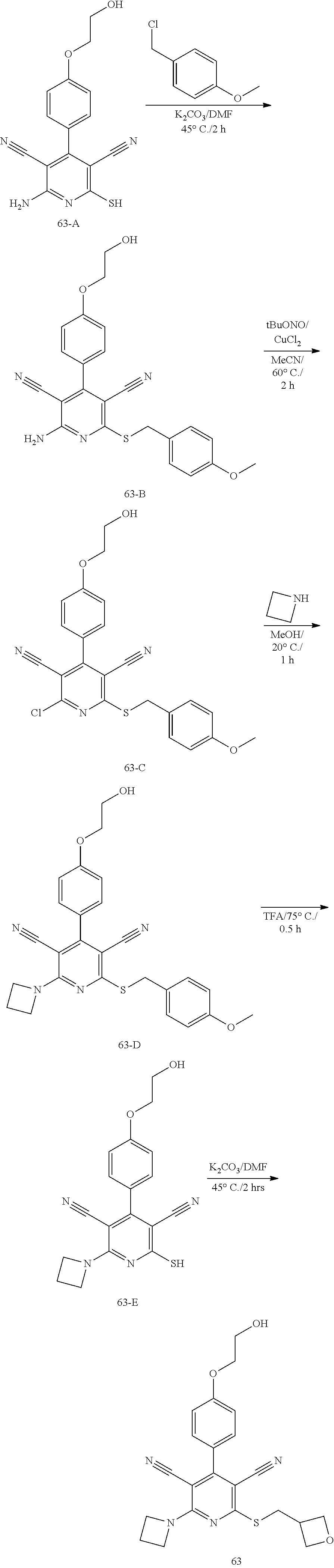

Step 1. 2-Amino-4-[4-(2-hydroxyethoxy)phenyl]-6-[(4-methoxyphenyl)methyl-sulfanyl]pyridine-3,5-dicarbonitrile (63-B). A mixture of 2-amino-4-[4-(2-hydroxyethoxy)phenyl]-6-sulfanyl-pyridine-3,5-dicarbonitrile (63-A, 500 mg, 60 mmol, 1 eq), 1-(chloromethyl)-4-methoxy-benzene (300.84 mg, 1.92 mmol, 261.60 μL, 1.2 eq), K₂CO₃ (442.48 mg, 3.20 mmol, 2 eq) in DMF (5 mL) was stirred at 45° C. for 2 h. To the reaction mixture was added H₂O (8 mL) and the reaction mixture was filtered, and the filter cake was collected to give 2-amino-4-[4-(2-hydroxyethoxy)phenyl]-6-[(4-methoxy-phenyl)methylsulfanyl]pyridine-3,5-dicarbonitrile (420 mg, 971.11 μmol, 60.66% yield) as a brown solid. ESI [M+H]=433.1.

Step 2. 2-Chloro-4-[4-(2-hydroxyethoxy)phenyl]-6-[(4-methoxyphenyl)methyl-sulfanyl]pyridine-3,5-dicarbonitrile (63-C). A mixture of 2-amino-4-[4-(2-hydroxyethoxy)phenyl]-6-[(4-methoxyphenyl)methylsulfanyl]-pyridine-3,5-dicarbonitrile (300 mg, 693.65 μmol, 1 eq), tert-butyl nitrite (143.06 mg, 1.39 mmol, 165.00 μL, 2 eq), CuCl₂ (186.52 mg, 1.39 mmol, 2 eq) in ACN (4 mL) was stirred at 60° C. for 2 h. The reaction mixture was concentrated under reduced pressure to give 2-chloro-4-[4-(2-hydroxyethoxy)phenyl]-6-[(4-methoxyphenyl)methylsulfanyl]pyridine-3,5-dicarbonitrile (250 mg, 553.19 μmol, 79.75% yield) as a green solid. ESI [M+H]=452.1.

Step 3. 2-(Azetidin-1-yl)-4-[4-(2-hydroxyethoxy)phenyl]-6-[(4-methoxyphenyl)-methylsulfanyl]pyridine-3,5-dicarbonitrile (63-D). A mixture of 2-chloro-4-[4-(2-hydroxyethoxy)phenyl]-6-[(4-methoxyphenyl)methylsulfanyl]-pyridine-3,5-dicarbonitrile (250 mg, 553.19 μmol, 1 eq), azetidine (63.17 mg, 1.11 mmol, 74.67 μL, 2 eq), in MeOH (3 mL) was stirred at 20° C. for 0.5 h. The reaction mixture was filtered, and the filtrate was collected. The residue was purified by prep-HPLC (column: Waters Xbridge Prep OBD C_{18 150} mm×40 mm×10 μm; mobile phase A: 10 mM NH₄HCO₃ in water, mobile phase B: ACN; 45%-75% over 8 min, to give 2-(azetidin-1-yl)-4-[4-(2-hydroxyethoxy)phenyl]-6-[(4-methoxyphenyl)-methylsulfanyl]pyridine-3,5-dicarbonitrile (150 mg, 317.42 μmol, 57.38% yield) as a white solid. ESI [M+H]=473.2.

Step 4. 2-(Azetidin-1-yl)-4-[4-(2-hydroxyethoxy)phenyl]-6-sulfanyl-pyridine-3,5-dicarbonitrile (63-E). A mixture of 2-(azetidin-1-yl)-4-[4-(2-hydroxyethoxy)phenyl]-6-[(4-methoxyphenyl)-methyl-sulfanyl]pyridine-3,5-dicarbonitrile (120 mg, 253.94 μmol, 1 eq) in TFA (3 mL) at 75° C. for 0.5 h. The reaction mixture was concentrated under reduced pressure to give 2-(azetidin-1-yl)-4-[4-(2-hydroxyethoxy)phenyl]-6-sulfanyl-pyridine-3,5-dicarbonitrile (80 mg, 227.01 μmol, 89.40% yield) as a yellow solid. ESI [M+H]=353.2.

Step 5. 2-(Azetidin-1-yl)-4-[4-(2-hydroxyethoxy)phenyl]-6-(oxetan-3-ylmethylsulfanyl)-pyridine-3,5-dicarbonitrile (63). A mixture of 2-(azetidin-1-yl)-4-[4-(2-hydroxyethoxy)phenyl]-6-sulfanyl-pyridine-3,5-dicarbonitrile (70 mg, 198.63 μmol, 1 eq), 3-(bromomethyl)oxetane (32.99 mg, 218.50 μmol, 1.1 eq), K₂CO₃ (54.90 mg, 397.27 μmol, 2 eq) in DMF (1 mL) was stirred at 45° C. for 2 h. The reaction mixture was filtered, and filtrate was collected. The residue was purified by prep-HPLC (column: Waters Xbridge Prep OBDC18 150 mm×40 mm×10 μm; mobile phase A: 10 mM NH₄HCO₃ in water, mobile phase B: ACN; 25%-55% over 8 min, to give 2-(azetidin-1-yl)-4-[4-(2-hydroxyethoxy)phenyl]-6-(oxetan-3-ylmethylsulfanyl)pyridine-3,5-dicarbonitrile (0.7 mg, 1.66 μmol, 0.83% yield, 100% purity) as white solid. ¹H-NMR (400 MHz, DMSO-d₆) δ=7.43 (d, J=8.8 Hz, 2H), 7.08 (d, J=8.8 Hz, 2H), 4.82-5.03 (m, 1H), 4.75-5.09 (m, 2H), 4.63 (dd, J=7.6, 6.4 Hz, 6H), 4.28-4.37 (m, 2H), 4.05 (t, J=4.8 Hz, 2H), 3.72 (t, J=4.4 Hz, 2H), 3.52 (d, J=7.2 Hz, 1H), 2.29-2.42 (m, 2H).

TABLE 3
The following compounds were synthesized in a similar manner, but with
different starting materials.

| Compound | Structure | Spectroscopic data |
|---|---|---|
| 62 | 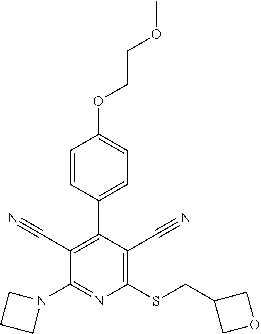 [SMILES: COCCOc1ccc(-c2c(C#N)c(SCC3COC3)nc(N3CCC3)c2C#N)cc1] | ¹H-NMR (400 MHz, DMSO-d₆) δ = 7.43 (d, J = 8.8 Hz, 2H), 7.08 (d, J = 8.8 Hz, 2H), 4.63 (t, J = 6.4 Hz, 2H), 4.27-4.47 (m, 5H), 4.12-4.22 (m, 2H), 3.63-3.72 (m, 2H), 3.48- 3.56 (m, 2H), 3.43-3.40 (m, 1H), 3.30 (br.s, 3H), 2.26-2.42 (m, 2H). ESI [M + H] = 437.1 |

### Protocol 4. 4-[4-(2-hydroxyethoxy)phenyl]-2-(oxetan-3-ylmethythio)pyridine-3,5-dicarbonitrile (61)

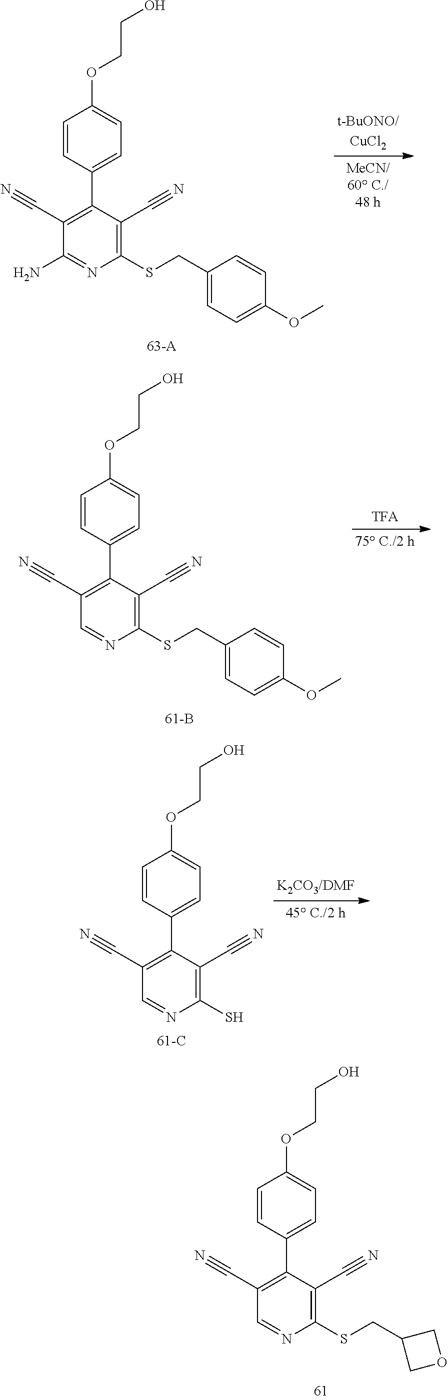 [SMILES: COc1ccc(CSc2nc(N)c(C#N)c(-c3ccc(OCCO)cc3)c2C#N)cc1 ; COc1ccc(CSc2ncc(C#N)c(-c3ccc(OCCO)cc3)c2C#N)cc1 ; N#Cc1cnc(S)c(C#N)c1-c1ccc(OCCO)cc1 ; N#Cc1cnc(SCC2COC2)c(C#N)c1-c1ccc(OCCO)cc1]

Step 1. 4-(4-(2-Hydroxyethoxy)phenyl)-2-((4-methoxybenzyl)thio)pyridine-3,5-dicarbonitrile (61-B). A mixture of 2-amino-4-[4-(2-hydroxyethoxy)phenyl]-6-[(4-methoxyphenyl)methyl-thio]pyridine-3,5-dicarbonitrile (0.88 g, 2.03 mmol, 1 eq), tert-butyl nitrite (1.19 g, 10.17 mmol, 1.37 mL, 5 eq), CuCl₂ (164.14 mg, 1.22 mmol, 0.6 eq) in MeCN (10 mL) was stirred at 60° C. for 48 h. The reaction mixture was filtered, and the filtrate was collected. The residue was purified by prep-HPLC (column: Kromasil C₁₈ (250 mm×50 mm×10 μm); mobile phase A: 10 mM NH₄HCO₃ in water, mobile phase B: ACN; 50%-75% over 10 min, to give 4-[4-(2-hydroxyethoxy)phenyl]-2-[(4-methoxyphenyl)-methylthio]pyridine-3,5-dicarbonitrile (220 mg, 526.97 μmol, 25.90% yield) as a white solid. ESI [M+H]=418.1.

Step 2. 4-[4-(2-Hydroxyethoxy)phenyl]-2-thiopyridine-3,5-dicarbonitrile (61-C). A mixture of 4-[4-(2-hydroxyethoxy)phenyl]-2-[(4-methoxyphenyl)methylthio]pyridine-3,5-dicarbonitrile (200 mg, 479.07 μmol, 1 eq) in TFA (3 mL) was stirred at 75° C. for 2 h. The reaction mixture was concentrated under reduced pressure to give 4-[4-(2-hydroxyethoxy)phenyl]-2-thiopyridine-3,5-dicarbonitrile (100 mg, 336.33 μmol, 70.20% yield) as a brown oil. ESI [M+H]=296.0.

Step 3. 4-[4-(2-Hydroxyethoxy)phenyl]-2-(oxetan-3-ylmethylthio)pyridine-3,5-dicarbonitrile (61). A mixture of 4-[4-(2-hydroxyethoxy)phenyl]-2-thiopyridine-3,5-dicarbonitrile (100 mg, 336.33 3 mol, 1 eq), 3-(bromomethyl)oxetane (60.94 mg, 403.59 mol, 1.2 eq), K₂CO₃ (92.96 mg, 672.65 μmol, 2 eq) in DMF (2 mL) was stirred at 45° C. for 1 h. The reaction mixture was filtered, and the filtrate was collected. The residue was purified by prep-HPLC (column: Waters Xbridge Prep OBD C18 150 mm×40 mm×10 μm; mobile phase A: 10 mM NH₄HCO₃ in water, mobile phase B: ACN; 25%-55% over 8 m, to give 4-[4-(2-hydroxyethoxy)phenyl]-2-(oxetan-3-ylmethylsulfanyl)pyridine-3,5-dicarbonitrile (53.8 mg, 146.43 μmol, 43.54% yield, 100% purity) as white solid. (H-NMR (400 MHz, DMSO-d₆) δ=9.11 (d, J=0.8 Hz, 1H), 7.58 (d, J=8.4 Hz, 2H), 7.14 (d, J=8.4 Hz, 2H), 4.91 (t, J=5.2 Hz, 1H), 4.61-4.68 (m, 2H), 4.34 (t, J=6.4 Hz, 2H), 4.04-4.11 (m, 2H), 3.58-3.80 (m, 4H), 3.32-3.37 (m, 1H). ESI [M+H]=368.0

TABLE 4
The following compounds were synthesized in a similar manner, but with
different starting materials.

| Compound | Structure | Spectroscopic data |
|---|---|---|
| 3 | 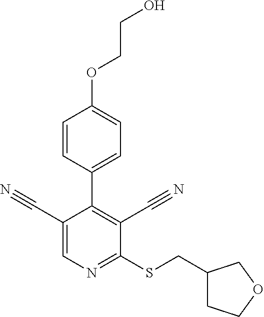 [SMILES: N#Cc1cnc(SCC2CCOC2)c(C#N)c1-c1ccc(OCCO)cc1] | ¹H-NMR (400 MHz, CD₃OD) δ = 8.92 (s, 1H), 7.59 (d, J = 8.8 Hz, 2H), 7.25-7.31 (m, 1H), 7.13-7.25 (m, 2H), 4.13-4.20 (m, 2H), 3.87- 3.96 (m, 4H), 3.78 (d, J = 7.2 Hz, 1H), 3.58- 3.63 (m, 1H), 3.47 (s, 2H), 2.64-2.79 (m, 1H), 2.10-2.27 (m, 1H), 1.71-1.89 (m, 1H). |
| 64 | 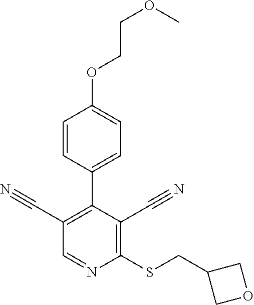 [SMILES: COCCOc1ccc(-c2c(C#N)cnc(SCC3COC3)c2C#N)cc1] | ¹H-NMR (400 MHz, DMSO-d₆) δ = 9.12 (s, 1H), 7.58 (d, J = 8.8 Hz, 2H), 7.15 (d, J = 8.4 Hz, 2H), 4.59-4.68 (m, 2H), 4.34 (t, J = 6.4 Hz, 2H), 4.15-4.23 (m, 2H), 3.64-3.69 (m, 4H), 3.31 (s, 4H). ESI [M + H] = 382.0 |

### Protocol 5. 4-[4-(2-Methoxyethoxy)phenyl]-2-(3-pyridylmethylthio)-pyridine-3,5-dicarbonitrile (83)

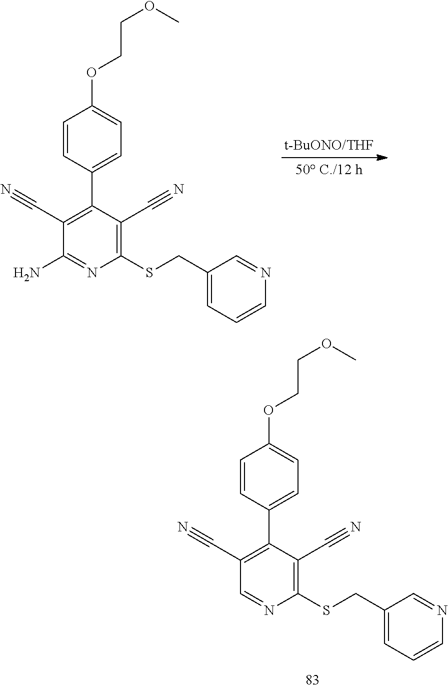 [SMILES: COCCOc1ccc(-c2c(C#N)c(N)nc(SCc3cccnc3)c2C#N)cc1 ; COCCOc1ccc(-c2c(C#N)cnc(SCc3cccnc3)c2C#N)cc1]

A mixture of 2-amino-4-[4-(2-methoxyethoxy)phenyl]-6-(3-pyridylmethylthio)pyridine-3,5-dicarbonitrile (200 mg, 479.06 μmol, 1 eq), t-BuONO (395.20 mg, 3.83 mmol, 455.83 μL, 8 eq) in THF (5 mL) was stirred at 50° C. for 12 h under N₂ atmosphere. The reaction mixture was filtered, and the filtrate was collected. The residue was purified by preparative HPLC (column: Waters Xbridge Prep OBD C18 150 mm×40 mm×10 μm; mobile phase A: 10 mM NH₄HCO₃ in water, mobile phase B: ACN; 30%-55% over 8 min, to give 4-[4-(2-methoxyethoxy)phenyl]-2-(3-pyridylmethyl-thio)pyridine-3,5-dicarbonitrile (147.5 mg, 340.83 μmol, 71.15% yield, 93.0% purity) as a white solid. ¹H-NMR (400 MHz, DMSO-d₆) δ=9.22 (s, 1H), 8.72 (d, J=2.0 Hz, 1H), 8.49 (dd, J=4.8, 1.2 Hz, 1H), 7.91 (dt, J=7.6, 1.6 Hz, 1H), 7.63 (d, J=8.8 Hz, 2H), 7.39 (dd, J=7.8, 4.8 Hz, 1H), 7.20 (d, J=8.8 Hz, 2H), 4.68 (s, 2H), 4.22

### Protocol 6. 2-Cyclopropyl-4-[4-(2-hydroxyethoxy)phenyl]-6-(3-pyridylmethylsulfanyl)-pyridine-3,5-dicarbonitrile (45)

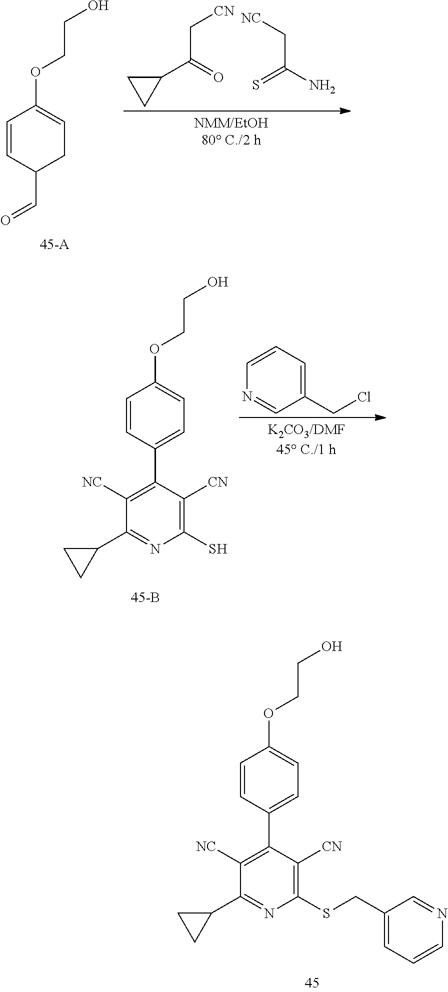

Step 1. 2-Cyclopropyl-4-[4-(2-hydroxyethoxy)phenyl]-6-sulfanyl-pyridine-3,5-dicarbonitrile (45-B). A mixture of 4-(2-hydroxyethoxy)benzaldehyde (300 mg, 1.81 mmol, 1 eq), 3-cyclopropyl-3-oxo-propanenitrile (197.01 mg, 1.81 mmol, 1 eq) and 2-cyanothioacetamide (180.79 mg, 1.81 mmol, 1 eq) and NMM (456.53 mg, 4.51 mmol, 496.22 μL, 2.5 eq) in EtOH (5 mL) was degassed at 80° C. for 2 h. The reaction mixture was added H₂O (20 mL) at 0° C., and then extracted with EtOAc (3×20 mL). The combined organic layers were dried over Na₂SO₄, filtered and concentrated under reduced pressure to give 2-cyclopropyl-4-[4-(2-hydroxyethoxy)phenyl]-6-sulfanyl-pyridine-3,5-dicarbonitrile (350 mg, crude) as a yellow oil. [M+H]=338.0.

Step 2. 2-Cyclopropyl-4-[4-(2-hydroxyethoxy)phenyl]-6-(3-pyridylmethyl-sulfanyl)pyridine-3,5-dicarbonitrile (45). A mixture of 2-cyclopropyl-4-[4-(2-hydroxyethoxy)phenyl]-6-sulfanyl-pyridine-3,5-dicarbonitrile (350 mg, 1.04 mmol, 1 eq), 3-(chloromethyl)pyridine (198.51 mg, 1.56 mmol, 1.5 eq) and K₂CO₃ (286.74 mg, 2.07 mmol, 2 eq) in DMF (3 mL) was stirred at 45° C. for 1 h. The reaction mixture was concentrated under reduced pressure to give a residue. The residue was purified by HPLC using a column Phenomenex Gemini-NX 80 mm×40 mm×3 μm; mobile phase A: 10 mM NH₄HCO₃ in water, mobile phase B: ACN; 30%-55% over 8 min, to give 2-cyclopropyl-4-[4-(2-hydroxyethoxy)phenyl]-6-(3-pyridylmethylsulfanyl)pyridine-3,5-dicarbonitrile (56.9 mg, 129.20 μmol, 12.45% yield, 97.3% purity) as a white solid. ¹H-NMR (400 MHz, DMSO-d₆) δ=8.60 (s, 1H) 8.45 (d, J=4.8 Hz, 1H) 7.79 (d, J=7.6 Hz, 1H) 7.55 (d, J=8.4 Hz, 2H) 7.35 (dd, J=7.6, 4.8 Hz, 1H) 7.36-7.08 (m, 1H) 7.12 (d, J=8.8 Hz, 1H) 4.91 (t, J=5.2 Hz, 1H) 4.55 (s, 2H) 4.06 (t, J=4.8 Hz, 2H) 3.72 (q, J=4.8 Hz, 2H) 2.44-2.40 (m, 1H) 1.28-1.20 (m, 2H) 1.16-1.08 (in, 2H). [M+H]=429.1.

TABLE 5
The following compounds were synthesized in a similar manner, but with
different starting materials.

| Compound | Structure | Spectroscopic data |
|---|---|---|
| 46 | 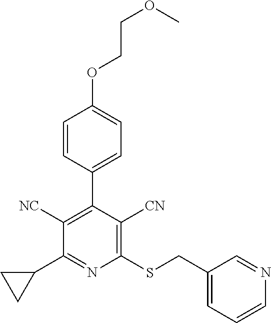 [SMILES: COCCOc1ccc(-c2c(C#N)c(SCc3cccnc3)nc(C3CC3)c2C#N)cc1] | ¹H-NMR (400 MHz, DMSO-d₆) δ = 8.60 (s, 1H) 8.45 (d, J = 4.8 Hz, 1H) 7.79 (d, J = 7.6 Hz, 1H) 7.55 (d, J = 8.4 Hz, 2H) 7.35 (dd, J = 7.6, 4.8 Hz, 1H) 7.13 (d, J = 8.4 Hz, 2H) 4.55 (s, 2H) 4.20-4.12 (m, 2H) 3.68- 3.64 (m, 2H) 3.29 (s, 3H) 2.40-2.42 (m, 1H) 1.28-1.08 (m, 4H). [M + H] = 443.15 |
| 233 | 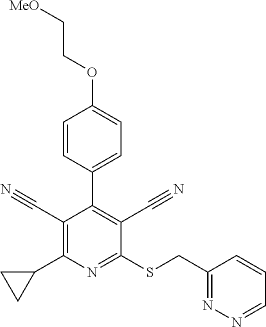 [SMILES: COCCOc1ccc(-c2c(C#N)c(SCc3cccnn3)nc(C3CC3)c2C#N)cc1] | ¹H NMR (400 MHz, DMSO-d₆) δ = 9.20- 9.14 (m, 1H), 7.82-7.75 (m, 1H), 7.73-7.66 (m, 1H), 7.58 (d, J = 8.8 Hz, 2H), 7.16 (d, J = 8.8 Hz, 2H), 4.86 (s, 2H), 4.23-4.18 (m, 2H), 3.72-3.67 (m, 2H), 3.33 (s, 3H), 2.44- 2.38 (m, 1H), 1.24-1.18 (m, 2H), 1.10-1.02 (m, 2H) [M + H] = 444.2 |
| 239 | 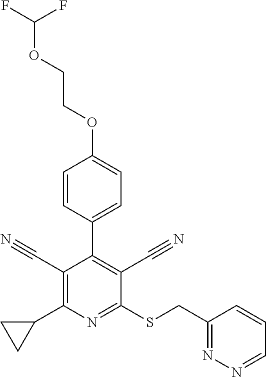 [SMILES: N#Cc1c(SCc2cccnn2)nc(C2CC2)c(C#N)c1-c1ccc(OCCOC(F)F)cc1] | ¹H NMR (400 MHz, DMSO-d₆) δ = 9.17 (dd, J = 4.8, 1.2 Hz, 1H), 7.82-7.76 (m, 1H), 7.70 (dd, J = 8.4, 4.8 Hz, 1H), 7.61 (d, J = 8.8 Hz, 2H), 7.19 (d, J = 8.8 Hz, 2H), 6.78 (t, J = 75.6 Hz, 1H), 4.86 (s, 2H), 4.35-4.20 (m, 4H), 2.46-2.38 (m, 1H), 1.22 (qd, J = 7.6, 3.6 Hz, 2H), 1.09-1.03 (m, 2H) [M + H] = 480.1 |
| 242 | 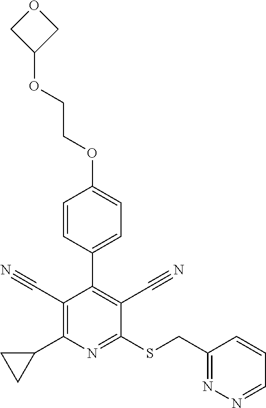 [SMILES: N#Cc1c(SCc2cccnn2)nc(C2CC2)c(C#N)c1-c1ccc(OCCOC2COC2)cc1] | ¹H NMR (400 MHz, DMSO-d₆) δ 9.19-9.12 (m, 1H), 7.77 (d, J = 1.2 Hz, 1H), 7.71-7.67 (m, 1H), 7.60 (d, J = 8.8 Hz, 2H), 7.01 (d, J = 8.8 Hz, 2H), 5.40 (t, J = 5.6 Hz, 1H), 4.96 (t, J = 6.8 Hz, 2H), 4.85 (s, 2H), 4.62-4.58 (m, 2H), 2.43-2.37 (m, 1H), 1.23-1.18 (m, 2H), 1.07-1.03 (m, 2H) |

### Protocol 7. 2-Amino-4-[4-(2-methoxyethoxy)phenyl]-6-methyl-pyridine-3,5-dicarbonitrile (39)

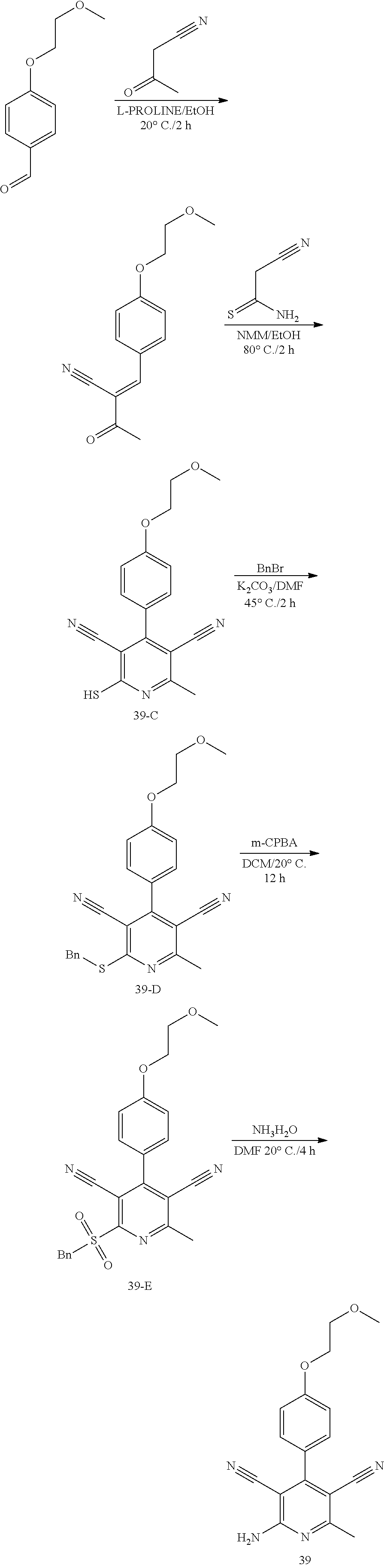

Step 1: (2Z)-2-[[4-(2-Methoxyethoxy)phenyl]methylene]-3-oxo-butanenitrile (39B). To a solution of 4-(2-methoxyethoxy)benzaldehyde (1 g, 5.55 mmol, 1 eq) in EtOH (10 mL) was added L-proline (638.90 mg, 5.55 mmol, 1 eq) and 3-oxobutanenitrile (461.09 mg, 5.55 mmol, 1 eq). The mixture was stirred at 20° C. for 2 h. The reaction mixture was filtered, and the filtrate was collected to give (2Z)-2-[[4-(2-methoxyethoxy)phenyl]methylene]-3-oxo-butanenitrile (800 mg, crude) as a white oil. [M+H]=246.1

Step 2: 4-[4-(2-Methoxyethoxy)phenyl]-2-methyl-6-sulfanyl-pyridine-3,5-dicarbonitrile (39-C). To a solution of (2E)-2-[[4-(2-methoxyethoxy)phenyl]methylene]-3-oxo-butanenitrile (700 mg, 2.85 mmol, 1 eq) in EtOH (10 mL) was added NMM (577.34 mg, 5.71 mmol, 627.54 μL, 2 eq) and 2-cyanothioacetamide (285.80 mg, 2.85 mmol, 1 eq). The mixture was stirred at 80° C. for 2 h. The reaction mixture was filtered, and the solid was collected to give 4-[4-(2-methoxyethoxy)phenyl]-2-methyl-6-sulfanyl-pyridine-3,5-dicarbonitrile (450 mg, crude) as white oil. ESI [M+H]=326.1

Step 3: 2-Benzylsulfanyl-4-[4-(2-methoxyethoxy)phenyl]-6-methyl-pyridine-3,5-dicarbonitrile (39-D). To a solution of 4-[4-(2-methoxyethoxy)phenyl]-2-methyl-6-sulfanyl-pyridine-3,5-dicarbonitrile (50 mg, 153.66 μmol, 1 eq) in DMF (1 mL) was added K₂CO₃ (42.47 mg, 307.33 μmol, 2 eq) and bromomethylbenzene (26.28 mg, 153.66 μmol, 18.25 μL, 1 eq). The mixture was stirred at 45° C. for 2 h. The reaction mixture was poured into 10 mL of H₂O and extracted with EtOAc (3×10 mL). The combined organic phase was extracted with H₂O (3×10 mL), then dried with anhydrous Na₂SO₄, filtered, and concentrated in vacuum to give 2-benzylsulfanyl-4-[4-(2-methoxyethoxy)phenyl]-6-methyl-pyridine-3,5-dicarbonitrile (50 mg, crude) as a white oil. ESI [M+H]=416.1.

Step 4: 2-Benzylsulfonyl-4-[4-(2-methoxyethoxy)phenyl]-6-methyl-pyridine-3,5-dicarbonitrile (39-E). To a solution of 2-benzylsulfanyl-4-[4-(2-methoxyethoxy)phenyl]-6-methyl-pyridine-3,5-dicarbonitrile (50 mg, 120.34 μmol, 1 eq) in THF (2 mL) and DMF (1 mL) was added m-CPBA (73.29 mg, 361.01 μmol, 85% purity, 3 eq). The mixture was stirred at 20° C. for 12 h. The reaction mixture was quenched by addition of saturated aqueous Na₂SO₃ (5 mL) at 0° C., and then extracted with EtOAc (3×5 mL). The combined organic layers were dried over Na₂SO₄, filtered, and concentrated under reduced pressure to give 2-benzylsulfonyl-4-[4-(2-methoxyethoxy)phenyl]-6-methyl-pyridine-3,5-dicarbonitrile (50 mg, crude) as a white oil. ESI [M+H]=448.1.

Step 5: 2-Amino-4-[4-(2-methoxyethoxy)phenyl]-6-methyl-pyridine-3,5-dicarbonitrile (39). To a solution of 2-benzylsulfonyl-4-[4-(2-methoxyethoxy)phenyl]-6-methyl-pyridine-3,5-dicarbonitrile (20 mg, 44.69 μmol, 1 eq) in DMF (2 mL) was added NH₃ in water (5.59 mg, 44.69 μmol, 6.15 μL, 28% purity, 1 eq). The mixture was stirred at 20° C. for 4 h. The reaction mixture was filtered, and the filtrate was collected. The crude was purified by preparative HPLC (column: Phenomenex Gemini-NX C18, 75 mm×30 mm×3 μm; mobile phase A: 0.05% NH₃+10 mM NH₄HCO₃ in water, mobile phase B: ACN; 20%-45% over 8 min, to give 2-amino-4-[4-(2-methoxyethoxy)phenyl]-6-methyl-pyridine-3,5-dicarbonitrile (1.9 mg, 5.92 μmol, 13.25% yield, 96.08% purity) as a white solid. ¹H-NMR (400 MHz, CDCl₃) δ=7.52 (d, J=8.4 Hz, 2H), 7.11 (d, J=8.4 Hz, 2H), 5.72 (s, 2H), 4.22 (t, J=9.6, 4.8 Hz, 2H), 3.81 (t, J=9.6, 4.8 Hz, 2H), 3.49 (s, 3H), 2.70 (m, 3H). [M+H]=309.1

### Protocol 8. 2-Amino-4-[4-(1,1-difluoro-2-hydroxy-ethoxy)phenyl]-6-(oxetan-3-ylmethylsulfanyl)pyridine-3,5-dicarbonitrile (311)

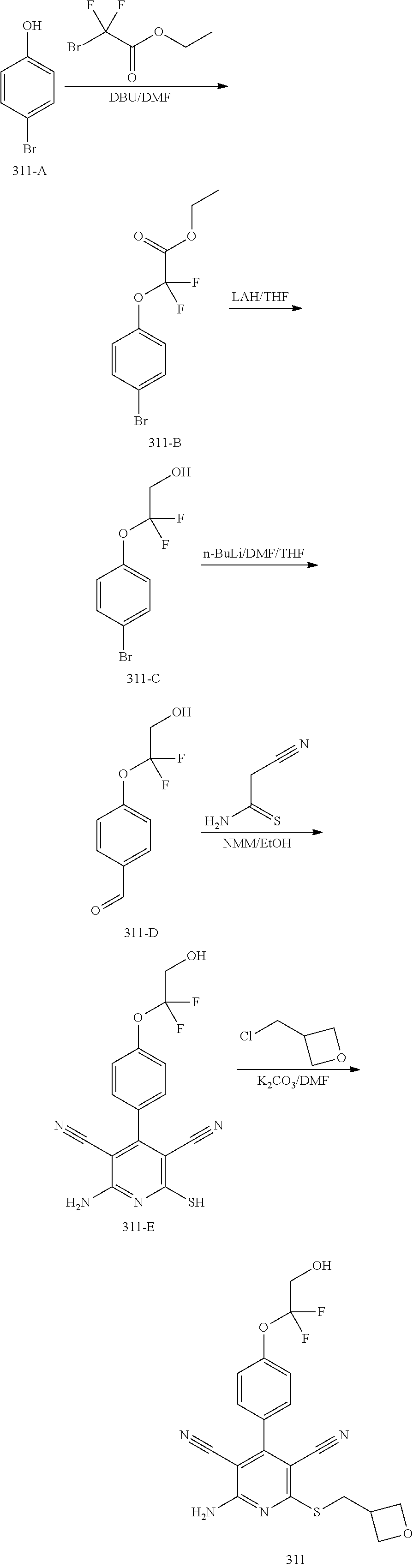

Step 1: Ethyl 2-(4-bromophenoxy)-2,2-difluoroacetate (311-B). To a mixture of 4-bro mophenol (10 g, 57.80 mmol, 1 eq) and ethyl 2-bromo-2,2-difluoroacetate (15.25 g, 75.14 m mol, 9.65 mL, 1.3 eq) in DMF (100 mL) was added DBU (11.44 g, 75.14 mmol, 11.33 mL, 1.3 eq) and the mixture was stirred at 70° C. for 12 hrs under N₂. The reaction mixture was filter ed. The residue was purified by column chromatography (SiO₂, Petroleum ether/Ethyl acetate=10/1 to 1/1,plate1), based on TLC (Plate 1: Petroleum ether:Ethyl acetate=3:1) to give ethyl 12-(4-bromophenoxy)-2,2-difluoroacetate (7 g, 23.72 mmol, 41.04% yield) as brown oil. [M+H]=294.1. ¹H-NMR (400 MHz, CD₃OD) δ ppm 7.46-7.36 (m, 2H), 7.04 (d, J=8.8 Hz, 2H), 4.32 (q, J=7.2 Hz, 2H), 1.30 (t, J=7.2 Hz, 3H)

Step 2: 2-(4-Bromophenoxy)-2,2-difluoro-ethanol (311-C). To a mixture of LiAlH₄ (1.67 g, 44.06 mmol, 2 eq) in THF (3 mL) was added ethyl 2-(4-bromophenoxy)-2,2-difluoroacetate (6.5 g, 22.03 mmol, 1 eq) and the mixture was stirred at 0° C. for 1 hr under N₂. The react ion mixture was quenched by addition Na₂SO₄.10H₂O₃ g at 0° C., and then diluted with THF 50 mL. The mixture was dried over Na₂SO₄, filtered and concentrated under reduced pressure to give a residue. The residue was purified by column chromatography (SiO₂, Petroleum ether/Ethyl acetate=10/1 to 1/1,plate1), based on TLC (Plate 1: Petroleum ether:Ethyl acetate=8:1) to give 2-(4-bromophenoxy)-2,2-difluoroethanol (3.9 g, 15.41 mmol, 69.97% yield) as Brown oil. [M+H]=252.8.

Step 3: 4-(1,1-Difluoro-2-hydroxy-ethoxy)benzaldehyde (311-D). To a mixture of 2-(4-bromophenoxy)-2,2-difluoroethanol (1.5 g, 5.93 mmol, 1 eq) in THF (50 mL) was added n-BuLi (2.5 M, 7.11 mL, 3 eq) at −78° C. for 0.5 hr, then was added DMF (866.58 mg, 11.86 mmol, 912.19 uL, 2 eq), the mixture was stirred at 20° C. for 1.5 hrs under N₂. The reaction mixture was diluted with sat.aq NH₄Cl 50 mL and extracted with EtOAc (15 mL×3). The combined organic layers were washed with sat.aq.NaCl (10 mL),dried over Na₂SO₄, filtered and concentrated under reduced pressure to give a residue. The residue was purified by column chromatography (SiO₂, Petroleum ether/Ethyl acetate=50/1 to 1/1,plate1), based on TLC (Plate 1: Petroleum ether:Ethyl acetate=8:1) to give 4-(1,1-difluoro-2-hydroxy-ethoxy)benzaldehyde (300 mg, 1.48 mmol, 25.03% yield) as Brown oil. [M+H]=203.0.

Step 4: 2-Amino-4-[4-(1,1-difluoro-2-hydroxy-ethoxy)phenyl]-6-sulfanyl-pyridine-3,5-dicarbonitrile (311-E). To a mixture of 4-(1,1-difluoro-2-hydroxy-ethoxy)benzaldehyde (270 mg, 1.34 mmol, 1 eq) and 2-cyanothioacetamide (294.25 mg, 2.94 mmol, 2.2 eq) in EtOH (5 mL) was added NMM (405.28 mg, 4.01 mmol, 440.52 uL, 3 eq), the mixture was stirred at 60° C. for 2 hrs. The reaction mixture was diluted with H₂O 10 mL and extracted with EtOAc (15 mL×3). The combined organic layers were washed with sat.aq.NaCl (10 mL),dried over Na₂SO₄, filtered and concentrated under reduced pressure to give a residue. to give 2-amino-4-[4-(1,1-difluoro-2-hydroxy-ethoxy)phenyl]-6-sulfanyl-pyridine-3,5-dicarbonitrile (300 mg, c rude) as Brown oil. [M+H]=349.2.

Step 5: 2-Amino-4-[4-(1,1-difluoro-2-hydroxy-ethoxy)phenyl]-6-(oxetan-3-ylmethylsulfanyl)pyridine-3,5-dicarbonitrile (311). To a solution of 2-amino-4-[4-(1,1-difluoro-2-hydroxy-ethoxy)phenyl]-6-sulfanyl-pyridine-3,5-dicarbonitrile (120 mg, 344.50 umol, 1 eq) and 3-(bromomethyl)oxetane (104.04 mg, 689.01 umol, 2 eq) in DMF (1 mL) was added K2CO3 (142.84 mg, 1.03 mmol, 3 eq), the mixture was stirred at 20° C. for 0.5 hr. The reaction mixture was filtered. The residue was purified by preparative HPLC (Phenomenex Gemini C₁₈ column (75×30 mm, 3 mm); flow rate: 25 mL/min; gradient: 25% 55% B over 8 min; mobile phase A: 10 mM aqueous NH₄HCO₃, mobile phase B: acetonitrile) to give 2-amino-4-[4-(1,1-difluoro-2-hydroxy-ethoxy)phenyl]-6-(oxetan-3-ylmethylsulfanyl)pyridine-3,5-dicarbonitrile (38.7 mg, 92.49 umol, 26.85% yield, 100% purity) as a brown oil. ¹H NMR (400 MHz, CD₃OD) δ=7.58 (d, J=8.4 Hz, 2H), 7.42 (d, J=8.4 Hz, 2H), 4.83 (d, J=7.6 Hz, 2H), 4.52 (t, J=6.0 Hz, 2H), 3.96 (t, J=9.6 Hz, 2H), 3.65 (d, J=7.6 Hz, 2H), 3.51-3.40 (m, 1H). [M+H]=419.2

TABLE 6
The following compounds were synthesized in a similar manner as described
for compound 311.

| Compound | Structure | Spectroscopic data |
|---|---|---|
| 309 | 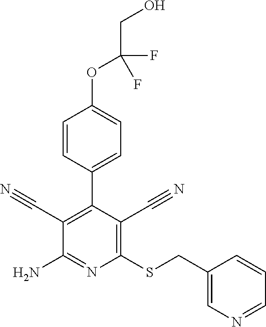 [SMILES: N#Cc1c(N)nc(SCc2cccnc2)c(C#N)c1-c1ccc(OC(F)(F)CO)cc1] | ¹H-NMR (400 MHz, CD₃OD) δ 9.05 (br s, 1H), 8.79-8.61 (m, 2H), 7.96 (br t, J = 6.4 Hz, 1H), 7.59-7.51 (m, 2H), 7.41 (d, J = 8.4 Hz, 2H), 4.69 (s, 2H), 3.96 (t, J = 9.6 Hz, 2H) |
| 310 | 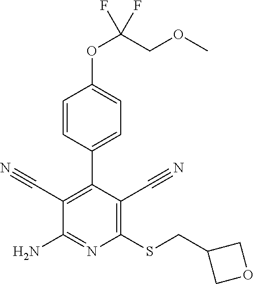 [SMILES: COCC(F)(F)Oc1ccc(-c2c(C#N)c(N)nc(SCC3COC3)c2C#N)cc1] | ¹H NMR (400 MHz, MeOD) δ = 7.63- 7.54 (m, 2H), 7.41 (d, J = 8.4 Hz, 2H), 4.86-4.80 (m, 2H), 4.52 (t, J = 6.0 Hz, 2H), 3.92 (t, J = 9.2 Hz, 2H), 3.65 (d, J = 7.6 Hz, 2H), 3.56 (s, 3H), 3.50-3.40 (m, 1H) [M + H] = 433.0 |
| 306 | 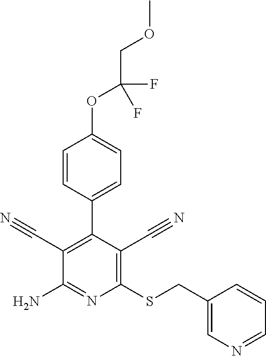 [SMILES: COCC(F)(F)Oc1ccc(-c2c(C#N)c(N)nc(SCc3cccnc3)c2C#N)cc1] | ¹H-NMR (400 MHz, DMSO-d₆) δ 8.78 (d, J = 1.6 Hz, 1H), 8.45 (dd, J = 4.8 1.50 Hz, 1H), 7.94 (dt, J = 8.0, 1.6 Hz, 1H), 7.63-7.55 (m, 2H), 7.41- 7.31 (m, 3H), 4.50 (s, 2 H), 3.95 (t, J = 10.0 Hz, 2H), 3.45 (s, 3 H) |

### Protocol 9. 2-Amino-4-(4-((1r,3r)-3-hydroxycyclobutoxy)phenyl)-6-((pyridin-3-ylmethyl)thio)pyridine-3,5-dicarbonitrile (295) and 2-Amino-4-(4-((1s,3s)-3-hydroxycyclobutoxy)phenyl)-6-((pyridin-3-ylmethyl)thio)pyridine-3,5-dicarbonitrile (296)

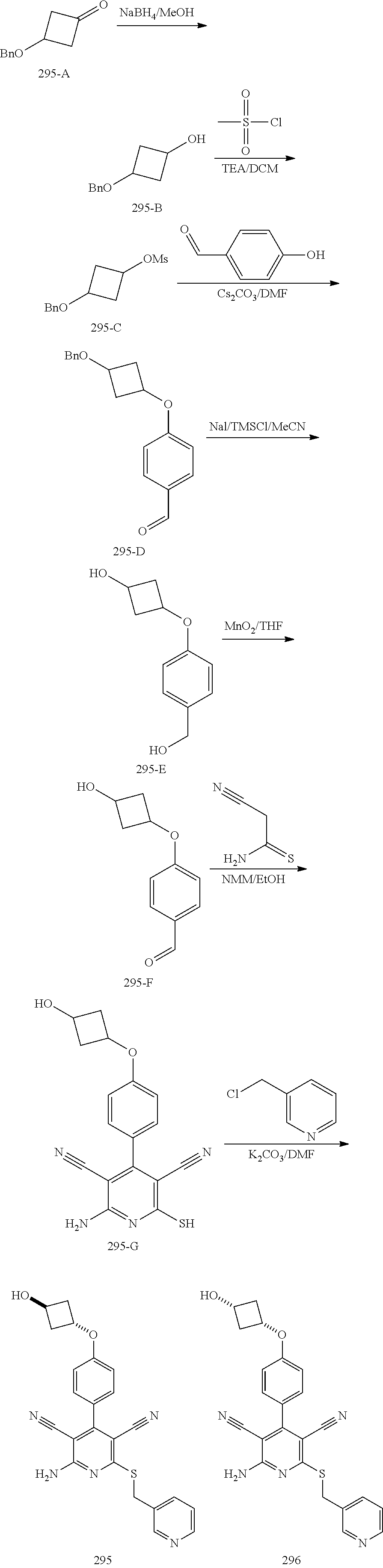

Step 1: 3-(Benzyloxy)cyclobutan-1-ol (295-B). To a mixture of 3-(benzyloxy)cyclobutan-1-one (5.0 g, 28.38 mmol, 1.0 eq) in MeOH (60.0 mL) was added NaBH₄ (1.29 g, 34.05 m mol, 1.2 eq) at 0° C. and the mixture was stirred at 20° C. for 1.5 hrs. TLC (Petroleum ether:Ethyl acetate=1:1) showed the reaction was completed and then quenched by addition of saturated aqueous NH₄Cl (100 mL) at 0° C. and extracted with EtOAc (100 mL×3). The combined organic layers were washed with brine (100 mL), dried over Na₂SO₄, filtered and concentrate d. The residue was purified by preparative TLC (SiO₂, Petroleum ether:Ethyl acetate=1:1) to give 3-(benzyloxy)cyclobutan-1-ol (3.6 g, 20.20 mmol, 71% yield) as yellow oil. ¹H NMR (400 MHz, CD₃OD) δ ppm 7.39-7.26 (m, 5H), 4.49-4.37 (m, 2H), 3.91-3.79 (m, 1H), 3.71-3.57 (m, 1H), 2.78-2.55 (m, 2H), 2.00-1.78 (m, 2H).

Step 2: 3-(Benzyloxy)cyclobutyl methanesulfonate (295-C). To a mixture of 3-(benzyl oxy)cyclobutan-1-ol (3.5 g, 19.64 mmol, 1.0 eq) in DCM (50.0 mL) was added TEA (5.96 g, 58.91 mmol, 8.20 mL, 3.0 eq) and methanesulfonyl chloride (3.37 g, 29.46 mmol, 1.5 eq) at 0° C. and the mixture was stirred at 25° C. for 1 hr. Then the mixture was quenched by addition of saturated aqueous Na₂CO₃ (100 mL) at 0° C. and extracted with EtOAc (100 mL×3). The combined organic layers were washed with brine (100 mL), dried over Na₂SO₄, filtered and concentrated. The residue was purified by preparative TLC (SiO₂, Petroleum ether:Ethyl acetate=1:1) to give 3-(benzyloxy)cyclobutyl methanesulfonate (4.5 g, 17.56 mmol, 89.40% yield) as yellow oil.

Step 3: 4-(3-(Benzyloxy)cyclobutoxy)benzaldehyde (295-D). To a mixture of 3-(benzyloxy)cyclobutyl methanesulfonate (4.5 g, 17.56 mmol, 1.0 eq) and 4-hydroxybenzaldehyde (20.14 g, 17.56 mmol, 1.0 eq) in DMF (25.0 mL) was added Cs₂CO₃ (11.44 g, 35.11 mmol, 2 eq) at 25° C., the mixture was stirred at 100° C. for 5 hrs. Then the mixture was filtered and the filtrate was concentrated under reduced pressure to give a residue. The residue was purified by preparative TLC (SiO₂, Petroleum ether:Ethyl acetate=3:1) to give 4-(3-(benzyloxy)cyclobutoxy)benzaldehyde (2.5 g, 8.85 mmol, 50.44% yield) as yellow oil. [M+H]=283.1

Step 4: 3-(4-(Hydroxymethyl)phenoxy)cyclobutan-1-ol (295-E). To a mixture of 4-(3-(benzyloxy)cyclobutoxy)benzaldehyde (1.0 g, 3.54 mmol, 1.0 eq) in MeCN (15.0 mL) was added NaI (1.59 g, 10.63 mmol, 3 eq) and TMSCl (1.15 g, 10.63 mmol, 3 eq) at 25° C. and the mixture was stirred at 60° C. for 1 hr. The mixture was filtered and the filtrate was concentrated under reduced pressure to give a residue. The residue was purified by preparative TLC (SiO₂, Petroleum ether:Ethyl acetate=1:1) to give 3-(4-(hydroxymethyl)phenoxy)cyclobutan-1-ol (400.0 mg, 2.06 mmol, 58.15% yield) as yellow solid. [M+H]=193.1. ¹H NMR (400 MHz, C D₃OD) δ ppm 9.86 (s, 1H), 7.34 (d, J=8.4 Hz, 2H), 6.87-6.76 (m, 2H), 5.34 (s, 1H), 4.91-4.80 (m, 1H), 4.59-4.45 (m, 1H), 2.53-2.33 (m, 4H)

Step 5: 4-(3-Hydroxycyclobutoxy)benzaldehyde (295-F). To a mixture of 3-(4-(hydroxymethyl)phenoxy)cyclobutan-1-ol (350.0 mg, 1.80 mmol, 1.0 eq) in THF (15.0 mL) was added MnO₂ (234.99 mg, 2.70 mmol, 1.5 eq) at 25° C. and the mixture was stirred at 60° C. for 1 hr. Then the mixture was filtered and the filtrate was concentrated under reduced pressure to give 4-(3-hydroxycyclobutoxy)benzaldehyde (300.0 mg, crude) as yellow solid. ¹H NMR (400 MHz, CD₃OD) δ ppm 9.85 (s, 1H), 7.88 (d, J=8.4 Hz, 2H), 6.99 (d, J=8.8 Hz, 2H), 5.03-4.96 (m, 1H), 4.54 (t, J=6.4 Hz, 1H), 2.55-2.41 (m, 4H).

Step 6: 2-Amino-4-(4-(3-hydroxycyclobutoxy)phenyl)-6-mercaptopyridine-3,5-dicarbonitrile (295-G). To a mixture of 4-(3-hydroxycyclobutoxy)benzaldehyde (300.0 mg, 1.56 mmol, 1.0 eq) and 2-cyanoethanethioamide (390.75 mg, 3.90 mmol, 2.5 eq) in EtOH (5.0 mL) was added NMM (394.67 mg, 3.90 mmol, 2.5 eq) at 25° C. and the mixture was stirred at 25° C. for 12 hrs. Then the mixture was filtered and the filtrate was concentrated under reduced pressure to give 2-amino-4-(4-(3-hydroxycyclobutoxy)phenyl)-6-mercaptopyridine-3,5-dicarbonitrile (230.0 mg, 679.70 umol, crude) as yellow solid. [M+H]=339.1

Step 7: 2-Amino-4-(4-((1r,3r)-3-hydroxycyclobutoxy)phenyl)-6-((pyridin-3-ylmethyl)thio)pyridine-3,5-dicarbonitrile (295) and 2-Amino-4-(4-((1s,3s)-3-hydroxycyclobutoxy)phenyl)-6-((pyridin-3-ylmethyl)thio)pyridine-3,5-dicarbonitrile (296). To a mixture of 2-amino-4-(4-(3-hydroxycyclobutoxy)phenyl)-6-mercaptopyridine-3,5-dicarbonitrile (220.0 mg, 650.15 umol, 1.0 eq) and 3-(chloromethyl)pyridine (95.98 mg, 585.14 umol, 0.9 eq) in DMF (4.0 mL) was added K₂CO₃ (179.71 mg, 1.30 mmol, 2 eq) at 25° C. and the mixture was stirred at 80° C. for 2 hrs The reaction mixture was filtered and the filtrate was concentrated under reduced pressure to give a residue. The residue was purified by preparative HPLC (column: Phenomenex Gemini C18 column (100×30 mm, 10 um); flow rate: 25 mL/min; gradient: 20%-50% B over 10 min; mobile phase A: 10 mM aqueous NH₄HCO₃, mobile phase B: acetonitrile) to give the product as a mixture of stereoisomers. The mixture was further separated by SFC (Instrument: Waters UPCC with PDA; Column: DAICEL CHIRALPAK IG (250 mm*30 mm,10 um); Mobile phase A: CO₂ B: 0.1% IPA in EtOH; Gradient: B %=45% isocratic elution mode; Flow rate: CO₂ (4.2 mg/min), B(3.4 mL/min); Wavelength:220 nm; Column temperature: 35° C.; System back pressure: 2000 Psi) to give 2-amino-4-(4-((1r,3r)-3-hydroxycyclobutoxy)phenyl)-6-((pyridin-3-ylmethyl)thio)pyridine-3,5-dicarbonitrile (47.9 mg, 105.34 umol, 16.20% yield, 94.45% purity, free), ¹H NMR (400 MHz, DMSO-d₆) δ ppm 8.78 (d, J=1.6 Hz, 1H), 8.45 (dd, J=4.8, 1.6 Hz, 1H), 7.95 (dt, J=7.6, 1.6 Hz, 1H), 7.48-7.44 (m, 2H), 7.35 (dd, J=7.6, 4.8 Hz, 1H), 6.98-6.93 (m, 2H), 5.20 (d, J=5.2 Hz, 1H), 4.90 (t, J=5.2 Hz, 1H), 4.50 (s, 2H), 4.43-4.34 (m, 1H), 2.34 (t, J=5.6 Hz, 4H), [M+H]=430.2, and 2-amino-4-(4-((1s,3s)-3-hydroxycyclobutoxy)phenyl)-6-((pyridin-3-ylmethyl)thio)pyridine-3,5-dicarbonitrile (8.7 mg, 20.07 umol, 3.09% yield, 99.07% purity), ¹H NMR (400 MHz, DMSO-d₆) δ ppm 8.80 (d, J=1.6 Hz, 1H), 8.45 (dd, J=4.4, 1.2 Hz, 1H), 7.96 (dt, J=7.2 Hz, 1H), 7.46 (d, J=8.8 Hz, 2H), 7.36 (dd, J=7.2, 4.8 Hz, 1H), 7.03-6.96 (m, 2H), 5.25 (d, J=6.4 Hz, 1H), 4.50 (s, J=5.2 Hz, 1H), 4.50 (s, 2H), 4.43-4.34 (m, 1H), 2.34 (t, J=5.6 Hz, 4H). [M+H]=430.2

TABLE 7
The following compounds were synthesized in a similar manner as described
for compounds 295 and 296.

| Compound | Structure | Spectroscopic data |
|---|---|---|
| 303 | 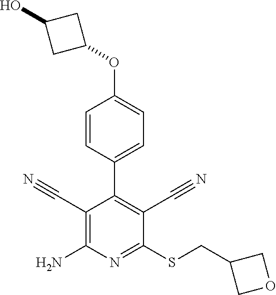 | ¹H NMR (400 MHz, DMSO-d₆) δ ppm 7.48 (d, J = 8.8 Hz, 2H), 6.98 (d, J = 8.8 Hz, 2H), 5.24 (d, J = 5.6 Hz, 1H), 4.92 (t, J = 5.2 Hz, 1H), 4.67 (dd, J = 7.6, 6.4 Hz, 2H), 4.47-4.30 (m, 3H), 3.56 (d, J = 7.6 Hz, 2H), 2.36 (t, J = 5.6 Hz, 4H). |
| 321 | 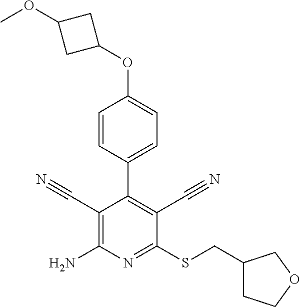 [SMILES: COC1CC(Oc2ccc(-c3c(C#N)c(N)nc(SCC4CCOC4)c3C#N)cc2)C1] | ¹H-NMR (400 MHz, DMSO-d₆) δ ppm 8.19-7.80 (m, 2 H), 7.48 (d, J = 8.4 Hz, 2 H), 7.08-6.94 (m, 2 H), 4.96-4.84 (m, 0.3 H), 4.48 (t, J = 6.8 Hz, 1 H), 4.13- 4.06 (m, 0.4 H), 3.84-3.78 (m, 1.6 H), 3.68-3.63 (m, 1.6 H), 3.45-3.40 (m, 2 H), 3.29 (s, 2 H), 3.22-3.13 (m, 3 H), 2.94-2.89 (m, 1 H), 2.62 (br s, 2 H), 2.46 (br dd, J = 7.2, 2.8 Hz, 1 H), 2.39- 2.28 (m, 1 H), 2.10-2.04 (m, 1 H), 1.97- 1.91 (m, 1 H), 1.74-1.57 (m, 1 H). [M + H] = 437.2 |
| 323 | 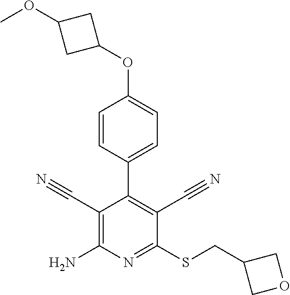 [SMILES: COC1CC(Oc2ccc(-c3c(C#N)c(N)nc(SCC4COC4)c3C#N)cc2)C1] | ¹H-NMR (400 MHz, DMSO-d₆) δ ppm 8.24-7.76 (m, 2 H), 7.48 (d, J = 8.8 Hz, 2 H), 7.08-6.95 (m, 2 H), 4.92 (m, J = 6.8, 4.4, 2.4 Hz, 0.3 H), 4.66 (dd, J = 7.6, 6.0 Hz, 2 H), 4.54-4.42 (m, 0.7 H), 4.36 (t, J = 6.0 Hz, 2 H), 4.15-4.03 (m, 0.3 H), 3.66 (t, J = 6.8 Hz, 0.7 H), 3.55 (d, J = 7.2 Hz, 2 H), 3.32 (br s, 2 H), 3.24- 3.12 (m, 3 H), 2.93-2.86 (m, 1 H), 2.40- 2.26 (m, 1 H), 1.94 (m, J = 7.2 Hz, 1 H). [M + H] = 423.2 |
| 324 | 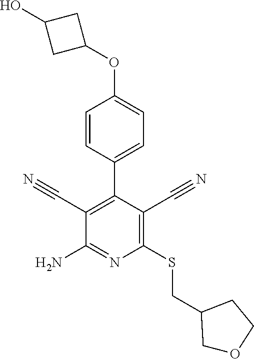 [SMILES: N#Cc1c(N)nc(SCC2CCOC2)c(C#N)c1-c1ccc(OC2CC(O)C2)cc1] | ¹H NMR (400 MHz, CD₃OD) δ = 7.39- 7.33 (m, 2H), 6.92-6.85 (m, 2H), 4.28 (t, J = 6.8Hz, 1H), 3.97-3.87 (m, 1H), 3.85-3.76 (m, 2H), 3.67 (q, J = 7.6 Hz, 1H), 3.49 (dd, J = 8.8, 5.6 Hz, 1H), 3.26 (dd, J = 7.6, 1.6 Hz, 3H), 2.93-2.78 (m, 2H), 2.57 (dt, J = 13.6, 6.8 Hz, 1H), 2.43- 2.29 (m, 1H), 2.07 (m, J = 12.4, 7.6, 7.6, 5.6 Hz, 1H), 2.01-1.95 (m, 1H), 1.75-1.61 (m, 1H). |
| 328 | 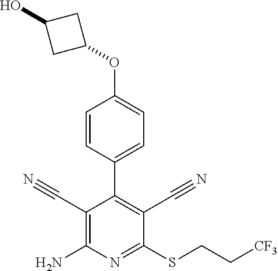 | ¹H-NMR (400 MHz, DMSO-d₆) δ ppm 8.36-7.81 (m, 2H), 7.50 (d, J = 8.8 Hz, 2H), 7.07-6.93 (m, 2H), 5.31-5.20 (m, 1H), 4.94 (q, J = 5.2 Hz, 1H), 4.49-4.29 (m, 1H), 3.38 (br s, 2H), 2.85-2.74 (m, 2H), 2.38 (t, J = 5.6 Hz, 4H). [M + H] = 435.1 |
| 329 | 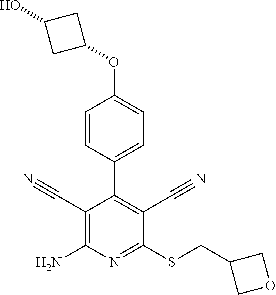 | ¹H-NMR (400 MHz, DMSO-d₆) δ ppm 7.46 (d, J = 8.8 Hz, 2H), 7.00 (d, J = 8.8 Hz, 2H), 5.24 (d, J = 6.8 Hz, 1H), 4.65 (dd, J = 7.6, 6.0 Hz, 2H), 4.43-4.23 (m, 3H), 3.94-3.74 (m, 1H), 3.54 (d, J = 7.2 Hz, 2H), 2.86 (dt, J = 9.2, 6.4, 6.4, 3.2 Hz, 2H), 2.00-1.85 (m, 2H). |

### Protocol 10. 2-Methoxy-6-((oxetan-3-ylmethyl)thio)-4-(4-(oxetan-3-yloxy)phenyl)pyridine-3,5-dicarbonitrile (294)

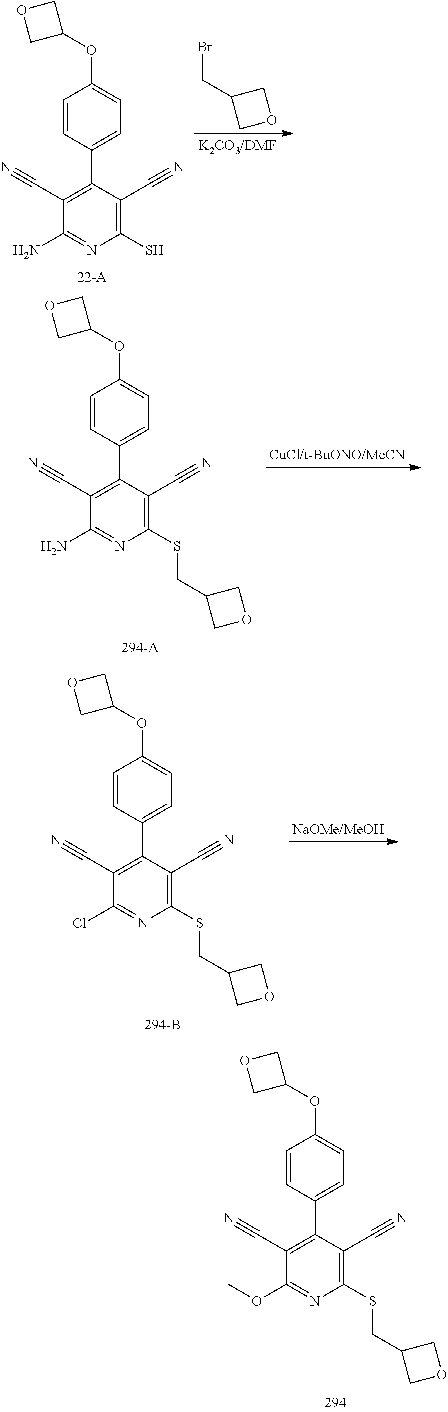 [SMILES: N#Cc1c(N)nc(S)c(C#N)c1-c1ccc(OC2COC2)cc1 ; BrCC1COC1 ; N#Cc1c(N)nc(SCC2COC2)c(C#N)c1-c1ccc(OC2COC2)cc1 ; N#Cc1c(Cl)nc(SCC2COC2)c(C#N)c1-c1ccc(OC2COC2)cc1 ; COc1nc(SCC2COC2)c(C#N)c(-c2ccc(OC3COC3)cc2)c1C#N]

Step 1: 2-Amino-6-((oxetan-3-ylmethyl)thio)-4-(4-(oxetan-3-yloxy)phenyl)pyridine-3,5-dicarbonitrile (294-A). To a mixture of 2-amino-6-mercapto-4-(4-(oxetan-3-yloxy)phenyl) pyridine-3,5-dicarbonitrile (1.0 g, 3.08 mmol, 1.0 eq) and 3-(bromomethyl)oxetane (465.54 mg, 3.08 mmol, 1.0 eq) in DMF (5.0 mL) was added K₂CO₃ (852.19 mg, 6.17 mmol, 2.0 eq) at 25° C. and the reaction mixture was stirred at 25° C. for 1 hr. The reaction mixture was filtered d and the filtrate was concentrated under reduced pressure to give a residue. The residue was purified by column chromatography on silica gel (Ethyl acetate:Petroleum ether=1:0 to 1:2) to give 2-amino-6-((oxetan-3-ylmethyl)thio)-4-(4-(oxetan-3-yloxy)phenyl)pyridine-3,5-dicarbonitrile (550.0 mg, 1.39 mmol, 45.23% yield) as brown solid. [M+H]=395.1

Step 2: 2-Chloro-6-((oxetan-3-ylmethyl)thio)-4-(4-(oxetan-3-yloxy)phenyl)pyridine-3,5-dicarbonitrile (294-B). To a mixture of 2-amino-4-[4-(oxetan-3-yloxy)phenyl]-6-(3-pyridyl methylsulfanyl)pyridine-3,5-dicarbonitrile (250 mg, 633.80 umol, 1.0 eq) in MeCN (3.0 mL) was added t-BuONO (98.04 mg, 950.70 umol, 113.08 ul, 1.5 eq) and CuCl (125.49 mg, 1.27 Mmol, 2.0 eq) at 25° C., the reaction mixture was stirred at 60° C. for 1 hr under N₂ atmosphere. The reaction mixture was filtered and the filtrate was concentrated under reduced pressure to give a residue. The residue was purified by preparative TLC (Petroleum ether:Ethyl acetate=0:1) to give 2-chloro-6-((oxetan-3-ylmethyl)thio)-4-(4-(oxetan-3-yloxy)phenyl)pyridine-3,5-dicarbonitrile (120.0 mg, crude) as green solid. [M+H]=414.1

Step 3: 2-Methoxy-6-((oxetan-3-ylmethyl)thio)-4-(4-(oxetan-3-yloxy)phenyl)pyridine-3,5-dicarbonitrile (294). To a mixture of 2-chloro-6-((oxetan-3-ylmethyl)thio)-4-(4-(oxetan-3-yloxy)phenyl)pyridine-3,5-dicarbonitrile (35.0 mg, 84.57 umol, 1.0 eq) in MeOH (1.0 mL) was added NaOMe (5.48 mg, 101.48 umol, 1.2 eq) at 25° C., the reaction mixture was stirred at 25° C. for 1 hr. The reaction mixture was filtered and the filtrate was concentrated under reduced pressure to give a residue. The residue was purified by preparative HPLC (column: Waters Xbridge BEH C18 (100×30 mm, 10 um); flow rate: 25 mL/min; gradient: 40%-70% B over 8 min; mobile phase A: 10 mM aqueous NH₄HCO₃, mobile phase B: acetonitrile) to give 2-methoxy-6-((oxetan-3-ylmethyl)thio)-4-(4-(oxetan-3-yloxy)phenyl)pyridine-3,5-dicarbonitrile (7.2 mg, 17.58 umol, 20.79% yield) as white solid. [M+H]=410.2. ¹H-NMR (400 MHz, DMSO-d₆) δ ppm 7.57 (d, 2H), 7.02 (d, 2H), 5.40 (t, 1H), 4.97 (t, 2H), 4.69 (dd, 2H), 4.61 (dd, 2H), 4.39 (t, 2H), 4.15 (s, 3H), 3.70 (d, 2H), 3.48-3.39 (m, 1H).

TABLE 8
The following compounds were synthesized in a similar manner as described
for compound 294.

| Compound | Structure | Spectroscopic Data |
|---|---|---|
| 281 | 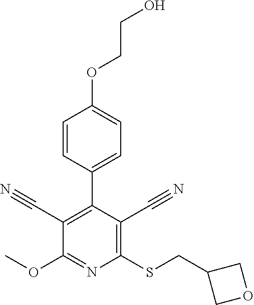 [SMILES: COc1nc(SCC2COC2)c(C#N)c(-c2ccc(OCCO)cc2)c1C#N] | ¹H NMR (400 MHz, DMSO-d₆) δ = 7.54 (d, J = 8.8 Hz, 2H), 7.15 (d, J = 8.8 Hz, 2H), 4.92 (t, J = 5.2 Hz, 1H), 4.68 (t, J = 6.8 Hz, 2H), 4.38 (t, J = 6.0 Hz, 2H), 4.14 (s, 3H), 4.09 (t, J = 4.8 Hz, 2H), 3.75 (q, J = 5.2 Hz, 2H), 3.69 (d, J = 7.2 Hz, 2H), 3.43 (td, J = 14.0, 6.8 Hz, 1H). [M + H] = 398.1 |
| 282 | 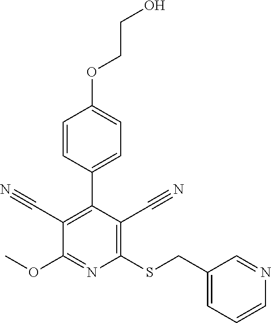 [SMILES: COc1nc(SCc2cccnc2)c(C#N)c(-c2ccc(OCCO)cc2)c1C#N] | ¹H-NMR (400 MHz, CD₃OD) δ = 8.70 (d, J = 1.6 Hz, 1H), 8.49 (dd, J = 4.8, 1.6 Hz, 1H), 8.00 (br d, J = 7.6 Hz, 1H), 7.58- 7.53 (m, 2H), 7.46 (dd, J = 7.6, 4.8 Hz, 1H), 7.19-7.14 (m, 2H), 4.72 (s, 2H), 4.19-4.15 (m, 5H), 3.93 (t, J = 4.8 Hz, 2H). [M + H] = 419.0 |
| 283 | 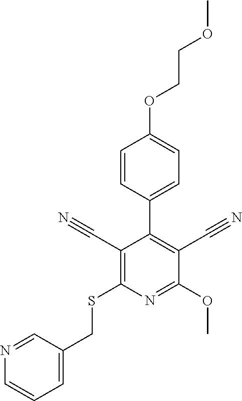 [SMILES: COCCOc1ccc(-c2c(C#N)c(OC)nc(SCc3cccnc3)c2C#N)cc1] | ¹H NMR (400 MHz, CD₃OD) δ = 8.69 (s, 1H) 8.48 (br d, J = 4.4 Hz, 1H) 7.99 (br d, J = 7.6 Hz, 1H) 7.55 (d, J = 8.8 Hz, 2H) 7.45 (dd, J = 7.6, 4.8 Hz, 1H) 7.15 (d, J = 8.8 Hz, 2H) 4.71 (s, 2H) 4.23 (dd, J = 5.2, 3.6 Hz, 2H) 4.17 (s, 3H) 3.80 (dd, J = 5.2, 3.6 Hz, 2 H) 3.45 (s, 3H). [M + H] = 433.2 |

### Protocol 11. 2-Amino-4-(4-(oxetan-3-yloxy)phenyl)-6-((tetrahydro-2H-pyran-3-yl)methoxy)pyridine-3,5-dicarbonitrile (300)

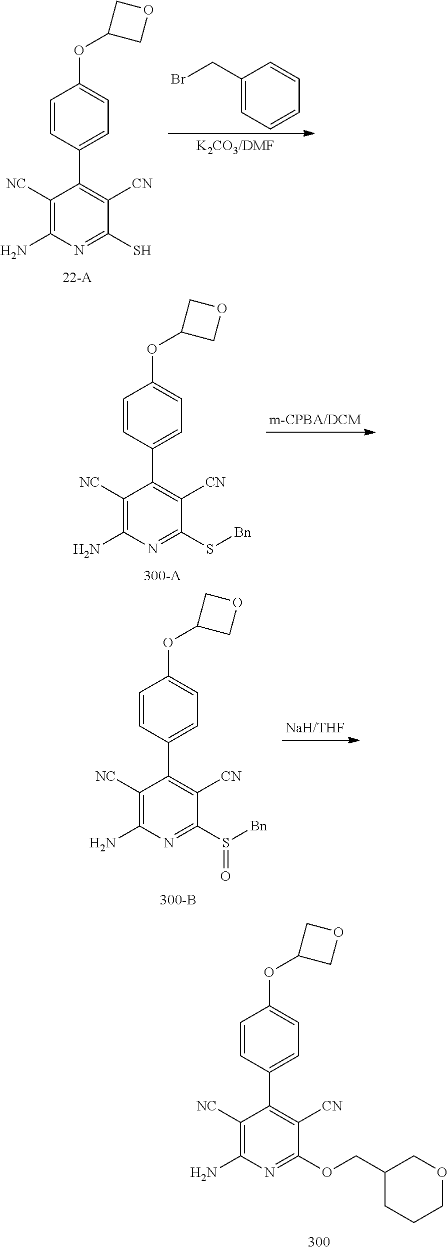

Step 1: 2-Amino-6-(benzylthio)-4-(4-(oxetan-3-yloxy)phenyl)pyridine-3,5-dicarbonitrile (300-A). To a mixture of 2-amino-6-mercapto-4-(4-(oxetan-3-yloxy)phenyl)pyridine-3,5-dicarbonitrile (2 g, 6.17 mmol, 1 eq) and (bromomethyl)benzene (1.05 g, 6.17 mmol, 732.37 uL, 1.00 eq) in DMF (15 mL) was added K₂CO₃ (1.70 g, 12.33 mmol, 2 eq) at 25° C., the mixture was stirred at 25° C. for 1 hr. The reaction mixture was concentrated under reduced pressure, then diluted with H₂O 30 mL and extracted with EtOAc 90 mL(30 mL×3). The combined organic layers were washed with brine (30 mL×2), dried over Na₂SO₄, filtered and concentrated under reduced pressure to 2-amino-6-(benzylthio)-4-(4-(oxetan-3-yloxy)phenyl)pyridine-3,5-dicarbonitrile (3.8 g, crude) as white oil. [M+H]=415.1.

Step 2: 2-Amino-6-(benzylsulfinyl)-4-(4-(oxetan-3-yloxy)phenyl)pyridine-3,5-dicarbonitrile (300-B). To a mixture of 2-amino-6-(benzylthio)-4-(4-(oxetan-3-yloxy)phenyl)pyridine-3,5-dicarbonitrile (3.8 g, 9.17 mmol, 1 eq) in DCM (20 mL) was added m-CPBA (1.12 g, 5.50 mmol, 85% purity, 0.6 eq) at 0° C. and the mixture was stirred at 25° C. for 5 hrs. The reaction mixture was quenched by addition saturated aqueous Na₂SO₃ (30 mL) at 0° C., and then extracted with DCM (30 mL×3). The combined organic layers were dried over Na₂SO₄, filtered and concentrated under reduced pressure to give a residue. The residue was purified by column chromatography on silica gel (Petroleum ether:Ethyl acetate=1:0 to 0:1) to give 2-amino-6-(benzylsulfinyl)-4-(4-(oxetan-3-yloxy)phenyl)pyridine-3,5-dicarbonitrile (600 mg, 1.39 mmol, 15.20% yield) as yellow solid. ¹H-NMR (400 MHz, DMSO-d₆) δ=7.50 (d, J=8.4 Hz, 2H), 7.44-7.37 (m, 3H), 7.25 (dd, J=6.4, 2.8 Hz, 2H), 7.05 (d, J=8.8 Hz, 2H), 5.45 (t, J=5.2 Hz, 1H), 5.03 (t, J=6.8 Hz, 2H), 4.66 (dd, J=7.2, 5.2 Hz, 2H), 4.46 (s, 2H). [M+H]=431.1.

Step 3: 2-Amino-4-(4-(oxetan-3-yloxy)phenyl)-6-((tetrahydro-2H-pyran-3-yl)methoxy)pyridine-3,5-dicarbonitrile (300). To a mixture of (tetrahydro-2H-pyran-3-yl)methanol (26.98 mg, 232.30 umol, 1 eq) in THF (2 mL) was added NaH (37.16 mg, 929.20 umol, 60% purity, 4 eq) at 0° C. The mixture was stirred at 25° C. for 1 hr, then 2-amino-6-(benzylsulfinyl)-4-(4-(oxetan-3-yloxy)phenyl)pyridine-3,5-dicarbonitrile (100 mg, 232.30 umol, 1 eq) was added to the mixture reaction at 0° C., the mixture was stirred at 25° C. for 1 hr. The reaction mixture was concentrated under reduced pressure to give a residue, and the filter cake was quenched by addition saturated aqueous H₂O (2 mL) at 0° C. The residue was purified by preparative HPLC (column: Phenomenex C18 column (75×30 mm, 3 um); flow rate: 25 mL/min; gradient: 20%-50% B over 8 min; mobile phase A: 10 mM aqueous NH₄HCO₃, mobile phase B: acetonitrile) to give 2-amino-4-(4-(oxetan-3-yloxy)phenyl)-6-((tetrahydro-2H-pyran-3-yl)methoxy)pyridine-3,5-dicarbonitrile (19.6 mg, 48.22 umol, 20.76% yield, 100.0% purity) as yellow solid. ¹H-NMR (400 MHz, DMSO-d₆) δ ppm 8.21-7.69 (m, 2H), 7.50 (d, J=8.8 Hz, 2H), 6.98 (d, J=8.8 Hz, 2H), 5.44-5.33 (m, 1H), 4.97 (t, J=6.8 Hz, 2H), 4.61 (dd, J=7.2, 5.2 Hz, 2H), 4.34-4.17 (m, 2H), 3.93-3.67 (m, 2H), 3.40-=3.35 (m, 1H), 3.28 (dd, J=11.2, 9.2 Hz, 1H), 2.12-1.96 (m, 1H), 1.90-1.77 (m, 1H), 1.69-1.46 (m, 2H), 1.45-1.33 (m, 1H). [M+H]=407.2

TABLE 9
The following compounds were synthesized in a similar manner as described
for compound 300.

| Compound | Structure | Spectroscopic data |
|---|---|---|
| 290 | 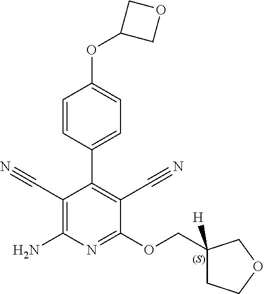 | ¹H-NMR (400 MHz, CD₃OD) δ ppm 7.53 (d, J = 8.8 Hz, 2 H), 6.97 (d, J = 8.8 Hz, 2 H), 5.40 (q, J = 5.2 Hz, 1 H), 5.08 (t, J = 6.4 Hz, 2 H), 4.76 (dd, J = 6.8, 5.2 Hz, 2 H), 4.57- 4.32 (m, 2 H), 4.00-3.86 (m, 2 H), 3.81 (q, J = 7.6 Hz, 1 H), 3.71 (dd, J = 8.8, 5.2 Hz, 1 H), 2.81 (dt, J = 13.2, 6.4 Hz, 1 H), 2.30- 2.05 (m, 1 H), 1.80 (dq, J = 13.2, 6.4 Hz, 1 H). [M + H] = 393.0 |
| 299 | 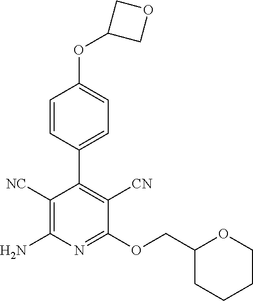 [SMILES: N#Cc1c(N)nc(OCC2CCCCO2)c(C#N)c1-c1ccc(OC2COC2)cc1] | ¹H-NMR (400 MHz, CD₃OD) δ ppm 7.54- 7.50 (m, 2 H), 6.94-6.99 (m, 2 H), 5.39 (t, J = 5.6 Hz, 1 H), 5.07 (t, J = 6.8 Hz, 2 H), 4.75 (dd, J = 7.2, 5.2 Hz, 2 H), 4.46-4.36 (m, 2 H), 4.00 (dd, J = 11.2, 3.2 Hz, 1 H), 3.77 (d, J = 8.8, 8.8, 4.0, 2.4 Hz, 1 H), 3.53 (td, J = 10.8, 3.2 Hz, 1 H), 1.97-1.89 (m, 1 H), 1.74 (br d, J = 12.0 Hz, 1 H), 1.66-1.55 (m, 3 H), 1.53-1.42 (m, 1 H). [M + H] = 407.1 |
| 298 | 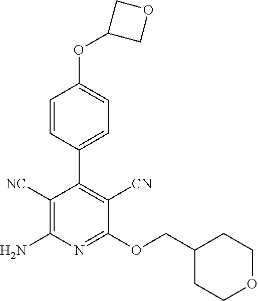 [SMILES: N#Cc1c(N)nc(OCC2CCOCC2)c(C#N)c1-c1ccc(OC2COC2)cc1] | ¹H NMR (400 MHz, DMSO-d₆) δ = 7.65- 8.66 (m, 2 H), 7.51 (d, J = 8.8 Hz, 2 H), 6.99 (d, J = 8.8 Hz, 2 H), 5.40 (q, J = 5.20 Hz, 1 H), 4.99 (t, J = 6.6 Hz, 2 H), 4.62 (dd, J = 7.2, 5.2 Hz, 2 H), 4.25 (d, J = 6.4 Hz, 2 H), 3.90 (br dd, J = 11.2, 3.2 Hz, 2 H), 3.30-3.38 (m, 2 H), 1.95-2.13 (m, 1 H), 1.54-1.70 (m, 2 H), 1.25-1.42 (m, 2 H). [M + H] = 407.1 |

### Protocol 12. 2-(azetidin-1-yl)-6-(oxetan-3-ylmethoxy)-4-(4-(oxetan-3-yloxy)phenyl)pyridine-3,5-dicarbonitrile (287)

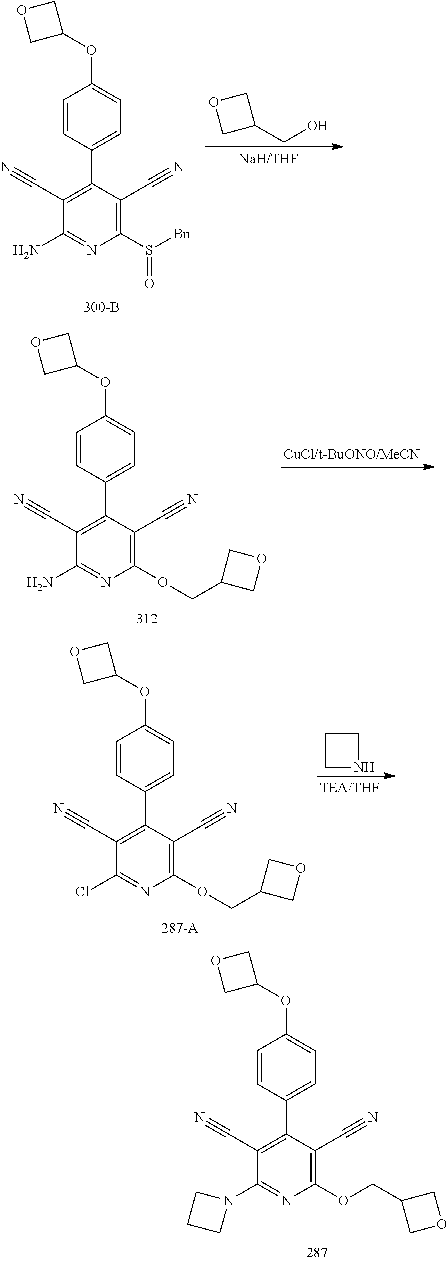

Step 1: 2-Amino-6-(oxetan-3-ylmethoxy)-4-(4-(oxetan-3-yloxy)phenyl)pyridine-3,5-dicarbonitrile (312). To a mixture of 2-amino-6-(benzylsulfinyl)-4-(4-(oxetan-3-yloxy)phenyl) pyridine-3,5-dicarbonitrile (600.0 mg, 1.39 mmol, 1.0 eq) in DMF (5.0 mL) was added NaH (278.73 mg, 6.97 mmol, 5.0 eq) at 0° C. and the reaction mixture was stirred at 20° C. for 1 hr. Then oxetan-3-ylmethanol (122.80 mg, 1.39 mmol, 1.0 eq) was added to the mixture reaction at 0° C., the reaction mixture was stirred at 20° C. for 1 hr under N₂ atmosphere. The reaction mixture was quenched by addition saturated aqueous H₂O (10 mL) at 0° C., and then extracted with EtOAc (10 mL×3). The combined organic layers were dried over Na₂SO₄, filtered and concentrated under reduced pressure to give a residue. The residue was purified by column chromatography on silica gel (Petroleum ether:Ethyl acetate=1:0 to 0:1) to give 2-amino-6-(oxetan-3-ylmethoxy)-4-(4-(oxetan-3-yloxy)phenyl)pyridine-3,5-dicarbonitrile (260.0 mg, 687.1 4 umol, 49.30% yield) as yellow solid. ¹H NMR (400 MHz, CD₃OD) δ=7.54 (d, J=8.8 Hz, 2H), 6.98 (d, J=8.8 Hz, 2H), 5.41 (t, J=5.2 Hz, 1H), 5.09 (t, J=6.8 Hz, 2H), 4.91-4.87 (m, 2H), 4.77 (dd, J=7.2, 5.2 Hz, 2H), 4.72 (d, J=6.4 Hz, 2H), 4.63 (t, J=6.0 Hz, 2H), 3.5 9-3.49 (m, 1H). [M+H]=379.4.

Step 2: 2-Chloro-6-(oxetan-3-ylmethoxy)-4-(4-(oxetan-3-yloxy)phenyl)pyridine-3,5-dicarbonitrile (287-A). To a mixture of 2-amino-6-(oxetan-3-ylmethoxy)-4-(4-(oxetan-3-yloxy) phenyl)pyridine-3,5-dicarbonitrile (200 mg, 528.57 umol, 1 eq) in MeCN (10.0 mL) was added t-BuONO (81.76 mg, 792.85 umol, 94.30 ul, 1.5 eq) and CuCl (156.98 mg, 1.59 mmol, 3.0 eq) at 25° C. and the reaction mixture was stirred at 60° C. for 5 hrs under N₂ atmosphere. T the reaction mixture was filtered and the filtrate was concentrated under reduced pressure to give a residue. The residue was purified by preparative TLC (Petroleum ether:Ethyl acetate=0:1) to give 2-chloro-6-(oxetan-3-ylmethoxy)-4-(4-(oxetan-3-yloxy)phenyl)pyridine-3,5-dicarbonitrile (120.0 mg, crude) as brown solid. [M+H]=398.2.

Step 3: 2-(Azetidin-1-yl)-6-(oxetan-3-ylmethoxy)-4-(4-(oxetan-3-yloxy)phenyl)pyridine-3,5-dicarbonitrile (287). To a mixture of 2-chloro-6-(oxetan-3-ylmethoxy)-4-(4-(oxetan-3-yloxy)phenyl)pyridine-3,5-dicarbonitrile (40.0 mg, 100.55 umol, 1.0 eq) in THF (1.0 mL) was added TEA (30.52 mg, 301.65 umol, 41.99 uL, 3 eq) and azetidine (17.22 mg, 184.09 umol, 1.83 eq) at 25° C. and the reaction mixture was stirred at 25° C. for 1 hr. The reaction mixture w as filtered and the filtrate was concentrated under reduced pressure to give a residue. The residue was purified by preparative HPLC (column: Waters Xbridge Prep OBD C₁₈ (150×40 m m, 10 um); flow rate: 25 mL/min; gradient: 25%-65% B over 8 min; mobile phase A: 10 m M aqueous NH₄HCO₃, mobile phase B: acetonitrile) to give 2-(azetidin-1-yl)-6-(oxetan-3-yl methoxy)-4-(4-(oxetan-3-yloxy)phenyl)pyridine-3,5-dicarbonitrile (26.3 mg, 61.37 umol, 61.04% yield, 97.6% purity) as white solid. ¹H-NMR (400 MHz, CD₃OD) δ ppm 7.48 (d, J=8.8 Hz, 2H), 6.95 (d, J=8.8 Hz, 2H), 5.46-5.33 (m, 1H), 5.13-5.01 (m, 2H), 4.93-4.88 (m, 2H), 4.87-4.84 (m, 1H), 4.80-4.72 (m, 2H), 4.71-4.66 (m, 2H), 4.64-4.58 (m, 2H), 4.57-4.44 (m, 3H), 3.57-3.47 (m, 1H), 2.53-2.41 (m, 2H). [M+H]=419.2.

TABLE 10
The following compounds were synthesized in a similar manner as
described for compound 287.

| Compound | Structure | Spectroscopic data |
|---|---|---|
| 307 | 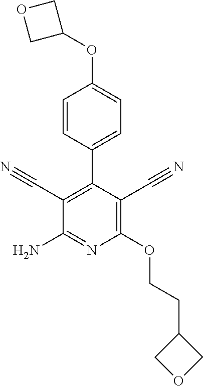 [SMILES: N#Cc1c(N)nc(OCCC2COC2)c(C#N)c1-c1ccc(OC2COC2)cc1] | ¹H NMR (400 MHz, CD₃OD) δ = 7.58-7.45 (m, 2 H), 6.96 (d, J = 8.4 Hz, 2H), 5.39 (t, J = 5.2 Hz, 1 H), 5.07 (t, J = 6.4 Hz, 2H), 4.87-4.82 (m, 2H), 4.75 (dd, J = 7.6, 5.2 Hz, 2 H), 4.56 (t, J = 6.4 Hz, 2H), 4.47 (t, J = 6.4 Hz, 2 H), 3.30-3.19 (m, 1H), 2.26-2.16 (m, 2H). [M + H] = 393.4 |
| 288 | 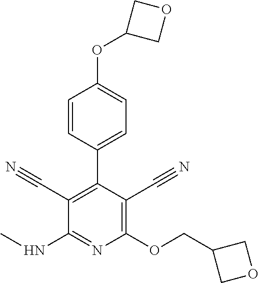 [SMILES: CNc1nc(OCC2COC2)c(C#N)c(-c2ccc(OC3COC3)cc2)c1C#N] | ¹H-NMR (400 MHz, CD₃OD) δ ppm 7.55-7.46 (m, 2H), 7.01-6.90 (m, 2H), 5.51-5.30 (m, 1H), 5.14-5.00 (m, 2H), 4.92-4.88 (m, 2H), 4.80- 4.71 (m, 4H), 4.67-4.50 (m, 2H), 3.61-3.48 (m, 1H), 3.10 (s, 3H). [M + H] = 393.1 |

### Protocol 13. 2-(Azetidin-1-yl)-4-(4-(1,1-difluoro-2-hydroxyethoxy)phenyl)-6-((pyridin-3-ylmethyl)thio)pyridine-3,5-dicarbonitrile (259)

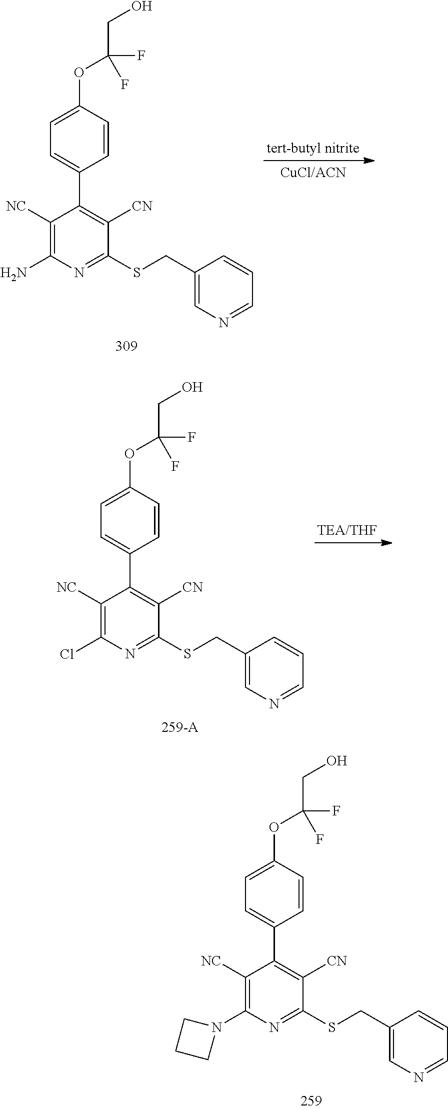 [SMILES: N#Cc1c(N)nc(SCc2cccnc2)c(C#N)c1-c1ccc(OC(F)(F)CO)cc1 ; N#Cc1c(Cl)nc(SCc2cccnc2)c(C#N)c1-c1ccc(OC(F)(F)CO)cc1 ; N#Cc1c(SCc2cccnc2)nc(N2CCC2)c(C#N)c1-c1ccc(OC(F)(F)CO)cc1]

Step 1: 2-Chloro-4-(4-(1,1-difluoro-2-hydroxyethoxy)phenyl)-6-((pyridin-3-ylmethyl)thio)pyridine-3,5-dicarbonitrile (259-A). To the mixture of 2-amino-4-[4-(1,1-difluoro-2-hydr oxy-ethoxy)phenyl]-6-(3-pyridylmethylsulfanyl)pyridine-3,5-dicarbonitrile (210 mg, 477.88 umol, 1 eq) in ACN (5 mL) was added tert-butyl nitrite (49.28 mg, 477.88 umol, 56.84 uL, 1 eq) and CuCl (47.31 mg, 477.88 umol, 11.43 uL, 1 eq), the mixture was stirred at 60° C. for 1 hr under N₂. The reaction mixture was filtered and concentrated to give 2-chloro-4-(4-(1,1-difluoro-2-hydroxyethoxy)phenyl)-6-((pyridin-3-ylmethyl)thio)pyridine-3,5-dicarbonitrile (200 mg, crude) as yellow solid. [M+H]=459.9

Step 2: 2-(Azetidin-1-yl)-4-(4-(1,1-difluoro-2-hydroxyethoxy)phenyl)-6-((pyridin-3-ylmethyl)thio)pyridine-3,5-dicarbonitrile (259). To a solution of 2-chloro-4-[4-(1,1-difluoro-2-hydroxy-ethoxy)phenyl]-6-(3-pyridylmethylsulfanyl)pyridine-3,5-dicarbonitrile (200 mg, 435.86 μmol, 1 eq) in THF (3 mL) was added TEA (132.31 mg, 1.31 mmol, 182.00 μL, 3 eq) and azetidine·hydrochloride (163.11 mg, 1.74 mmol, 4 eq). The mixture was stirred at 25° C. for 1 hr. The reaction mixture was filtered and the filtrate was concentrated. The residue was purified by preparative HPLC(Phenomenex Luna C18 column (75×30 mm, 3 um); flow rate: 25 mL/min; gradient: 15%-55% B over 8 min; mobile phase A: 0.2% aqueous FA, mobile phase B: acetonitrile) to give 2-(azetidin-1-yl)-4-(4-(1,1-difluoro-2-hydroxyethoxy)phenyl)-6-((pyridin-3-ylmethyl)thio)pyridine-3,5-dicarbonitrile (26.6 mg, 50.46 μmol, 11.58% yield, 99.7% purity, FA) as yellow solid. ¹H NMR (400 MHz, DMSO) δ=8.65 (d, J=1.6 Hz, 1H), 8.47 (d, J=3.6 Hz, 1H), 7.84 (br d, J=8.0 Hz, 1H), 7.57 (d, J=8.4 Hz, 2H), 7.43-7.34 (m, 3H), 5.94 (t, J=6.8 Hz, 1H), 4.54 (s, 2H), 4.43 (br d, J=1.2 Hz, 4H), 3.88 (dt, J=10.0, 6.8 Hz, 2H), 3.34 (br s, 1H), 2.41-2.30 (m, 2H). [M+H]=480.5.

TABLE 11
The following compounds were synthesized in a similar manner as
described for compound 259.

| Compound | Structure | Spectroscopic data |
|---|---|---|
| 254 | 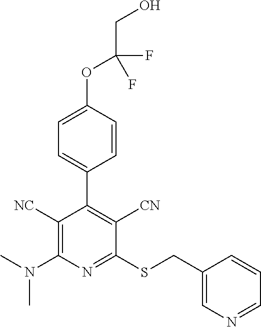 [SMILES: CN(C)c1nc(SCc2cccnc2)c(C#N)c(-c2ccc(OC(F)(F)CO)cc2)c1C#N] | ¹H NMR (400 MHz, DMSO-d₆) δ = 9.75- 8.03 (m, 2H), 7.88 (br s, 1H), 7.64- 7.37 (m, 5H), 5.95 (br s, 1H), 4.62 (br s, 2H), 3.89 (br s, 2H), 3.34-3.27 (m, 6H) [M + H] = 468.1 |
| 260 | 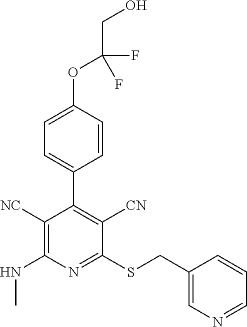 [SMILES: CNc1nc(SCc2cccnc2)c(C#N)c(-c2ccc(OC(F)(F)CO)cc2)c1C#N] | ¹H-NMR (400 MHz, CD₃OD) δ = 8.68 (d, J = 1.6 Hz, 1H), 8.47 (dd, J = 4.8, 1.6 Hz, 1H), 8.01-7.96 (m, 1H), 7.60-7.55 (m, 2H), .41 (d, J = 8.8 Hz, 3H), 5.55 (s, 1H), 4.60 (br s, 2H), 3.96 (s, 2H), 3.10 (s, 3H). [M + H] = 454.1 |
| 255 | 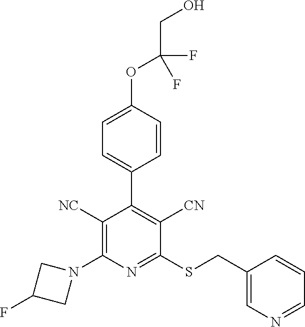 [SMILES: N#Cc1c(SCc2cccnc2)nc(N2CC(F)C2)c(C#N)c1-c1ccc(OC(F)(F)CO)cc1] | ¹H-NMR (400)δ ppm 8.66 (d, J = 1.6 Hz, 1H), 8.47 (dd, J = 4.8, 1.2 Hz, 1H), 7.97 (dt, J = 8.0, 1.6 Hz, 1H), 7.61-7.53 (m, 2H), 7.47-7.38 (m, 3H), 5.57-5.37 (m, 1H), 4.85-4.70 (m, 2H), 4.64-4.42 (m, 4H), 3.96 (t, J = 9.6 Hz, 2H). [M + H] = 498.1 |
| 256 | 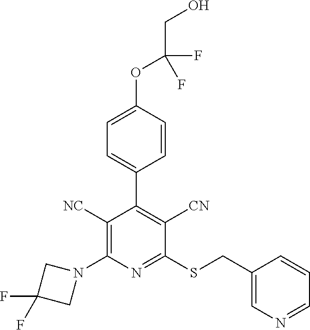 [SMILES: N#Cc1c(SCc2cccnc2)nc(N2CC(F)(F)C2)c(C#N)c1-c1ccc(OC(F)(F)CO)cc1] | ¹H-NMR (400 MHz, CD₃OD) δ ppm 8.74-8.63 (m, 1H), 8.49 (br s, 1H), 7.99 (br d, J = 7.6 Hz, 1H), 7.60 (d, J = 8.4 Hz, 2H), 7.49-7.42 (m, 3H), 4.89-4.82 (m, 4H), 4.64 (s, 2H), 3.98 (t, J = 9.6 Hz, 2H). [M + H] = 516.0 |
| 261 | 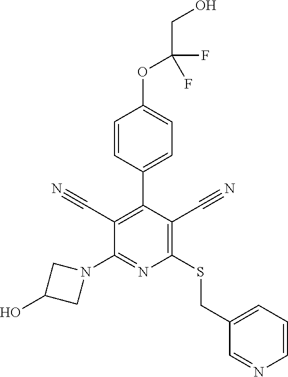 [SMILES: N#Cc1c(SCc2cccnc2)nc(N2CC(O)C2)c(C#N)c1-c1ccc(OC(F)(F)CO)cc1] | ¹H NMR (400 MHz, CD₃OD) δ ppm 8.65 (s, 1H), 8.46 (br d, J = 4.4 Hz, 1H), 7.96 (br d, J = 8.0 Hz, 1H), 7.58-7.52 (m, 2H), 7.45-7.38 (m, 3H), 4.70 (br s, 3H), 4.59 (s, 1H), 4.59-4.58 (m, 1H), 4.23 (br s, 2H), 3.95 (t, J = 9.6 Hz, 2H). [M + H] = 496.1 |
| 263 | 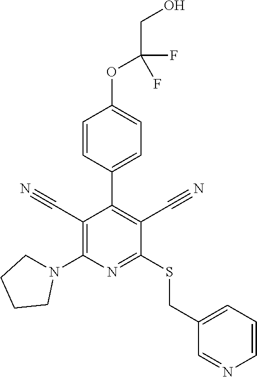 [SMILES: N#Cc1c(SCc2cccnc2)nc(N2CCCC2)c(C#N)c1-c1ccc(OC(F)(F)CO)cc1] | ¹H NMR (400 MHz, CD₃OD) δ 8.65 (s, 1H), 8.46 (d, J = 4.8 Hz, 1H), 7.96 (br d, J = 8.0 Hz, 1H), 7.56 (d, J = 8.8 Hz, 2H), 7.45-7.39 (m, 3H), 4.63 (s, 2H), 3.96 (t, J = 9.2 Hz, 2H), 3.88 (br s, 4H), 3.34-3.32 (m, 5H), 2.06-2.02 (m, 4H). [M + H] = 494.1 |

### Protocol 14. 2-Amino-4-(4-(2-(difluoromethoxy)-1,1-difluoroethoxy)phenyl)-6-((oxetan-3-ylmethyl)thio)pyridine-3,5-dicarbonitrile (289)

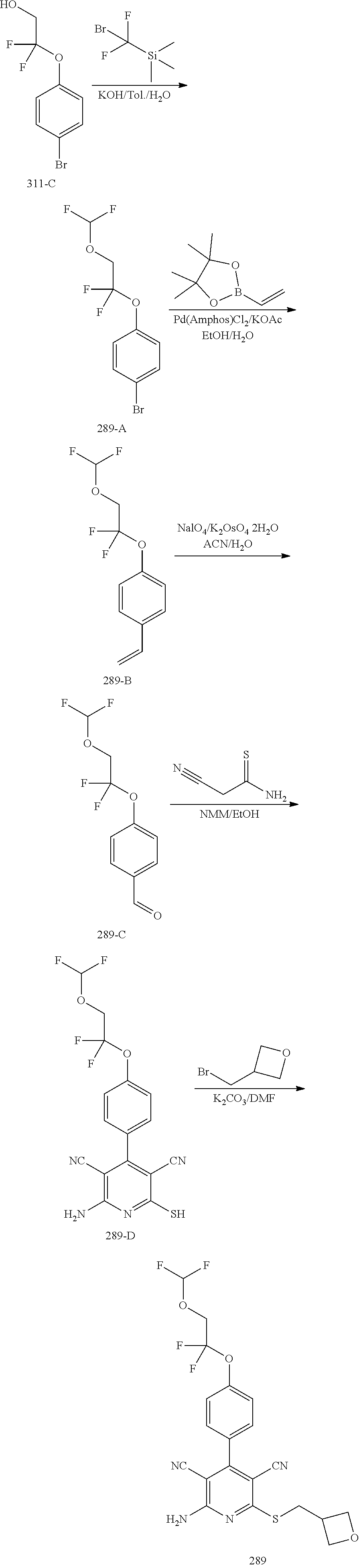

Step 1: 1-Bromo-4-(2-(difluoromethoxy)-1,1-difluoroethoxy)benzene (289-A). To a mixture of 2-(4-bromophenoxy)-2,2-difluoroethane-1-ol (2 g, 7.90 mmol, 1 eq) and KOH (2.00 g, 35.57 mmol, 4.5 eq) in Tol. (20 mL) and H₂O (6 mL) was stirred at 20° C. for 10 mins, then (bromodifluoromethyl)trimethylsilane (3.21 g, 15.81 mmol, 2 eq) was added to the mixture reaction at −78° C., the mixture was stirred at 60° C. for 2 hrs under N₂ atmosphere. The reaction mixture was filtered and the filtrate was concentrated under reduced pressure to give a residue. The residue was purified by flash column chromatography (SiO₂, Petroleum ether:Ethyl acetate=1:0 to 1:1) to give 1-bromo-4-(2-(difluoromethoxy)-1,1-difluoroethoxy)benzene (780 mg, 2.57 mmol, 32.56% yield) as yellow oil.

Step 2: 1-(2-(Difluoromethoxy)-1,1-difluoroethoxy)-4-vinylbenzene (289-B). To a mixture of 1-bromo-4-(2-(difluoromethoxy)-1,1-difluoroethoxy)benzene (750 mg, 2.47 mmol, 1 eq) and 4,4,5,5-tetramethyl-2-vinyl-1,3,2-dioxaborolane (457.39 mg, 2.97 mmol, 503.74 uL, 1.2 eq) in EtOH (3 mL) and H₂O (0.3 mL) was added 4-ditert-butylphosphanyl-N,N-dimethyl-aniline;dichloropalladium (175.24 mg, 247.49 umol, 175.24 uL, 0.1 eq) and KOAc (485.78 mg, 4.95 mmol, 2 eq) at 25° C., the reaction mixture was stirred at 80° C. for 2 hrs under the N₂ atmosphere. The reaction mixture was filtered and the filtrate was concentrated under reduced pressure to give a residue. The residue was purified by preparative TLC (SiO₂, Petroleum ether:Ethyl acetate=5:1) to give 1-(2-(difluoromethoxy)-1,1-difluoroethoxy)-4-vinylbenzene (320 mg, 1.28 mmol, 51.68% yield) as white solid. ¹H-NMR (400 MHz, CD₃OD) (ppm 7.39-7.31 (m, 2H), 7.06 (d, J=8.8 Hz, 2H), 6.64 (dd, J=10.4, 7.2 Hz, 1H), 6.60-6.26 (m, 1H), 5.66 (dd, J=17.6, 0.63 Hz, 1H), 5.21-5.07 (m, 1H), 4.21 (t, J=8.8 Hz, 2H).

Step 3: 4-(2-(Difluoromethoxy)-1,1-difluoroethoxy)benzaldehyde (289-C). To a mixture of 1-(2-(difluoromethoxy)-1,1-difluoroethoxy)-4-vinylbenzene (300 mg, 1.20 mmol, 1 eq) in ACN (2 mL) and H₂O (2 mL) was added K₂OsO₄·2H₂O (44.18 mg, 119.91 umol, 0.1 eq) at 0° C. and the reaction mixture was stirred at 25° C. for 0.5 hr, then NaIO₄ (1.03 g, 4.80 mmol, 265.78 uL, 4 eq) was added to the reaction mixture and the reaction mixture was stirred at 25° C. for 2 hrs. The reaction mixture was filtered and the filtrate was concentrated under reduced pressure to give a residue. The residue was purified by preparative TLC (SiO₂, Petroleum ether:Ethyl acetate=5:1) to give 4-(2-(difluoromethoxy)-1,1-difluoroethoxy)benzaldehyde (155 mg, 614.68 umol, 51.26% yield) as white solid. ¹H-NMR (400 MHz, CD₃OD) (ppm 10.01 (s, 1H), 8.01 (d, J=8.4 Hz, 2H), 7.46 (d, J=8.4 Hz, 2H), 6.97-6.33 (m, 1H), 4.42 (t, J=8.8 Hz, 2H).

Step 3: 2-Amino-4-(4-(2-(difluoromethoxy)-1,1-difluoroethoxy)phenyl)-6-mercaptopyridine-3,5-dicarbonitrile (289-D). To a mixture of 4-(2-(difluoromethoxy)-1,1-difluoroethoxy)benzaldehyde (140 mg, 555.20 umol, 1 eq) and 2-cyanoethanethioamide (111.20 mg, 1.11 mmol, 2 eq) in EtOH (4 mL) was added NMM (112.31 mg, 1.11 mmol, 122.08 uL, 2 eq) at 25° C. and the reaction mixture was stirred at 25° C. for 24 hrs. The reaction mixture was filtered and the filtrate was concentrated under reduced pressure to give a residue. The residue was purified by preparative HPLC (column: Phenomenex C₁₈ column (75×30 mm, 3 um); flow rate: 25 mL/min; gradient: 20%-50% B over 8 min; mobile phase A: 10 mM aqueous NH₄HCO₃, mobile phase B: acetonitrile) to give 2-amino-4-(4-(2-(difluoromethoxy)-1,1-difluoroethoxy)phenyl)-6-mercaptopyridine-3,5-dicarbonitrile (70 mg, 175.73 umol, 31.65% yield) as brown solid. [M−H]=397.1.

Step 4: 2-Amino-4-(4-(2-(difluoromethoxy)-1,1-difluoroethoxy)phenyl)-6-((oxetan-3-ylmethyl)thio)pyridine-3,5-dicarbonitrile (289). To a mixture of 2-amino-4-(4-(2-(difluoromethoxy)-1,1-difluoroethoxy)phenyl)-6-mercaptopyridine-3,5-dicarbonitrile (35 mg, 87.87 umol, 1 eq) and 3-(bromomethyl)oxetane (15.92 mg, 105.44 umol, 1.2 eq) in DMF (1.5 mL) was added K₂CO₃ (24.29 mg, 175.73 umol, 2 eq) and the mixture was stirred at 25° C. for 1 hr. The reaction mixture was filtered and the filtrate was concentrated under reduced pressure to give a residue. The residue was purified by preparative HPLC (column: Waters Xbridge BEH C18 column (100×30 mm, 10 um); flow rate: 25 mL/min; gradient: 35%-65% B over 8 min; mobile phase A: 10 mM aqueous NH₄HCO₃, mobile phase B: acetonitrile) to give 2-amino-4-(4-(2-(difluoromethoxy)-1,1-difluoroethoxy)phenyl)-6-((oxetan-3-ylmethyl)thio)pyridine-3,5-dicarbonitrile (14.7 mg, 31.38 umol, 35.72% yield) as yellow solid. [M+H]=469.1. ¹H-NMR (400 MHz, CD₃OD) (ppm 7.60 (d, J=8.8 Hz, 2H), 7.43 (d, J=8.4 Hz, 2H), 6.61 (t, J=73.6 Hz, 1H), 4.84 (dd, J=7.6, 6.4 Hz, 2H), 4.52 (t, J=6.0 Hz, 2H), 4.40 (t, J=8.8 Hz, 2H), 3.65 (d, J=7.6 Hz, 2H), 3.53-3.39 (m, 1H).

### Protocol 15. 2-(3-Fluoroazetidin-1-yl)-4-(4-(2-methoxyethoxy)phenyl)-6-((pyrimidin-4-ylmethyl)thio)pyridine-3,5-dicarbonitrile (334)

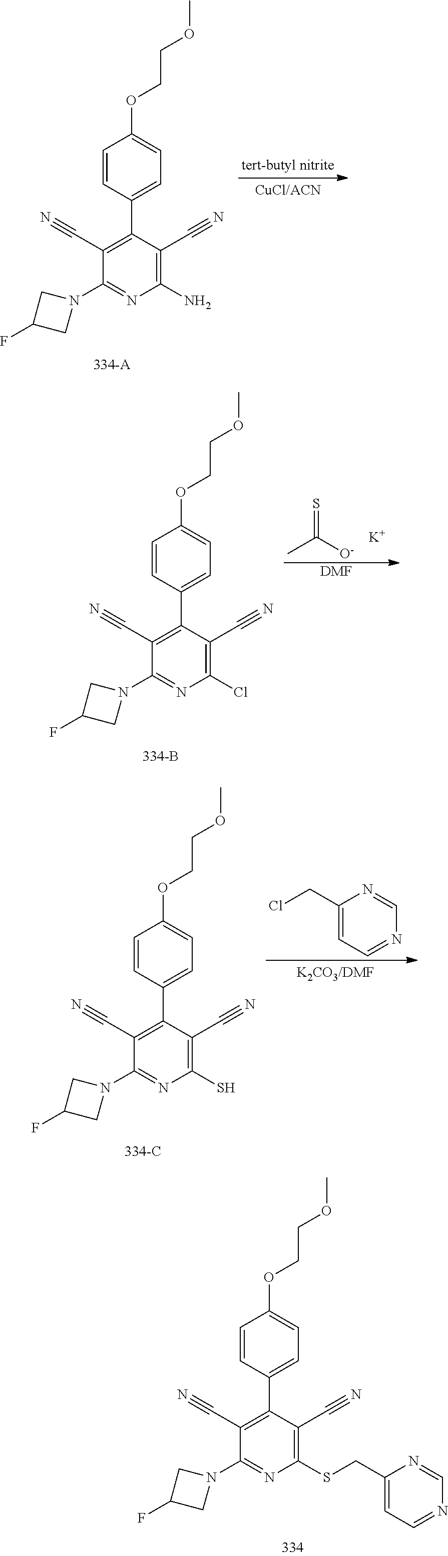

Step 1: 2-Chloro-6-(3-fluoroazetidin-1-yl)-4-(4-(2-methoxyethoxy)phenyl)pyridine-3,5-dicarbonitrile (334-B). To a solution of 2-amino-6-(3-fluoroazetidin-1-yl)-4-(4-(2-methoxyethoxy)phenyl)pyridine-3,5-dicarbonitrile (250 mg, 680.50 μmol, 1 eq) in ACN (5 mL) was added tert-butyl nitrite (140.35 mg, 1.36 mmol, 161.88 μL, 2 eq) and CuCl (134.74 mg, 1.36 mmol, 32.55 μL, 2 eq). The mixture was stirred at 60° C. for 1 hr under N₂, then filtered and the filtrate was concentrated under reduced pressure to give a residue. The residue was purified by column chromatography on silica gel (Petroleum ether:Ethyl acetate=3:1 to 1:1) to give 2-chloro-6-(3-fluoroazetidin-1-yl)-4-(4-(2-methoxyethoxy)phenyl)pyridine-3,5-dicarbonitrile (160 mg, 413.64 μmol, 60.79% yield) as a yellow oil. [M+H]=387.2

Step 2: 2-(3-Fluoroazetidin-1-yl)-6-mercapto-4-(4-(2-methoxyethoxy)phenyl)pyridine-3,5-dicarbonitrile (334-C). To a solution of 2-chloro-6-(3-fluoroazetidin-1-yl)-4-(4-(2-methoxyethoxy)phenyl)pyridine-3,5-dicarbonitrile (160 mg, 413.64 μmol, 1 eq) in DMF (2 mL) was added potassium ethanethioate (94.48 mg, 827.29 μmol, 2 eq) at 0° C. The mixture was warmed to 25° C. and stirred for 2 hrs. The reaction mixture was added to the sat.aq. NaClO, then the reaction mixture was partitioned between EtOAc (10 mL) and H₂O (5 mL), the water phase was extracted with EtOAc (15 mL×3), the combined organic phase was dried over Na₂SO₄, filtered and concentrated to give 2-(3-fluoroazetidin-1-yl)-6-mercapto-4-(4-(2-methoxyethoxy)phenyl)pyridine-3,5-dicarbonitrile (150 mg, crude) as a black solid. [M+H]=385.1

Step 3: 2-(3-Fluoroazetidin-1-yl)-4-(4-(2-methoxyethoxy)phenyl)-6-((pyrimidin-4-ylmethyl)thio)pyridine-3,5-dicarbonitrile (334). To a solution of 2-(3-fluoroazetidin-1-yl)-6-mercapto-4-(4-(2-methoxyethoxy)phenyl)pyridine-3,5-dicarbonitrile (150 mg, 390.19 μmol, 1 eq) in DMF (2 mL) was added K₂CO₃ (107.85 mg, 780.38 μmol, 2 eq) and 4-(chloromethyl)pyrimidine (50.16 mg, 390.19 μmol, 1 eq). The mixture was stirred at 25° C. for 0.5 hr, then filtered. The residue was purified by preparative HPLC (Waters Xbridge BEH C18(100×30 mm, 10 um); flow rate: 25 mL/min; gradient: 25%-60% B over 8 min; mobile phase A:10 mM aqueous NH₄HCO₃, mobile phase B: acetonitrile) to give 2-(3-fluoroazetidin-1-yl)-4-(4-(2-methoxyethoxy)phenyl)-6-((pyrimidin-4-ylmethyl)thio)pyridine-3,5-dicarbonitrile (37.0 mg, 76.87 μmol, 19.70% yield, 99.0% purity) as a white solid. ¹H NMR (400 MHz, DMSO-d₆) δ=9.14 (d, J=1.2 Hz, 1H), 8.77 (d, J=5.2 Hz, 1H), 7.66 (d, J=4.2 Hz, 1H), 7.47 (d, J=8.8 Hz, 2H), 7.12 (d, J=8.8 Hz, 2H), 5.56-5.36 (m, 1H), 4.70-4.57 (m, 4H), 4.41-4.27 (m, 2H), 4.20-4.16 (m, 2H), 3.70-3.67 (m, 2H), 3.32 (s, 3H) [M+H]=477.0.

### Example 2: Biological Activity

### Material and Methods: Biological Materials

SPA ³⁵S-GTPγS experiments were conducted with Epics Therapeutics membrane preparations. cAMP HTRF assays were conducted with recombinant cell lines. Receptor accession numbers and cellular backgrounds are shown in Table 12.

TABLE 12
Cell lines used for in vitro assays

| | Accession | | |
|---|---|---|---|
| Receptor | Number | Cell line | Reference agonist |
| human A1 | NP_000665.1 | CHO-K1 | CPA |
| human A2A | NP_000666.2 | HEK293 | NECA |
| human A2B | NP_000667.1 | HEK293 | NECA |

Conditions for the GTPγS scintillation proximity assay are shown in Table 13.

TABLE 13
GTPγS scintillation proximity assay (96 w)

| | | | | | | Incubation | |
|---|---|---|---|---|---|---|---|
| | Reference | NaCl | MgCl₂ | GDP | BSA | time (min)/ | Mb |
| Receptor | Agonist | (mM) | (mM) | (μM) | (%) | T° | (μg/well) |
| human A1 | CPA | 100 | 1 | 10 | 0 | 15/RT | 3 |

RT = room temperature

Assay buffer: 20 mM HEPES pH 7.4; 100 mM NaCl, 10 μg/mL saponin, MgCl₂ at optimized concentration for the specific receptor.
Membranes: membrane extracts prepared from CHO-K1 cells expressing recombinant human Adenosine A1 receptor, thawed on ice, diluted in assay buffer and kept on ice.
GDP: diluted in assay buffer at the optimized concentration for the specific receptor Beads: PVT-WGA (Perkin Elmer, RPNQ001), diluted in assay buffer at 50 mg/mL (0.5 mg/10 μL)
GTPγ³⁵S: (Perkin Elmer, NEG030X), diluted in assay buffer for a final 0.1 nM assay concentration

Protocol for agonist testing: membranes are mixed with GDP (volume:volume). In parallel, GTPγ[³⁵S] is mixed with the beads (volume:volume) just before starting the reaction. The following reagents are successively added in the wells of an Optiplate (Perkin Elmer): 50 μL of test or reference ligand, 25 μL of the membranes:GDP mix, and 25 μL of the GTPγ[³⁵S]:beads mix. The plates are covered with a top seal, mixed on an orbital shaker for 2 min, and then incubated for 1 hour at room temperature. Then the plates are centrifuged for 10 min at 2000 rpm, incubated at room temperature and counted for 30 sec/well with a PerkinElmer TopCount™ reader.
Conditions for the GTPγS scintillation proximity assay are shown in Table 14.

TABLE 14
cAMP HTRF assays

| | Reference | | Incubation time | Cells/ |
|---|---|---|---|---|
| Receptor | Agonist | Buffer | (min)/ T° | well |
| human A2A | NECA | KRH - 25 μM | 30 / RT | 1000 |
| | | Rolipram | | |
| human A2B | NECA | KRH - 25 μM | 30 / RT | 2500 |
| | | Rolipram | | |

RT = room temperature

cAMP HTRF assay for Gs coupled receptor: HEK293 cells expressing recombinant receptor grown prior to the test in media without antibiotic are detached by gentle flushing with PBS-EDTA (5 mM EDTA), recovered by centrifugation and resuspended in assay buffer (KRH: 5 mM KCl, 1.25 mM MgSO₄, 124 mM NaCl, 25 mM HEPES, 13.3 mM glucose, 1.25 mM KH₂PO₄, 1.45 mM CaCl₂, 0.5 g/L BSA, supplemented with 1 mM IBMX or 25 μM rolipram).

Dose response curves are performed in parallel with the reference compounds.

For agonist test (96 well): 12 μL of cells are mixed with 12 μL of the test compound at increasing concentrations and then incubated 30 min at room temperature. After addition of the lysis buffer containing cAMP-d2 and anti-cAMP cryptate detection reagents, plates are incubated 1 hour incubation at room temperature, and fluorescence ratios are measured according to the manufacturer specification, with the HTRF kit.

Results are provided in Table 15 below.

TABLE 15
Adenosine Agonist Activity

| | A1AR EC50 | A2BAR EC50 |
|---|---|---|
| | (nM) | (nM) |
| 1 | >10,000 | 5850 |
| 2 | >10,000 | 0.28 |
| 4 | >10,000 | 2410 |
| 5 | >10,000 | 13.4 |
| 6 | 586 | 213 |
| 7 | >10,000 | 150 |
| 8 | 37.1 | 37.7 |
| 9 | 884 | 2490 |
| 10 | >10,000 | 3760 |
| 12 | 483 | >10,000 |
| 13 | >10,000 | 272 |
| 14 | 29.2 | 81.7 |
| 15 | 1050 | 142 |
| 16 | 7600 | >10,000 |
| 17 | 99.1 | 5950 |
| 18 | 69.5 | 299 |
| 20 | 14.1 | 3.0 |
| 21 | 3520 | >10,000 |
| 22 | 3.5 | >10,000 |
| 23 | 100 | 420 |
| 24 | 9.8 | >10,000 |
| 25 | 1.1 | >10,000 |
| 27 | 31.4 | 65.9 |
| 28 | 52.4 | >10,000 |
| 29 | 79 | 6120 |
| 30 | 3090 | >10,000 |
| 31 | 596 | >10,000 |
| 32 | 890 | >10,000 |
| 33 | 7.9 | 563 |
| 34 | 245 | >10,000 |
| 35 | 238 | >10,000 |
| 36 | 124 | >10,000 |
| 37 | 139 | >10,000 |
| 39 | 862 | >10,000 |
| 40 | 21.6 | >10,000 |
| 44 | 34.3 | >10,000 |
| 45 | 34.3 | 8.2 |
| 46 | 110 | >10,000 |
| 47 | >10,000 | 36.1 |
| 48 | >10,000 | 42.6 |
| 49 | 311 | 65 |
| 50 | 294 | 94.2 |
| 51 | 38 | 5.1 |
| 52 | 94.2 | 5.3 |
| 53 | 7.2 | 0.7 |
| 56 | 8.6 | 0.08 |
| 61 | 620 | >10,000 |
| 62 | 350 | 10,000 |
| 63 | 91 | 2960 |
| 64 | 281 | >10,000 |
| 65 | 309 | 1.9 |
| 66 | 2.5 | 0.2 |
| 67 | 17.6 | >10,000 |
| 68 | >10,000 | 4230 |
| 69 | 201 | 26.3 |
| 70 | 469 | 209 |
| 74 | >10,000 | 8100 |
| 76 | >10,000 | 4370 |
| 77 | >10,000 | 2230 |
| 78 | 643 | >10,000 |
| 79 | 1790 | 45 |
| 83 | 282 | >10,000 |
| 84 | 44 | 6.8 |
| 89 | 819 | >10,000 |
| 90 | 347 | 869 |
| 93 | 109 | 64 |
| 94 | 1010 | >10,000 |
| 95 | 276 | 459 |
| 96 | >10,000 | 97 |
| 104 | 1790 | >10,000 |
| 106 | 502 | >10,000 |
| 107 | 97 | >10,000 |
| 110 | 253 | 177 |
| 112 | 944 | >10,000 |
| 113 | 316 | 10,000 |
| 114 | 7.6 | 18 |
| 115 | 31 | >10,000 |
| 116 | 2.9 | 3.9 |
| 117 | 997 | >10,000 |
| 119 | 161 | 925 |
| 120 | 1000 | >10,000 |
| 121 | 327 | 576 |
| 122 | 1020 | >10,000 |
| 123 | 72 | >10,000 |
| 125 | >10,000 | 468 |
| 127 | 91 | >10,000 |
| 128 | >10,000 | 255 |
| 129 | 121 | 6 |
| 132 | >10,000 | 645 |
| 136 | 768 | 87 |
| 138 | 891 | 2460 |
| 139 | 87 | 4.5 |
| 140 | 7500 | >10,000 |
| 141 | 569 | 149 |
| 146 | 177 | 59 |
| 147 | 55 | 8560 |
| 150 | 12 | 4.2 |
| 151 | 61 | 28 |
| 152 | 92 | >10,000 |
| 159 | 41 | 3.3 |
| 160 | 130 | 56 |
| 161 | 368 | 159 |
| 162 | 11 | 0.7 |
| 172 | 12 | 3.9 |
| 174 | 8.1 | 0.5 |
| 175 | 20 | 0.9 |
| 176 | 34 | 7.1 |
| 177 | 57 | 18 |
| 178 | 1.5 | 0.06 |
| 183 | 2.0 | 0.1 |
| 191 | 567 | >10,000 |
| 192 | 25 | >10,000 |
| 193 | 49 | >10,000 |
| 194 | 71 | >10,000 |
| 195 | 9.7 | >10,000 |
| 197 | 52 | >10,000 |
| 198 | 80 | >10,000 |
| 206 | 981 | 495 |
| 207 | 739 | 108 |
| 208 | 500 | 98 |
| 212 | 7,600 | >10,000 |
| 213 | >10,000 | >10,000 |
| 214 | >10,000 | >10,000 |
| 215 | >10,000 | >10,000 |
| 216 | >10,000 | >10,000 |
| 217 | >10,000 | >10,000 |
| 218 | 353 | >10,000 |
| 219 | 154 | >10,000 |
| 220 | >10,000 | >10,000 |
| 221 | >10,000 | >10,000 |
| 222 | 32 | >10,000 |
| 223 | 1.5 | 401 |
| 224 | 230 | >10,000 |
| 225 | 0.75 | 118 |
| 226 | 22 | >10,000 |
| 227 | 326 | >10,000 |
| 228 | 1460 | 7550 |
| 229 | >10,000 | 11 |
| 230 | 5.2 | >10,000 |
| 232 | 1.51 | >10,000 |
| 233 | >10,000 | >10,000 |
| 234 | 1.22 | 2.02 |
| 235 | 3280 | 1970 |
| 236 | 164 | 49.3 |
| 237 | 1.62 | 5360 |
| 238 | >10,000 | >10,000 |
| 239 | 840 | >10,000 |
| 240 | 0.903 | 0.335 |
| 241 | 73.2 | >10,000 |
| 242 | 905 | 749 |
| 243 | >10,000 | 6450 |
| 244 | >10,000 | >10,000 |
| 245 | 2.05 | >10,000 |
| 246 | >10,000 | >10,000 |
| 247 | 1.19 | >10,000 |
| 248 | 276 | >10,000 |
| 249 | >10,000 | >10,000 |
| 250 | 33.2 | >10,000 |
| 251 | >10,000 | >10,000 |
| 252 | >10,000 | >10,000 |
| 253 | >10,000 | >10,000 |
| 254 | >10,000 | >10,000 |
| 255 | >10,000 | 435 |
| 256 | >10,000 | >10,000 |
| 257 | >10,000 | >10,000 |
| 258 | >10,000 | >10,000 |
| 259 | >10,000 | 198 |
| 260 | 3.38 | 33.7 |
| 261 | >10,000 | >10,000 |
| 262 | >10,000 | >10,000 |
| 263 | >10,000 | >10,000 |
| 264 | >10,000 | >10,000 |
| 265 | 12.5 | >10,000 |
| 266 | 1070 | >10,000 |
| 267 | 77 | >10,000 |
| 268 | 1.16 | 56.7 |
| 269 | 615 | >10,000 |
| 270 | 205 | >10,000 |
| 271 | 30.3 | 116 |
| 272 | 22 | 311 |
| 273 | 295 | >10,000 |
| 274 | 75.2 | >10,000 |
| 275 | 55.9 | >10,000 |
| 276 | 98.8 | >10,000 |
| 277 | 23.9 | >10,000 |
| 278 | 13.9 | >10,000 |
| 281 | 827 | >10,000 |
| 282 | 61.5 | 20 |
| 283 | >10,000 | >10,000 |
| 284 | >10,000 | >10,000 |
| 285 | 958 | >10,000 |
| 286 | >10,000 | >10,000 |
| 287 | 807 | >10,000 |
| 288 | 308 | >10,000 |
| 289 | >10,000 | >10,000 |
| 290 | 265 | >10,000 |
| 291 | 208 | 9890 |
| 292 | 93 | >10,000 |
| 293 | 73.2 | >10,000 |
| 294 | 25.2 | >10,000 |
| 295 | 1.6 | 9.79 |
| 296 | 20.6 | 2.47 |
| 297 | 1100 | >10,000 |
| 298 | 229 | >10,000 |
| 299 | >10,000 | >10,000 |
| 300 | 1150 | >10,000 |
| 301 | 228 | >10,000 |
| 302 | 64.8 | 894 |
| 303 | 78.5 | >10,000 |
| 304 | 167 | >10,000 |
| 305 | 140 | 1120 |
| 306 | 353 | >10,000 |
| 307 | >10,000 | >10,000 |
| 308 | >10,000 | 5570 |
| 309 | 6.48 | 0.962 |
| 310 | >10,000 | >10,000 |
| 311 | 7.18 | >10,000 |
| 312 | 204 | >10,000 |
| 313 | 55 | >10,000 |
| 314 | 5.16 | >10,000 |
| 315 | 81.8 | >10,000 |
| 316 | 10.8 | 305 |
| 317 | 88.7 | >10,000 |
| 318 | 32.1 | >10,000 |
| 319 | 1.53 | 401 |
| 320 | 230 | >10,000 |
| 321 | >10,000 | >10,000 |
| 322 | 0.75 | 118 |
| 323 | 227 | >10,000 |
| 324 | 21.3 | >10,000 |
| 325 | >10,000 | >10,000 |
| 326 | >10,000 | >10,000 |
| 327 | 22.1 | >10,000 |
| 328 | >10,000 | >10,000 |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject application have been discussed, the above specification is illustrative and not restrictive. Many variations of the subject of the application will become apparent to those skilled in the art upon review of this specification and the claims below. The full scope of the application should be determined by reference to the claims, along with their full scope of equivalents, and the specification, along with such variations.

1. A compound of formula (I) 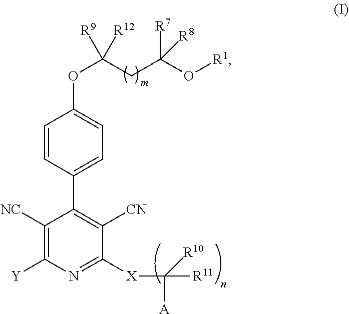
or a pharmaceutically acceptable salt thereof, wherein:
Y is selected from hydrogen, C₁₋₆ alkyl, C₃₋₆ cycloalkyl, OR¹³, and NR²R³,
R² and R³ each independently is selected from hydrogen and C₁₋₆alkyl; or R² and R³ taken together with the nitrogen to which they are attached form a 4- or 5-membered heterocyclyl ring, wherein the 4- or 5-membered heterocyclyl ring is optionally substituted with one or more substitutent independently selected from halogen or hydroxyl;
R¹ is selected from hydrogen, C₁₋₆ alkyl, and C₁₋₆ haloalkyl;
R⁷ and R⁸ each independently is selected from hydrogen, C₁₋₆alkyl, and C₁₋₆ haloalkyl;
R⁹ is hydrogen or halogen, or R¹ and R⁹ taken together with the atoms to which they are attached form a 4-, 5-, or 6-membered heterocyclyl ring, or R⁹ and R⁸ taken together with the atoms to which they are attached form a cyclopropyl ring system when m is 0 or a cyclobutyl or cyclopentyl ring system when m is 1;
R¹⁰ and R¹¹ each independently is selected from hydrogen, hydroxyl, halogen, C₁₋₆alkyl, and C₁₋₆ haloalkyl;
R¹² is hydrogen or halogen;
X is selected from a bond, S, O, and NR⁴, wherein R⁴ is hydrogen or C₁₋₆alkyl (e.g., methyl);
R¹³ is selected from hydrogen, C₁₋₆ alkyl, and C₁₋₆ haloalkyl;
A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, optionally substituted aryl, and optionally substituted heteroaryl;
R⁵ and R⁶ each independently is selected from hydrogen and C₁₋₆alkyl;
m is 0 or 1, provided that when m is 1, R⁸ and R⁹ are taken together with the carbons to which they are attached to form a cyclobutyl or cyclopentyl ring system; and
n is 0, 1 or 2;
provided that when Y is NH₂, X is S, n is 1, R¹ is hydrogen or methyl, and each of R⁷, R⁸ and R⁹ is hydrogen, then A is not substituted thiazole;
when Y is NH₂, X is S, n is 1, R⁸ is methyl, and each of R¹, R⁷ and R⁹ is hydrogen, then A is not substituted thiazole;
when Y is NR²R³, R² and R³ are both C₁₋₆alkyl (e.g., ethyl), X is S, n is 1, and each of R¹, R⁷, R⁸ and R⁹ is hydrogen, then A is not substituted thiazole;
when Y is NR²R³, R² and R³ taken together with the nitrogen to which they are attached form a 4- or 5-membered heterocyclyl ring, X is S, n is 1, and each of R¹, R⁷, R⁸ and R⁹ is hydrogen, then A is not substituted thiazole;
when Y is NH₂, X is S, n is 1, R¹ is methyl, and each of R⁷, R⁸, R⁹, R¹⁰, and R¹¹ is hydrogen, then A is not 2-pyridinyl or 3-pyridinyl;
when Y is NH₂, X is S, n is 1, R⁷ is methyl, R⁸ is methyl, and each of R¹ and R⁹ is hydrogen, then A is not substituted 4-pyridyl;
when Y is NH₂, X is S, n is 1, and each of R¹, R⁷, R⁸ and R⁹ is hydrogen, then A is not substituted 3-pyridyl;
when Y is hydrogen or NR²R³, R² is hydrogen, R³ is hydrogen or C₁₋₆alkyl, X is S, n is 1, and each of R¹, R⁷, R⁸ and R⁹ is hydrogen, then A is not substituted 4-pyridyl;
when Y is NH₂ or pyrrolidine, X is S, n is 1, and each of R¹, R⁷, R⁸, R⁹, R¹⁰ and R¹¹ is hydrogen, then A is not substituted phenyl;
when Y is NH₂, X is S, n is 1, R¹ is hydrogen or methyl, each of R⁷, R⁸, R⁹, and R¹¹ is hydrogen, and R¹⁰ is methyl, then A is not substituted phenyl; when Y is H or NH₂, X is S, n is 0, and each of R¹, R⁷, R⁸ and R⁹ is hydrogen, then A is not optionally substituted aryl (e.g., aryl, such as phenyl);
when Y is NH₂, X is S or O, n is 1, and each of R¹, R⁷, R⁸ and R⁹ is hydrogen, then A is not substituted oxazole;
when Y is NH₂, X is 0, n is 1, and each of R¹, R⁷, R⁸ and R⁹ is hydrogen, then A is not substituted phenyl, or substituted 4-pyridyl;
when Y is OR¹³, R¹³ is methyl, X is S, n is 1, and each of R¹, R⁷, R⁸, R⁹, R¹⁰ and R¹¹ is hydrogen, then A is not substituted phenyl, substituted oxazole, imidazole, or 2-pyridyl;
when Y is OR¹³, R¹³ is ethyl, X is S, n is 1, and each of R¹, R⁷, R⁸, R⁹, R¹⁰ and R¹¹ is hydrogen, then A is not substituted oxazole; and
when Y is OR¹³, R¹³ is methyl, ethyl, or sec-butyl, X is S, n is 1, and each of R¹, R⁷, R⁸, R⁹, R¹⁰ and R¹¹ is hydrogen, then A is not substituted thiazole.

2. The compound of claim 1, or a pharmaceutically acceptable salt thereof, wherein A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, aryl (e.g., phenyl), and optionally substituted heteroaryl.

3. The compound of claim 1, or a pharmaceutically acceptable salt thereof, wherein A is selected from cyano, hydroxyl, optionally substituted C₁₋₆alkyl, optionally substituted C₁₋₆ alkoxy, —C(O)NR⁵R⁶, —NR⁵R⁶, —NR⁵C(O)C₁₋₆alkyl, optionally substituted heterocyclyl, optionally substituted aryl, optionally substituted indole, optionally substituted pyrazine, optionally substituted pyrimidine, optionally substituted pyrrolopyridine, optionally substituted pyridazine, optionally substituted indazole, optionally substituted benzofuran, optionally substituted benzoimidazole, optionally substituted benzothiazole, and optionally substituted benzothiophene.

4. The compound of claim 1, or a pharmaceutically acceptable salt thereof, wherein A is selected from cyano, hydroxyl, optionally substituted C₁₋₆alkyl, optionally substituted C₁₋₆ alkoxy, —C(O)NR⁵R⁶, —NR⁵R⁶, —NR⁵C(O)C₁₋₆alkyl, optionally substituted heterocyclyl, optionally substituted aryl, optionally substituted indole, optionally substituted pyrazine, optionally substituted pyrimidine, optionally substituted pyrrolopyridine, optionally substituted pyridazine, optionally substituted indazole, optionally substituted benzofuran, optionally substituted benzoimidazole, optionally substituted benzothiazole, optionally substituted benzothiophene, oxazole, and thiazole.

5. The compound of claim 1, or a pharmaceutically acceptable salt thereof, wherein A is selected from cyano, hydroxyl, optionally substituted C₁₋₆alkyl, optionally substituted C₁₋₆alkoxy, —C(O)NR⁵R⁶, —NR⁵R⁶, —NR⁵C(O)C₁₋₆alkyl, optionally substituted heterocyclyl, optionally substituted indole, optionally substituted pyrazine, optionally substituted pyrimidine, optionally substituted pyrrolopyridine, optionally substituted pyridazine, optionally substituted indazole, optionally substituted benzofuran, optionally substituted benzoimidazole, optionally substituted benzothiazole, and optionally substituted benzothiophene.

6. The compound of claim 1, or a pharmaceutically acceptable salt thereof, wherein A is selected from cyano, hydroxyl, optionally substituted C₁₋₆alkyl, optionally substituted C₁₋₆ alkoxy, —C(O)NR⁵R⁶, —NR⁵R⁶, —NR⁵C(O)C₁₋₆alkyl, and optionally substituted heterocyclyl.

7. The compound of claim 1, or a pharmaceutically acceptable salt thereof, wherein A is selected from cyano, hydroxyl, optionally substituted C₁₋₆alkyl, optionally substituted C₁₋₆ alkoxy, —C(O)NR⁵R⁶, —NR⁵R⁶, —NR⁵C(O)C₁₋₆alkyl, optionally substituted heterocyclyl, and optionally substituted heteroaryl.

8. The compound of claim 1, or a pharmaceutically acceptable salt thereof, wherein A is selected from cyano, hydroxyl, optionally substituted C₁₋₆alkyl, optionally substituted C₁₋₆alkoxy, —C(O)NR⁵R⁶, —NR⁵R⁶, —NR⁵C(O)C₁₋₆alkyl, optionally substituted heterocyclyl, aryl, and optionally substituted heteroaryl.

9. The compound of claim 1, or a pharmaceutically acceptable salt thereof, wherein A is optionally substituted heterocyclyl

10. The compound of claim 9, or a pharmaceutically acceptable salt thereof, wherein A is optionally substituted oxetane.

11. The compound of claim 9, or a pharmaceutically acceptable salt thereof, wherein A is optionally substituted tetrahydrofuran.

12. The compound of any preceding claim, or a pharmaceutically acceptable salt thereof, wherein Y is NR²R³ and R² and R³ each independently is selected from hydrogen and C₁₋₆alkyl.

13. The compound of claim 12, or a pharmaceutically acceptable salt thereof, wherein Y is NH₂.

14. The compound of claim 12, or a pharmaceutically acceptable salt thereof, wherein Y is NMe₂.

15. The compound of claim 12, or a pharmaceutically acceptable salt thereof, wherein Y is NHMe.

16. The compound of any one of claims 1-11, or a pharmaceutically acceptable salt thereof, wherein Y is NR²R³ and R² and R³ taken together with the nitrogen to which they are attached form a 4- or 5-membered heterocyclyl ring.

17. The compound of any one of claims 1-11, or a pharmaceutically acceptable salt thereof, wherein Y is hydrogen.

18. The compound of any one of claims 1-11, or a pharmaceutically acceptable salt thereof, wherein Y is C₁₋₆ alkyl.

19. The compound of any one of claims 1-11, or a pharmaceutically acceptable salt thereof, wherein Y is C₃₋₆ cycloalkyl.

20. The compound of any preceding claim, or a pharmaceutically acceptable salt thereof, wherein X is S.

21. The compound of any preceding claim, or a pharmaceutically acceptable salt thereof, wherein n is 1.

22. The compound of any preceding claim, or a pharmaceutically acceptable salt thereof, wherein R¹⁰ and R¹¹ are both hydrogen.

23. The compound of any preceding claim, or a pharmaceutically acceptable salt thereof, wherein R⁷ and R⁸ are both hydrogen.

23. The compound of any one of claims 1-22, or a pharmaceutically acceptable salt thereof, wherein R⁷ and R⁸ are both C₁₋₆alkyl.

24. The compound of any one of claims 1-22, or a pharmaceutically acceptable salt thereof, wherein R⁷ is hydrogen and and R⁸ is C₁₋₆alkyl or C₁₋₆ haloalkyl.

25. The compound of any preceding claim, or a pharmaceutically acceptable salt thereof, wherein R⁹ is hydrogen.

26. The compound of any preceding claim, or a pharmaceutically acceptable salt thereof, wherein R¹ is hydrogen.

27. The compound of any one of claims 1-24, or a pharmaceutically acceptable salt thereof, wherein R¹ and R⁹ taken together with the atoms to which they are attached form a 4- or 5-membered heterocyclyl ring

28. The compound of claim 27, or a pharmaceutically acceptable salt thereof, wherein R¹ and R⁹ taken together with the atoms to which they are attached form oxetan-3-yl.

29. A compound selected from any one of compounds 1-10, 12-18, 20-25, 27-37, 39, 40, 44-53, 56, 61-70, 74, 76-79, 83, 84, 89, 90, 93-96, 104, 106, 107, 110, 112-117, 119-123, 125, 127-129, 132, 136, 138-141, 146-147, 150-152, 159-162, 172, 174-178, 183, 191-195, 197, 198, 206-208, 212-230, and 232-339, and pharmaceutically acceptable salts thereof.

30. A pharmaceutical composition comprising (a) a compound of any preceding claim; and (b) a pharmaceutically acceptable excipient.

31. A compound of any one of claims 1-29 or a pharmaceutical composition of claim 30 for use as a medicament.

32. A method of treating Rett syndrome and/or one or more symptoms associated with Rett syndrome in a subject, comprising administering to a subject in need thereof an effective amount of at least one compound of any one of claims 1-29 or a pharmaceutical composition of claim 30.

33. A method of treating Rett syndrome and/or one or more symptoms associated with Rett syndrome in a subject, comprising administering to a subject in need thereof an effective amount of a compound of formula (Ia) 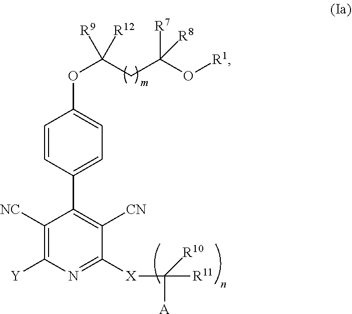
or a pharmaceutically acceptable salt thereof, wherein:
Y is selected from hydrogen, C₁₋₆ alkyl, C₃₋₆ cycloalkyl, O, and NR²R³,
R² and R³ each independently is selected from hydrogen and C₁₋₆alkyl; or R² and R³ taken together with the nitrogen to which they are attached form a 4- or 5-membered heterocyclyl ring, wherein the 4- or 5-membered heterocyclyl ring is optionally substituted with one or more substitutent independently selected from halogen or hydroxyl;
R¹ is selected from hydrogen, C₁₋₆ alkyl, and C₁₋₆ haloalkyl;
R⁷ and R⁸ each independently is selected from hydrogen, C₁₋₆alkyl, and C₁₋₆ haloalkyl;
R⁹ is hydrogen or halogen, or R¹ and R⁹ taken together with the atoms to which they are attached form a 4-, 5-, or 6-membered heterocyclyl ring, or R⁹ and R⁸ taken together with the atoms to which they are attached form a cyclopropyl ring system when m is 0 or a cyclobutyl or cyclopentyl ring system when m is 1;
R¹⁰ and R¹¹ each independently is selected from hydrogen, hydroxyl, halogen, C₁₋₆alkyl, and C₁₋₆ haloalkyl;
R¹² is hydrogen or halogen;
X is selected from a bond, S, O, and NR⁴, wherein R⁴ is hydrogen or C₁₋₆alkyl (e.g., methyl);
A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, optionally substituted aryl, and optionally substituted heteroaryl;
R⁵ and R⁶ each independently is selected from hydrogen and C₁₋₆alkyl;
m is 0 or 1, provided that when m is 1, R⁸ and R⁹ are taken together with the carbons to which they are attached to form a cyclobutyl or cyclopentyl ring system; and
n is 0, 1 or 2.

34. The method of claim 32, wherein the compound is selected from any one of compounds 1-10, 12-18, 20-25, 27-37, 39, 40, 44-53, 56, 61-70, 74, 76-79, 83, 84, 89, 90, 93-96, 104, 106, 107, 110, 112-117, 119-123, 125, 127-129, 132, 136, 138-141, 146-147, 150-152, 159-162, 172, 174-178, 183, 191-195, 197, 198, 206-208, 212-230, and 232-339, and pharmaceutically acceptable salts thereof.

35. The method of any one of claims 32-34, wherein the one or more symptoms associated with Rett syndrome is selected from sleep disturbances; sleep apnea; seizures; breathing disorders; irregular heartbeat; intellectual disabilities; and autism.

36. Use of a compound of any one of claims 1-29 or a pharmaceutical composition of claim 30, in the preparation of a medicament for the treatment of Rett syndrome and/or one or more symptoms associated with Rett syndrome.

37. Use of a compound of compound of formula (Ia) 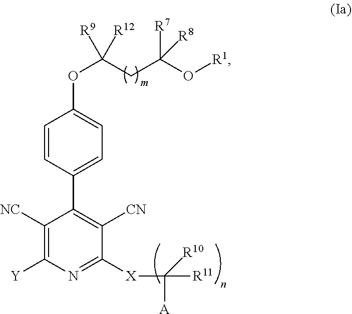
or a pharmaceutically acceptable salt thereof, wherein:
Y is selected from hydrogen, C₁₋₆ alkyl, C₃₋₆ cycloalkyl, O, and NR²R³,
R² and R³ each independently is selected from hydrogen and C₁₋₆alkyl; or R² and R³ taken together with the nitrogen to which they are attached form a 4- or 5-membered heterocyclyl ring, wherein the 4- or 5-membered heterocyclyl ring is optionally substituted with one or more substitutent independently selected from halogen or hydroxyl;
R¹ is selected from hydrogen, C₁₋₆ alkyl, and C₁₋₆ haloalkyl;
R⁷ and R⁸ each independently is selected from hydrogen, C₁₋₆alkyl, and C₁₋₆ haloalkyl;
R⁹ is hydrogen or halogen, or R¹ and R⁹ taken together with the atoms to which they are attached form a 4-, 5-, or 6-membered heterocyclyl ring, or R⁹ and R⁸ taken together with the atoms to which they are attached form a cyclopropyl ring system when m is 0 or a cyclobutyl or cyclopentyl ring system when m is 1;
R¹⁰ and R¹¹ each independently is selected from hydrogen, hydroxyl, halogen, C₁₋₆alkyl, and C₁₋₆ haloalkyl;
R¹² is hydrogen or halogen;
X is selected from a bond, S, O, and NR⁴, wherein R⁴ is hydrogen or C₁₋₆alkyl (e.g., methyl);
A is selected from cyano, hydroxyl, optionally substituted C₁₋₆alkyl, optionally substituted C₁₋₆alkoxy, hydroxycarbonyl, C₁₋₆alkoxycarbonyl, —C(O)NR⁵R⁶, —NR⁵R⁶, —NR⁵C(O)C₁₋₆alkyl, optionally substituted heterocyclyl, optionally substituted aryl, and optionally substituted heteroaryl;
R⁵ and R⁶ each independently is selected from hydrogen and C₁₋₆alkyl;
m is 0 or 1, provided that when m is 1, R⁸ and R⁹ are taken together with the carbons to which they are attached to form a cyclobutyl or cyclopentyl ring system; and
n is 0, 1 or 2,
in the preparation of a medicament for the treatment of Rett syndrome and/or one or more symptoms associated with Rett syndrome.

38. The use of claim 37, wherein the compound is selected from any one of compounds 1-10, 12-18, 20-25, 27-37, 39, 40, 44-53, 56, 61-70, 74, 76-79, 83, 84, 89, 90, 93-96, 104, 106, 107, 110, 112-117, 119-123, 125, 127-129, 132, 136, 138-141, 146-147, 150-152, 159-162, 172, 174-178, 183, 191-195, 197, 198, 206-208, 212-230, and 232-339, and pharmaceutically acceptable salts thereof.

39. The use of any one of claims 36-38, wherein the one or more symptoms associated with Rett syndrome is selected from sleep disturbances; sleep apnea; seizures; breathing disorders; irregular heartbeat; intellectual disabilities; and autism.
